(19) United States
(12) Patent Application Publication
(10) Pub. No.: US 2024/0182599 A1
(43) Pub. Date: Jun. 6, 2024

(54) ANTI-IGE ANTIBODIES

(71) Applicant: Abwiz Bio, Inc., San Diego, CA (US)

(72) Inventors: Toshiaki Maruyama, La Jolla, CA (US); Shigeru CJ Maruyama, San Diego, CA (US); Jiro Kitaura, Tokyo (JP); Tomoaki Ando, Tokyo (JP); Hexing Wang, Tokyo (JP)

(21) Appl. No.: 18/378,125

(22) Filed: Oct. 9, 2023

Related U.S. Application Data
(60) Provisional application No. 63/414,414, filed on Oct. 7, 2022.

(51) Int. Cl. C07K 16/42

(52) U.S. Cl. CPC C07K 16/4291; C07K 2317/24; C07K 2317/31; C07K 2317/52; C07K 2317/55; C07K 2317/565; C07K 2317/76; C07K 2317/92

(57) ABSTRACT

This application concerns a set of anti-IgE antibodies to a novel region of Cε (β5-helix region of Cε2) and their use in the blocking of IgE to Fcε receptors (FcεRI and CD23) for the treatment of diseases mediated by IgE such as allergic airway diseases including asthma. Of note, these anti-IgE antibodies not only bind but also remove pre-bound IgE from Fcε receptors.

### BACKGROUND OF THE INVENTION

Immunoglobulin E (IgE) is a sensor molecule that plays a pivotal role in inducing the immediate reaction. IgE binds its high affinity receptor FcεRI and confers a versatile recognizing ability to mast cell and basophils, which are the main effector cells in the immediate allergic reaction. Upon binding of allergens, mast cells and basophils are quickly activated, and they release a wide variety of inflammation mediator molecules including histamine, proteases, lipid mediators, cytokines, and chemokines.

This application contains a sequence listing which is electronically filed concurrently herewith as 119897-0004UT01, file size 448 KB with a production date of Feb. 8, 2024, which is incorporated by reference herein.

### SUMMARY OF THE INVENTION

According to one aspect of the disclosure, an antigen binding polypeptide is provided wherein the polypeptide exhibits specific binding to an IgE.

In some embodiments, the binding of the antigen binding polypeptide to the IgE disrupts interaction between the IgE and at least one Fcε receptor.

In some embodiments, the disrupted interaction comprises blocking an unbound IgE from binding to the at least one Fcε receptor. In some embodiments, the disrupted interaction comprises dissociating a bound IgE from the at least one Fcε receptor.

In some embodiments, the disrupted interaction results in suppression of degranulation.

In some embodiments, the at least one Fcε receptor comprises FcεRI. In some embodiments, the at least one Fcε receptor comprises CD23. In some embodiments, the at least one Fcε receptor comprises FcεRI and CD23.

In some embodiments, the antigen binding polypeptide specifically binds to at least one amino acid residue in β5-helix region of the Cε2, wherein the β5-helix region is the combination of helix, β5-helix joint, and lower half of β5 that connects to helix of Cε2.

In some embodiments, the antigen binding polypeptide specifically binds to at least one amino acid residue in the helix of Cε2. In some embodiments, the antigen binding polypeptide specifically binds to at least one amino acid residue in the β5-helix joint of Cε2. In some embodiments, the antigen binding polypeptide specifically binds to at least one amino acid residue in the lower half of β5 that connects to helix of Cε2.

In some embodiments, the antigen binding polypeptide does not bind to amino acid residue T298 of Cε2.

In some embodiments, the antigen binding polypeptide binds to at least two amino acid residues in the β5-Cε2 helix region.

In some embodiments, the antigen binding polypeptide does not bind to β3 region of Cε2. In some embodiments, the antigen binding polypeptide does not bind to β4 region of Cε2. In some embodiments, the antigen binding polypeptide does not bind to β3 or β4 region of Cε2.

In some embodiments, the antigen binding polypeptide comprises at least one amino acid sequence selected from SEQ ID Nos 1-25. In some embodiments, the antigen binding polypeptide comprises at least one amino acid sequence selected from SEQ ID Nos 26-50.

In some embodiments, the antigen binding polypeptide comprises at least one amino acid sequence selected from SEQ ID Nos 51-200.

According to another aspect of the disclosure, an antibody or a fragment thereof is provided. In some embodiments, the antibody or fragment has a Fab region that specifically binds to an IgE.

In some embodiments, the binding of the Fab to the IgE disrupts interaction between the IgE and at least one Fe receptor.

In some embodiments, the disrupted interaction comprises blocking an unbound IgE from binding to the at least one Fe receptor. In some embodiments, the disrupted interaction comprises dissociating a bound IgE from the at least one Fe receptor.

In some embodiments, the disrupted interaction results in suppression of degranulation.

In some embodiments, the at least one Fe receptor comprises FcεRI. In some embodiments, the at least one Fe receptor comprises CD23. In some embodiments, the at least one Fe receptor comprises FcεRI and CD23.

In some embodiments, the Fab region specifically binds to at least one amino acid residue in β5-helix region of the Cε2, wherein the β5-helix region is the combination of helix, β5-helix joint, and lower half of β5 that connects to helix of Cε2.

In some embodiments, the Fab region specifically binds to at least one amino acid residue in the helix of Cε2. In some embodiments, the Fab region specifically binds to at least one amino acid residue in the β5-helix joint of Cε2. In some embodiments, the Fab region specifically binds to at least one amino acid residue in the lower half of β5 that connects to helix of Cε2.

In some embodiments, the Fab region does not bind to amino acid residue T298 of Cε2.

In some embodiments, the Fab region binds to at least two amino acid residues in the β5-Cε2 helix region.

In some embodiments, the Fab region does not bind to β3 region of Cε2. In some embodiments, the Fab region does not bind to β4 region of Cε2. In some embodiments, the Fab region does not bind to β3 or β4 region of Cε2.

In some embodiments, the antibody is a bispecific antibody or a binding fragment thereof.

In some embodiments, the antibody comprises a monovalent Fab′, a divalent Fab2, a single-chain variable fragment (scFv), a diabody, a minibody, a nanobody, a single-domain antibody (sdAb), or a camelid antibody or binding fragment thereof.

In some embodiments, the Fab region comprises at least one heavy chain sequence selected from SEQ ID Nos 1-25. In some embodiments, the Fab region comprises at least one light chain sequence selected from SEQ ID Nos 26-50. In some embodiments, the Fab region comprises at least one complementarity determining region (CDR) selected from SEQ ID Nos 51-150, DAS, DVS, LSS, KAS, GAS, RAS, TTS, EAS, KTS, and SEQ ID Nos 176-200.

In some embodiments, the antibody exhibits a KD of less than 1 nM, 1.2 nM, 2 nM, 5 nM, 10 nM, 13.5 nM, 15 nM, 20 nM, 25 nM, or 30 nM.

In some embodiments, the antibody comprises a humanized antibody.

According to another aspect of the disclosure, an antibody or a fragment thereof having Fab region comprising specific heavy chain sequence, light chain sequence and/or CDR sequence is provided.

In some embodiments, the Fab region comprises at least one heavy chain sequence selected from SEQ ID Nos 1-25. In some embodiments, the Fab region comprises at least one light chain sequence selected from SEQ ID Nos 26-50. In some embodiments, the Fab region comprises at least one heavy chain sequence selected from SEQ ID Nos 1-25 and at least one light chain sequence selected from SEQ ID Nos 26-50.

In some embodiments, the Fab region comprises a heavy chain sequence of SEQ ID No 1 and a light chain sequence of SEQ ID No 26.

In some embodiments, the Fab region comprises a heavy chain sequence of SEQ ID No 2 and a light chain sequence of SEQ ID No 27.

In some embodiments, the Fab region comprises a heavy chain sequence of SEQ ID No 3 and a light chain sequence of SEQ ID No 28.

In some embodiments, the Fab region comprises a heavy chain sequence of SEQ ID No 4 and a light chain sequence of SEQ ID No 29.

In some embodiments, the Fab region comprises a heavy chain sequence of SEQ ID No 5 and a light chain sequence of SEQ ID No 30.

In some embodiments, the Fab region comprises a heavy chain sequence of SEQ ID No 6 and a light chain sequence of SEQ ID No 31.

In some embodiments, the Fab region comprises a heavy chain sequence of SEQ ID No 7 and a light chain sequence of SEQ ID No 32.

In some embodiments, the Fab region comprises a heavy chain sequence of SEQ ID No 8 and a light chain sequence of SEQ ID No 33.

In some embodiments, the Fab region comprises a heavy chain sequence of SEQ ID No 9 and a light chain sequence of SEQ ID No 34.

In some embodiments, the Fab region comprises a heavy chain sequence of SEQ ID No 10 and a light chain sequence of SEQ ID No 35.

In some embodiments, the Fab region comprises a heavy chain sequence of SEQ ID No 11 and a light chain sequence of SEQ ID No 36.

In some embodiments, the Fab region comprises a heavy chain sequence of SEQ ID No 12 and a light chain sequence of SEQ ID No 37.

In some embodiments, the Fab region comprises a heavy chain sequence of SEQ ID No 13 and a light chain sequence of SEQ ID No 38.

In some embodiments, the Fab region comprises a heavy chain sequence of SEQ ID No 14 and a light chain sequence of SEQ ID No 39.

In some embodiments, the Fab region comprises a heavy chain sequence of SEQ ID No 15 and a light chain sequence of SEQ ID No 40.

In some embodiments, the Fab region comprises a heavy chain sequence of SEQ ID No 16 and a light chain sequence of SEQ ID No 41.

In some embodiments, the Fab region comprises a heavy chain sequence of SEQ ID No 17 and a light chain sequence of SEQ ID No 42.

In some embodiments, the Fab region comprises a heavy chain sequence of SEQ ID No 18 and a light chain sequence of SEQ ID No 43.

In some embodiments, the Fab region comprises a heavy chain sequence of SEQ ID No 19 and a light chain sequence of SEQ ID No 44.

In some embodiments, the Fab region comprises a heavy chain sequence of SEQ ID No 20 and a light chain sequence of SEQ ID No 45.

In some embodiments, the Fab region comprises a heavy chain sequence of SEQ ID No 21 and a light chain sequence of SEQ ID No 46.

In some embodiments, the Fab region comprises a heavy chain sequence of SEQ ID No 22 and a light chain sequence of SEQ ID No 47.

In some embodiments, the Fab region comprises a heavy chain sequence of SEQ ID No 23 and a light chain sequence of SEQ ID No 48.

In some embodiments, the Fab region comprises a heavy chain sequence of SEQ ID No 24 and a light chain sequence of SEQ ID No 49.

In some embodiments, the Fab region comprises a heavy chain sequence of SEQ ID No 25 and a light chain sequence of SEQ ID No 50.

In some embodiments, the Fab region comprises an HCDR1 selected from SEQ ID Nos 51-75.

In some embodiments, the Fab region comprises an HCDR2 selected from SEQ ID Nos 76-100.

In some embodiments, the Fab region comprises an HCDR3 selected from SEQ ID Nos 101-125.

In some embodiments, the Fab region comprises an LCDR1 selected from SEQ ID Nos 126-150.

In some embodiments, the Fab region comprises an LCDR2 selected from SEQ ID Nos 151-175.

In some embodiments, the Fab region comprises an LCDR3 selected from SEQ ID Nos 176-200.

In some embodiments, the antibody exhibits a KD of less than 1 nM, 1.2 nM, 2 nM, 5 nM, 10 nM, 13.5 nM, 15 nM, 20 nM, 25 nM, or 30 nM.

In some embodiments, the antibody comprises a humanized antibody. In some embodiments, the humanized antibody comprises a heavy chain variable region selected from SEQ ID Nos 201-202. In some embodiments, the humanized antibody comprises a light chain variable region selected from SEQ ID Nos 203-206. In some embodiments, the humanized antibody comprises a heavy chain variable region selected from SEQ ID Nos 201-202, and a light chain variable region selected from SEQ ID Nos 203-206.

In some embodiments, the humanized antibody comprises a heavy chain variable region selected from SEQ ID Nos 201, and a light chain variable region selected from SEQ ID Nos 203-204. In some embodiments, the humanized antibody comprises a heavy chain variable region selected from SEQ ID Nos 202, and a light chain variable region selected from SEQ ID Nos 205-206.

In some embodiments, the humanized antibody comprises a heavy chain full sequence selected from SEQ ID Nos 207-208. In some embodiments, the humanized antibody comprises a light chain full sequence selected from SEQ ID Nos 209-212. In some embodiments, rein the humanized antibody comprises a heavy chain full sequence selected from SEQ ID Nos 207-208, and a light chain variable region selected from SEQ ID Nos 209-212.

In some embodiments, the humanized antibody comprises a heavy chain full sequence selected from SEQ ID Nos 207, and a light chain variable region selected from SEQ ID Nos 209-210. In some embodiments, the humanized antibody comprises a heavy chain full sequence selected from SEQ ID Nos 208, and a light chain variable region selected from SEQ ID Nos 211-212.

According to another aspect of the disclosure, a complex comprising the disclosed antigen binding polypeptide or the disclosed antibody or fragment is provided, wherein the complex comprises the polypeptide or antibody bound to IgE protein.

According to another aspect of the disclosure, a method is provided to disrupt an interaction between IgE and at least one Fe receptor by contacting a cell expressing the at least one Fe receptor with an antigen binding polypeptide that specifically binds to β5-helix region of the Cε2, wherein the β5-helix region is the combination of helix, β5-helix joint, and lower half of β5 that connects to helix of Cε2.

In some embodiments, the antigen binding polypeptide specifically binds to at least one amino acid residue in the helix of Cε2. In some embodiments, the antigen binding polypeptide specifically binds to at least one amino acid residue in the β5-helix joint of Cε2. In some embodiments, the antigen binding polypeptide specifically binds to at least one amino acid residue in the lower half of β5 that connects to helix of Cε2.

In some embodiments, the disrupted interaction comprises blocking an unbound IgE from binding to the at least one Fe receptor. In some embodiments, the disrupted interaction comprises dissociating a bound IgE from the at least one Fe receptor.

In some embodiments, the disrupted interaction results in suppression of degranulation.

In some embodiments, the at least one Fe receptor comprises FcεRI. In some embodiments, the at least one Fe receptor comprises CD23. In some embodiments, the at least one Fe receptor comprises FcεRI and CD23.

In some embodiments, the Fab region does not bind to amino acid residue T298 of Cε2.

In some embodiments, the Fab region binds to at least two amino acid residues in the β5-Cε2 helix region.

In some embodiments, the antigen binding polypeptide does not bind to β3 region of Cε2. In some embodiments, the antigen binding polypeptide does not bind to β4 region of Cε2. In some embodiments, the antigen binding polypeptide does not bind to β3 or β4 region of Cε2.

In some embodiments, the antigen binding polypeptide is an antibody or a binding fragment thereof. In some embodiments, the antibody comprises a monovalent Fab′, a divalent Fab2, a single-chain variable fragment (scFv), a diabody, a minibody, a nanobody, a single-domain antibody (sdAb), or a camelid antibody or binding fragment thereof.

In some embodiments, the Fab region comprises at least one heavy chain sequence selected from SEQ ID Nos 1-25.

In some embodiments, the Fab region comprises at least one light chain sequence selected from SEQ ID Nos 26-50.

In some embodiments, the Fab region comprises at least one complementarity determining region (CDR) selected from SEQ ID Nos 51-150, DAS, DVS, LSS, KAS, GAS, RAS, TTS, EAS, KTS, and SEQ ID Nos 176-200.

In some embodiments, the antibody exhibits a KD of less than 1 nM, 1.2 nM, 2 nM, 5 nM, 10 nM, 13.5 nM, 15 nM, 20 nM, 25 nM, or 30 nM.

In some embodiments, the antibody comprises a humanized antibody.

In some embodiments, the interaction between IgE and at least one Fcε receptor is associated with an allergic condition.

In some embodiments, the allergic condition is selected from asthma, chronic idiopathic urticaria, nasal polyps, hay fever, or food allergy.

### BRIEF DESCRIPTION OF THE DRAWINGS

Various aspects of the disclosure are set forth with particularity in the appended claims. The file of this patent contains at least one drawing/photograph executed in color. Copies of this patent with color drawing(s)/photograph(s) will be provided by the Office upon request and payment of the necessary fee. A better understanding of the features and advantages of the present disclosure will be obtained by reference to the following detailed description that sets forth illustrative embodiments, in which the principles of the disclosure are utilized, and the accompanying drawings of which:

FIG. 1 illustrates binding of IgE to its high affinity receptor FcεRI according to some aspect of the disclosure, particularly showing the role of Cε2 and Cε3.

FIG. 2 illustrates a production process for an anti-human IgE Fab library according to some aspect of the disclosure.

FIG. 3 illustrates competition and removal assays according to some aspect of the disclosure.

FIG. 4 illustrates binding of selected Fabs tested in an ELISA according to some aspect of the disclosure. The purified Fabs were serially diluted and tested against a recombinant Cε2-4 protein coated at 2 μg/mL.

FIG. 5 illustrates blocking activities of the selected Fabs tested by flow cytometry according to some aspect of the disclosure. 0.5 μg/mL of IgE was incubated with different molar ratios of the Fabs and IgG at 37° C. for 1 h. The BaF/3 cell line transduced with human FcεRIα, β, and γ chains (BaF3-hFcεRI) was incubated with the Fab-treated IgE at 37° C. for 2 h. The binding of IgE to the cells was assessed as the binding capacity of the fluorescently labeled antigens.

FIG. 6 illustrates removal of the human IgEs on human FcεRIα-expressing mouse bone marrow-derived mast cells (BMMCs-hFcεRI) (A) and suppression of antigen-induced degranulation (B) according to some aspect of the disclosure. BMMCs-hFcεRI were incubated with 0.5 μg/mL of human IgE overnight. After washes, the cells were incubated with various concentrations of indicated Fab for 48 h. After washes, the cells were splitted into two experiments. In one experiment, the cells were incubated with fluorescently labeled antigens on ice, and the fluorescence levels, which represent the IgE amount on the cells, were measured by flow cytometry (A). In the other experiment, the cells were stimulated with antigens in the presence of anti-human CD63 antibodies, and the CD63 positive cells were considered as degranulating cells (B). Conc, concentration; MFI, mean fluorescence intensity.

FIG. 7 illustrates the competition (A) and removal (B) activities on IgE binding to human CD23 (hCD23) according to some aspect of the disclosure. (A) 2 μg/mL of IgE was incubated with 40 μg/mL of the indicated Fabs at 37° C. for 1 h. BaF/3 cell line transduced with human CD23 (BaF3-hCD23) was incubated with the Fab-treated IgE for 2 h on ice. After washes, the cells were incubated with fluorescently labeled antigens on ice, and the fluorescence was measured by flow cytometry. The MFIs were normalized to that of the cells incubated with non-treated IgE. (B) BaF-hCD23 cells were incubated with 2 μg/mL of human IgE overnight. After washes, the cells were incubated with 10 μg/mL of indicated Fab or omalizumab (Xolair) at 37° C. for 1 h. After washes, the cells were incubated with the fluorescently labeled antigen and the fluorescence levels were measured by flow cytometry. For Xolair, the same mass and molecular (mol) concentrations were used for comparison.

FIG. 8 illustrates the binding region of the three Fabs assessed by ELISA according to some aspect of the disclosure. (A,B) Recombinant his-tagged Cε2 protein fragment was treated with PNGaseF under the denaturing (A) or native (B) conditions. The Cε2 preparations were coated onto a microplate, and the binding of Fabs was assessed. (C) Human or mouse IgEs were coated on a microplate and detected by indicated Fabs or antibodies by ELISA. (D) The schematic representation of the Cε2 fragment of human IgE. Cε2 consists of seven β-sheets (numbered), one α-helix, and joining hinges. (E) Recombinant Cε2 mutants with human to mouse replacement in indicated regions were coated on a microplate. The binding of indicated Fabs and IgGs were assessed by ELISA. The readings were normalized to those of the anti-human IgE polyclonal antibody.

FIG. 9 illustrates the binding amino acids of the three Fabs in β5-Helix region assessed by ELISA according to some aspect of the disclosure. (A) Amino acid sequences of human and mouse Cε2 β5-Helix region. Gray-boxed amino acids are common for human and mouse IgE. Orange-underlined amino acids were replaced with those of mouse IgE in the indicated mutant IgE. (B) The wild-type (WT) and mutant chimera IgEs with the indicated regions of human to mouse replacements were coated onto a microplate. The binding levels of Fabs and anti-human IgE antibody were assessed by ELISA.

FIG. 10 illustrates the binding and stability of the mutant IgEs on the cell surface FcεRI assessed by flow cytometry according to some aspect of the disclosure. BaF3-hFcεRI cells were incubated with indicated human IgE mutants overnight. After washes, the cells were settled on ice for indicated time (A, 0 h; B, 3 h; C, 6 h). After washes, the cells were incubated with indicated concentrations of fluorescently labeled antigens on ice and subjected to the flow cytometry.

FIG. 11 illustrates the competitive activities of the three Fabs on the mutated IgEs assessed by flow cytometry according to some aspect of the disclosure. The human IgE mutants were incubated with the indicated Fabs or IgGs at 37° C. for 1 h. The BaF3-hFcεRI cells were incubated with the pretreated IgE mutants at 37° C. for 2 h. The binding of IgEs was assessed by the fluorescent antigen binding.

FIG. 12 illustrates the removal activities of the three Fabs (A) or the IgGs having the Fab regions (B) on the FcεRI-bound mutated IgEs assessed by flow cytometry according to some aspect of the disclosure. The cells were incubated with 0.5 μg/mL of the IgE mutants overnight. After washes, the cells were incubated with 10 μg/mL of Fabs or IgGs at 37° C. The remaining IgE amount was assessed as fluorescent antigen binding at indicated time points by flow cytometry.

FIG. 13 illustrates dose response of a standard in a degranulation suppression assay according to some aspect of the disclosure.

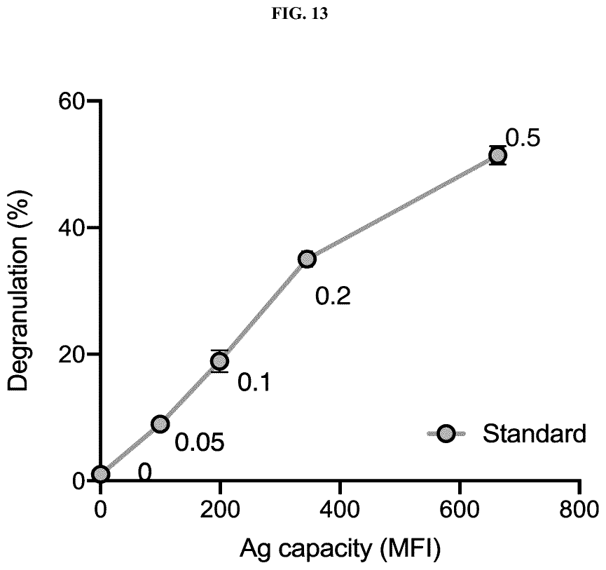

FIG. 14 illustrates dose response of a Fab (BH3), a control and a standard in a degranulation suppression assay according to some aspect of the disclosure. The Fab exhibits dose-dependent suppression of degranulation.

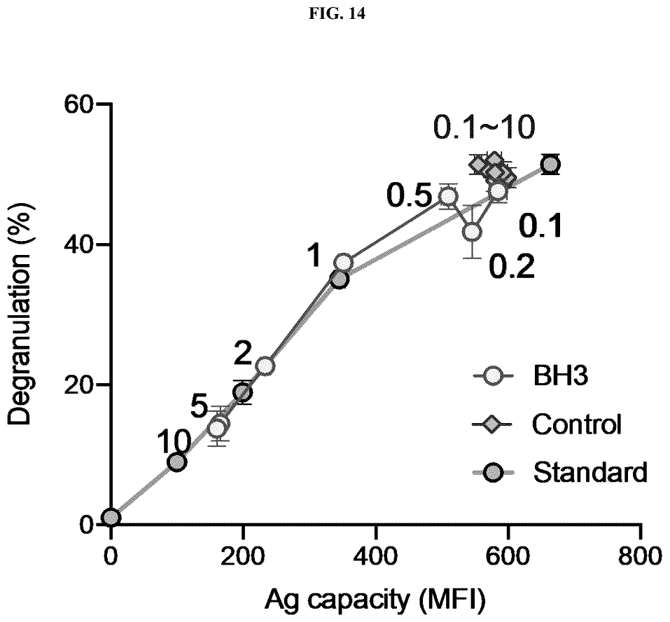

FIG. 15 illustrates dose response of omalizumab (Xolair) and a standard in a degranulation suppression assay according to some aspect of the disclosure.

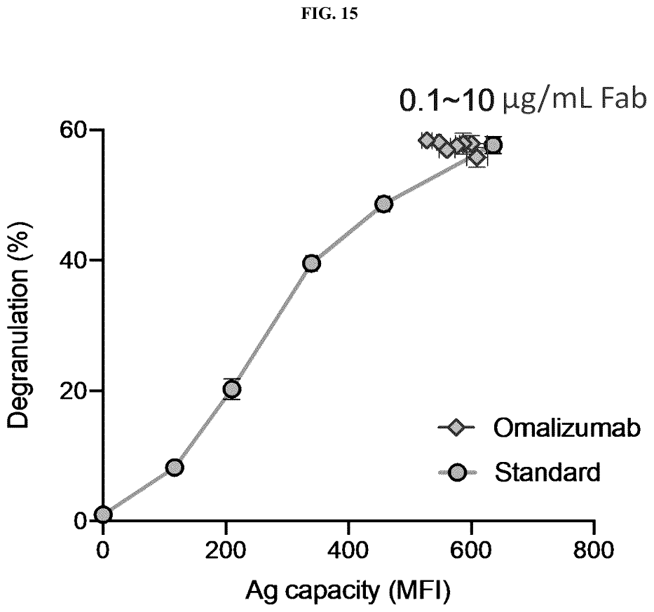

FIG. 16 illustrates binding of rabbit Fabs of BC48 and humanized Fabs of BC48 to Cε2-4 by ELISA according to some aspect of the disclosure.

FIG. 17 illustrates binding of rabbit Fabs of BH3 and humanized Fabs of BH3 to Cε2-4 by ELISA according to some aspect of the disclosure.

### DETAILED DESCRIPTION OF THE INVENTION

IgEs are produced by B cells and plasma cells, either centrally or peripherally at the site of inflammation, and are distributed throughout the human body through circulation at a very low concentration. Once picked up by mast cells, the IgE stays on FcεRI for weeks. Therefore, targeting the unbound free IgE cannot remove already bound IgEs on FcεRI for a long time. However, because of the low production, it is not difficult to neutralize unbound IgEs using antibodies such as omalizumab.

Omalizumab (Xolair®) has been developed to neutralize the free IgEs in sera and to block their binding to FcεRI and low-affinity IgE receptors, CD23. Omalizumab is a recombinant humanized IgG1κ monoclonal antibody that mainly binds Cε3 domain of human IgE. The efficacy of omalizumab has been reported for many allergic diseases including allergic asthma 1-5, chronic urticaria 6, 7, nasal polyposis 8, 9, and pollinosis 10, and approved for the treatment of moderate to severe persistent allergic asthma, chronic idiopathic urticaria (CIU) and nasal polyps in the USA. It is also approved for severe pollinosis in Japan. In addition, its benefit in an adjunctive use with immunotherapy has been reported 11. One of the major drawbacks of omalizumab is its slow action. Since omalizumab does not affect the FcεRI-bound IgEs, the reduction of IgE on mast cells takes longer (˜70 days) than that on basophils (˜7 days), depending on their half-lives 12, 13. Therefore, the improvement of allergic disease requires more than two weeks after initiation of the therapy 13.

To improve efficacy, several kinds of IgE-associating molecules have been developed, including ligelizumab 14-18, aεFab 19, MEDI4212 20, 21, DARPins (designed ankyrin repeat proteins) 22-25, Single-domain antibody (sdab) 026 26, quilizumab 27-29, AIMab7195 (former XmAb7195) 30, bispecific IgE/CD3 antibody (bsc-IgE/CD3) 31, and DNA aptamers 321, 33, 34. Most of the substances directly targeting IgE Cε3 to block its binding to IgE receptors, as IgE binds FcεRI and CD23 using Cε3 portion. However, the mode of action varies among the substances; the steric hindrance, allosteric hindrance, and facilitated dissociation mechanisms have been proposed for the blocking and removal activities 35. For example, the binding of omalizumab blocks IgE's binding to CD23 by steric hindrance, caused by a direct overlap of the footprints of omalizumab and human CD23 36, 37. The way omalizumab blocks IgE's binding to FcεRI is different; the binding of omalizumab alters IgE's 3D-conformation to the one that cannot bind FcεRI. This mode is called allosteric hindrance 37. The best example of the facilitated dissociation is DARPin E2_79. DARPin E2_79 shares a small portion of the footprint with FcεRI on IgE molecule, and this competition at that small part is considered to accelerate the dissociation of FcεRI 23. Therefore, the IgE blocking substances may have different blocking and/or removal activities depending on the binding sites.

In addition to Cε3, the Cε2 portion of human IgE has a role in stabilizing the IgE's binding to FcεRI 38. A modified version of omalizumab Fab called FabXol3 has contact with some of Cε2 amino acids 37. An Fab portion of an antibody against mouse Cε2 (clone 6HD5) reduces mast cell activation in vivo and in vitro 39. However, the binding epitope for Fab-6HD5 inside Cε2 has not been elucidated. Furthermore, the homology of the amino acid sequences between human and mouse IgE Cε2-Cε4 is quite low (55% for Cε2-Cε4, and 63% for Cε2), and human IgE does not bind murine FcεRI. Therefore, this cannot be directly extrapolated into human IgE, and it has not been well understood whether antibodies targeting human Cε2 can block and/or remove IgE's binding to its receptors.

In this invention, we have devised several Fab clones that bind mainly to Cε2 of human IgE. The Fab clones were able to not only block IgE's binding to FcεRI and CD23, but also remove already bound IgEs from their receptors. These Fabs had common binding epitopes inside the Cε2 portion that were not described before. Our invention adds another strategy to target IgE, in a faster way than the omalizumab does.

IgEs play a pivotal role in the immediate allergic reaction. In addition, successful applications of an anti-human IgE antibody, omalizumab, to human diseases have proved essential roles of IgEs in various allergic disorders including asthma, chronic idiopathic urticaria, nasal polyps, hay fever, and food allergy.

The Cε2 region of human IgE has been implicated in the stability of IgE on FcεRI. However, it was not clear whether an Fab or IgG targeting Cε2 can inhibit the binding of human IgE to FcεRI and CD23.

In this invention, we first prepared Fab libraries against human IgE that were produced from a rabbit immunized with a recombinant human IgE Cε2-4 fragment and selected by phage display. The Fab fragments of selected clones against Cε2 region were purified and subjected to competition, removal, and degranulation inhibition assays. The binding sites of the selected three Fabs were investigated using a partial chimera fragment of human and mouse Cε2, and further narrowed down using chimera IgEs. The competition and removal activities against human CD23 on cell surface were also assessed.

As a result, 24 clones against Cε2 were isolated from a rabbit Fab library. All these Fab clones competitively inhibited the binding of human IgE to FcεRI. The removal activities were highly correlated with the competitive inhibition activities. Of these, three highly active clones were selected. These did not bind mouse IgEs. Using a recombinant human Cε2 of which the amino acid sequence was partially substituted with that of mouse Cε2, β5-helix region was found to be the common binding region of these Fabs. Further investigation revealed that especially the latter half of the β5-helix region was the essential portion for the inhibitory binding. These Fabs competitively inhibited the binding of human IgEs to human CD23, and removed IgEs bound to CD23. Using mouse bone marrow-derived mast cells expressing human FcεRIα, the removal of IgEs from the mast cell surface was shown to reduce mast cell activation. In summary, Fab clones against human IgE Cε2 region were obtained that elicit inhibition activities on binding of human IgE to its receptors.

### Antibody Production

In some embodiments, anti-IgE antibodies are raised by standard protocol by injecting a production animal with an antigenic composition. See, e.g., Harlow and Lane, Antibodies: A Laboratory Manual, Cold Spring Harbor Laboratory, 1988. When utilizing an entire protein, or a larger section of the protein, antibodies may be raised by immunizing the production animal with the protein and a suitable adjuvant (e.g., Freund's, Freund's complete, oil-in-water emulsions, etc.). When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the protein target, a superior immune response may be elicited if the polypeptide is joined to a carrier protein, such as ovalbumin, BSA or KLH.

Polyclonal or monoclonal anti-IgE antibodies can be produced from animals which have been genetically altered to produce human immunoglobulins. A transgenic animal can be produced by initially producing a “knock-out” animal which does not produce the animal's natural antibodies, and stably transforming the animal with a human antibody locus (e.g., by the use of a human artificial chromosome). In such cases, only human antibodies are then made by the animal. Techniques for generating such animals, and deriving antibodies therefrom, are described in U.S. Pat. Nos. 6,162,963 and 6,150,584, incorporated fully herein by reference. Such antibodies can be referred to as human xenogenic antibodies.

Alternatively, anti-IgE antibodies can be produced from phage libraries containing human variable regions. See U.S. Pat. No. 6,174,708, incorporated fully herein by reference.

In some aspects of any of the embodiments disclosed herein, an anti-IGE antibody is produced by a hybridoma.

For monoclonal anti-IgE antibodies, hybridomas may be formed by isolating the stimulated immune cells, such as those from the spleen of the inoculated animal. These cells can then be fused to immortalized cells, such as myeloma cells or transformed cells, which are capable of replicating indefinitely in cell culture, thereby producing an immortal, immunoglobulin-secreting cell line. The immortal cell line utilized can be selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT).

In addition, the anti-IgE antibody may be produced by genetic engineering.

Anti-IgE antibodies disclosed herein can have a reduced propensity to induce an undesired immune response in humans, for example, anaphylactic shock, and can also exhibit a reduced propensity for priming an immune response which would prevent repeated dosage with an antibody therapeutic or imaging agent (e.g., the human-anti-murine-antibody “HAMA” response). Such anti-IgE antibodies include, but are not limited to, humanized, chimeric, or xenogenic human anti-IgE antibodies.

Chimeric anti-IgE antibodies can be made, for example, by recombinant means by combining the murine variable light and heavy chain regions (VK and VH), obtained from a murine (or other animal-derived) hybridoma clone, with the human constant light and heavy chain regions, in order to produce an antibody with predominantly human domains. The production of such chimeric antibodies is well known in the art, and may be achieved by standard means (as described, e.g., in U.S. Pat. No. 5,624,659, incorporated fully herein by reference).

The term “humanized” as applies to a non-human (e.g. rodent or primate) antibodies are hybrid immunoglobulins, immunoglobulin chains or fragments thereof which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance and minimize immunogenicity when introduced into a human body. In some examples, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin.

Humanized antibodies can be engineered to contain human-like immunoglobulin domains, and incorporate only the complementarity-determining regions of the animal-derived antibody. This can be accomplished by carefully examining the sequence of the hyper-variable loops of the variable regions of a monoclonal antigen binding unit or monoclonal antibody, and fitting them to the structure of a human antigen binding unit or human antibody chains. See, e.g., U.S. Pat. No. 6,187,287, incorporated fully herein by reference.

Methods for humanizing non-human antibodies are well known in the art. “Humanized” antibodies are antibodies in which at least part of the sequence has been altered from its initial form to render it more like human immunoglobulins. In some versions, the heavy (H) chain and light (L) chain constant (C) regions are replaced with human sequence. This can be a fusion polypeptide comprising a variable (V) region and a heterologous immunoglobulin C region. In some versions, the complementarity determining regions (CDRs) comprise non-human antibody sequences, while the V framework regions have also been converted to human sequences. See, for example, EP 0329400. In some versions, V regions are humanized by designing consensus sequences of human and mouse V regions, and converting residues outside the CDRs that are different between the consensus sequences.

In principle, a framework sequence from a humanized antibody can serve as the template for CDR grafting; however, it has been demonstrated that straight CDR replacement into such a framework can lead to significant loss of binding affinity to the antigen. Glaser et al. (1992) J. Immunol. 149:2606; Tempest et al. (1992) Biotechnology 9:266; and Shalaby et al. (1992) J. Exp. Med. 17:217. The more homologous a human antibody (HuAb) is to the original murine antibody (muAb), the less likely that the human framework will introduce distortions into the murine CDRs that could reduce affinity. Based on a sequence homology search against an antibody sequence database, the HuAb IC4 provides good framework homology to muM4TS.22, although other highly homologous HuAbs would be suitable as well, especially kappa L chains from human subgroup I or H chains from human subgroup III. Kabat et al. (1987). Various computer programs such as ENCAD (Levitt et al. (1983) J. Mol. Biol. 168:595) are available to predict the ideal sequence for the V region. The invention thus encompasses HuAbs with different variable (V) regions. It is within the skill of one in the art to determine suitable V region sequences and to optimize these sequences. Methods for obtaining antibodies with reduced immunogenicity are also described in U.S. Pat. No. 5,270,202 and EP 699,755.

Humanized antibodies can be prepared by a process of analysis of the parental sequences and various conceptual humanized products using three dimensional models of the parental and humanized sequences. Three dimensional immunoglobulin models are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved.

A process for humanization of subject antigen binding units can be as follows. The best-fit germline acceptor heavy and light chain variable regions are selected based on homology, canonical structure and physical properties of the human antibody germlines for grafting. Computer modeling of mVH/VL versus grafted hVH/VL is performed and prototype humanized antibody sequence is generated. If modeling indicated a need for framework back-mutations, second variant with indicated FW changes is generated. DNA fragments encoding the selected germline frameworks and murine CDRs are synthesized. The synthesized DNA fragments are subcloned into IgG expression vectors and sequences are confirmed by DNA sequencing. The humanized antibodies are expressed in cells, such as 293F and the proteins are tested, for example in MDM phagocytosis assays and antigen binding assays. The humanized antigen binding units are compared with parental antigen binding units in antigen binding affinity, for example, by FACS on cells expressing the target antigen. If the affinity is greater than 2-fold lower than parental antigen binding unit, a second round of humanized variants can be generated and tested as described above.

As noted above, an anti-IgE antibody can be either “monovalent” or “multivalent.” Whereas the former has one binding site per antigen-binding unit, the latter contains multiple binding sites capable of binding to more than one antigen of the same or different kind. Depending on the number of binding sites, antigen binding units may be bivalent (having two antigen-binding sites), trivalent (having three antigen-binding sites), tetravalent (having four antigen-binding sites), and so on.

Multivalent anti-IgE antibodies can be further classified on the basis of their binding specificities. A “monospecific” anti-IgE antibody is a molecule capable of binding to one or more antigens of the same kind. A “multispecific” anti-IgE antibody is a molecule having binding specificities for at least two different antigens. While such molecules normally will only bind two distinct antigens (i.e. bispecific anti-IgE antibodies), antibodies with additional specificities such as trispecific antibodies are encompassed by this expression when used herein. This disclosure further provides multispecific anti-IgE antibodies. Multispecific anti-IgE antibodies are multivalent molecules capable of binding to at least two distinct antigens, e.g., bispecific and trispecific molecules exhibiting binding specificities to two and three distinct antigens, respectively.

### Polynucleotides and Vectors

In some embodiments, the present disclosure provides isolated nucleic acids encoding any of the anti-IgE antibodies disclosed herein. In another embodiment, the present disclosure provides vectors comprising a nucleic acid sequence encoding any anti-IgE antibody disclosed herein. In some embodiments, this invention provides isolated nucleic acids that encode a light-chain CDR and a heavy-chain CDR of an anti-IgE antibody disclosed herein.

The subject anti-IgE antibodies can be prepared by recombinant DNA technology, synthetic chemistry techniques, or a combination thereof. For instance, sequences encoding the desired components of the anti-IgE antibodies, including light chain CDRs and heavy chain CDRs are typically assembled cloned into an expression vector using standard molecular techniques know in the art. These sequences may be assembled from other vectors encoding the desired protein sequence, from PCR-generated fragments using respective template nucleic acids, or by assembly of synthetic oligonucleotides encoding the desired sequences. Expression systems can be created by transfecting a suitable cell with an expressing vector which comprises an anti-IgE antibody of interest.

Nucleotide sequences corresponding to various regions of light or heavy chains of an existing antibody can be readily obtained and sequenced using convention techniques including but not limited to hybridization, PCR, and DNA sequencing. Hybridoma cells that produce monoclonal antibodies serve as a preferred source of antibody nucleotide sequences. A vast number of hybridoma cells producing an array of monoclonal antibodies may be obtained from public or private repositories. The largest depository agent is American Type Culture Collection (atcc.org), which offers a diverse collection of well-characterized hybridoma cell lines.

Alternatively, antibody nucleotides can be obtained from immunized or non-immunized rodents or humans, and form organs such as spleen and peripheral blood lymphocytes. Specific techniques applicable for extracting and synthesizing antibody nucleotides are described in Orlandi et al. (1989) Proc. Natl. Acad. Sci. U.S.A 86: 3833-3837; Larrick et al. (1989) Biochem. Biophys. Res. Commun. 160:1250-1255; Sastry et al. (1989) Proc. Natl. Acad. Sci., U.S.A. 86: 5728-5732; and U.S. Pat. No. 5,969,108.

Polynucleotides encoding anti-IgE antibodies can also be modified, for example, by substituting the coding sequence for human heavy and light chain constant regions in place of the homologous non-human sequences. In that manner, chimeric antibodies are prepared that retain the binding specificity of the original anti-IgE antibody.

### Host Cells

In some embodiments, the present disclosure provides host cells expressing any one of the anti-IgE antibodies disclosed herein. A subject host cell typically comprises a nucleic acid encoding any one of the anti-IgE antibodies disclosed herein.

The invention provides host cells transfected with the polynucleotides, vectors, or a library of the vectors described above. The vectors can be introduced into a suitable prokaryotic or eukaryotic cell by any of a number of appropriate means, including electroporation, microprojectile bombardment; lipofection, infection (where the vector is coupled to an infectious agent), transfection employing calcium chloride, rubidium chloride, calcium phosphate, DEAE-dextran, or other substances. The choice of the means for introducing vectors will often depend on features of the host cell.

For most animal cells, any of the above-mentioned methods is suitable for vector delivery. Preferred animal cells are vertebrate cells, preferably mammalian cells, capable of expressing exogenously introduced gene products in large quantity, e.g. at the milligram level. Non-limiting examples of preferred cells are NIH3T3 cells, COS, HeLa, and CHO cells.

Once introduced into a suitable host cell, expression of the anti-IgE antibodies can be determined using any nucleic acid or protein assay known in the art. For example, the presence of transcribed mRNA of light chain CDRs or heavy chain CDRs, or the anti-IGE antibody can be detected and/or quantified by conventional hybridization assays (e.g. Northern blot analysis), amplification procedures (e.g. RT-PCR), SAGE (U.S. Pat. No. 5,695,937), and array-based technologies (see e.g. U.S. Pat. Nos. 5,405,783, 5,412,087 and 5,445,934), using probes complementary to any region of a polynucleotide that encodes the anti-IGE antibody.

Expression of the vector can also be determined by examining the expressed anti-IgE antibody. A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

### NON-LIMITING EMBODIMENTS

The present disclosure is also described and demonstrated by way of the following non-limiting embodiments. However, the use of these and other examples anywhere in the specification is illustrative only and in no way limits the scope and meaning of the disclosure or of any exemplified term. Likewise, the disclosure is not limited to any particular preferred embodiment or aspect described herein. Indeed, suitable modifications and variations may be apparent to those skilled in the art upon reading this specification, and such variations can be made without departing from the invention in spirit or in scope.
1. An antigen binding polypeptide, wherein the polypeptide exhibits specific binding to an IgE.
2. The antigen binding polypeptide of embodiment 1, wherein the binding of the antigen binding polypeptide to the IgE disrupts interaction between the IgE and at least one Fcε receptor.
3. The antigen binding polypeptide of embodiment 2, wherein the disrupted interaction comprises blocking an unbound IgE from binding to the at least one Fcε receptor.
4. The antigen binding polypeptide of embodiment 2-3, wherein the disrupted interaction comprises dissociating a bound IgE from the at least one Fcε receptor.
5. The antigen binding polypeptide of embodiment 2-4, wherein the disrupted interaction results in suppression of degranulation.
6. The antigen binding polypeptide of embodiment 2-5, wherein the at least one Fcε receptor comprises FcεRI.
7. The antigen binding polypeptide of embodiment 2-5, wherein the at least one Fcε receptor comprises CD23.
8. The antigen binding polypeptide of embodiment 2-5, wherein the at least one Fcε receptor comprises FcεRI and CD23.
9. The antigen binding polypeptide of embodiment 1-8, wherein the antigen binding polypeptide specifically binds to at least one amino acid residue in β5-helix region of the Cε2, wherein the β5-helix region is the combination of helix, β5-helix joint, and lower half of β5 that connects to helix of Cε2.
10. The antigen binding polypeptide of embodiment 9, wherein the antigen binding polypeptide specifically binds to at least one amino acid residue in the helix of Cε2.
11. The antigen binding polypeptide of embodiment 9-10, wherein the antigen binding polypeptide specifically binds to at least one amino acid residue in the β5-helix joint of Cε2.
12. The antigen binding polypeptide of embodiment 9-11, wherein the antigen binding polypeptide specifically binds to at least one amino acid residue in the lower half of β5 that connects to helix of Cε2.
13. The antigen binding polypeptide of embodiment 9-12, wherein the antigen binding polypeptide does not bind to amino acid residue T298 of Cε2.
14. The antigen binding polypeptide of embodiment 9-13, wherein the antigen binding polypeptide binds to at least two amino acid residues in the β5-Cε2 helix region.
15. The antigen binding polypeptide of embodiment 1-14, wherein the antigen binding polypeptide does not bind to β3 region of Cε2.
16. The antigen binding polypeptide of embodiment 1-14, wherein the antigen binding polypeptide does not bind to β4 region of Cε2.
17. The antigen binding polypeptide of embodiment 1-14, wherein the antigen binding polypeptide does not bind to β3 or β4 region of Cε2.
18. The antigen binding polypeptide of embodiment 1-17, comprising at least one amino acid sequence selected from SEQ ID Nos 1-25.
19. The antigen binding polypeptide of embodiment 1-18, comprising at least one amino acid sequence selected from SEQ ID Nos 26-50.
20. The antigen binding polypeptide of embodiment 1-19, comprising at least one amino acid sequence selected from SEQ ID Nos 51-150, DAS, DVS, LSS, KAS, GAS, RAS, TTS, EAS, KTS, and SEQ ID Nos 176-200.
21. An antibody or a fragment thereof, comprising a Fab region that specifically binds to an IgE.
22. The antibody or fragment of embodiment 21, wherein the binding of the Fab to the IgE disrupts interaction between the IgE and at least one Fcε receptor.
23. The antibody or fragment of embodiment 22, wherein the disrupted interaction comprises blocking an unbound IgE from binding to the at least one Fcε receptor.
24. The antibody or fragment of embodiment 22-23, wherein the disrupted interaction comprises dissociating a bound IgE from the at least one Fcε receptor.
25. The antibody or fragment of embodiment 22-24, wherein the disrupted interaction results in suppression of degranulation.
26. The antibody or fragment of embodiment 22-25, wherein the at least one Fcε receptor comprises FcεRI.
27. The antigen binding polypeptide of embodiment 22-25, wherein the at least one Fcε receptor comprises CD23.
28. The antibody or fragment of embodiment 22-25, wherein the at least one Fcε receptor comprises FcεRI and CD23.
29. The antibody or fragment of embodiment 21-28, wherein the Fab region specifically binds to at least one amino acid residue in β5-helix region of the Cε2, wherein the β5-helix region is the combination of helix, β5-helix joint, and lower half of β5 that connects to helix of Cε2.
30. The antibody or fragment of embodiment 29, wherein the Fab region specifically binds to at least one amino acid residue in the helix of Cε2.
31. The antibody or fragment of embodiment 29-30, wherein the Fab region specifically binds to at least one amino acid residue in the β5-helix joint of Cε2.
32. The antibody or fragment of embodiment 29-31, wherein the Fab region specifically binds to at least one amino acid residue in the lower half of β5 that connects to helix of Cε2.
33. The antibody or fragment of embodiment 29-32, wherein the Fab region does not bind to amino acid residue T298 of Cε2.
34. The antibody or fragment of embodiment 29-33, wherein the Fab region binds to at least two amino acid residues in the β5-Cε2 helix region.
35. The antibody or fragment of embodiment 21-34, wherein the Fab region does not bind to 33 region of Cε2.
36. The antibody or fragment of embodiment 21-34, wherein the Fab region does not bind to β4 region of Cε2.
37. The antibody or fragment of embodiment 21-34, wherein the Fab region does not bind to β or β4 region of Cε2.
38. The antibody or fragment of embodiment 21-37, wherein the antibody is a bispecific antibody or a binding fragment thereof.
39. The antibody or fragment of embodiment 21-38, wherein the antibody comprises a monovalent Fab′, a divalent Fab2, a single-chain variable fragment (scFv), a diabody, a minibody, a nanobody, a single-domain antibody (sdAb), or a camelid antibody or binding fragment thereof.
40. The antibody or fragment of embodiment 21-39, wherein the Fab region comprises at least one heavy chain sequence selected from SEQ ID Nos 1-25.
41. The antibody or fragment of embodiment 21-40, wherein the Fab region comprises at least one light chain sequence selected from SEQ ID Nos 26-50.
42. The antibody or fragment of embodiment 21-41, wherein the Fab region comprises at least one complementarity determining region (CDR) selected from SEQ ID Nos 51-150, DAS, DVS, LSS, KAS, GAS, RAS, TTS, EAS, KTS, and SEQ ID Nos 176-200.
43. The antibody or fragment of embodiment 21-42, wherein the antibody exhibits a K_{D} of less than 1 nM, 1.2 nM, 2 nM, 5 nM, 10 nM, 13.5 nM, 15 nM, 20 nM, 25 nM, or 30 nM.
44. The antibody or fragment of embodiment 21-43, wherein the antibody comprises a humanized antibody.
45. An antibody or a fragment thereof, having Fab region comprising at least one heavy chain sequence selected from SEQ ID Nos 1-25.
46. An antibody or a fragment thereof, having Fab region comprising at least one light chain sequence selected from SEQ ID Nos 26-50.
47. An antibody or a fragment thereof, having Fab region comprising at least one heavy chain sequence selected from SEQ ID Nos 1-25 and at least one light chain sequence selected from SEQ ID Nos 26-50.
48. An antibody or a fragment thereof, having Fab region comprising a heavy chain sequence of SEQ ID No 1 and a light chain sequence of SEQ ID No 26.
49. An antibody or a fragment thereof, having Fab region comprising a heavy chain sequence of SEQ ID No 2 and a light chain sequence of SEQ ID No 27.
50. An antibody or a fragment thereof, having Fab region comprising a heavy chain sequence of SEQ ID No 3 and a light chain sequence of SEQ ID No 28.
51. An antibody or a fragment thereof, having Fab region comprising a heavy chain sequence of SEQ ID No 4 and a light chain sequence of SEQ ID No 29.
52. An antibody or a fragment thereof, having Fab region comprising a heavy chain sequence of SEQ ID No 5 and a light chain sequence of SEQ ID No 30.
53. An antibody or a fragment thereof, having Fab region comprising a heavy chain sequence of SEQ ID No 6 and a light chain sequence of SEQ ID No 31.
54. An antibody or a fragment thereof, having Fab region comprising a heavy chain sequence of SEQ ID No 7 and a light chain sequence of SEQ ID No 32.
55. An antibody or a fragment thereof, having Fab region comprising a heavy chain sequence of SEQ ID No 8 and a light chain sequence of SEQ ID No 33.
56. An antibody or a fragment thereof, having Fab region comprising a heavy chain sequence of SEQ ID No 9 and a light chain sequence of SEQ ID No 34.
57. An antibody or a fragment thereof, having Fab region comprising a heavy chain sequence of SEQ ID No 10 and a light chain sequence of SEQ ID No 35.
58. An antibody or a fragment thereof, having Fab region comprising a heavy chain sequence of SEQ ID No 11 and a light chain sequence of SEQ ID No 36.
59. An antibody or a fragment thereof, having Fab region comprising a heavy chain sequence of SEQ ID No 12 and a light chain sequence of SEQ ID No 37.
60. An antibody or a fragment thereof, having Fab region comprising a heavy chain sequence of SEQ ID No 13 and a light chain sequence of SEQ ID No 38.
61. An antibody or a fragment thereof, having Fab region comprising a heavy chain sequence of SEQ ID No 14 and a light chain sequence of SEQ ID No 39.
62. An antibody or a fragment thereof, having Fab region comprising a heavy chain sequence of SEQ ID No 15 and a light chain sequence of SEQ ID No 40.
63. An antibody or a fragment thereof, having Fab region comprising a heavy chain sequence of SEQ ID No 16 and a light chain sequence of SEQ ID No 41.
64. An antibody or a fragment thereof, having Fab region comprising a heavy chain sequence of SEQ ID No 17 and a light chain sequence of SEQ ID No 42.
65. An antibody or a fragment thereof, having Fab region comprising a heavy chain sequence of SEQ ID No 18 and a light chain sequence of SEQ ID No 43.
66. An antibody or a fragment thereof, having Fab region comprising a heavy chain sequence of SEQ ID No 19 and a light chain sequence of SEQ ID No 44.
67. An antibody or a fragment thereof, having Fab region comprising a heavy chain sequence of SEQ ID No 20 and a light chain sequence of SEQ ID No 45.
68. An antibody or a fragment thereof, having Fab region comprising a heavy chain sequence of SEQ ID No 21 and a light chain sequence of SEQ ID No 46.
69. An antibody or a fragment thereof, having Fab region comprising a heavy chain sequence of SEQ ID No 22 and a light chain sequence of SEQ ID No 47.
70. An antibody or a fragment thereof, having Fab region comprising a heavy chain sequence of SEQ ID No 23 and a light chain sequence of SEQ ID No 48.
71. An antibody or a fragment thereof, having Fab region comprising a heavy chain sequence of SEQ ID No 24 and a light chain sequence of SEQ ID No 49.
72. An antibody or a fragment thereof, having Fab region comprising a heavy chain sequence of SEQ ID No 25 and a light chain sequence of SEQ ID No 50.
73. An antibody or a fragment thereof, having Fab region comprising an HCDR1 selected from SEQ ID Nos 51-75.
74. The antibody or fragment of embodiment 73, having Fab region comprising an HCDR2 selected from SEQ ID Nos 76-100.
75. The antibody or fragment of embodiment 73-74, having Fab region comprising an HCDR3 selected from SEQ ID Nos 101-125.
76. The antibody or fragment of embodiment 73-75, having Fab region comprising an LCDR1 selected from SEQ ID Nos 126-150.
77. The antibody or fragment of embodiment 73-76, having Fab region comprising an LCDR2 selected from DAS, DVS, LSS, KAS, GAS, RAS, TTS, EAS, and KTS.
78. The antibody or fragment of embodiment 73-77, having Fab region comprising an LCDR3 selected from SEQ ID Nos 176-200.
79. The antibody or fragment of embodiment 45-78, wherein the antibody exhibits a K_{D} of less than 1 nM, 1.2 nM, 2 nM, 5 nM, 10 nM, 13.5 nM, 15 nM, 20 nM, 25 nM, or 30 nM.
80. The antibody or fragment of embodiment 45-79, wherein the antibody comprises a humanized antibody.
81. A humanized antibody or a fragment thereof, wherein the humanized antibody comprises a heavy chain variable region selected from SEQ ID Nos 201-202.
82. A humanized antibody or a fragment thereof, wherein the humanized antibody comprises a light chain variable region selected from SEQ ID Nos 203-206.
83. A humanized antibody or a fragment thereof, wherein the humanized antibody comprises a heavy chain variable region selected from SEQ ID Nos 201-202, and a light chain variable region selected from SEQ ID Nos 203-206.
84. A humanized antibody or a fragment thereof, wherein the humanized antibody comprises a heavy chain variable region selected from SEQ ID Nos 201, and a light chain variable region selected from SEQ ID Nos 203-204.
85. A humanized antibody or a fragment thereof, wherein the humanized antibody comprises a heavy chain variable region selected from SEQ ID Nos 202, and a light chain variable region selected from SEQ ID Nos 205-206.
86. A humanized antibody or a fragment thereof, wherein the humanized antibody comprises a heavy chain full sequence selected from SEQ ID Nos 207-208.
87. A humanized antibody or a fragment thereof, wherein the humanized antibody comprises a light chain full sequence selected from SEQ ID Nos 209-212.
88. A humanized antibody or a fragment thereof, wherein the humanized antibody comprises a heavy chain full sequence selected from SEQ ID Nos 207-208, and a light chain variable region selected from SEQ ID Nos 209-212.
89. A humanized antibody or a fragment thereof, wherein the humanized antibody comprises a heavy chain full sequence selected from SEQ ID Nos 207, and a light chain variable region selected from SEQ ID Nos 209-210.
90. A humanized antibody or a fragment thereof, wherein the humanized antibody comprises a heavy chain full sequence selected from SEQ ID Nos 208, and a light chain variable region selected from SEQ ID Nos 211-212.
91. A complex comprising the antigen binding polypeptide of embodiment 1-20 or the antibody or fragment of embodiment 21-90, wherein the complex comprises the polypeptide or antibody bound to IgE protein.
92. A method of disrupting an interaction between IgE and at least one Fcε receptor, comprising:
   contacting a cell expressing the at least one Fcε receptor with an antigen binding polypeptide that specifically binds to β5-helix region of the Cε2, wherein the β5-helix region is the combination of helix, β5-helix joint, and lower half of β5 that connects to helix of Cε2.
93. The method of embodiment 92, wherein the antigen binding polypeptide specifically binds to at least one amino acid residue in the helix of Cε2.
94. The method of embodiment 92-93, wherein the antigen binding polypeptide specifically binds to at least one amino acid residue in the β5-helix joint of Cε2.
95. The method of embodiment 92-94, wherein the antigen binding polypeptide specifically binds to at least one amino acid residue in the lower half of β5 that connects to helix of Cε2.
96. The method of embodiment 92-95, wherein the disrupted interaction comprises blocking an unbound IgE from binding to the at least one Fcε receptor.
97. The method of embodiment 92-96, wherein the disrupted interaction comprises dissociating a bound IgE from the at least one Fcε receptor.
98. The method of embodiment 96-97, wherein the disrupted interaction results in suppression of degranulation.
99. The method of embodiment 92-98, wherein the at least one Fcε receptor comprises FcεRI.
100. The method of embodiment 92-98, wherein the at least one Fcε receptor comprises CD23.
101. The method of embodiment 92-98, wherein the at least one Fcε receptor comprises FcεRI and CD23.
102. The method of embodiment 92-101, wherein the Fab region does not bind to amino acid residue T298 of Cε2.
103. The method of embodiment 92-102, wherein the Fab region binds to at least two amino acid residues in the β5-Cε2 helix region.
104. The method of embodiment 92-103, wherein the antigen binding polypeptide does not bind to β3 region of Cε2.
105. The method of embodiment 92-103, wherein the antigen binding polypeptide does not bind to β4 region of Cε2.
106. The method of embodiment 92-103, wherein the antigen binding polypeptide does not bind to β3 or β4 region of Cε2.
107. The method of embodiment 92-106, wherein the antigen binding polypeptide is an antibody or a binding fragment thereof.
108. The method of embodiment 107, wherein the antibody comprises a monovalent Fab′, a divalent Fab2, a single-chain variable fragment (scFv), a diabody, a minibody, a nanobody, a single-domain antibody (sdAb), or a camelid antibody or binding fragment thereof.
109. The method of embodiment 107-108, wherein the Fab region comprises at least one heavy chain sequence selected from SEQ ID Nos 1-25.
110. The method of embodiment 107-109, wherein the Fab region comprises at least one light chain sequence selected from SEQ ID Nos 26-50.
111. The method of embodiment 107-110, wherein the Fab region comprises at least one complementarity determining region (CDR) selected from SEQ ID Nos 51-150, DAS, DVS, LSS, KAS, GAS, RAS, TTS, EAS, KTS, and SEQ ID Nos 176-200.
112. The method of embodiment 107-111, wherein the antibody exhibits a K_{D} of less than 1 nM, 1.2 nM, 2 nM, 5 nM, 10 nM, 13.5 nM, 15 nM, 20 nM, 25 nM, or 30 nM.
113. The method of embodiment 107-112, wherein the antibody comprises a humanized antibody.
114. The method of embodiment 92-113, wherein the interaction between IgE and at least one Fcε receptor is associated with an allergic condition.
115. The method of embodiment 114, wherein the allergic condition is selected from asthma, chronic idiopathic urticaria, nasal polyps, hay fever, or food allergy.

### NON-LIMITING EXAMPLES

The present disclosure is also described and demonstrated by way of the following non-limiting examples. However, the use of these and other examples anywhere in the specification is illustrative only and in no way limits the scope and meaning of the disclosure or of any exemplified term. Likewise, the disclosure is not limited to any particular preferred example or aspect described herein. Indeed, suitable modifications and variations may be apparent to those skilled in the art upon reading this specification, and such variations can be made without departing from the invention in spirit or in scope.

### Example 1—The Development of Anti-IgE Antibodies from an Immunized Rabbit

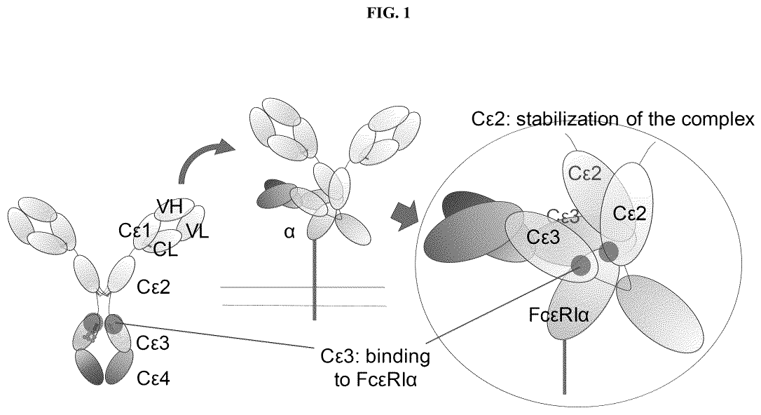

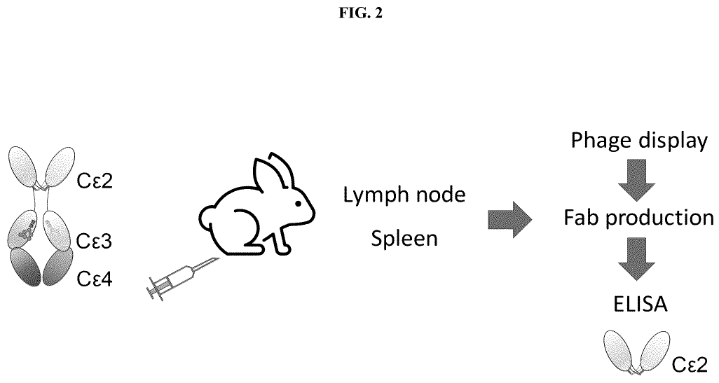

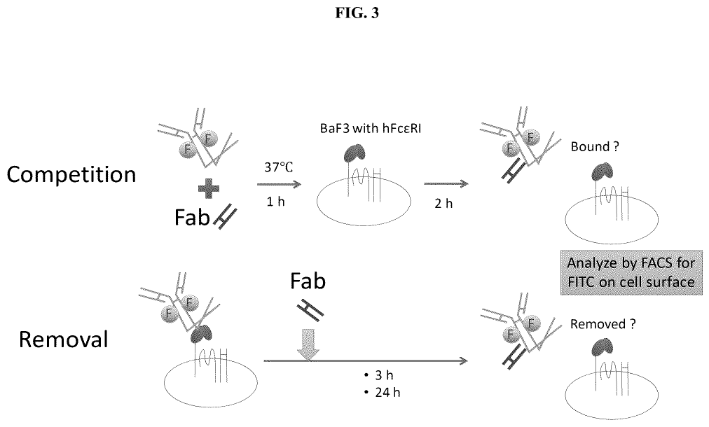

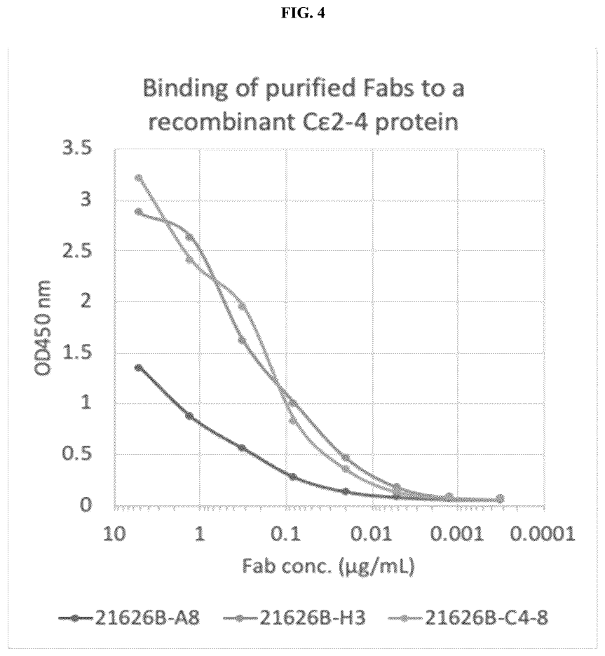

New Zealand White rabbits were immunized with a recombinant IgE Cε2-4 protein. Phage display libraries were constructed from the bone marrow and spleen and selected against a recombinant IgE Cε2-4 protein. The clones that showed specific binding to IgE Cε2-4 protein by ELISA were sequenced and their complementarity determining regions (CDRs) were analyzed (FIGS. 1-4). These clones were expressed as Fab with 6×His tag in HEK293 cells and purified using Ni-NTA column chromatography.

### Example 2—Three Fabs Inhibit Binding of Human IgE to Human FcεRI

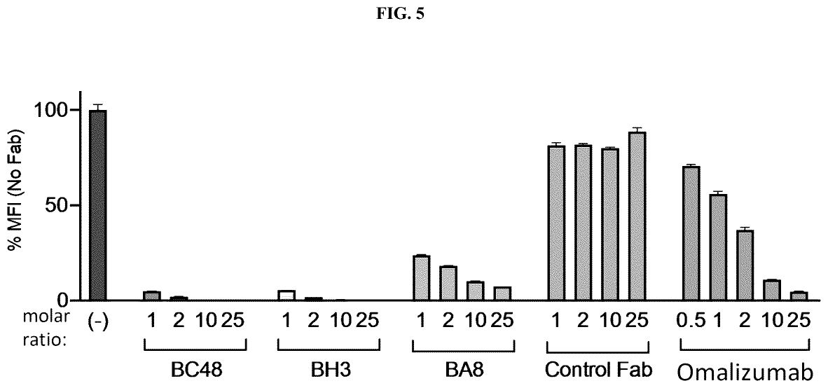

We investigated whether the newly invented Fabs can inhibit the binding of human IgE to human FcεRI. Human FcεRI is a heterotetrameric protein, which is composed of one α-chain, one β-chain, and two γ-chains. Using a BaF/3 cell line retrovirally transduced with these α, β, and γ-chains of human FcεRI, we evaluated the binding of human IgEs preincubated with three Fabs: 21626B-C4-8 (BC48)(SEQ ID No. 24, 49, 74, 99, 124, 149, KTS, and SEQ ID No. 199), 21626B-H3 (BH3)(SEQ ID No. 20, 45, 70, 95, 120, 145, RAS, and SEQ ID No.195), and 21626B-A8 (BA8)(SEQ ID No. 21, 46, 71, 96, 121EAS, 171, and SEQ ID No. 196) or Xolair to the cell surface human FcεRI. To avoid the potential interference of the Fabs with the bound IgE detection, we used fluorescently labeled antigen to measure the IgEs amount. Interestingly, all the three Fabs inhibited the binding of IgE to the cell surface human FcεRI, at a lower molar ratio compared to the commercially available Xolair (FIG. 5).

### Example 3—Three Fabs Remove Human IgE and Suppress Antigen-Stimulated Degranulation of Human FcεRIα-Transgenic Murine Bone Marrow-Derived Mast Cells (BMMCs)

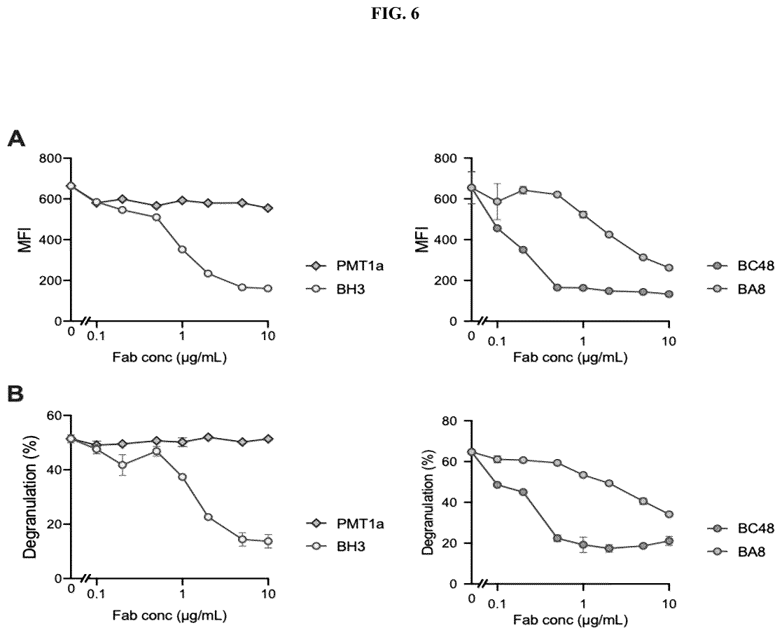

We further evaluated whether our Fabs can remove IgEs already bound to human FcεRI on cell surface. The three Fabs, but not the control Fabs that does not target human IgE, could remove IgEs in a dose dependent manner from the cell surface of human FcεRIα-transgenic BMMCs (FIG. 6A). Furthermore, the removal coincided with the reduced antigen-dependent activation of BMMCs (FIG. 6B), indicating that the three Fabs can suppress the mast cell reaction through removal of the bound IgEs

### Example 4—Three Fabs Exhibit Inhibition and Removal Activities on IgE's Binding to Low-Affinity Receptor CD2

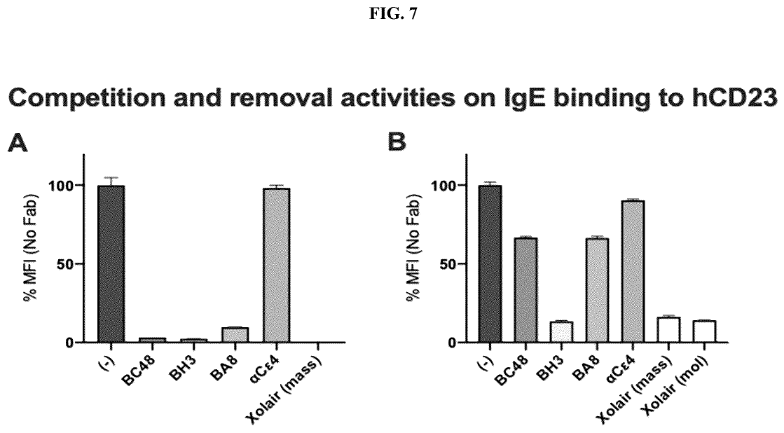

To invest the effects of the Fabs to IgE's binding to the low-affinity IgE receptor, CD23, we performed competition and removal assays using a BaF/3 cell line retrovirally transduced with human CD23. The amount of IgEs on cell surface was measured by the binding capacity of the fluorescently labeled antigens. We found that the preincubation of human IgE with the three Fabs strongly inhibited the binding to cell surface CD23 (FIG. 7A). Furthermore, the Fabs showed various removal activities within 1 h on the already bound IgE (FIG. 7B)

### Example 5—the Three Fabs Bind β5-Helix Region of Cε2 in a Tertiary Structure-Dependent Manner

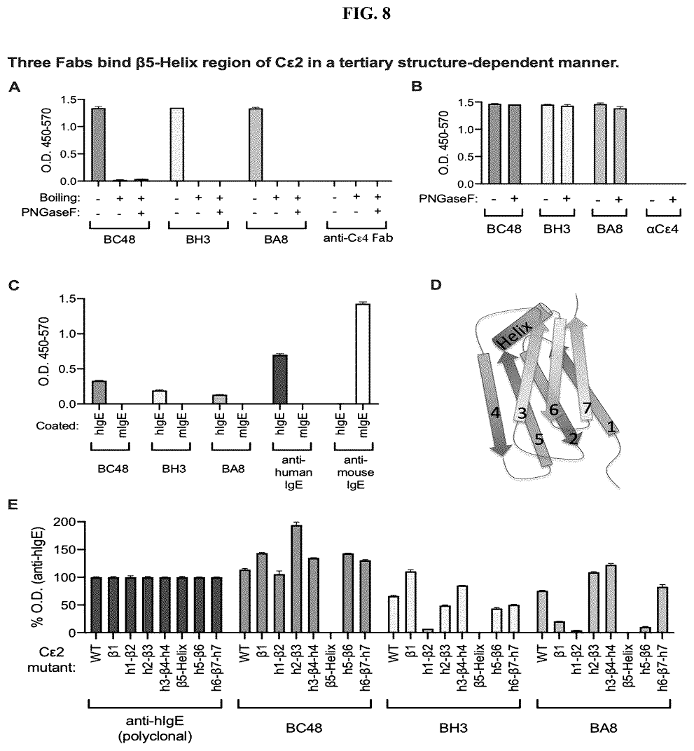

To find the binding sites of the three Fabs, we prepared recombinant His6-tagged Cε2 protein of human IgE and evaluated binding of the Fabs to the raw, boiled, and PNGaseF-treated boiled Cε2 by ELISA. The bindings of Fabs were abolished by boiling, suggesting that the tertiary structure is important for the binding of Fabs to the Cε2 (FIG. 8A). In addition, the treatment of recombinant Cε2 with PNGaseF did not affect the binding of Fabs to Cε2, suggesting that the PNGaseF-accessible N-glycan chains are not involved in the binding sites (FIG. 8B). To further narrow down the Fab binding sites in Cε2, we tried to substitute parts of the human Cε2 with the corresponding part of murine Cε2. Importantly, the three Fabs did not bind murine IgE (FIG. 8C). Because Cε2 of the two species are composed of 7 β-sheets and one α-helix (FIG. 8D), we expressed 7 chimera Cε2 proteins in HEK293T cells and evaluated the binding of Fabs to them. Interestingly, substitution of β5-helix region of human Cε2 with that of murine β5-helix abolished all three Fabs' binding (FIG. 8E). These results indicate that the three Fabs have critical binding sites at β5-helix region in common.

### Example 6—The Detailed Binding Sites in the β5-Helix Region

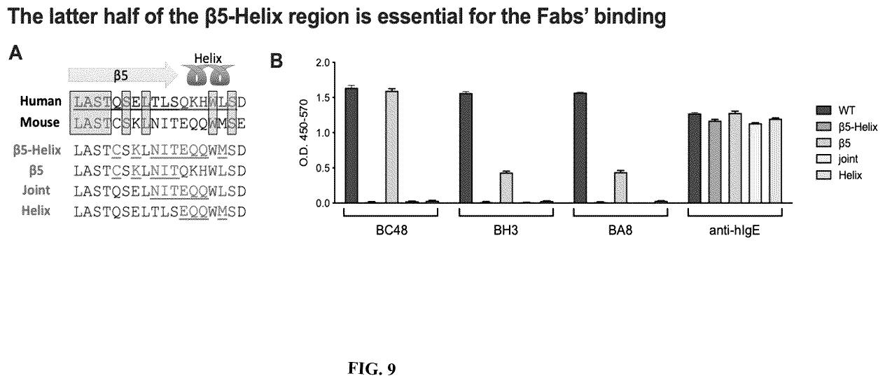

There were 9 amino acids that were different between human and mouse β5-helix region of Cε2 (FIG. 9). Therefore, we prepared chimera human IgEs, in which the parts of β5-helix region were substituted with those of murine IgEs (FIG. 9A). The binding of the three Fabs were abolished by the mutation of the middle to C-terminal of the β5-helix region (FIG. 9B). In addition, the binding of two Fabs were partially reduced by the mutation in β5 (FIG. 9B). There results indicate that the critical binding sites inside β5-helix region includes at least the α-helix region, although β5-region may also be involved.

### Example 7—Mutations of β5-Helix Regions Did not Affect IgE's Functions

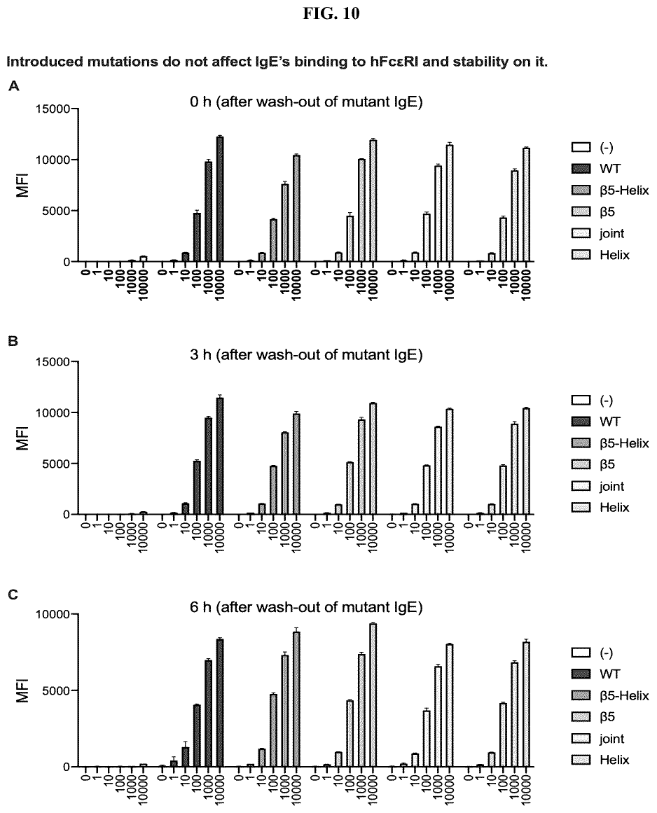

Because binding of the three Fabs removed human IgEs from the cell surface FcεRI, and because human IgEs does not bind murine FcεRI, we tested whether the substitution of the human β5-helix region of Cε2 with murine sequences can affect the binding functions of human IgE. All the human chimera IgEs evaluated in FIG. 9 bound to cell-surface FcεRI and captured different doses of fluorescently labeled antigens at the similar levels as wild-type IgEs (FIG. 10A). The antigen binding levels were similar 3 and 6 h after washing out the IgEs, indicating that the stability of IgEs on the cell surface FcεRI is also unaffected by the introduced mutations (FIGS. 10B and C). These results suggest that the β5-helix substitution does not affect IgE's intrinsic binding functions to bind to FcεRI.

### Example 8—the Inhibition of IgE's Binding to the FcεRI is Fab-Binding Dependent

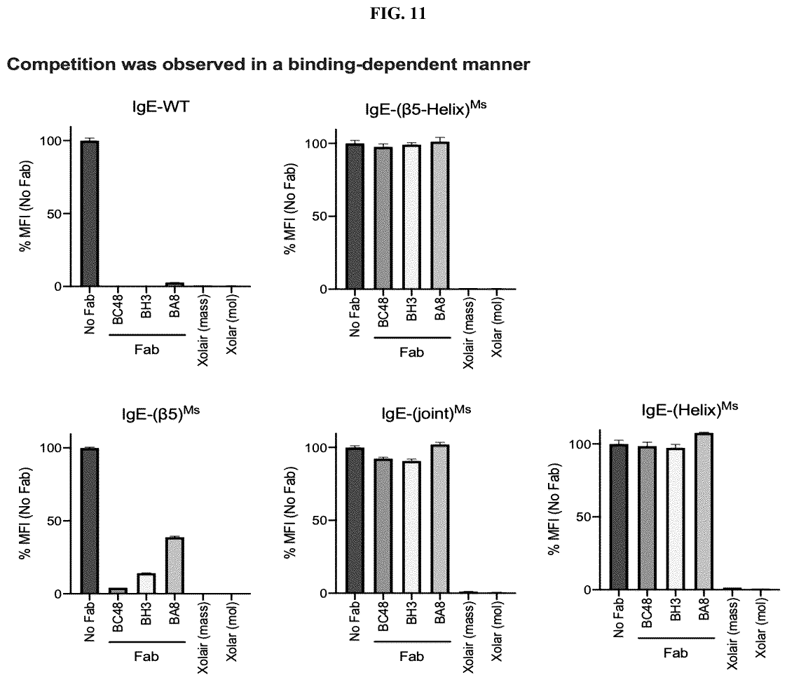

Using these IgEs, we tested the Fab's ability to inhibit the binding to the cell surface FcεRI. As expected, the mutations of Fabs' critical binding sites abolished the competitive inhibition of IgE's binding to cell surface FcεRI (FIG. 11). Furthermore, for the β5-mutated IgEs, the competition activities were correlated with the binding affinity (FIGS. 9B and 11). These results indicated that the binding inhibition effects were Fab-binding dependent.

### Example 9—the Removal of Pre-Bound IgEs on Cell Surface FcεRI is Fab-Binding Dependent

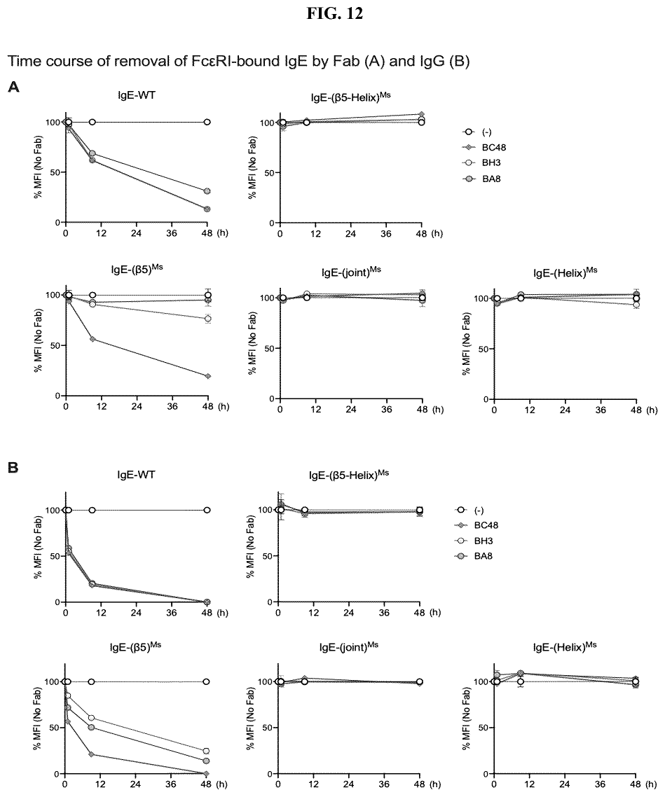

We further determined whether the Fabs' removal effects are dependent on the Fab binding. Again, the mutations of Fabs' critical binding sites abolished the removal of pre-bound IgEs on cell surface FcεRI (FIG. 12A). The similar, but stronger effects were observed when the IgG versions of the Fab clones were used (FIG. 12B). In both cases, the removal activities for the j5 region were correlated with the binding affinity (FIGS. 9B and 11). These results indicated that the removal effects were Fab-binding dependent.

### Example 10—Humanized Anti-IgEs Antibodies (BC48 and BH3)

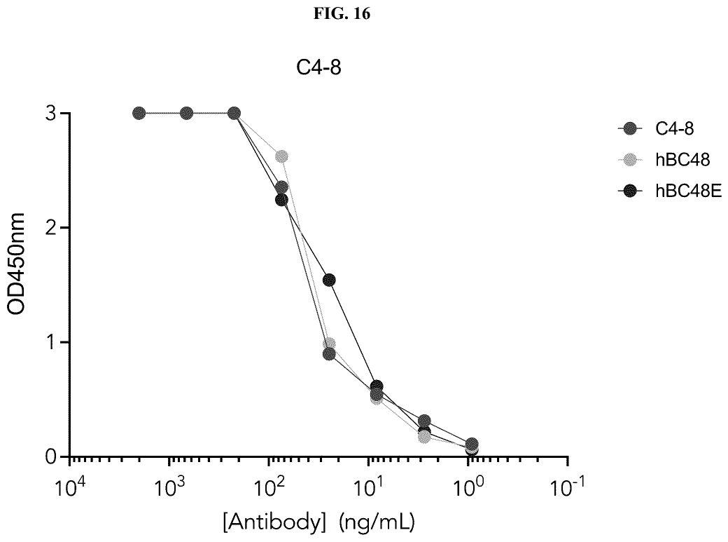

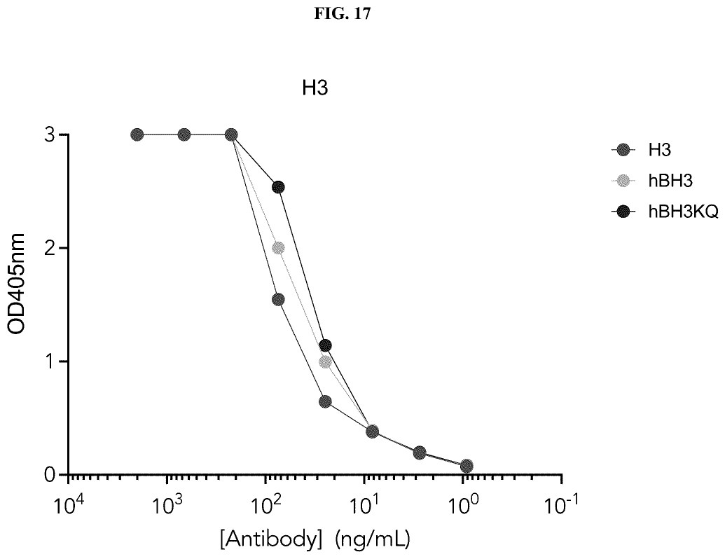

BC48 and BH3 were humanized by grafting CDRs into human germline genes. Humanized Fabs were expressed in HEK293 cells as a 6×his tag protein and purified using a Ni-NTA column. Recombinant Cε2-4 was coated at 1 μg/mL in PBS at 4° C. overnight. The wells were washed 3 times with PBS and blocked with 1% BSA/PBS. The purified Fabs were serially diluted and incubated with the antigen at RT for 1 hour. The wells were washed and the bound rabbit Fabs were detected with peroxidase conjugated goat anti-rabbit IgG F(ab′)2 (ThermoFisher Scientific 31461) and human Fabs were detected with peroxidase conjugated goat anti-human IgG F(ab′)2 (Jackson Immuno Research 109-035-097). Humanized Fabs showed equivalent binding to Cε2-4, as shown in FIGS. 16 and 17.

### Sequence Listings

### Heavy Chain Sequences

Listed below are heavy chain sequences of certain non-limiting anti-IgE antibodies, identified by name (e.g. “21626S-D5”) and ID number (e.g. “SEQ ID No. 1”).

| 21626S-D5 SEQ ID No. 1, 213, 214 | | |
|---|---|---|
| 1: | ValGlnLeuGlnGlnProGlyAlaGluLeuValLysProGlyAlaSerValLysLeuSer | |
| | GTCCAACTGCAGCAGCCTGGAGCTGAACTGGTGAAACCCGGGGCATCAGTGAAGCTGTCC | |
| | ---------!---------!---------!---------!---------!---------! | 60 |
| 1 | CAGGTTGACGTCGTCGGACCTCGACTTGACCACTTTGGGCCCCGTAGTCACTTCGACAGG | |
| 1: | CysLysAlaSerGlyAspThrPheThrGluTyrIleIleHisTrpValLysGlnArgSer | |
| | TGCAAGGCTTCTGGCGACACCTTCACTGAGTATATTATACACTGGGTAAAGCAGAGGTCT | |
| 61 | ---------!---------!---------!---------!---------!---------! | 120 |
| | ACGTTCCGAAGACCGCTGTGGAAGTGACTCATATAATATGTGACCCATTTCGTCTCCAGA | |
| 1: | GlyGlnGlyLeuGluTrpIleGlyTrpLeuSerProGlySerGlyAsnIleLysTyrAsn | |
| | GGACAGGGTCTTGAGTGGATTGGGTGGTTATCCCCTGGAAGTGGTAATATAAAGTATAAT | |
| 121 | ---------!---------!---------!---------!---------!---------! | 180 |
| | CCTGTCCCAGAACTCACCTAACCCACCAATAGGGGACCTTCACCATTATATTTCATATTA | |
| 1: | GluLysPheLysAspLysAlaThrLeuThrAlaAspLysSerSerSerThrValTyrMet | |
| | GAGAAATTCAAGGACAAGGCCACATTGACTGCGGACAAATCCTCCAGCACAGTCTATATG | |
| 181 | ---------!---------!---------!---------!---------!---------! | 240 |
| | CTCTTTAAGTTCCTGTTCCGGTGTAACTGACGCCTGTTTAGGAGGTCGTGTCAGATATAC | |
| 1: | GluLeuSerArgLeuThrSerGluAspSerAlaValTyrPheCysAlaArgHisGluVal | |
| | GAGCTTAGTAGATTGACATCTGAAGACTCTGCGGTCTATTTCTGTGCAAGACACGAAGTG | |
| | ---------!---------!---------!---------!---------!---------! | |
| 241 | CTCGAATCATCTAACTGTAGACTTCTGAGACGCCAGATAAAGACACGTTCTGTGCTTCAC | 300 |
| 1: | GlySerTyrAlaMetAspTyrTrpGlyGlnGlyThrSerValThrValSerSer | |
| | GGGTCTTATGCTATGGACTACTGGGGTCAAGGAACCTCAGTCACCGTCTCCTCA | |
| 301 | ---------!---------!---------!---------!---------!---- | 354 |
| | CCCAGAATACGATACCTGATGACCCCAGTTCCTTGGAGTCAGTGGCAGAGGAGT | |
| 21626B-B9 SEQ ID No. 2, 215, 216 | | |
| 1: | GlnThrValLysGluSerGluGlyArgLeuValThrProGlyThrProLeuThrLeuThr | |
| | CAGACGGTGAAGGAGTCCGAGGGTCGCCTGGTCACGCCTGGGACACCCCTGACACTGACC | |
| 1 | ---------!---------!---------!---------!---------!---------! | 60 |
| | GTCTGCCACTTCCTCAGGCTCCCAGCGGACCAGTGCGGACCCTGTGGGGACTGTGACTGG | |
| 1: | CysThrValSerGlyPheSerLeuSerAsnTyrAlaMetThrTrpValArgGlnAlaPro | |
| | TGCACAGTCTCTGGATTCTCCCTCAGTAACTATGCAATGACCTGGGTCCGCCAGGCTCCA | |
| 61 | ---------!---------!---------!---------!---------!---------! | 120 |
| | ACGTGTCAGAGACCTAAGAGGGAGTCATTGATACGTTACTGGACCCAGGCGGTCCGAGGT | |
| 1: | GlyLysGlyLeuGluTrpIleGlyThrIleSerSerGlyGlySerThrTyrTyrAlaSer | |
| | GGGAAGGGGCTGGAATGGATCGGAACCATTAGTAGTGGTGGTAGTACATACTACGCGAGC | |
| 121 | ---------!---------!---------!---------!---------!---------! | 180 |
| | CCCTTCCCCGACCTTACCTAGCCTTGGTAATCATCACCACCATCATGTATGATGCGCTCG | |
| 1: | GlyAlaLysGlyArgPheThrIleSerLysThrSerThrThrValAspLeuLysIleThr | |
| | GGGGCGAAAGGCCGATTCACCATCTCCAAGACCTCGACCACGGTGGATCTGAAGATCACC | |
| 181 | ---------!---------!---------!---------!---------!---------! | 240 |
| | CCCCGCTTTCCGGCTAAGTGGTAGAGGTTCTGGAGCTGGTGCCACCTAGACTTCTAGTGG | |
| 1: | SerProThrThrGluAspThrAlaThrTyrPheCysAlaArgGlyValGlyGlyGlyAsn | |
| | AGTCCGACAACCGAGGACACGGCCACCTATTTCTGTGCCAGAGGTGTTGGTGGTGGTAAT | |
| 241 | ---------!---------!---------!---------!---------!---------! | 300 |
| | TCAGGCTGTTGGCTCCTGTGCCGGTGGATAAAGACACGGTCTCCACAACCACCACCATTA | |
| 1: | TyrTyrGluHisPheAsnIleTrpGlyProGlyThrLeuValThrValSerLeu | |
| | TATTATGAGCACTTTAACATCTGGGGCCCAGGCACCCTGGTCACCGTCTCCTTA | |
| 301 | ---------!---------!---------!---------!---------!---- | 354 |
| | ATAATACTCGTGAAATTGTAGACCCCGGGTCCGTGGGACCAGTGGCAGAGGAAT | |
| 21626B-H8 SEQ ID No. 3, 217, 218 | | |
| 1: | GlnSerValGluGluSerGlyGlyArgLeuValThrProGlyThrProLeuThrLeuThr | |
| | CAGTCGGTGGAGGAGTCCGGGGGTCGCCTGGTCACGCCTGGGACACCCCTGACACTCACC | |
| 1 | ---------!---------!---------!---------!---------!---------! | 60 |
| | GTCAGCCACCTCCTCAGGCCCCCAGCGGACCAGTGCGGACCCTGTGGGGACTGTGAGTGG | |
| 1: | CysThrAlaSerGlyPheSerLeuSerThrTyrSerMetSerTrpValArgGlnAlaPro | |
| | TGCACAGCCTCTGGATTCTCCCTCAGTACCTACTCCATGAGCTGGGTCCGCCAGGCTCCA | |
| 61 | ---------!---------!---------!---------!---------!---------! | 120 |
| | ACGTGTCGGAGACCTAAGAGGGAGTCATGGATGAGGTACTCGACCCAGGCGGTCCGAGGT | |
| 1: | GlyLysGlyLeuGluTrpIleGlyIleIleSerSerSerGlyAsnThrTyrTyrAlaSer | |
| | GGGAAGGGGCTGGAATGGATCGGAATCATTAGTAGTAGTGGTAACACATACTACGCGAGC | |
| 121 | ---------!---------!---------!---------!---------!---------! | 180 |
| | CCCTTCCCCGACCTTACCTAGCCTTAGTAATCATCATCACCATTGTGTATGATGCGCTCG | |
| 1: | TrpAlaLysGlyArgPheThrIleSerLysSerSerThrThrValAspLeuLysIleThr | |
| | TGGGCGAAAGGCCGATTCACCATCTCCAAGTCCTCGACCACGGTGGATCTGAAAATCACC | |
| 181 | ---------!---------!---------!---------!---------!---------! | 240 |
| | ACCCGCTTTCCGGCTAAGTGGTAGAGGTTCAGGAGCTGGTGCCACCTAGACTTTTAGTGG | |
| 1: | SerProThrThrGluAspThrAlaThrTyrPheCysAlaArgGlyValGlyTyrAsnAsn | |
| | AGTCCGACAACCGAGGACACGGCCACCTATTTCTGTGCCAGAGGTGTTGGTTATAATAAT | |
| 241 | ---------!---------!---------!---------!---------!---------! | 300 |
| | TCAGGCTGTTGGCTCCTGTGCCGGTGGATAAAGACACGGTCTCCACAACCAATATTATTA | |
| 1: | TyrTyrGluHisPheAsnIleTrpGlyProGlyThrLeuValThrValSerLeu | |
| | TATTATGAGCACTTTAACATCTGGGGCCCAGGCACCCTGGTCACCGTCTCCTTA | |
| 301 | ---------!---------!---------!---------!---------!---- | 354 |
| | ATAATACTCGTGAAATTGTAGACCCCGGGTCCGTGGGACCAGTGGCAGAGGAAT | |
| 21626S-E6 SEQ ID No. 4, 219, 220 | | |
| 1: | GlnSerValLysGluSerGlyGlyArgLeuValThrProGlyThrProLeuThrLeuThr | |
| | CAGTCGGTGAAGGAGTCCGGGGGTCGCCTGGTCACGCCTGGGACACCCCTGACACTCACC | |
| 1 | ---------!---------!---------!---------!---------!---------! | 60 |
| | GTCAGCCACTTCCTCAGGCCCCCAGCGGACCAGTGCGGACCCTGTGGGGACTGTGAGTGG | |
| 1: | CysThrValSerGlyPheSerLeuSerAsnTyrAlaMetSerTrpValArgGlnAlaPro | |
| | TGCACAGTCTCTGGATTCTCCCTCAGTAACTATGCAATGAGCTGGGTCCGCCAGGCTCCA | |
| 61 | ---------!---------!---------!---------!---------!---------! | 120 |
| | ACGTGTCAGAGACCTAAGAGGGAGTCATTGATACGTTACTCGACCCAGGCGGTCCGAGGT | |
| 1: | GlyLysGlyLeuGluTrpIleGlyIleIleSerSerGlyAlaSerThrTyrTyrAlaSer | |
| | GGGAAGGGGCTGGAATGGATCGGAATCATTAGTAGTGGTGCTAGCACATACTACGCGAGC | |
| 121 | ---------!---------!---------!---------!---------!---------! | 180 |
| | CCCTTCCCCGACCTTACCTAGCCTTAGTAATCATCACCACGATCGTGTATGATGCGCTCG | |
| 1: | TrpAlaLysGlyArgPheThrIleSerArgThrSerThrThrValAspLeuLysIleThr | |
| | TGGGCGAAAGGCCGATTCACCATCTCCAGAACCTCGACCACGGTGGATCTGAAAATCACC | |
| 181 | ---------!---------!---------!---------!---------!---------! | 240 |
| | ACCCGCTTTCCGGCTAAGTGGTAGAGGTCTTGGAGCTGGTGCCACCTAGACTTTTAGTGG | |
| 1: | SerProThrThrGluAspThrAlaThrTyrPheCysAlaArgGlyTyrGlyGlyAlaAsn | |
| | AGTCCGACAACCGAGGACACGGCCACCTATTTCTGTGCCAGAGGTTATGGTGGTGCTAAT | |
| 241 | ---------!---------!---------!---------!---------!---------! | 300 |
| | TCAGGCTGTTGGCTCCTGTGCCGGTGGATAAAGACACGGTCTCCAATACCACCACGATTA | |
| 1: | TyrTyrGluHisPheAsnIleTrpGlyProGlyThrLeuValThrValSerLeu | |
| | TATTATGAGCACTTTAACATCTGGGGCCCAGGCACCCTGGTCACCGTCTCCTTA | |
| 301 | ---------!---------!---------!---------!---------!---- | 354 |
| | ATAATACTCGTGAAATTGTAGACCCCGGGTCCGTGGGACCAGTGGCAGAGGAAT | |
| 21626S-E12 SEQ ID No. 5, 221, 222 | | |
| 1: | GlnThrValLysGluSerGlyGlyArgLeuValThrProGlyThrProLeuThrLeuThr | |
| | CAGACAGTGAAGGAGTCCGGGGGTCGCCTGGTCACGCCTGGGACACCCCTGACACTCACC | |
| 1 | ---------!---------!---------!---------!---------!---------! | 60 |
| | GTCTGTCACTTCCTCAGGCCCCCAGCGGACCAGTGCGGACCCTGTGGGGACTGTGAGTGG | |
| 1: | CysThrValSerGlyPheSerLeuSerAsnTyrAlaMetThrTrpValArgGlnAlaPro | |
| | TGCACAGTCTCTGGATTCTCCCTCAGTAACTATGCAATGACCTGGGTCCGCCAGGCTCCG | |
| 61 | ---------!---------!---------!---------!---------!---------! | 120 |
| | ACGTGTCAGAGACCTAAGAGGGAGTCATTGATACGTTACTGGACCCAGGCGGTCCGAGGC | |
| 1: | GlyLysGlyLeuGluTrpIleGlyIleIleSerSerGlyGlyTyrThrTyrTyrAlaSer | |
| | GGGAAGGGGCTGGAATGGATCGGAATCATTAGTAGTGGTGGTTATACATACTACGCGAGC | |
| 121 | ---------!---------!---------!---------!---------!---------! | 180 |
| | CCCTTCCCCGACCTTACCTAGCCTTAGTAATCATCACCACCAATATGTATGATGCGCTCG | |
| 1: | TrpAlaLysGlyArgPheThrIleSerLysThrSerThrThrValAspLeuLysIleThr | |
| | TGGGCGAAAGGCCGATTCACCATCTCCAAAACCTCGACCACGGTGGATCTGAAAATCACC | |
| 181 | ---------!---------!---------!---------!---------!---------! | 240 |
| | ACCCGCTTTCCGGCTAAGTGGTAGAGGTTTTGGAGCTGGTGCCACCTAGACTTTTAGTGG | |
| 1: | SerProThrThrGluAspThrAlaThrTyrPheCysAlaArgGlyTyrGlyGlyGlyAsn | |
| | AGTCCGACAACCGAGGACACGGCCACCTATTTCTGTGCCAGAGGTTATGGTGGTGGTAAC | |
| 241 | ---------!---------!---------!---------!---------!---------! | 300 |
| | TCAGGCTGTTGGCTCCTGTGCCGGTGGATAAAGACACGGTCTCCAATACCACCACCATTG | |
| 1: | TyrTyrGluHisTyrAsnIleTrpGlyProGlyThrLeuValThrValSerLeu | |
| | TATTATGAGCACTATAACATCTGGGGCCCAGGCACCCTGGTCACCGTCTCCTTA | |
| 301 | ---------!---------!---------!---------!---------!---- | 354 |
| | ATAATACTCGTGATATTGTAGACCCCGGGTCCGTGGGACCAGTGGCAGAGGAAT | |
| 21626B-F7 SEQ ID No. 6, 223, 224 | | |
| 1: | GlnSerLeuGluGluSerGlyGlyArgLeuValThrProGlyThrProLeuThrLeuThr | |
| | CAGTCGCTGGAGGAGTCCGGGGGTCGCCTGGTCACGCCTGGGACACCCCTGACACTCACC | |
| 1 | ---------!---------!---------!---------!---------!---------! | 60 |
| | GTCAGCGACCTCCTCAGGCCCCCAGCGGACCAGTGCGGACCCTGTGGGGACTGTGAGTGG | |
| 1: | CysThrValSerGlyPheSerLeuSerAlaTyrThrMetThrTrpValArgGlnAlaPro | |
| | TGCACAGTCTCTGGATTCTCCCTCAGTGCCTATACAATGACCTGGGTCCGCCAGGCTCCA | |
| 61 | ---------!---------!---------!---------!---------!---------! | 120 |
| | ACGTGTCAGAGACCTAAGAGGGAGTCACGGATATGTTACTGGACCCAGGCGGTCCGAGGT | |
| 1: | GlyLysGlyLeuGluTrpIleGlyAlaIleSerThrGlyGlySerThrTyrTyrAlaAsn | |
| | GGGAAGGGGCTGGAATGGATCGGAGCCATTAGTACTGGTGGTAGCACATACTACGCGAAC | |
| 121 | ---------!---------!---------!---------!---------!---------! | 180 |
| | CCCTTCCCCGACCTTACCTAGCCTCGGTAATCATGACCACCATCGTGTATGATGCGCTTG | |
| 1: | TrpAlaLysGlyArgPheThrIleSerLysThrSerThrThrValAspLeuLysIleThr | |
| | TGGGCGAAAGGCCGATTCACCATCTCCAAAACCTCGACCACGGTGGATCTGAAAATCACC | |
| 181 | ---------!---------!---------!---------!---------!---------! | 240 |
| | ACCCGCTTTCCGGCTAAGTGGTAGAGGTTTTGGAGCTGGTGCCACCTAGACTTTTAGTGG | |
| 1: | SerProThrThrGluAspThrAlaThrTyrPheCysAlaArgGlyTyrGlySerAsnAsn | |
| | AGTCCGACAACCGAGGACACGGCCACCTATTTCTGTGCCAGAGGTTATGGTAGTAATAAT | |
| 241 | ---------!---------!---------!---------!---------!---------! | 300 |
| | TCAGGCTGTTGGCTCCTGTGCCGGTGGATAAAGACACGGTCTCCAATACCATCATTATTA | |
| 1: | TyrTyrGluHisPheLysIleTrpGlyProGlyThrLeuValThrValSerLeu | |
| | TATTATGAGCACTTTAAGATCTGGGGCCCAGGCACCCTGGTCACCGTCTCCTTA | |
| 301 | ---------!---------!---------!---------!---------!---- | 354 |
| | ATAATACTCGTGAAATTCTAGACCCCGGGTCCGTGGGACCAGTGGCAGAGGAAT | |
| 21626B-F5 SEQ ID No. 7, 225, 226 | | |
| 1: | GlnGluGlnLeuMetGluSerGlyGlyArgLeuValThrProGlyThrProLeuThrLeu | |
| | CAGGAGCAGCTGATGGAGTCCGGGGGTCGCCTGGTCACGCCTGGGACACCCCTGACACTC | |
| 1 | ---------!---------!---------!---------!---------!---------! | 60 |
| | GTCCTCGTCGACTACCTCAGGCCCCCAGCGGACCAGTGCGGACCCTGTGGGGACTGTGAG | |
| 1: | ThrCysThrValSerGlyPheSerLeuSerSerTyrAlaMetThrTrpValArgGlnAla | |
| | ACCTGCACAGTCTCTGGATTCTCCCTCAGTAGCTATGCAATGACCTGGGTCCGCCAGGCT | |
| 61 | ---------!---------!---------!---------!---------!---------! | 120 |
| | TGGACGTGTCAGAGACCTAAGAGGGAGTCATCGATACGTTACTGGACCCAGGCGGTCCGA | |
| 1: | ProGlyLysGlyLeuGluTrpIleGlyIleIleSerThrAsnGlyAsnThrTyrTyrAla | |
| | CCAGGGAAGGGGCTGGAATGGATCGGAATCATTAGTACTAATGGTAACACATACTACGCG | |
| 121 | ---------!---------!---------!---------!---------!---------! | 180 |
| | GGTCCCTTCCCCGACCTTACCTAGCCTTAGTAATCATGATTACCATTGTGTATGATGCGC | |
| 1: | SerTrpAlaLysGlyArgPheAlaIleSerLysThrSerThrThrValAspLeuLysIle | |
| | AGCTGGGCGAAAGGCCGATTCGCCATCTCCAAAACCTCGACCACGGTGGATCTGAAAATC | |
| 181 | ---------!---------!---------!---------!---------!---------! | 240 |
| | TCGACCCGCTTTCCGGCTAAGCGGTAGAGGTTTTGGAGCTGGTGCCACCTAGACTTTTAG | |
| 1: | ThrSerProThrThrGluAspThrAlaThrTyrPheCysAlaArgGlyTyrGlySerAsn | |
| | ACCAGTCCGACAACCGAGGACACGGCCACCTATTTCTGTGCCAGAGGTTATGGTAGTAAT | |
| 241 | ---------!---------!---------!---------!---------!---------! | 300 |
| | TGGTCAGGCTGTTGGCTCCTGTGCCGGTGGATAAAGACACGGTCTCCAATACCATCATTA | |
| 1: | AsnTyrTyrGluHisPheAsnIleTrpGlyProGlyThrLeuValThrValSerLeu | |
| | AATTATTATGAGCACTTTAACATCTGGGGCCCAGGCACCCTGGTCACCGTCTCCTTA | |
| 301 | ---------!---------!---------!---------!---------!------- | 357 |
| | TTAATAATACTCGTGAAATTGTAGACCCCGGGTCCGTGGGACCAGTGGCAGAGGAAT | |
| 21626B-C5 SEQ ID No. 8, 227, 228 | | |
| 1: | GlnGluGlnLeuLysGluSerGlyGlyArgLeuValThrProGlyThrProLeuThrLeu | |
| | CAGGAGCAGCTGAAGGAGTCCGGGGGTCGCCTGGTCACGCCTGGGACACCCCTGACACTC | |
| 1 | ---------!---------!---------!---------!---------!---------! | 60 |
| | GTCCTCGTCGACTTCCTCAGGCCCCCAGCGGACCAGTGCGGACCCTGTGGGGACTGTGAG | |
| 1: | ThrCysThrValSerGlyPheSerLeuSerAsnTyrAlaMetSerTrpValArgGlnAla | |
| | ACCTGCACAGTCTCTGGATTCTCCCTCAGTAACTATGCAATGAGCTGGGTCCGCCAGGCT | |
| 61 | ---------!---------!---------!---------!---------!---------! | 120 |
| | TGGACGTGTCAGAGACCTAAGAGGGAGTCATTGATACGTTACTCGACCCAGGCGGTCCGA | |
| 1: | ProGlyLysGlyLeuGluTrpIleGlyIleIleSerSerSerGlyAsnThrTyrTyrAla | |
| | CCAGGGAAGGGGCTGGAATGGATCGGAATCATTAGTAGTAGTGGTAACACATACTACGCG | |
| 121 | ---------!---------!---------!---------!---------!---------! | 180 |
| | GGTCCCTTCCCCGACCTTACCTAGCCTTAGTAATCATCATCACCATTGTGTATGATGCGC | |
| 1: | SerTrpAlaLysGlyArgPheThrIleSerLysThrSerThrThrValAspLeuLysIle | |
| | AGCTGGGCGAAAGGCCGATTCACCATCTCCAAAACCTCGACCACGGTGGATCTGAAAATC | |
| 181 | ---------!---------!---------!---------!---------!---------! | 240 |
| | TCGACCCGCTTTCCGGCTAAGTGGTAGAGGTTTTGGAGCTGGTGCCACCTAGACTTTTAG | |
| 1: | ThrSerProThrThrGluAspThrAlaThrTyrPheCysAlaArgGlyTyrGlyTyrAsn | |
| | ACCAGTCCGACAACCGAGGACACGGCCACCTATTTCTGTGCCAGAGGTTATGGTTATAAT | |
| 241 | ---------!---------!---------!---------!---------!---------! | 300 |
| | TGGTCAGGCTGTTGGCTCCTGTGCCGGTGGATAAAGACACGGTCTCCAATACCAATATTA | |
| 1: | AsnTyrTyrGluHisPheAsnIleTrpGlyProGlyThrLeuValThrValSerLeu | |
| | AATTATTATGAGCACTTTAACATCTGGGGCCCAGGCACCCTGGTCACCGTCTCCTTA | |
| 301 | ---------!---------!---------!---------!---------!------- | 357 |
| | TTAATAATACTCGTGAAATTGTAGACCCCGGGTCCGTGGGACCAGTGGCAGAGGAAT | |
| 21626S-G9 SEQ ID No. 9, 229, 230 | | |
| 1: | GlnGluGlnLeuGluGluSerGlyGlyArgLeuValThrProGlyThrProLeuThrLeu | |
| | CAGGAGCAGCTGGAGGAGTCCGGGGGTCGCCTGGTCACGCCTGGGACACCCCTGACACTC | |
| 1 | ---------!---------!---------!---------!---------!---------! | 60 |
| | GTCCTCGTCGACCTCCTCAGGCCCCCAGCGGACCAGTGCGGACCCTGTGGGGACTGTGAG | |
| 1: | ThrCysThrAlaSerGlyPheSerLeuSerAlaTyrSerMetSerTrpValArgGlnAla | |
| | ACCTGCACAGCCTCTGGATTCTCCCTCAGTGCCTACTCCATGAGCTGGGTCCGCCAGGCT | |
| 61 | ---------!---------!---------!---------!---------!---------! | 120 |
| | TGGACGTGTCGGAGACCTAAGAGGGAGTCACGGATGAGGTACTCGACCCAGGCGGTCCGA | |
| 1: | ProGlyLysGlyLeuGluTrpIleGlyIleIleSerSerSerGlyTyrThrTyrTyrAla | |
| | CCAGGGAAGGGGCTGGAATGGATCGGAATCATTAGTAGCAGTGGTTACACATACTACGCG | |
| 121 | ---------!---------!---------!---------!---------!---------! | 180 |
| | GGTCCCTTCCCCGACCTTACCTAGCCTTAGTAATCATCGTCACCAATGTGTATGATGCGC | |
| 1: | SerTrpAlaLysGlyArgPheThrIleSerLysThrSerThrThrValAspLeuGluIle | |
| | AGCTGGGCGAAAGGCCGATTCACCATCTCCAAAACCTCGACCACGGTGGATCTGGAGATC | |
| 181 | ---------!---------!---------!---------!---------!---------! | 240 |
| | TCGACCCGCTTTCCGGCTAAGTGGTAGAGGTTTTGGAGCTGGTGCCACCTAGACCTCTAG | |
| 1: | ThrSerProThrThrGluAspThrAlaThrTyrPheCysAlaArgGlyTyrGlyTyrAsn | |
| | ACCAGTCCGACAACCGAGGACACGGCCACCTATTTCTGTGCCAGAGGTTATGGTTATAAT | |
| 241 | ---------!---------!---------!---------!---------!---------! | 300 |
| | TGGTCAGGCTGTTGGCTCCTGTGCCGGTGGATAAAGACACGGTCTCCAATACCAATATTA | |
| 1: | AsnTyrTyrGluHisPheAsnMetTrpGlyProGlyThrLeuValThrValSerLeu | |
| | AATTATTATGAGCACTTTAACATGTGGGGCCCAGGCACCCTGGTCACCGTCTCCTTA | |
| 301 | ---------!---------!---------!---------!---------!------- | 357 |
| | TTAATAATACTCGTGAAATTGTACACCCCGGGTCCGTGGGACCAGTGGCAGAGGAAT | |
| 21626S-F8 SEQ ID No. 10, 231, 232 | | |
| 1: | GlnSerValLysGluSerGluGlyArgLeuValThrProGlyThrProLeuThrLeuThr | |
| | CAGTCAGTGAAGGAGTCCGAGGGTCGCCTGGTCACGCCTGGGACACCCCTGACACTCACC | |
| 1 | ---------!---------!---------!---------!---------!---------! | 60 |
| | GTCAGTCACTTCCTCAGGCTCCCAGCGGACCAGTGCGGACCCTGTGGGGACTGTGAGTGG | |
| 1: | CysThrValAlaGlyPheSerLeuSerHisTyrHisMetSerTrpValArgGlnAlaPro | |
| | TGCACAGTCGCTGGATTCTCCCTCAGCCACTACCATATGAGCTGGGTCCGCCAGGCTCCA | |
| 61 | ---------!---------!---------!---------!---------!---------! | 120 |
| | ACGTGTCAGCGACCTAAGAGGGAGTCGGTGATGGTATACTCGACCCAGGCGGTCCGAGGT | |
| 1: | GlyLysGlyLeuGluTrpIleGlyIleIleGlySerSerGlyAsnThrTrpTyrAlaSer | |
| | GGGAAGGGGCTGGAATGGATCGGAATCATTGGTAGTAGTGGAAACACATGGTACGCGAGC | |
| 121 | ---------!---------!---------!---------!---------!---------! | 180 |
| | CCCTTCCCCGACCTTACCTAGCCTTAGTAACCATCATCACCTTTGTGTACCATGCGCTCG | |
| 1: | TrpAlaLysGlyArgPheThrIleSerLysThrSerThrThrValAspLeuArgIleThr | |
| | TGGGCGAAAGGCCGATTCACCATCTCCAAGACCTCGACCACGGTGGATCTGAGAATCACC | |
| 181 | ---------!---------!---------!---------!---------!---------! | 240 |
| | ACCCGCTTTCCGGCTAAGTGGTAGAGGTTCTGGAGCTGGTGCCACCTAGACTCTTAGTGG | |
| 1: | SerProThrThrGluAspThrAlaThrTyrPheCysAlaArgAlaGlyAlaSerAsnAsn | |
| | AGTCCGACAACCGAGGACACGGCCACCTATTTCTGTGCCAGAGCTGGTGCTAGTAACAAT | |
| 241 | ---------!---------!---------!---------!---------!---------! | 300 |
| | TCAGGCTGTTGGCTCCTGTGCCGGTGGATAAAGACACGGTCTCGACCACGATCATTGTTA | |
| 1: | TyrTyrAsnTyrPheAsnValTrpGlyProGlyThrLeuValThrValSerLeu | |
| | TATTATAATTACTTTAACGTCTGGGGCCCAGGCACCCTGGTCACCGTCTCCTTA | |
| 301 | ---------!---------!---------!---------!---------!---- | 354 |
| | ATAATATTAATGAAATTGCAGACCCCGGGTCCGTGGGACCAGTGGCAGAGGAAT | |
| 21626B-F10 SEQ ID No. 11, 233, 234 | | |
| 1: | GlnSerValLysGluSerGlyGlyArgLeuValThrProGlyThrProLeuThrLeuThr | |
| | CAGTCGGTGAAGGAGTCCGGGGGTCGCCTGGTCACGCCTGGGACACCCCTGACACTCACC | |
| 1 | ---------!---------!---------!---------!---------!---------! | 60 |
| | GTCAGCCACTTCCTCAGGCCCCCAGCGGACCAGTGCGGACCCTGTGGGGACTGTGAGTGG | |
| 1: | CysThrValAlaGlyPheSerLeuSerArgTyrTyrMetSerTrpValArgGlnAlaPro | |
| | TGCACAGTCGCTGGATTCTCCCTCAGCCGCTACTATATGAGCTGGGTCCGCCAGGCTCCA | |
| 61 | ---------!---------!---------!---------!---------!---------! | 120 |
| | ACGTGTCAGCGACCTAAGAGGGAGTCGGCGATGATATACTCGACCCAGGCGGTCCGAGGT | |
| 1: | GlyLysGlyLeuGluTrpIleGlyIleIleSerSerSerGlySerThrTyrTyrAlaSer | |
| | GGGAAGGGGCTGGAATGGATCGGAATCATTAGTAGTAGTGGTAGCACATACTACGCGAGC | |
| 121 | ---------!---------!---------!---------!---------!---------! | 180 |
| | CCCTTCCCCGACCTTACCTAGCCTTAGTAATCATCATCACCATCGTGTATGATGCGCTCG | |
| 1: | TrpAlaLysGlyArgPheThrIleSerLysThrSerThrThrValAspLeuArgIleThr | |
| | TGGGCGAAAGGCCGATTCACCATCTCCAAAACCTCGACCACGGTGGATCTGAGAATCACC | |
| 181 | ---------!---------!---------!---------!---------!---------! | 240 |
| | ACCCGCTTTCCGGCTAAGTGGTAGAGGTTTTGGAGCTGGTGCCACCTAGACTCTTAGTGG | |
| 1: | SerProThrThrGluAspThrAlaThrTyrPheCysAlaArgGlyGlyAlaSerAsnAsn | |
| | AGTCCGACAACCGAGGACACGGCCACCTATTTCTGTGCCAGAGGTGGTGCTAGTAATAAT | |
| 241 | ---------!---------!---------!---------!---------!---------! | 300 |
| | TCAGGCTGTTGGCTCCTGTGCCGGTGGATAAAGACACGGTCTCCACCACGATCATTATTA | |
| 1: | TyrTyrAsnTyrPheAsnIleTrpGlyProGlyThrLeuValThrValSerLeu | |
| | TATTATAATTACTTTAACATCTGGGGCCCAGGCACCCTGGTCACCGTCTCCTTA | |
| 301 | ---------!---------!---------!---------!---------!---- | 354 |
| | ATAATATTAATGAAATTGTAGACCCCGGGTCCGTGGGACCAGTGGCAGAGGAAT | |
| 21626B-G12 SEQ ID No. 12, 235, 236 | | |
| 1: | GlnSerLeuGluGluSerGlyGlyGlyLeuPheLysProThrAspThrLeuThrLeuThr | |
| | CAGTCGTTGGAGGAGTCCGGGGGAGGTCTCTTCAAGCCAACGGATACCCTGACTCTCACC | |
| 1 | ---------!---------!---------!---------!---------!---------! | 60 |
| | GTCAGCAACCTCCTCAGGCCCCCTCCAGAGAAGTTCGGTTGCCTATGGGACTGAGAGTGG | |
| 1: | CysThrValSerGlyPheProLeuSerThrTyrGlyIleThrTrpValArgGlnAlaPro | |
| | TGCACAGTCTCTGGATTCCCCCTCAGTACCTATGGAATAACCTGGGTCCGCCAGGCTCCA | |
| 61 | ---------!---------!---------!---------!---------!---------! | 120 |
| | ACGTGTCAGAGACCTAAGGGGGAGTCATGGATACCTTATTGGACCCAGGCGGTCCGAGGT | |
| 1: | GlyAsnGlyLeuGluTrpIleGlyIleIleSerSerGlyGlySerThrHisTyrAlaSer | |
| | GGGAACGGGCTGGAATGGATCGGAATCATTAGTAGTGGTGGTAGCACACACTACGCGAGC | |
| 121 | ---------!---------!---------!---------!---------!---------! | 180 |
| | CCCTTGCCCGACCTTACCTAGCCTTAGTAATCATCACCACCATCGTGTGTGATGCGCTCG | |
| 1: | TrpAlaLysSerArgSerThrIleThrArgAspThrAsnGluAsnThrValThrLeuLys | |
| | TGGGCGAAAAGCCGATCCACCATCACCAGAGACACCAACGAGAACACGGTGACTCTGAAA | |
| 181 | ---------!---------!---------!---------!---------!---------! | 240 |
| | ACCCGCTTTTCGGCTAGGTGGTAGTGGTCTCTGTGGTTGCTCTTGTGCCACTGAGACTTT | |
| 1: | MetThrSerLeuThrValAlaAspThrAlaThrTyrPheCysAlaArgAlaProTyrPhe | |
| | ATGACCAGTCTGACAGTCGCGGACACGGCCACCTATTTCTGTGCGAGAGCCCCCTACTTT | |
| 241 | ---------!---------!---------!---------!---------!---------! | 300 |
| | TACTGGTCAGACTGTCAGCGCCTGTGCCGGTGGATAAAGACACGCTCTCGGGGGATGAAA | |
| 1: | ArgTrpAspPheAsnTrpAspLeuSerAlaPheAspProTrpGlyProGlyThrLeuVal | |
| | AGGTGGGATTTTAATTGGGATCTTTCCGCTTTTGATCCCTGGGGCCCAGGCACCCTGGTC | |
| 301 | ---------!---------!---------!---------!---------!---------! | 360 |
| | TCCACCCTAAAATTAACCCTAGAAAGGCGAAAACTAGGGACCCCGGGTCCGTGGGACCAG | |
| 1: | ThrValSerSer | |
| | ACCGTCTCCTCA | |
| 361 | ---------!-- | 372 |
| | TGGCAGAGGAGT | |
| 21626B-A11 SEQ ID No. 13, 237, 238 | | |
| 1: | GlnSerLeuGlyGluSerGlyGlyArgLeuValLysProAspGluThrLeuThrLeuThr | |
| | CAGTCGCTGGGGGAGTCCGGGGGTCGCCTGGTCAAGCCTGACGAAACCCTGACACTCACC | |
| 1 | ---------!---------!---------!---------!---------!---------! | 60 |
| | GTCAGCGACCCCCTCAGGCCCCCAGCGGACCAGTTCGGACTGCTTTGGGACTGTGAGTGG | |
| 1: | CysThrAlaSerGlyPheSerLeuSerAsnTyrAspMetGlyTrpValArgGlnAlaPro | |
| | TGCACAGCCTCTGGATTCTCCCTCAGTAACTACGACATGGGCTGGGTCCGCCAGGCTCCA | |
| 61 | ---------!---------!---------!---------!---------!---------! | 120 |
| | ACGTGTCGGAGACCTAAGAGGGAGTCATTGATGCTGTACCCGACCCAGGCGGTCCGAGGT | |
| 1: | GlyGluGlyLeuGluTyrIleGlyTrpIleThrThrGlyGlySerAlaTyrTyrAlaAsn | |
| | GGGGAGGGGCTGGAATATATCGGATGGATTACTACTGGTGGTAGCGCATACTACGCGAAC | |
| 121 | ---------!---------!---------!---------!---------!---------! | 180 |
| | CCCCTCCCCGACCTTATATAGCCTACCTAATGATGACCACCATCGCGTATGATGCGCTTG | |
| 1: | TrpAlaLysGlyArgPheThrIleSerArgThrSerThrThrValAspLeuLysIleSer | |
| | TGGGCAAAAGGCCGATTCACCATCTCCAGAACCTCGACCACGGTGGATCTGAAAATCAGC | |
| 181 | ---------!---------!---------!---------!---------!---------! | 240 |
| | ACCCGTTTTCCGGCTAAGTGGTAGAGGTCTTGGAGCTGGTGCCACCTAGACTTTTAGTCG | |
| 1: | SerProThrSerGluAspThrAlaThrTyrPheCysAlaArgAspGlyGlyArgGlyTyr | |
| | AGTCCGACAAGTGAGGACACGGCCACCTATTTCTGTGCCAGAGATGGTGGCCGTGGTTAT | |
| 241 | ---------!---------!---------!---------!---------!---------! | 300 |
| | TCAGGCTGTTCACTCCTGTGCCGGTGGATAAAGACACGGTCTCTACCACCGGCACCAATA | |
| 1: | GlyTyrSerPheSerIleTrpGlyProGlyThrLeuValThrValSerSer | |
| | GGTTATTCATTTAGCATCTGGGGCCCAGGCACCCTGGTCACCGTCTCCTCA | |
| 301 | ---------!---------!---------!---------!---------!- | 351 |
| | CCAATAAGTAAATCGTAGACCCCGGGTCCGTGGGACCAGTGGCAGAGGAGT | |
| 21626B-B5 SEQ ID No. 14, 239, 240 | | |
| 1: | GlnThrValLysGluSerGlyGlyArgLeuValThrProGlyThrProLeuThrLeuThr | |
| | CAGACGGTGAAGGAGTCCGGGGGTCGCCTGGTCACGCCTGGGACACCCCTGACACTCACC | |
| 1 | ---------!---------!---------!---------!---------!---------! | 60 |
| | GTCTGCCACTTCCTCAGGCCCCCAGCGGACCAGTGCGGACCCTGTGGGGACTGTGAGTGG | |
| 1: | CysThrAlaSerGlyPheSerPheSerGlyTrpSerValThrTrpValArgGInAlaPro | |
| | TGCACAGCCTCTGGATTCTCCTTCAGTGGCTGGTCGGTGACCTGGGTCCGCCAGGCTCCA | |
| 61 | ---------!---------!---------!---------!---------!---------! | 120 |
| | ACGTGTCGGAGACCTAAGAGGAAGTCACCGACCAGCCACTGGACCCAGGCGGTCCGAGGT | |
| 1: | GlyLysGlyLeuGluTrpIleGlyTyrIleTyrProArgSerGlySerThrAsnTyrAla | |
| | GGGAAGGGGCTGGAGTGGATCGGATACATTTATCCTCGGAGTGGTAGCACAAACTACGCG | |
| 121 | ---------!---------!---------!---------!---------!---------! | 180 |
| | CCCTTCCCCGACCTCACCTAGCCTATGTAAATAGGAGCCTCACCATCGTGTTTGATGCGC | |
| 1: | ArgTrpValArgGlyArgPheThrIleSerAlaThrSerThrThrValAspLeuLysLeu | |
| | AGGTGGGTGAGAGGCCGATTCACCATCTCCGCAACCTCGACCACGGTGGATCTGAAACTC | |
| 181 | ---------!---------!---------!---------!---------!---------! | 240 |
| | TCCACCCACTCTCCGGCTAAGTGGTAGAGGCGTTGGAGCTGGTGCCACCTAGACTTTGAG | |
| 1: | ThrSerProThrThrGluAspThrAlaThrTyrPheCysAlaArgGlyGlyTyrGlyGly | |
| | ACCAGTCCGACAACCGAGGACACGGCCACTTATTTCTGTGCCAGAGGAGGTTACGGTGGT | |
| 241 | ---------!---------!---------!---------!---------!---------! | 300 |
| | TGGTCAGGCTGTTGGCTCCTGTGCCGGTGAATAAAGACACGGTCTCCTCCAATGCCACCA | |
| 1: | IleAspTrpGlySerMetAspIleTrpGlyProGlyThrLeuValThrValSerLeu | |
| | ATTGACTGGGGTAGCATGGACATCTGGGGCCCAGGCACCCTGGTCACCGTCTCCTTA | |
| 301 | ---------!---------!---------!---------!---------!------- | 357 |
| | TAACTGACCCCATCGTACCTGTAGACCCCGGGTCCGTGGGACCAGTGGCAGAGGAAT | |
| 21626B-C2 SEQ ID No. 15, 241, 242 | | |
| 1: | GlnThrValLysGluSerGlyGlyArgLeuValThrProGlyThrProLeuThrLeuThr | |
| | CAGACAGTGAAGGAGTCCGGGGGTCGCCTGGTCACGCCTGGGACACCCCTGACACTCACC | |
| 1 | ---------!---------!---------!---------!---------!---------! | 60 |
| | GTCTGTCACTTCCTCAGGCCCCCAGCGGACCAGTGCGGACCCTGTGGGGACTGTGAGTGG | |
| 1: | CysThrAlaSerGlyPheSerLeuAsnSerAspAspMetThrTrpValArgGlnAlaPro | |
| | TGCACAGCCTCTGGATTCTCCCTCAACAGCGACGACATGACCTGGGTCCGCCAGGCTCCA | |
| 61 | ---------!---------!---------!---------!---------!---------! | 120 |
| | ACGTGTCGGAGACCTAAGAGGGAGTTGTCGCTGCTGTACTGGACCCAGGCGGTCCGAGGT | |
| 1: | GlyMetGlyLeuGluTrpIleGlyThrMetHisAlaSerGlyPheThrTyrTyrAlaSer | |
| | GGGATGGGGCTGGAATGGATTGGAACCATGCATGCTAGCGGTTTCACATACTACGCGAGC | |
| 121 | ---------!---------!---------!---------!---------!---------! | 180 |
| | CCCTACCCCGACCTTACCTAACCTTGGTACGTACGATCGCCAAAGTGTATGATGCGCTCG | |
| 1: | TrpAlaGlnGlyArgPheThrIleSerGlyThrSerThrThrValAspLeuLysIleThr | |
| | TGGGCGCAAGGCCGATTCACCATCTCCGGAACCTCGACCACGGTGGATCTGAAAATCACC | |
| 181 | ---------!---------!---------!---------!---------!---------! | 240 |
| | ACCCGCGTTCCGGCTAAGTGGTAGAGGCCTTGGAGCTGGTGCCACCTAGACTTTTAGTGG | |
| 1: | SerProThrThrGluAspThrAlaThrTyrPheCysAlaArgGlyAlaProGlyTyrThr | |
| | AGTCCGACAACCGAGGACACGGCCACCTATTTCTGTGCCAGAGGTGCTCCTGGTTATACT | |
| 241 | ---------!---------!---------!---------!---------!---------! | 300 |
| | TCAGGCTGTTGGCTCCTGTGCCGGTGGATAAAGACACGGTCTCCACGAGGACCAATATGA | |
| 1: | IleAspAsnIleTrpGlyProGlyThrLeuValThrValSerLeu | |
| | ATTGATAACATCTGGGGCCCAGGCACCCTGGTCACCGTCTCCTTA | |
| 301 | ---------!---------!---------!---------!----- | 345 |
| | TAACTATTGTAGACCCCGGGTCCGTGGGACCAGTGGCAGAGGAAT | |
| 21626B-H4 SEQ ID No. 16, 243, 244 | | |
| 1: | GlnSerLeuGluGluSerGlyGlyArgLeuValThrProGlyThrProLeuThrLeuThr | |
| | CAGTCGTTGGAGGAGTCCGGGGGTCGCCTGGTCACGCCTGGGACACCCCTGACACTCACC | |
| 1 | ---------!---------!---------!---------!---------!---------! | 60 |
| | GTCAGCAACCTCCTCAGGCCCCCAGCGGACCAGTGCGGACCCTGTGGGGACTGTGAGTGG | |
| 1: | CysThrValSerGlyPheSerLeuSerAspHisAlaMetIleTrpValArgGlnAlaPro | |
| | TGCACAGTCTCTGGATTCTCCCTCAGTGACCATGCAATGATCTGGGTCCGCCAGGCTCCA | |
| 61 | ---------!---------!---------!---------!---------!---------! | 120 |
| | ACGTGTCAGAGACCTAAGAGGGAGTCACTGGTACGTTACTAGACCCAGGCGGTCCGAGGT | |
| 1: | GlyGluGlyLeuGluTrpValGlyIleIleTyrProSerGlySerThrTyrTyrAlaAsn | |
| | GGCGAGGGGCTGGAATGGGTCGGGATCATTTATCCTAGTGGTAGCACATACTACGCGAAC | |
| 121 | ---------!---------!---------!---------!---------!---------! | 180 |
| | CCGCTCCCCGACCTTACCCAGCCCTAGTAAATAGGATCACCATCGTGTATGATGCGCTTG | |
| 1: | GlyAlaLysGlyArgPheThrIleSerLysThrSerThrThrValAspLeuLysMetThr | |
| | GGGGCGAAAGGCCGATTCACCATCTCCAAAACCTCGACCACGGTGGATCTGAAAATGACC | |
| 181 | ---------!---------!---------!---------!---------!---------! | 240 |
| | CCCCGCTTTCCGGCTAAGTGGTAGAGGTTTTGGAGCTGGTGCCACCTAGACTTTTACTGG | |
| 1: | SerLeuIleIleGluAspThrAlaThrTyrPheCysAlaArgAspTyrAspSerGlyTrp | |
| | AGCCTGATAATCGAGGACACGGCCACGTATTTCTGTGCCAGAGACTATGATAGTGGCTGG | |
| 241 | ---------!---------!---------!---------!---------!---------! | 300 |
| | TCGGACTATTAGCTCCTGTGCCGGTGCATAAAGACACGGTCTCTGATACTATCACCGACC | |
| 1: | GlyAlaAspIleTrpGlyProGlyThrProValThrValSerLeu | |
| | GGTGCGGATATCTGGGGCCCAGGCACCCCGGTCACCGTCTCCTTG | |
| 301 | ---------!---------!---------!---------!----- | 345 |
| | CCACGCCTATAGACCCCGGGTCCGTGGGGCCAGTGGCAGAGGAAC | |
| 21626B-C8 SEQ ID No. 17, 245, 246 | | |
| 1: | GlnThrValLysGluSerGluGlyGlyLeuPheLysProThrAspThrLeuThrLeuThr | |
| | CAGACAGTGAAGGAGTCCGAGGGAGGTCTCTTCAAGCCAACGGATACCCTGACACTCACC | |
| 1 | ---------!---------!---------!---------!---------!---------! | 60 |
| | GTCTGTCACTTCCTCAGGCTCCCTCCAGAGAAGTTCGGTTGCCTATGGGACTGTGAGTGG | |
| 1: | CysThrValSerGlyPheSerLeuSerIleTyrGlyValSerTrpValArgGlnAlaPro | |
| | TGCACAGTCTCTGGATTCTCCCTCAGTATCTATGGAGTGAGCTGGGTCCGCCAGGCTCCA | |
| 61 | ---------!---------!---------!---------!---------!---------! | 120 |
| | ACGTGTCAGAGACCTAAGAGGGAGTCATAGATACCTCACTCGACCCAGGCGGTCCGAGGT | |
| 1: | GlyAsnGlyLeuGluTrpIleGlySerIleSerAlaThrGlyIleThrTyrTyrAlaSer | |
| | GGGAACGGGCTGGAATGGATCGGGTCCATTAGTGCTACTGGTATCACATACTACGCGAGC | |
| 121 | ---------!---------!---------!---------!---------!---------! | 180 |
| | CCCTTGCCCGACCTTACCTAGCCCAGGTAATCACGATGACCATAGTGTATGATGCGCTCG | |
| 1: | TrpAlaLysSerArgSerThrValThrArgAsnThrAsnLeuAsnThrValThrLeuLys | |
| | TGGGCGAAAAGCCGATCCACCGTCACCAGAAACACCAACCTGAACACGGTGACTCTGAAA | |
| 181 | ---------!---------!---------!---------!---------!---------! | 240 |
| | ACCCGCTTTTCGGCTAGGTGGCAGTGGTCTTTGTGGTTGGACTTGTGCCACTGAGACTTT | |
| 1: | MetThrSerLeuThrAlaAlaAspThrAlaThrTyrPheCysThrArgGlyHisThrTyr | |
| | ATGACCAGTCTGACAGCCGCGGACACGGCCACCTATTTCTGTACTAGAGGTCATACTTAT | |
| 241 | ---------!---------!---------!---------!---------!---------! | 300 |
| | TACTGGTCAGACTGTCGGCGCCTGTGCCGGTGGATAAAGACATGATCTCCAGTATGAATA | |
| 1: | SerThrProAspIleTrpGlyProGlyThrLeuValThrValSerSer | |
| | TCTACCCCTGACATCTGGGGCCCAGGCACCCTGGTCACCGTCTCCTCA | |
| 301 | ---------!---------!---------!---------!-------- | 348 |
| | AGATGGGGACTGTAGACCCCGGGTCCGTGGGACCAGTGGCAGAGGAGT | |
| 21626B-E6 SEQ ID No. 18, 247, 248 | | |
| 1: | GlnSerLeuGluGluSerGlyGlyGlyLeuIleLysProThrAspThrLeuThrLeuThr | |
| | CAGTCGCTGGAGGAGTCCGGGGGAGGCCTGATCAAGCCAACGGATACCCTGACACTCACC | |
| 1 | ---------!---------!---------!---------!---------!---------! | 60 |
| | GTCAGCGACCTCCTCAGGCCCCCTCCGGACTAGTTCGGTTGCCTATGGGACTGTGAGTGG | |
| 1: | CysThrValSerGlyPheSerLeuSerArgTyrGlyValIleTrpValArgGlnAlaPro | |
| | TGCACAGTCTCTGGATTCTCCCTCAGTAGGTATGGAGTGATCTGGGTCCGCCAGGCTCCA | |
| 61 | ---------!---------!---------!---------!---------!---------! | 120 |
| | ACGTGTCAGAGACCTAAGAGGGAGTCATCCATACCTCACTAGACCCAGGCGGTCCGAGGT | |
| 1: | GlySerGlyLeuGluTrpIleGlyAlaIleThrAlaThrGlyIleThrTyrTyrAlaSer | |
| | GGGAGTGGCCTGGAATGGATCGGAGCCATTACTGCTACTGGTATCACATACTACGCGAGC | |
| 121 | ---------!---------!---------!---------!---------!---------! | 180 |
| | CCCTCACCGGACCTTACCTAGCCTCGGTAATGACGATGACCATAGTGTATGATGCGCTCG | |
| 1: | TrpAlaLysSerArgSerThrIleThrArgAsnThrAsnLeuAsnThrValThrLeuLys | |
| | TGGGCGAAAAGCCGATCCACCATCACCAGAAACACCAACCTGAACACGGTGACTCTGAAA | |
| 181 | ---------!---------!---------!---------!---------!---------! | 240 |
| | ACCCGCTTTTCGGCTAGGTGGTAGTGGTCTTTGTGGTTGGACTTGTGCCACTGAGACTTT | |
| 1: | MetThrSerLeuThrAlaAlaAspThrAlaThrTyrPheCysAlaGlyGlyHisThrPhe | |
| | ATGACCAGTCTGACAGCCGCGGACACGGCCACCTATTTCTGTGCGGGGGGTCATACTTTT | |
| 241 | ---------!---------!---------!---------!---------!---------! | 300 |
| | TACTGGTCAGACTGTCGGCGCCTGTGCCGGTGGATAAAGACACGCCCCCCAGTATGAAAA | |
| 1: | SerThrAspValGlyAspIleTrpGlyProGlyThrProValThrValSerSer | |
| | AGTACTGATGTCGGGGACATCTGGGGCCCAGGCACCCCGGTCACCGTCTCCTCA | |
| 301 | ---------!---------!---------!---------!---------!---- | 354 |
| | TCATGACTACAGCCCCTGTAGACCCCGGGTCCGTGGGGCCAGTGGCAGAGGAGT | |
| 21626B-G5 SEQ ID No. 19, 249, 250 | | |
| 1: | GlnSerValLysGluSerGluGlyGlyLeuPheLysProAlaAspThrLeuThrLeuThr | |
| | CAGTCGGTGAAGGAGTCCGAGGGAGGTCTCTTCAAGCCAGCGGATACCCTGACACTCACC | |
| 1 | ---------!---------!---------!---------!---------!---------! | 60 |
| | GTCAGCCACTTCCTCAGGCTCCCTCCAGAGAAGTTCGGTCGCCTATGGGACTGTGAGTGG | |
| 1: | CysThrValSerGlyPheSerLeuAsnSerTyrThrIleSerTrpValArgGlnAlaPro | |
| | TGCACAGTCTCTGGATTCTCCCTCAATAGCTATACAATAAGCTGGGTCCGCCAGGCTCCA | |
| 61 | ---------!---------!---------!---------!---------!---------! | 120 |
| | ACGTGTCAGAGACCTAAGAGGGAGTTATCGATATGTTATTCGACCCAGGCGGTCCGAGGT | |
| 1: | GlyAsnGlyLeuGluTrpIleGlyIleIleAsnThrTyrGlyThrThrAsnTyrAlaSer | |
| | GGGAACGGGCTGGAATGGATCGGAATCATTAATACTTATGGTACCACTAACTACGCGAGC | |
| 121 | ---------!---------!---------!---------!---------!---------! | 180 |
| | CCCTTGCCCGACCTTACCTAGCCTTAGTAATTATGAATACCATGGTGATTGATGCGCTCG | |
| 1: | TrpAlaLysSerArgSerThrIleThrArgAsnThrAsnGluAsnThrValThrLeuLys | |
| | TGGGCGAAAAGCCGATCCACCATCACCAGAAACACCAACGAGAACACGGTGACTCTGAAA | |
| 181 | ---------!---------!---------!---------!---------!---------! | 240 |
| | ACCCGCTTTTCGGCTAGGTGGTAGTGGTCTTTGTGGTTGCTCTTGTGCCACTGAGACTTT | |
| 1: | MetThrSerLeuThrAlaAlaAspThrAlaThrTyrPheCysAlaSerLeuTyrThrThr | |
| | ATGACCAGTCTGACAGCCGCGGACACGGCCACCTATTTCTGTGCGAGCTTGTATACAACA | |
| 241 | ---------!---------!---------!---------!---------!---------! | 300 |
| | TACTGGTCAGACTGTCGGCGCCTGTGCCGGTGGATAAAGACACGCTCGAACATATGTTGT | |
| 1: | GlnThrAsnIleTrpGlyProGlyThrLeuValThrValSerSer | |
| | CAGACTAACATTTGGGGCCCAGGCACCCTGGTCACCGTCTCCTCA | |
| 301 | ---------!---------!---------!---------!----- | 345 |
| | GTCTGATTGTAAACCCCGGGTCCGTGGGACCAGTGGCAGAGGAGT | |
| 21626B-H3 SEQ ID No. 20, 251, 252 | | |
| 1: | GlnSerLeuGlyGluSerArgGlyArgLeuValThrProGlyGlySerLeuThrLeuThr | |
| | CAGTCGCTGGGGGAGTCCAGGGGTCGCCTGGTAACGCCTGGAGGATCCCTGACACTCACC | |
| 1 | ---------!---------!---------!---------!---------!---------! | 60 |
| | GTCAGCGACCCCCTCAGGTCCCCAGCGGACCATTGCGGACCTCCTAGGGACTGTGAGTGG | |
| 1: | CysThrAlaSerGlyPheSerLeuSerSerTyrAlaMetGlyTrpValArgGlnAlaPro | |
| | TGCACAGCCTCTGGATTCTCCCTCAGTAGCTATGCAATGGGCTGGGTCCGCCAGGCTCCA | |
| 61 | ---------!---------!---------!---------!---------!---------! | 120 |
| | ACGTGTCGGAGACCTAAGAGGGAGTCATCGATACGTTACCCGACCCAGGCGGTCCGAGGT | |
| 1: | GlyLysGlyLeuGluTyrIleGlyTrpIleSerAlaGlyGlyThrThrTyrTyrAlaSer | |
| | GGGAAGGGGCTGGAATACATCGGATGGATTAGTGCTGGTGGTACCACATACTACGCGAGC | |
| 121 | ---------!---------!---------!---------!---------!---------! | 180 |
| | CCCTTCCCCGACCTTATGTAGCCTACCTAATCACGACCACCATGGTGTATGATGCGCTCG | |
| 1: | TrpValAsnSerArgPheThrIleSerArgThrSerThrThrValAspLeuGluMetThr | |
| | TGGGTGAATAGTCGATTCACCATCTCCAGAACCTCGACCACGGTGGATCTGGAAATGACC | |
| 181 | ---------!---------!---------!---------!---------!---------! | 240 |
| | ACCCACTTATCAGCTAAGTGGTAGAGGTCTTGGAGCTGGTGCCACCTAGACCTTTACTGG | |
| 1: | SerLeuThrThrGluAspThrAlaThrTyrPheCysAlaArgGluGlyThrGlyTrpGly | |
| | AGTCTGACAACCGAGGACACGGCCACCTATTTCTGTGCCAGAGAGGGTACTGGCTGGGGT | |
| 241 | ---------!---------!---------!---------!---------!---------! | 300 |
| | TCAGACTGTTGGCTCCTGTGCCGGTGGATAAAGACACGGTCTCTCCCATGACCGACCCCA | |
| 1: | AlaTyrAspIleTrpGlyProGlyThrLeuValThrValSerLeu | |
| | GCCTATGACATCTGGGGCCCAGGCACCCTGGTCACCGTCTCCTTA | |
| 301 | ---------!---------!---------!---------!----- | 345 |
| | CGGATACTGTAGACCCCGGGTCCGTGGGACCAGTGGCAGAGGAAT | |
| 21626B-A8 SEQ ID No. 21, 253, 254 | | |
| 1: | GlnLeuGluGluSerGlyGlyArgLeuValThrProGlyThrProLeuThrLeuThrCys | |
| | CAGCTGGAGGAGTCCGGGGGTCGCCTGGTCACGCCTGGGACACCCCTGACACTCACCTGC | |
| 1 | ---------!---------!---------!---------!---------!---------! | 60 |
| | GTCGACCTCCTCAGGCCCCCAGCGGACCAGTGCGGACCCTGTGGGGACTGTGAGTGGACG | |
| 1: | ThrValSerGlyIleAspLeuSerSerAspSerIleGlyTrpValArgGlnAlaProGly | |
| | ACCGTCTCTGGAATCGACCTCAGTAGCGACAGTATTGGCTGGGTCCGCCAGGCTCCAGGG | |
| 61 | ---------!---------!---------!---------!---------!---------! | 120 |
| | TGGCAGAGACCTTAGCTGGAGTCATCGCTGTCATAACCGACCCAGGCGGTCCGAGGTCCC | |
| 1: | LysGlyLeuGluTrpIleGlyIleIleTyrProSerGlyAsnValTyrTyrAlaSerTrp | |
| | AAGGGGCTGGAATGGATCGGAATCATTTATCCTAGTGGTAATGTATACTACGCGAGCTGG | |
| 121 | ---------!---------!---------!---------!---------!---------! | 180 |
| | TTCCCCGACCTTACCTAGCCTTAGTAAATAGGATCACCATTACATATGATGCGCTCGACC | |
| 1: | AlaLysGlyArgLeuThrIleSerLysThrSerThrThrValGluLeuLysMetThrSer | |
| | GCGAAAGGCCGACTCACCATCTCCAAAACCTCGACCACGGTGGAGCTGAAAATGACCAGT | |
| 181 | ---------!---------!---------!---------!---------!---------! | 240 |
| | CGCTTTCCGGCTGAGTGGTAGAGGTTTTGGAGCTGGTGCCACCTCGACTTTTACTGGTCA | |
| 1: | LeuThrThrGluAspThrAlaThrTyrPheCysAlaArgAspTyrTyrSerThrThrLeu | |
| | CTGACAACCGAGGACACGGCCACCTATTTTTGTGCCAGAGATTACTATAGTACTACCTTA | |
| 241 | ---------!---------!---------!---------!---------!---------! | 300 |
| | GACTGTTGGCTCCTGTGCCGGTGGATAAAAACACGGTCTCTAATGATATCATGATGGAAT | |
| 1: | AspIleTrpGlyProGlyThrLeuValThrValSerLeu | |
| | GACATCTGGGGCCCAGGCACCCTGGTCACCGTCTCCTTG | |
| 301 | ---------!---------!---------!--------- | 339 |
| | CTGTAGACCCCGGGTCCGTGGGACCAGTGGCAGAGGAAC | |
| 21626B-D1 SEQ ID No. 22, 255, 256 | | |
| 1: | GlnGluGlnLeuLysGluSerArgGlyArgLeuValThrProGlyGlySerLeuThrLeu | |
| | CAGGAGCAGCTGAAGGAGTCCCGGGGTCGCCTGGTAACGCCTGGAGGATCCCTGACACTC | |
| 1 | ---------!---------!---------!---------!---------!---------! | 60 |
| | GTCCTCGTCGACTTCCTCAGGGCCCCAGCGGACCATTGCGGACCTCCTAGGGACTGTGAG | |
| 1: | ThrCysThrValSerGlyIleAspLeuSerSerAsnAlaMetSerTrpValArgGlnAla | |
| | ACCTGCACAGTCTCTGGAATCGACCTCAGTAGCAATGCAATGAGCTGGGTCCGCCAGGCT | |
| 61 | ---------!---------!---------!---------!---------!---------! | 120 |
| | TGGACGTGTCAGAGACCTTAGCTGGAGTCATCGTTACGTTACTCGACCCAGGCGGTCCGA | |
| 1: | ProGlyGluGlyLeuGluTrpIleGlyThrIleSerGlyGlySerGlySerThrTrpTyr | |
| | CCAGGGGAGGGGCTGGAATGGATCGGAACCATTAGTGGTGGTAGTGGTAGCACATGGTAC | |
| 121 | ---------!---------!---------!---------!---------!---------! | 180 |
| | GGTCCCCTCCCCGACCTTACCTAGCCTTGGTAATCACCACCATCACCATCGTGTACCATG | |
| 1: | AlaSerTrpAlaLysGlyArgPheThrIleSerLysThrSerThrThrValTyrLeuLys | |
| | GCGAGCTGGGCGAAAGGCCGATTCACCATCTCCAAGACCTCGACCACGGTGTATCTGAAA | |
| 181 | ---------!---------!---------!---------!---------!---------! | 240 |
| | CGCTCGACCCGCTTTCCGGCTAAGTGGTAGAGGTTCTGGAGCTGGTGCCACATAGACTTT | |
| 1: | MetThrSerProThrThrGluAspThrAlaThrTyrPheCysAlaArgGlyTrpAspGly | |
| | ATGACCAGTCCGACAACCGAGGACACGGCCACCTATTTCTGTGCCAGAGGTTGGGACGGC | |
| 241 | ---------!---------!---------!---------!---------!---------! | 300 |
| | TACTGGTCAGGCTGTTGGCTCCTGTGCCGGTGGATAAAGACACGGTCTCCAACCCTGCCG | |
| 1: | PheSerThrTrpGlyProGlyThrLeuValThrValSerLeu | |
| | TTTAGCACCTGGGGCCCAGGCACCCTGGTCACCGTCTCCTTA | |
| 301 | ---------!---------!---------!---------!-- | 342 |
| | AAATCGTGGACCCCGGGTCCGTGGGACCAGTGGCAGAGGAAT | |
| 21626B-G1 SEQ ID No. 23, 257, 258 | | |
| 1: | GlnGluGlnLeuValGluSerGlyGlyArgLeuValThrProGlySerProLeuThrLeu | |
| | CAGGAGCAGCTGGTGGAGTCCGGGGGTCGCCTGGTCACGCCTGGGTCACCCCTGACACTC | |
| 1 | ---------!---------!---------!---------!---------!---------! | 60 |
| | GTCCTCGTCGACCACCTCAGGCCCCCAGCGGACCAGTGCGGACCCAGTGGGGACTGTGAG | |
| 1: | ThrCysThrValSerGlyPheSerLeuSerSerTyrTrpMetSerTrpValArgGlnAla | |
| | ACCTGCACAGTCTCTGGATTCTCCCTCAGTAGCTACTGGATGAGCTGGGTCCGCCAAGCT | |
| 61 | ---------!---------!---------!---------!---------!---------! | 120 |
| | TGGACGTGTCAGAGACCTAAGAGGGAGTCATCGATGACCTACTCGACCCAGGCGGTTCGA | |
| 1: | ProGlyLysGlyLeuGluTrpMetGlyAspIleSerAsnThrGlyLysIleTyrTyrAla | |
| | CCAGGGAAGGGGCTGGAGTGGATGGGAGATATTAGTAATACTGGTAAGATATATTACGCG | |
| 121 | ---------!---------!---------!---------!---------!---------! | 180 |
| | GGTCCCTTCCCCGACCTCACCTACCCTCTATAATCATTATGACCATTCTATATAATGCGC | |
| 1: | AsnTrpAlaLysGlyArgPheThrIleSerLysThrSerThrThrValAspLeuThrIle | |
| | AACTGGGCGAAAGGCCGATTCACCATCTCCAAAACCTCGACCACGGTGGATCTGACAATC | |
| 181 | ---------!---------!---------!---------!---------!---------! | 240 |
| | TTGACCCGCTTTCCGGCTAAGTGGTAGAGGTTTTGGAGCTGGTGCCACCTAGACTGTTAG | |
| 1: | ThrSerProThrThrGluAspThrAlaThrTyrPheCysAlaArgGlyMetAsnValTyr | |
| | ACCAGTCCGACAACGGAGGACACGGCCACCTATTTCTGTGCCAGAGGTATGAATGTTTAT | |
| 241 | ---------!---------!---------!---------!---------!---------! | 300 |
| | TGGTCAGGCTGTTGCCTCCTGTGCCGGTGGATAAAGACACGGTCTCCATACTTACAAATA | |
| 1: | AspIleTrpGlyProGlyThrLeuValThrValSerLeu | |
| | GACATCTGGGGCCCAGGCACCCTGGTCACCGTCTCCTTA | |
| 301 | ---------!---------!---------!--------- | 339 |
| | CTGTAGACCCCGGGTCCGTGGGACCAGTGGCAGAGGAAT | |
| 21626B-C4-8 SEQ ID No. 24, 259, 260 | | |
| 1: | GlnSerLeuGluGluSerGlyGlyArgLeuValThrProGlyThrProLeuThrLeuThr | |
| | CAGTCGTTGGAGGAGTCCGGGGGTCGCCTGGTCACGCCTGGGACACCCCTGACACTCACC | |
| 1 | ---------!---------!---------!---------!---------!---------! | 60 |
| | GTCAGCAACCTCCTCAGGCCCCCAGCGGACCAGTGCGGACCCTGTGGGGACTGTGAGTGG | |
| 1: | CysThrAlaSerGlyPheSerLeuSerSerTyrTyrMetSerTrpValArgGlnAlaPro | |
| | TGCACAGCCTCTGGATTCTCCCTCAGTAGCTACTACATGAGCTGGGTCCGCCAGGCTCCA | |
| 61 | ---------!---------!---------!---------!---------!---------! | 120 |
| | ACGTGTCGGAGACCTAAGAGGGAGTCATCGATGATGTACTCGACCCAGGCGGTCCGAGGT | |
| 1: | GluLysGlyLeuGluTrpIleGlyIleIleTyrProSerGlyAsnThrTyrTyrAlaAsn | |
| | GAAAAGGGGCTGGAATGGATCGGAATCATTTATCCTAGTGGTAACACATACTACGCGAAC | |
| 121 | ---------!---------!---------!---------!---------!---------! | 180 |
| | CTTTTCCCCGACCTTACCTAGCCTTAGTAAATAGGATCACCATTGTGTATGATGCGCTTG | |
| 1: | TrpAlaAsnGlyArgPheAlaIleSerArgThrSerThrThrValAspLeuLysIleThr | |
| | TGGGCGAATGGTCGATTCGCCATCTCCAGAACCTCGACCACGGTGGATCTGAAGATCACC | |
| 181 | ---------!---------!---------!---------!---------!---------! | 240 |
| | ACCCGCTTACCAGCTAAGCGGTAGAGGTCTTGGAGCTGGTGCCACCTAGACTTCTAGTGG | |
| 1: | SerProThrThrGluAspThrAlaThrTyrPheCysAlaArgAspSerGlyTyrProAsp | |
| | AGTCCGACAACCGAGGACACGGCCACCTATTTCTGCGCCAGAGATTCTGGTTATCCTGAC | |
| 241 | ---------!---------!---------!---------!---------!---------! | 300 |
| | TCAGGCTGTTGGCTCCTGTGCCGGTGGATAAAGACGCGGTCTCTAAGACCAATAGGACTG | |
| 1: | IleTrpGlyProGlyThrLeuValThrValSerLeu | |
| | ATCTGGGGCCCAGGCACCCTGGTCACCGTCTCCTTA | |
| 301 | ---------!---------!---------!------ | 339 |
| | TAGACCCCGGGTCCGTGGGACCAGTGGCAGAGGAAT | |
| 21626S-A1 SEQ ID No. 25, 261, 262 | | |
| 1: | GlnGluGlnLeuLysGluSerGlyGlyArgLeuValThrProGlyThrProLeuThrLeu | |
| | CAGGAGCAGCTGAAGGAGTCCGGGGGTCGCCTGGTCACGCCTGGGACACCCCTGACACTC | |
| 1 | ---------!---------!---------!---------!---------!---------! | 60 |
| | GTCCTCGTCGACTTCCTCAGGCCCCCAGCGGACCAGTGCGGACCCTGTGGGGACTGTGAG | |
| 1: | ThrCysThrAlaSerGlyPheSerLeuSerAlaTyrSerMetSerTrpValArgGlnAla | |
| | ACCTGCACAGCCTCTGGATTCTCCCTCAGTGCCTACTCCATGAGCTGGGTCCGCCAGGCT | |
| 61 | ---------!---------!---------!---------!---------!---------! | 120 |
| | TGGACGTGTCGGAGACCTAAGAGGGAGTCACGGATGAGGTACTCGACCCAGGCGGTCCGA | |
| 1: | ProGlyLysGlyLeuGluTrpIleGlyIleIleSerSerSerGlyTyrThrTyrTyrAla | |
| | CCAGGGAAGGGGCTGGAATGGATCGGAATCATTAGTAGCAGTGGTTACACATACTACGCG | |
| 121 | ---------!---------!---------!---------!---------!---------! | 180 |
| | GGTCCCTTCCCCGACCTTACCTAGCCTTAGTAATCATCGTCACCAATGTGTATGATGCGC | |
| 1: | SerTrpAlaLysGlyArgPheThrIleSerLysThrSerThrThrValAspLeuGluIle | |
| | AGCTGGGCGAAAGGCCGATTCACCATCTCCAAAACCTCGACCACGGTGGATCTGGAGATC | |
| 181 | ---------!---------!---------!---------!---------!---------! | 240 |
| | TCGACCCGCTTTCCGGCTAAGTGGTAGAGGTTTTGGAGCTGGTGCCACCTAGACCTCTAG | |
| 1: | ThrSerProThrThrGluAspThrAlaThrTyrPheCysAlaArgGlyTyrGlyTyrAsn | |
| | ACCAGTCCGACAACCGAGGACACGGCCACCTATTTCTGTGCCAGAGGTTATGGTTATAAT | |
| 241 | ---------!---------!---------!---------!---------!---------! | 300 |
| | TGGTCAGGCTGTTGGCTCCTGTGCCGGTGGATAAAGACACGGTCTCCAATACCAATATTA | |
| 1: | AsnTyrTyrGluHisPheAsnMetTrpGlyProGlyThrLeuValThrValSerLeu | |
| | AATTATTATGAGCACTTTAACATGTGGGGCCCAGGCACCCTGGTCACCGTCTCCTTA | |
| 301 | ---------!---------!---------!---------!---------!------- | 357 |
| | TTAATAATACTCGTGAAATTGTACACCCCGGGTCCGTGGGACCAGTGGCAGAGGAAT | |

### Light Chain Sequences

Listed below are light chain sequences of certain non-limiting anti-IgE antibodies, identified by name (e.g. “21626S-D5”) and ID number (e.g. “SEQ ID No. 26”).

| 21626S-D5 SEQ ID No. 26, 263, 264 | | |
|---|---|---|
| 1: | AlaGlnValMetAlaGlnThrProAlaSerValSerGluProValGlyGlyThrValThr | |
| | GCCCAAGTGATGGCCCAGACTCCAGCCTCCGTGTCTGAACCTGTCGGAGGCACAGTCACC | |
| 1 | ---------!---------!---------!---------!---------!---------! | 60 |
| | CGGGTTCACTACCGGGTCTGAGGTCGGAGGCACAGACTTGGACAGCCTCCGTGTCAGTGG | |
| 1: | IleLysCysGlnAlaSerGlnSerIleGlyAsnAlaLeuAlaTrpTyrGlnGlnLysPro | |
| | ATCAAGTGCCAGGCCAGTCAGAGCATTGGCAATGCATTAGCCTGGTATCAGCAGAAACCA | |
| 61 | ---------!---------!---------!---------!---------!---------! | 120 |
| | TAGTTCACGGTCCGGTCAGTCTCGTAACCGTTACGTAATCGGACCATAGTCGTCTTTGGT | |
| 1: | GlyGlnArgProSerLeuLeuIleTyrAspAlaSerThrLeuAlaSerGlyValSerSer | |
| | GGGCAGCGTCCCTCGCTCCTGATCTATGATGCATCCACTCTGGCATCTGGGGTCTCATCG | |
| 121 | ---------!---------!---------!---------!---------!---------! | 180 |
| | CCCGTCGCAGGGAGCGAGGACTAGATACTACGTAGGTGAGACCGTAGACCCCAGAGTAGC | |
| 1: | ArgPheLysGlySerArgSerGlyThrGluTyrThrLeuThrIleSerAspLeuGluCys | |
| | CGGTTCAAAGGCAGTAGATCTGGGACAGAGTACACTCTCACCATCAGCGACCTGGAGTGT | |
| 181 | ---------!---------!---------!---------!---------!---------! | 240 |
| | GCCAAGTTTCCGTCATCTAGACCCTGTCTCATGTGAGAGTGGTAGTCGCTGGACCTCACA | |
| 1: | AlaAspAlaAlaThrTyrTyrCysGlnCysAlaTyrTyrGlySerArgTyrAlaGlyGly | |
| | GCCGATGCTGCCACTTACTACTGTCAGTGTGCTTACTATGGTAGCCGTTATGCTGGAGGG | |
| 241 | ---------!---------!---------!---------!---------!---------! | 300 |
| | CGGCTACGACGGTGAATGATGACAGTCACACGAATGATACCATCGGCAATACGACCTCCC | |
| 1: | SerAlaPheGlyGlyGlyThrGluValValValLys | |
| | AGTGCTTTCGGCGGAGGGACCGAGGTGGTGGTCAAA | |
| 301 | ---------!---------!---------!------ | 336 |
| | TCACGAAAGCCGCCTCCCTGGCTCCACCACCAGTTT | |
| 21626B-B9 SEQ ID No. 27, 265, 266 | | |
| 1: | AlaIleAspLeuThrGlnThrProAlaSerValSerGluProValGlyGlyThrValThr | |
| | GCCATCGATCTGACCCAGACTCCAGCCTCCGTGTCTGAACCTGTGGGAGGCACAGTCACC | |
| 1 | ---------!---------!---------!---------!---------!---------! | 60 |
| | CGGTAGCTAGACTGGGTCTGAGGTCGGAGGCACAGACTTGGACACCCTCCGTGTCAGTGG | |
| 1: | IleLysCysGlnAlaSerGlnSerIleGlyAsnAlaLeuAlaTrpTyrGlnGlnLysPro | |
| | ATCAAGTGCCAGGCCAGTCAGAGCATTGGCAATGCATTAGCCTGGTATCAGCAGAAACCA | |
| 61 | ---------!---------!---------!---------!---------!---------! | 120 |
| | TAGTTCACGGTCCGGTCAGTCTCGTAACCGTTACGTAATCGGACCATAGTCGTCTTTGGT | |
| 1: | GlyGlnProProLysLeuLeuIleHisAspAlaSerThrLeuAlaSerGlyValSerSer | |
| | GGGCAGCCTCCCAAGCTCCTGATCCATGATGCATCCACTCTGGCATCTGGGGTCTCATCG | |
| 121 | ---------!---------!---------!---------!---------!---------! | 180 |
| | CCCGTCGGAGGGTTCGAGGACTAGGTACTACGTAGGTGAGACCGTAGACCCCAGAGTAGC | |
| 1: | ArgPheLysGlySerArgSerGlyThrGluTyrThrLeuThrIleSerAspLeuGluCys | |
| | CGGTTCAAAGGCAGTAGATCTGGGACAGAGTACACTCTCACCATCAGCGACCTGGAGTGT | |
| 181 | ---------!---------!---------!---------!---------!---------! | 240 |
| | GCCAAGTTTCCGTCATCTAGACCCTGTCTCATGTGAGAGTGGTAGTCGCTGGACCTCACA | |
| 1: | AlaAspAlaAlaThrTyrTyrCysGlnCysAlaTyrTyrGlySerArgTyrAlaGlyGly | |
| | GCCGATGCTGCCACTTACTACTGTCAGTGTGCTTATTATGGTAGCCGTTATGCTGGAGGG | |
| 241 | ---------!---------!---------!---------!---------!---------! | 300 |
| | CGGCTACGACGGTGAATGATGACAGTCACACGAATAATACCATCGGCAATACGACCTCCC | |
| 1: | AsnAlaPheGlyGlyGlyThrGluValValValLys | |
| | AATGCTTTCGGCGGAGGGACCGAGGTGGTGGTCAAA | |
| 301 | ---------!---------!---------!------ | 336 |
| | TTACGAAAGCCGCCTCCCTGGCTCCACCACCAGTTT | |
| 21626B-H8 SEQ ID No. 28, 265, 266 | | |
| 1: | AspGlyValMetThrGlnThrProAlaSerValSerGluProValGlyGlyThrValThr | |
| | GATGGTGTGATGACCCAGACTCCAGCCTCCGTGTCTGAACCTGTGGGAGGCACAGTCACC | |
| 1 | ---------!---------!---------!---------!---------!---------! | 60 |
| | CTACCACACTACTGGGTCTGAGGTCGGAGGCACAGACTTGGACACCCTCCGTGTCAGTGG | |
| 1: | IleLysCysGlnAlaSerGlnSerIleGlyAsnAlaLeuAlaTrpTyrGlnGlnLysPro | |
| | ATCAAGTGCCAGGCCAGTCAGAGCATTGGCAATGCATTAGCCTGGTATCAGCAGAAACCA | |
| 61 | ---------!---------!---------!---------!---------!---------! | 120 |
| | TAGTTCACGGTCCGGTCAGTCTCGTAACCGTTACGTAATCGGACCATAGTCGTCTTTGGT | |
| 1: | GlyGlnProProLysLeuLeuIleHisAspAlaSerThrLeuAlaSerGlyValSerSer | |
| | GGGCAGCCTCCCAAGCTCCTGATCCATGATGCATCCACTCTGGCATCCGGGGTCTCATCG | |
| 121 | ---------!---------!---------!---------!---------!---------! | 180 |
| | CCCGTCGGAGGGTTCGAGGACTAGGTACTACGTAGGTGAGACCGTAGGCCCCAGAGTAGC | |
| 1: | ArgPheLysGlySerArgSerGlyThrGluTyrThrLeuThrIleSerAspLeuGluCys | |
| | CGGTTCAAAGGCAGTAGATCTGGGACAGAGTACACTCTCACCATCAGCGACCTGGAGTGT | |
| 181 | ---------!---------!---------!---------!---------!---------! | 240 |
| | GCCAAGTTTCCGTCATCTAGACCCTGTCTCATGTGAGAGTGGTAGTCGCTGGACCTCACA | |
| 1: | AlaAspAlaAlaThrTyrTyrCysGlnCysAlaTyrTyrGlySerArgTyrAlaGlyGly | |
| | GCCGATGCTGCCACTTACTACTGTCAGTGTGCTTATTATGGTAGCCGTTATGCTGGAGGG | |
| 241 | ---------!---------!---------!---------!---------!---------! | 300 |
| | CGGCTACGACGGTGAATGATGACAGTCACACGAATAATACCATCGGCAATACGACCTCCC | |
| 1: | AsnAlaPheGlyGlyGlyThrGluValValValLys | |
| | AATGCTTTCGGCGGAGGGACCGAGGTGGTGGTCAAA | |
| 301 | ---------!---------!---------!------ | 336 |
| | TTACGAAAGCCGCCTCCCTGGCTCCACCACCAGTTT | |
| 21626S-E6 SEQ ID No. 29, 269, 270 | | |
| 1: | AspValValMetThrGlnThrProAlaSerAlaSerGluProValGlyGlyThrValThr | |
| | GATGTTGTGATGACCCAGACTCCAGCCTCCGCGTCTGAACCTGTGGGAGGCACAGTCACC | |
| 1 | ---------!---------!---------!---------!---------!---------! | 60 |
| | CTACAACACTACTGGGTCTGAGGTCGGAGGCGCAGACTTGGACACCCTCCGTGTCAGTGG | |
| 1: | IleLysCysGlnAlaSerGlnSerIleGlyAsnAlaLeuAlaTrpTyrGlnGlnLysPro | |
| | ATCAAGTGCCAGGCCAGTCAGAGCATTGGCAATGCATTAGCCTGGTATCAGCAGAAACCA | |
| 61 | ---------!---------!---------!---------!---------!---------! | 120 |
| | TAGTTCACGGTCCGGTCAGTCTCGTAACCGTTACGTAATCGGACCATAGTCGTCTTTGGT | |
| 1: | GlyGlnProProLysLeuLeuIleHisAspAlaSerThrLeuAlaSerGlyValSerSer | |
| | GGGCAGCCTCCCAAGCTCCTGATCCATGATGCATCCACTCTGGCATCTGGGGTCTCATCG | |
| 121 | ---------!---------!---------!---------!---------!---------! | 180 |
| | CCCGTCGGAGGGTTCGAGGACTAGGTACTACGTAGGTGAGACCGTAGACCCCAGAGTAGC | |
| 1: | ArgPheLysGlySerArgSerGlyThrGluTyrThrLeuThrIleSerAspLeuGluCys | |
| | CGGTTCAAAGGCAGTAGATCTGGGACAGAGTACACTCTCACCATCAGCGACCTGGAGTGT | |
| 181 | ---------!---------!---------!---------!---------!---------! | 240 |
| | GCCAAGTTTCCGTCATCTAGACCCTGTCTCATGTGAGAGTGGTAGTCGCTGGACCTCACA | |
| 1: | AlaAspAlaAlaThrTyrTyrCysGlnCysAlaTyrTyrGlySerArgTyrAlaGlyGly | |
| | GCCGATGCTGCCACTTACTACTGTCAGTGTGCTTATTATGGTAGCCGTTATGCTGGAGGG | |
| 241 | ---------!---------!---------!---------!---------!---------! | 300 |
| | CGGCTACGACGGTGAATGATGACAGTCACACGAATAATACCATCGGCAATACGACCTCCC | |
| 1: | AsnAlaPheGlyGlyGlyThrGluValValValLys | |
| | AATGCTTTCGGCGGAGGGACCGAGGTGGTGGTCAAA | |
| 301 | ---------!---------!---------!------ | 336 |
| | TTACGAAAGCCGCCTCCCTGGCTCCACCACCAGTTT | |
| 21626S-E12 SEQ ID No. 30, 269, 270 | | |
| 1: | AspGlyValMetThrGlnThrProAlaSerValSerGluProValGlyGlyThrValThr | |
| | GATGGTGTGATGACCCAGACTCCAGCCTCCGTGTCTGAACCTGTGGGAGGCACAGTCACC | |
| 1 | ---------!---------!---------!---------!---------!---------! | 60 |
| | CTACCACACTACTGGGTCTGAGGTCGGAGGCACAGACTTGGACACCCTCCGTGTCAGTGG | |
| 1: | IleLysCysGlnAlaSerGlnSerIleGlyAsnAlaLeuAlaTrpTyrGlnGlnLysPro | |
| | ATCAAGTGCCAGGCCAGTCAGAGCATTGGCAATGCATTAGCCTGGTATCAGCAGAAACCA | |
| 61 | ---------!---------!---------!---------!---------!---------! | 120 |
| | TAGTTCACGGTCCGGTCAGTCTCGTAACCGTTACGTAATCGGACCATAGTCGTCTTTGGT | |
| 1: | GlyGlnArgProSerLeuLeuIleTyrAspAlaSerThrLeuAlaSerGlyValSerSer | |
| | GGGCAGCGTCCCTCGCTCCTGATCTATGATGCATCCACTCTGGCATCTGGGGTCTCATCG | |
| 121 | ---------!---------!---------!---------!---------!---------! | 180 |
| | CCCGTCGCAGGGAGCGAGGACTAGATACTACGTAGGTGAGACCGTAGACCCCAGAGTAGC | |
| 1: | ArgPheLysGlySerArgSerGlyThrGluTyrThrLeuThrIleSerAspLeuGluCys | |
| | CGGTTCAAAGGCAGTAGATCTGGGACAGAGTACACTCTCACCATCAGCGACCTGGAGTGT | |
| 181 | ---------!---------!---------!---------!---------!---------! | 240 |
| | GCCAAGTTTCCGTCATCTAGACCCTGTCTCATGTGAGAGTGGTAGTCGCTGGACCTCACA | |
| 1: | AlaAspAlaAlaThrTyrTyrCysGlnCysAlaTyrTyrGlySerArgTyrAlaGlyGly | |
| | GCCGATGCTGCCACTTACTACTGTCAGTGTGCTTACTATGGTAGCCGTTATGCTGGAGGG | |
| 241 | ---------!---------!---------!---------!---------!---------! | 300 |
| | CGGCTACGACGGTGAATGATGACAGTCACACGAATGATACCATCGGCAATACGACCTCCC | |
| 1: | AsnAlaPheGlyGlyGlyThrGluValValValLys | |
| | AATGCTTTCGGCGGAGGGACCGAGGTGGTGGTCAAA | |
| 301 | ---------!---------!---------!------ | 336 |
| | TTACGAAAGCCGCCTCCCTGGCTCCACCACCAGTTT | |
| 21626B-F7 SEQ ID No. 31, 273, 274 | | |
| 1: | AspValValMetThrGlnThrProSerSerValSerGluProValGlyGlyThrValThr | |
| | GATGTCGTGATGACCCAGACTCCGTCCTCCGTGTCTGAACCTGTGGGAGGCACAGTCACC | |
| 1 | ---------!---------!---------!---------!---------!---------! | 60 |
| | CTACAGCACTACTGGGTCTGAGGCAGGAGGCACAGACTTGGACACCCTCCGTGTCAGTGG | |
| 1: | IleLysCysGlnAlaSerGlnSerIleGlyAsnAlaLeuAlaTrpTyrGlnGlnLysPro | |
| | ATCAAGTGCCAGGCCAGTCAGAGCATTGGCAATGCATTAGCCTGGTATCAGCAGAAACCA | |
| 61 | ---------!---------!---------!---------!---------!---------! | 120 |
| | TAGTTCACGGTCCGGTCAGTCTCGTAACCGTTACGTAATCGGACCATAGTCGTCTTTGGT | |
| 1: | GlyGlnArgProLysLeuLeuIleHisAspAlaSerThrLeuAlaSerGlyValSerSer | |
| | GGGCAGCGTCCCAAGCTCCTGATCCATGATGCATCCACTCTGGCATCTGGGGTCTCATCG | |
| 121 | ---------!---------!---------!---------!---------!---------! | 180 |
| | CCCGTCGCAGGGTTCGAGGACTAGGTACTACGTAGGTGAGACCGTAGACCCCAGAGTAGC | |
| 1: | ArgPheLysGlyIleArgSerGlyThrGluTyrThrLeuThrIleSerAspLeuGluCys | |
| | CGGTTCAAAGGCATTAGATCCGGGACAGAGTACACTCTCACCATCAGCGACCTGGAGTGT | |
| 181 | ---------!---------!---------!---------!---------!---------! | 240 |
| | GCCAAGTTTCCGTAATCTAGGCCCTGTCTCATGTGAGAGTGGTAGTCGCTGGACCTCACA | |
| 1: | AlaAspAlaAlaThrTyrTyrCysGlnCysAlaTyrTyrGlyAsnArgTyrAlaGlyGly | |
| | GCCGATGCTGCCACTTACTACTGTCAGTGTGCTTATTATGGTAACCGTTATGCTGGAGGG | |
| 241 | ---------!---------!---------!---------!---------!---------! | 300 |
| | CGGCTACGACGGTGAATGATGACAGTCACACGAATAATACCATTGGCAATACGACCTCCC | |
| 1: | AsnAlaPheGlyGlyGlyThrGluValValValLys | |
| | AATGCTTTCGGCGGAGGGACCGAGGTGGTGGTCAAA | |
| 301 | ---------!---------!---------!------ | 336 |
| | TTACGAAAGCCGCCTCCCTGGCTCCACCACCAGTTT | |
| 21626B-F5 SEQ ID No. 32, 275, 276 | | |
| 1: | AspGlyValMetThrGlnThrProAlaSerValSerGluProValGlyGlyThrValThr | |
| | GATGGCGTGATGACCCAGACTCCAGCCTCCGTGTCTGAACCTGTGGGAGGCACAGTCACC | |
| 1 | ---------!---------!---------!---------!---------!---------! | 60 |
| | CTACCGCACTACTGGGTCTGAGGTCGGAGGCACAGACTTGGACACCCTCCGTGTCAGTGG | |
| 1: | IleLysCysGlnAlaSerGlnSerIleGlyAsnAlaLeuAlaTrpTyrGlnGlnLysPro | |
| | ATCAAGTGCCAGGCCAGTCAGAGCATTGGCAATGCATTAGCCTGGTATCAGCAGAAACCA | |
| 61 | ---------!---------!---------!---------!---------!---------! | 120 |
| | TAGTTCACGGTCCGGTCAGTCTCGTAACCGTTACGTAATCGGACCATAGTCGTCTTTGGT | |
| 1: | GlyGlnProProLysLeuLeuIleHisAspAlaSerThrLeuAlaSerGlyValSerSer | |
| | GGGCAGCCTCCCAAGCTCCTGATCCATGATGCATCCACTCTGGCATCTGGGGTCTCATCG | |
| 121 | ---------!---------!---------!---------!---------!---------! | 180 |
| | CCCGTCGGAGGGTTCGAGGACTAGGTACTACGTAGGTGAGACCGTAGACCCCAGAGTAGC | |
| 1: | ArgPheLysGlySerArgSerGlyThrGluTyrThrLeuThrIleSerAspLeuGluCys | |
| | CGGTTCAAAGGCAGTAGATCTGGGACAGAGTACACTCTCACCATCAGCGACCTGGAGTGT | |
| 181 | ---------!---------!---------!---------!---------!---------! | 240 |
| | GCCAAGTTTCCGTCATCTAGACCCTGTCTCATGTGAGAGTGGTAGTCGCTGGACCTCACA | |
| 1: | AlaAspAlaAlaThrTyrTyrCysGlnCysAlaTyrTyrGlySerArgTyrAlaGlyGly | |
| | GCCGATGCTGCCACTTACTACTGTCAGTGTGCTTATTATGGTAGCCGTTATGCTGGAGGG | |
| 241 | ---------!---------!---------!---------!---------!---------! | 300 |
| | CGGCTACGACGGTGAATGATGACAGTCACACGAATAATACCATCGGCAATACGACCTCCC | |
| 1: | AsnAlaPheGlyGlyGlyThrGluValValValLys | |
| | AATGCTTTCGGCGGAGGGACCGAGGTGGTGGTCAAA | |
| 301 | ---------!---------!---------!------ | 336 |
| | TTACGAAAGCCGCCTCCCTGGCTCCACCACCAGTTT | |
| 21626B-C5 SEQ ID No. 33, 275, 276 | | |
| 1: | AlaGlnGlyMetThrGlnThrProSerSerValSerGluProValGlyGlyThrValThr | |
| | GCCCAAGGGATGACCCAGACTCCATCCTCCGTGTCTGAACCTGTGGGAGGCACAGTCACC | |
| 1 | ---------!---------!---------!---------!---------!---------! | 60 |
| | CGGGTTCCCTACTGGGTCTGAGGTAGGAGGCACAGACTTGGACACCCTCCGTGTCAGTGG | |
| 1: | IleLysCysGlnAlaSerGlnSerIleGlyAsnAlaLeuAlaTrpTyrGlnGlnLysPro | |
| | ATCAAGTGCCAGGCCAGTCAGAGCATTGGCAATGCATTAGCCTGGTATCAGCAGAAACCA | |
| 61 | ---------!---------!---------!---------!---------!---------! | 120 |
| | TAGTTCACGGTCCGGTCAGTCTCGTAACCGTTACGTAATCGGACCATAGTCGTCTTTGGT | |
| 1: | GlyGlnArgProLysLeuLeuIleHisAspAlaSerThrLeuAlaSerGlyValSerSer | |
| | GGGCAGCGTCCCAAGCTCCTGATCCATGATGCATCCACTCTGGCATCTGGGGTCTCATCG | |
| 121 | ---------!---------!---------!---------!---------!---------! | 180 |
| | CCCGTCGCAGGGTTCGAGGACTAGGTACTACGTAGGTGAGACCGTAGACCCCAGAGTAGC | |
| 1: | ArgPheLysGlyIleArgSerGlyThrGluTyrThrLeuThrIleSerAspLeuGluCys | |
| | CGGTTCAAAGGCATTAGATCTGGGACAGAGTACACTCTCACCATCAGCGACCTGGAGTGT | |
| 181 | ---------!---------!---------!---------!---------!---------! | 240 |
| | GCCAAGTTTCCGTAATCTAGACCCTGTCTCATGTGAGAGTGGTAGTCGCTGGACCTCACA | |
| 1: | AlaAspAlaAlaThrTyrTyrCysGlnCysAlaTyrTyrGlyAsnArgTyrAlaGlyGly | |
| | GCCGATGCTGCCACTTACTACTGTCAGTGTGCTTATTATGGTAACCGTTATGCTGGAGGG | |
| 241 | ---------!---------!---------!---------!---------!---------! | 300 |
| | CGGCTACGACGGTGAATGATGACAGTCACACGAATAATACCATTGGCAATACGACCTCCC | |
| 1: | AsnAlaPheGlyGlyGlyThrGluValValValLys | |
| | AATGCTTTCGGCGGAGGGACCGAGGTGGTGGTCAAA | |
| 301 | ---------!---------!---------!------ | 336 |
| | TTACGAAAGCCGCCTCCCTGGCTCCACCACCAGTTT | |
| 21626S-G9 SEQ ID No. 34, 275, 276 | | |
| 1: | AlaGlnGlyMetThrGlnThrProAlaSerValSerGluProValGlyGlyThrValThr | |
| | GCCCAAGGGATGACCCAGACTCCAGCCTCCGTGTCTGAACCTGTGGGAGGCACAGTCACC | |
| 1 | ---------!---------!---------!---------!---------!---------! | 60 |
| | CGGGTTCCCTACTGGGTCTGAGGTCGGAGGCACAGACTTGGACACCCTCCGTGTCAGTGG | |
| 1: | IleLysCysGlnAlaSerGlnSerIleGlyAsnAlaLeuAlaTrpTyrGlnGlnLysPro | |
| | ATCAAGTGCCAGGCCAGTCAGAGCATTGGCAATGCATTAGCCTGGTATCAGCAGAAACCA | |
| 61 | ---------!---------!---------!---------!---------!---------! | 120 |
| | TAGTTCACGGTCCGGTCAGTCTCGTAACCGTTACGTAATCGGACCATAGTCGTCTTTGGT | |
| 1: | GlyGlnProProLysLeuLeuIleHisAspAlaSerThrLeuAlaSerGlyValSerSer | |
| | GGGCAGCCTCCCAAGCTCCTGATCCATGATGCATCCACTCTGGCATCTGGGGTCTCATCG | |
| 121 | ---------!---------!---------!---------!---------!---------! | 180 |
| | CCCGTCGGAGGGTTCGAGGACTAGGTACTACGTAGGTGAGACCGTAGACCCCAGAGTAGC | |
| 1: | ArgPheLysGlySerArgSerGlyThrGluTyrThrLeuThrIleSerAspLeuGluCys | |
| | CGGTTCAAAGGCAGTAGATCTGGGACAGAGTACACTCTCACCATCAGCGACCTGGAGTGT | |
| 181 | ---------!---------!---------!---------!---------!---------! | 240 |
| | GCCAAGTTTCCGTCATCTAGACCCTGTCTCATGTGAGAGTGGTAGTCGCTGGACCTCACA | |
| 1: | AlaAspAlaAlaThrTyrTyrCysGlnCysAlaTyrTyrGlySerArgTyrAlaGlyGly | |
| | GCCGATGCTGCCACTTACTACTGTCAGTGTGCTTATTATGGTAGCCGTTATGCTGGAGGG | |
| 241 | ---------!---------!---------!---------!---------!---------! | 300 |
| | CGGCTACGACGGTGAATGATGACAGTCACACGAATAATACCATCGGCAATACGACCTCCC | |
| 1: | AsnAlaPheGlyGlyGlyThrGluValValValLys | |
| | AATGCTTTCGGCGGAGGGACCGAGGTGGTGGTCAAA | |
| 301 | ---------!---------!---------!------ | 336 |
| | TTACGAAAGCCGCCTCCCTGGCTCCACCACCAGTTT | |
| 21626S-F8 SEQ ID No. 35, 275, 276 | | |
| 1: | AspValValMetThrGlnThrProAlaSerValSerGluProValGlyGlyThrAlaThr | |
| | GATGTTGTGATGACCCAGACTCCAGCCTCCGTGTCTGAACCTGTGGGAGGCACAGCCACC | |
| 1 | ---------!---------!---------!---------!---------!---------! | 60 |
| | CTACAACACTACTGGGTCTGAGGTCGGAGGCACAGACTTGGACACCCTCCGTGTCGGTGG | |
| 1: | IleLysCysGlnAlaSerGlnSerIleGlyAsnAlaLeuAlaTrpTyrGlnGlnLysPro | |
| | ATCAAGTGCCAGGCCAGTCAGAGCATTGGCAATGCATTAGCCTGGTATCAGCAGAAACCA | |
| 61 | ---------!---------!---------!---------!---------!---------! | 120 |
| | TAGTTCACGGTCCGGTCAGTCTCGTAACCGTTACGTAATCGGACCATAGTCGTCTTTGGT | |
| 1: | GlyGlnProProLysLeuLeuMetHisAspValSerThrLeuAlaSerGlyValSerSer | |
| | GGGCAGCCTCCCAAGCTCCTGATGCATGATGTATCCACTCTGGCATCTGGGGTCTCATCG | |
| 121 | ---------!---------!---------!---------!---------!---------! | 180 |
| | CCCGTCGGAGGGTTCGAGGACTACGTACTACATAGGTGAGACCGTAGACCCCAGAGTAGC | |
| 1: | ArgPheLysGlySerArgSerGlyThrGluPheThrLeuThrIleSerAspLeuGluCys | |
| | CGGTTCAAAGGCAGTAGATCTGGGACAGAGTTCACTCTCACCATCAGCGACCTGGAGTGT | |
| 181 | ---------!---------!---------!---------!---------!---------! | 240 |
| | GCCAAGTTTCCGTCATCTAGACCCTGTCTCAAGTGAGAGTGGTAGTCGCTGGACCTCACA | |
| 1: | AlaAspAlaAlaThrTyrTyrCysGlnCysAlaTyrTyrGlySerArgTyrAlaGlyGly | |
| | GCCGATGCTGCCACTTACTACTGTCAGTGTGCTTATTATGGTAGCCGTTATGCTGGAGGG | |
| 241 | ---------!---------!---------!---------!---------!---------! | 300 |
| | CGGCTACGACGGTGAATGATGACAGTCACACGAATAATACCATCGGCAATACGACCTCCC | |
| 1: | AsnAlaPheGlyGlyGlyThrGluValValValLys | |
| | AATGCTTTCGGCGGAGGGACCGAGGTGGTGGTCAAA | |
| 301 | ---------!---------!---------!------ | 336 |
| | TTACGAAAGCCGCCTCCCTGGCTCCACCACCAGTTT | |
| 21626B-F10 SEQ ID No. 36, 283, 284 | | |
| 1: | AlaAlaValMetThrGlnThrProAlaSerValSerGluProValGlyGlyThrValThr | |
| | GCAGCCGTGATGACCCAGACTCCAGCCTCCGTGTCTGAACCTGTGGGAGGCACAGTCACC | |
| 1 | ---------!---------!---------!---------!---------!---------! | 60 |
| | CGTCGGCACTACTGGGTCTGAGGTCGGAGGCACAGACTTGGACACCCTCCGTGTCAGTGG | |
| 1: | IleLysCysGlnAlaSerGlnSerIleGlyAsnAlaLeuAlaTrpTyrGlnGlnLysPro | |
| | ATCAAGTGCCAGGCCAGTCAGAGCATTGGCAATGCATTAGCCTGGTATCAGCAGAAACCA | |
| 61 | ---------!---------!---------!---------!---------!---------! | 120 |
| | TAGTTCACGGTCCGGTCAGTCTCGTAACCGTTACGTAATCGGACCATAGTCGTCTTTGGT | |
| 1: | GlyGlnProProLysLeuLeuIleHisAspAlaSerThrLeuAlaSerGlyValSerSer | |
| | GGGCAGCCTCCCAAGCTCCTGATCCATGATGCATCCACTCTGGCATCTGGGGTCTCATCG | |
| 121 | ---------!---------!---------!---------!---------!---------! | 180 |
| | CCCGTCGGAGGGTTCGAGGACTAGGTACTACGTAGGTGAGACCGTAGACCCCAGAGTAGC | |
| 1: | ArgPheLysGlySerArgSerGlyThrGluTyrThrLeuThrIleSerAspLeuGluCys | |
| | CGGTTCAAAGGCAGTAGATCTGGGACAGAGTACACTCTCACCATCAGCGACCTGGAGTGT | |
| 181 | ---------!---------!---------!---------!---------!---------! | 240 |
| | GCCAAGTTTCCGTCATCTAGACCCTGTCTCATGTGAGAGTGGTAGTCGCTGGACCTCACA | |
| 1: | AlaAspAlaAlaThrTyrTyrCysGlnCysAlaTyrTyrGlySerArgTyrAlaGlyGly | |
| | GCCGATGCTGCCACTTACTACTGTCAGTGTGCTTATTATGGTAGCCGTTATGCTGGAGGG | |
| 241 | ---------!---------!---------!---------!---------!---------! | 300 |
| | CGGCTACGACGGTGAATGATGACAGTCACACGAATAATACCATCGGCAATACGACCTCCC | |
| 1: | AsnAlaPheGlyGlyGlyThrGluValValValLys | |
| | AATGCTTTCGGCGGAGGGACCGAGGTGGTGGTCAAA | |
| 301 | ---------!---------!---------!------ | 336 |
| | TTACGAAAGCCGCCTCCCTGGCTCCACCACCAGTTT | |
| 21626B-G12 SEQ ID No. 37, 283, 284 | | |
| 1: | AlaGlnValLeuThrGlnThrProAlaSerValSerAlaAlaValGlyGlyThrValThr | |
| | GCCCAAGTGCTGACCCAGACTCCAGCCTCCGTGTCTGCAGCTGTGGGAGGCACAGTCACC | |
| 1 | ---------!---------!---------!---------!---------!---------! | 60 |
| | CGGGTTCACGACTGGGTCTGAGGTCGGAGGCACAGACGTCGACACCCTCCGTGTCAGTGG | |
| 1: | IleSerCysGlnSerSerGluSerValTyrLysAsnAsnTyrLeuSerTrpTyrGlnGln | |
| | ATCAGTTGCCAGTCCAGTGAGAGTGTTTATAAGAACAACTACTTATCCTGGTATCAGCAG | |
| 61 | ---------!---------!---------!---------!---------!---------! | 120 |
| | TAGTCAACGGTCAGGTCACTCTCACAAATATTCTTGTTGATGAATAGGACCATAGTCGTC | |
| 1: | LysProGlyGlnProProLysGlyLeuIleTyrLeuSerSerThrLeuAlaSerGlyVal | |
| | AAACCAGGGCAGCCTCCCAAAGGCCTGATCTATCTTTCATCCACTCTGGCATCTGGGGTC | |
| 121 | ---------!---------!---------!---------!---------!---------! | 180 |
| | TTTGGTCCCGTCGGAGGGTTTCCGGACTAGATAGAAAGTAGGTGAGACCGTAGACCCCAG | |
| 1: | ProSerArgPheSerGlySerGlySerGlyThrGlnPheThrLeuThrIleSerAspLeu | |
| | CCATCGCGGTTCAGCGGCAGTGGATCTGGGACACAGTTCACTCTCACCATCAGCGACCTG | |
| 181 | ---------!---------!---------!---------!---------!---------! | 240 |
| | GGTAGCGCCAAGTCGCCGTCACCTAGACCCTGTGTCAAGTGAGAGTGGTAGTCGCTGGAC | |
| 1: | GluCysSerAspAlaAlaThrTyrTyrCysAlaGlyGlyTyrSerGlyAsnIleGlyAla | |
| | GAGTGTAGCGATGCTGCCACTTACTACTGTGCAGGCGGTTATAGTGGTAATATTGGCGCT | |
| 241 | ---------!---------!---------!---------!---------!---------! | 300 |
| | CTCACATCGCTACGACGGTGAATGATGACACGTCCGCCAATATCACCATTATAACCGCGA | |
| 1: | PheGlyGlyGlyThrGluValValValLys | |
| | TTCGGCGGAGGGACCGAGGTGGTGGTCAAA | |
| 301 | ---------!---------!---------!------ | 336 |
| | AAGCCGCCTCCCTGGCTCCACCACCAGTTT | |
| 21626B-A11 SEQ ID No. 38, 287, 288 | | |
| 1: | AlaLeuValMetThrGlnThrProAlaSerValGluAlaAlaValGlyGlyThrValThr | |
| | GCGCTGGTGATGACCCAGACTCCAGCCTCCGTGGAGGCAGCTGTGGGAGGCACAGTCACC | |
| 1 | ---------!---------!---------!---------!---------!---------! | 60 |
| | CGCGACCACTACTGGGTCTGAGGTCGGAGGCACCTCCGTCGACACCCTCCGTGTCAGTGG | |
| 1: | IleAsnCysGlnAlaSerGlnAsnIleGlyAsnTyrLeuSerTrpPheGlnGlnLysPro | |
| | ATCAATTGCCAGGCCAGCCAGAACATTGGTAATTACTTATCCTGGTTTCAACAGAAACCA | |
| 61 | ---------!---------!---------!---------!---------!---------! | 120 |
| | TAGTTAACGGTCCGGTCGGTCTTGTAACCATTAATGAATAGGACCAAAGTTGTCTTTGGT | |
| 1: | GlyGlnArgProLysLeuLeuIleTyrLysAlaSerThrLeuAlaSerGlyValSerSer | |
| | GGGCAGCGTCCCAAGCTCCTGATCTACAAGGCTTCCACTCTGGCATCTGGGGTCTCATCG | |
| 121 | ---------!---------!---------!---------!---------!---------! | 180 |
| | CCCGTCGCAGGGTTCGAGGACTAGATGTTCCGAAGGTGAGACCGTAGACCCCAGAGTAGC | |
| 1: | ArgPheLysGlySerGlySerGlyThrGlnPheThrLeuThrIleSerGlyValGluCys | |
| | CGGTTCAAAGGCAGTGGATCTGGGACACAGTTCACTCTCACCATCAGCGGCGTGGAGTGT | |
| 181 | ---------!---------!---------!---------!---------!---------! | 240 |
| | GCCAAGTTTCCGTCACCTAGACCCTGTGTCAAGTGAGAGTGGTAGTCGCCGCACCTCACA | |
| 1: | AlaAspAlaAlaThrTyrTyrCysGlnGlnAspAlaAlaValIleAsnValAspAsnThr | |
| | GCCGATGCTGCCACTTACTACTGTCAACAAGATGCTGCTGTTATTAATGTTGATAATACT | |
| 241 | ---------!---------!---------!---------!---------!---------! | 300 |
| | CGGCTACGACGGTGAATGATGACAGTTGTTCTACGACGACAATAATTACAACTATTATGA | |
| 1: | PheGlyGlyGlyThrGluValValValLys | |
| | TTCGGCGGAGGGACCGAGGTGGTGGTCAAA | |
| 301 | ---------!---------!---------! | 330 |
| | AAGCCGCCTCCCTGGCTCCACCACCAGTTT | |
| 21626B-B5 SEQ ID No. 39, 289, 290 | | |
| 1: | AlaValValLeuThrGlnThrProSerSerSerSerAlaAlaValGlyGlyThrValThr | |
| | GCCGTCGTGCTGACCCAGACTCCATCATCCTCGTCTGCAGCTGTGGGAGGCACAGTCACC | |
| 1 | ---------!---------!---------!---------!---------!---------! | 60 |
| | CGGCAGCACGACTGGGTCTGAGGTAGTAGGAGCAGACGTCGACACCCTCCGTGTCAGTGG | |
| 1: | IleAsnCysGlnAlaSerGlnSerValTyrAsnAsnAsnTyrLeuSerTrpTyrGlnGln | |
| | ATCAATTGCCAGGCCAGTCAGAGTGTTTATAATAACAACTACTTATCCTGGTATCAGCAA | |
| 61 | ---------!---------!---------!---------!---------!---------! | 120 |
| | TAGTTAACGGTCCGGTCAGTCTCACAAATATTATTGTTGATGAATAGGACCATAGTCGTT | |
| 1: | LysProGlyGlnProProLysLeuLeuIleTyrGlyAlaSerLysLeuAlaSerGlyVal | |
| | AAACCAGGGCAGCCTCCCAAGCTCCTGATCTATGGTGCATCCAAACTGGCATCTGGGGTC | |
| 121 | ---------!---------!---------!---------!---------!---------! | 180 |
| | TTTGGTCCCGTCGGAGGGTTCGAGGACTAGATACCACGTAGGTTTGACCGTAGACCCCAG | |
| 1: | ProAlaArgPheSerGlySerArgSerGlyThrAspPheThrLeuThrIleAsnAspLeu | |
| | CCAGCGCGGTTCAGCGGCAGTAGATCTGGGACAGACTTCACTCTCACCATCAACGACCTG | |
| 181 | ---------!---------!---------!---------!---------!---------! | 240 |
| | GGTCGCGCCAAGTCGCCGTCATCTAGACCCTGTCTGAAGTGAGAGTGGTAGTTGCTGGAC | |
| 1: | GluCysAlaAspAlaAlaThrTyrTyrCysGlnSerThrTyrGlySerAsnSerAsnGly | |
| | GAGTGTGCCGATGCTGCCACTTACTACTGTCAAAGTACTTATGGTAGTAATAGTAATGGT | |
| 241 | ---------!---------!---------!---------!---------!---------! | 300 |
| | CTCACACGGCTACGACGGTGAATGATGACAGTTTCATGAATACCATCATTATCATTACCA | |
| 1: | TrpAspAsnAlaPheGlyGlyGlyThrGluValValValArg | |
| | TGGGATAATGCTTTCGGCGGAGGGACCGAGGTGGTCGTCAGA | |
| 301 | ---------!---------!---------!---------!-- | 342 |
| | ACCCTATTACGAAAGCCGCCTCCCTGGCTCCACCAGCAGTCT | |
| 21626B-C2 SEQ ID No. 40, 291, 292 | | |
| 1: | AlaGlnValLeuThrGlnThrAlaSerProValSerAlaAlaValGlyGlyThrValThr | |
| | GCCCAAGTGCTGACCCAGACTGCATCCCCTGTGTCTGCAGCTGTGGGAGGCACAGTCACC | |
| 1 | ---------!---------!---------!---------!---------!---------! | 60 |
| | CGGGTTCACGACTGGGTCTGACGTAGGGGACACAGACGTCGACACCCTCCGTGTCAGTGG | |
| 1: | IleAsnCysGlnSerSerGlnSerIleTyrGlyAsnAsnTrpLeuGlyTrpTyrGlnGln | |
| | ATCAATTGTCAGTCCAGTCAGAGCATTTATGGTAACAACTGGTTAGGCTGGTATCAGCAG | |
| 61 | ---------!---------!---------!---------!---------!---------! | 120 |
| | TAGTTAACAGTCAGGTCAGTCTCGTAAATACCATTGTTGACCAATCCGACCATAGTCGTC | |
| 1: | LysProGlyGlnProProLysGlnLeuIleTyrLysAlaSerThrLeuAlaSerGlyVal | |
| | AAACCAGGGCAGCCTCCCAAGCAACTGATCTACAAGGCTTCCACTCTGGCATCTGGGGTC | |
| 121 | ---------!---------!---------!---------!---------!---------! | 180 |
| | TTTGGTCCCGTCGGAGGGTTCGTTGACTAGATGTTCCGAAGGTGAGACCGTAGACCCCAG | |
| 1: | SerSerArgPheLysGlySerGlySerGlyThrGlnPheThrLeuThrIleSerAspVal | |
| | TCATCGCGGTTCAAAGGCAGTGGATCTGGGACACAGTTCACTCTCACCATCAGCGACGTG | |
| 181 | ---------!---------!---------!---------!---------!---------! | 240 |
| | AGTAGCGCCAAGTTTCCGTCACCTAGACCCTGTGTCAAGTGAGAGTGGTAGTCGCTGCAC | |
| 1: | GlnCysAspAspAlaAlaThrTyrTyrCysLeuGlyGluPheSerCysThrSerGlyAsp | |
| | CAGTGTGACGATGCTGCCACTTACTACTGTCTAGGCGAATTTAGTTGTACTAGTGGTGAT | |
| 241 | ---------!---------!---------!---------!---------!---------! | 300 |
| | GTCACACTGCTACGACGGTGAATGATGACAGATCCGCTTAAATCAACATGATCACCACTA | |
| 1: | CysTyrThrPheGlyGlyGlyThrGluValValValLys | |
| | TGTTATACTTTCGGCGGAGGGACCGAGGTGGTGGTCAAA | |
| 301 | ---------!---------!---------!--------- | 339 |
| | ACAATATGAAAGCCGCCTCCCTGGCTCCACCACCAGTTT | |
| 21626B-H4 SEQ ID No. 41, 293, 294 | | |
| 1: | AlaLeuValMetThrGlnThrProAlaSerValSerAlaAlaValGlyGlyThrValThr | |
| | GCCCTCGTGATGACCCAGACTCCAGCCTCCGTGTCTGCAGCTGTGGGAGGCACAGTCACC | |
| 1 | ---------!---------!---------!---------!---------!---------! | 60 |
| | CGGGAGCACTACTGGGTCTGAGGTCGGAGGCACAGACGTCGACACCCTCCGTGTCAGTGG | |
| 1: | IleAsnCysGlnAlaSerGlnSerIleHisLysTyrLeuSerTrpTyrGlnGlnLysPro | |
| | ATCAATTGCCAGGCCAGTCAGAGCATTCATAAGTACTTATCCTGGTATCAGCAGAAACCA | |
| 61 | ---------!---------!---------!---------!---------!---------! | 120 |
| | TAGTTAACGGTCCGGTCAGTCTCGTAAGTATTCATGAATAGGACCATAGTCGTCTTTGGT | |
| 1: | GlyGlnProProLysLeuLeuIleTyrArgAlaSerThrLeuAlaSerGlyValProSer | |
| | GGGCAGCCTCCCAAACTCCTGATTTACAGGGCATCCACTCTGGCATCTGGGGTCCCGTCG | |
| 121 | ---------!---------!---------!---------!---------!---------! | 180 |
| | CCCGTCGGAGGGTTTGAGGACTAAATGTCCCGTAGGTGAGACCGTAGACCCCAGGGCAGC | |
| 1: | ArgPheLysGlySerGlySerGlyThrGluPheThrLeuThrIleSerAspLeuGluCys | |
| | CGGTTCAAAGGCAGTGGATCTGGGACAGAGTTCACTCTCACCATTAGCGACCTGGAGTGT | |
| 181 | ---------!---------!---------!---------!---------!---------! | 240 |
| | GCCAAGTTTCCGTCACCTAGACCCTGTCTCAAGTGAGAGTGGTAATCGCTGGACCTCACA | |
| 1: | AlaAspAlaAlaThrTyrTyrCysGlnGlnAspAlaAlaValIleAsnIleAspAsnSer | |
| | GCCGATGCTGCCACTTACTACTGTCAACAGGATGCTGCCGTTATTAATATTGATAACAGT | |
| 241 | ---------!---------!---------!---------!---------!---------! | 300 |
| | CGGCTACGACGGTGAATGATGACAGTTGTCCTACGACGGCAATAATTATAACTATTGTCA | |
| 1: | PheGlyGlyGlyThrGluValValValLys | |
| | TTCGGCGGAGGGACCGAGGTGGTCGTCAAA | |
| 301 | ---------!---------!---------!------ | 336 |
| | AAGCCGCCTCCCTGGCTCCACCAGCAGTTT | |
| 21626B-C8 SEQ ID No. 42, 295, 296 | | |
| 1: | AlaLeuValLeuThrGlnThrProSerSerLysSerValProValGlyAspThrValThr | |
| | GCGCTCGTGCTGACCCAGACTCCATCTTCCAAGTCTGTCCCTGTGGGAGACACAGTCACC | |
| 1 | ---------!---------!---------!---------!---------!---------! | 60 |
| | CGCGAGCACGACTGGGTCTGAGGTAGAAGGTTCAGACAGGGACACCCTCTGTGTCAGTGG | |
| 1: | IleAsnCysGlnAlaSerGluSerValTyrAsnLysAsnTyrLeuAlaTrpPheGlnGln | |
| | ATCAATTGCCAGGCCAGTGAGAGTGTTTATAATAAGAACTACTTAGCCTGGTTTCAGCAG | |
| 61 | ---------!---------!---------!---------!---------!---------! | 120 |
| | TAGTTAACGGTCCGGTCACTCTCACAAATATTATTCTTGATGAATCGGACCAAAGTCGTC | |
| 1: | LysProGlyGlnProProLysLeuLeuIleTyrLysAlaSerThrLeuAlaSerGlyVal | |
| | AAACCAGGGCAGCCTCCCAAGCTCCTGATCTACAAGGCTTCCACTCTGGCATCTGGGGTC | |
| 121 | ---------!---------!---------!---------!---------!---------! | 180 |
| | TTTGGTCCCGTCGGAGGGTTCGAGGACTAGATGTTCCGAAGGTGAGACCGTAGACCCCAG | |
| 1: | ProSerArgPheLysGlySerGlySerGlyThrGlnPheThrLeuThrIleAsnAspVal | |
| | CCATCGCGGTTCAAAGGCAGTGGATCTGGGACACAGTTCACTCTCACCATCAACGATGTG | |
| 181 | ---------!---------!---------!---------!---------!---------! | 240 |
| | GGTAGCGCCAAGTTTCCGTCACCTAGACCCTGTGTCAAGTGAGAGTGGTAGTTGCTACAC | |
| 1: | ValCysAspAspAlaAlaThrTyrPheCysAlaGlyTyrLysAspAlaAlaThrAspGly | |
| | GTGTGTGACGATGCTGCCACTTACTTTTGTGCAGGATATAAAGATGCTGCTACTGATGGT | |
| 241 | ---------!---------!---------!---------!---------!---------! | 300 |
| | CACACACTGCTACGACGGTGAATGAAAACACGTCCTATATTTCTACGACGATGACTACCA | |
| 1: | AsnAlaPheGlyGlyGlyThrGluValValValLys | |
| | AATGCTTTCGGCGGAGGGACCGAGGTGGTGGTCAAA | |
| 301 | ---------!---------!---------!------ | 336 |
| | TTACGAAAGCCGCCTCCCTGGCTCCACCACCAGTTT | |
| 21626B-E6 SEQ ID No. 43, 297, 298 | | |
| 1: | AlaLeuValLeuThrGlnThrProSerSerLysSerValProValGlyAspThrValThr | |
| | GCGCTGGTGCTGACCCAGACTCCATCTTCCAAGTCTGTCCCTGTGGGAGACACAGTCACC | |
| 1 | ---------!---------!---------!---------!---------!---------! | 60 |
| | CGCGACCACGACTGGGTCTGAGGTAGAAGGTTCAGACAGGGACACCCTCTGTGTCAGTGG | |
| 1: | IleAsnCysGlnAlaSerGluSerValTrpAsnLysAsnTyrLeuAlaTrpPheGlnGln | |
| | ATCAATTGCCAGGCCAGTGAGAGTGTTTGGAATAAGAACTACTTAGCCTGGTTTCAACAG | |
| 61 | ---------!---------!---------!---------!---------!---------! | 120 |
| | TAGTTAACGGTCCGGTCACTCTCACAAACCTTATTCTTGATGAATCGGACCAAAGTTGTC | |
| 1: | LysProGlyGlnProProLysLeuLeuIleTyrLysAlaSerThrLeuAlaSerGlyVal | |
| | AAACCAGGGCAGCCTCCCAAGCTCCTGATCTACAAGGCTTCCACTCTGGCATCTGGGGTC | |
| 121 | ---------!---------!---------!---------!---------!---------! | 180 |
| | TTTGGTCCCGTCGGAGGGTTCGAGGACTAGATGTTCCGAAGGTGAGACCGTAGACCCCAG | |
| 1: | ProSerArgPheLysGlySerGlySerGlyThrGlnPheThrLeuThrIleAsnAspVal | |
| | CCATCGCGGTTCAAAGGCAGTGGATCTGGGACACAATTCACTCTCACCATCAACGATGTG | |
| 181 | ---------!---------!---------!---------!---------!---------! | 240 |
| | GGTAGCGCCAAGTTTCCGTCACCTAGACCCTGTGTTAAGTGAGAGTGGTAGTTGCTACAC | |
| 1: | ValCysGlyAspAlaAlaThrTyrPheCysAlaGlyTyrLysAspIleAspIleAspGly | |
| | GTGTGTGGCGATGCTGCCACTTACTTTTGTGCAGGATATAAAGATATTGATATTGATGGT | |
| 241 | ---------!---------!---------!---------!---------!---------! | 300 |
| | CACACACCGCTACGACGGTGAATGAAAACACGTCCTATATTTCTATAACTATAACTACCA | |
| 1: | AsnAlaPheGlyGlyGlyThrGluValValValLys | |
| | AATGCTTTCGGCGGAGGGACCGAGGTGGTGGTCAAA | |
| 301 | ---------!---------!---------!------ | 336 |
| | TTACGAAAGCCGCCTCCCTGGCTCCACCACCAGTTT | |
| 21626B-G5 SEQ ID No. 44, 299, 300 | | |
| 1: | AspProValMetThrGlnThrProSerSerThrSerAlaAlaValGlyGlyThrValThr | |
| | GACCCTGTGATGACCCAGACTCCATCTTCCACGTCTGCAGCTGTGGGAGGCACAGTCACC | |
| 1 | ---------!---------!---------!---------!---------!---------! | 60 |
| | CTGGGACACTACTGGGTCTGAGGTAGAAGGTGCAGACGTCGACACCCTCCGTGTCAGTGG | |
| 1: | IleAsnCysGlnAlaSerGlnSerValAlaThrLysAsnGlnLeuAlaTrpTyrGlnGln | |
| | ATCAATTGCCAGGCCAGTCAAAGTGTTGCTACTAAGAACCAATTAGCCTGGTATCAGCAG | |
| 61 | ---------!---------!---------!---------!---------!---------! | 120 |
| | TAGTTAACGGTCCGGTCAGTTTCACAACGATGATTCTTGGTTAATCGGACCATAGTCGTC | |
| 1: | LysProGlyGlnProProLysLeuLeuIleTyrThrThrSerArgLeuAlaSerGlyVal | |
| | AAACCAGGGCAGCCTCCCAAGCTCCTGATCTATACTACATCCAGACTGGCATCTGGGGTC | |
| 121 | ---------!---------!---------!---------!---------!---------! | 180 |
| | TTTGGTCCCGTCGGAGGGTTCGAGGACTAGATATGATGTAGGTCTGACCGTAGACCCCAG | |
| 1: | ProSerArgPheSerGlySerGlySerGlyAlaGInPheThrLeuThrIleSerGlyVal | |
| | CCATCGCGGTTCAGCGGCAGTGGATCTGGGGCGCAGTTCACTCTCACCATCAGTGGTGTG | |
| 181 | ---------!---------!---------!---------!---------!---------! | 240 |
| | GGTAGCGCCAAGTCGCCGTCACCTAGACCCCGCGTCAAGTGAGAGTGGTAGTCACCACAC | |
| 1: | GlnCysAspAspAlaAlaThrTyrTyrCysLeuGlyThrTyrAlaAsnProIleTyrThr | |
| | CAGTGTGACGATGCTGCCACTTACTACTGTCTAGGAACATATGCTAATCCTATTTATACT | |
| 241 | ---------!---------!---------!---------!---------!---------! | 300 |
| | GTCACACTGCTACGACGGTGAATGATGACAGATCCTTGTATACGATTAGGATAAATATGA | |
| 1: | PheGlyGlyGlyThrGluValValValLys | |
| | TTCGGCGGAGGGACTGAGGTGGTCGTCAAG | |
| 301 | ---------!---------!---------! | 330 |
| | AAGCCGCCTCCCTGACTCCACCAGCAGTTC | |
| 21626B-H3 SEQ ID No. 45, 301, 302 | | |
| 1: | AlaLeuValMetThrGlnThrProAlaSerValSerAlaAlaValGlyGlyThrValThr | |
| | GCCCTTGTGATGACCCAGACTCCAGCCTCCGTGTCTGCAGCTGTGGGAGGCACAGTCACC | |
| 1 | ---------!---------!---------!---------!---------!---------! | 60 |
| | CGGGAACACTACTGGGTCTGAGGTCGGAGGCACAGACGTCGACACCCTCCGTGTCAGTGG | |
| 1: | IleAsnCysGlnAlaSerGlnSerIleHisLysTyrLeuSerTrpTyrGlnGlnLysPro | |
| | ATCAACTGCCAGGCCAGTCAGAGCATTCATAAGTACTTATCCTGGTATCAGCAGAAACCA | |
| 61 | ---------!---------!---------!---------!---------!---------! | 120 |
| | TAGTTGACGGTCCGGTCAGTCTCGTAAGTATTCATGAATAGGACCATAGTCGTCTTTGGT | |
| 1: | GlyGlnProProLysLeuLeuIleTyrArgAlaSerThrLeuAlaSerGlyValProSer | |
| | GGGCAGCCTCCCAAACTCCTGATTTACAGGGCATCCACTCTGGCATCTGGGGTCCCGTCG | |
| 121 | ---------!---------!---------!---------!---------!---------! | 180 |
| | CCCGTCGGAGGGTTTGAGGACTAAATGTCCCGTAGGTGAGACCGTAGACCCCAGGGCAGC | |
| 1: | ArgPheLysGlySerGlySerGlyThrGlnPheThrLeuThrIleSerAspLeuGluCys | |
| | CGGTTCAAAGGCAGTGGATCTGGGACACAGTTCACTCTCACCATTAGCGACCTGGAGTGT | |
| 181 | ---------!---------!---------!---------!---------!---------! | 240 |
| | GCCAAGTTTCCGTCACCTAGACCCTGTGTCAAGTGAGAGTGGTAATCGCTGGACCTCACA | |
| 1: | AspAspAlaAlaThrTyrTyrCysGlnGlnAspAlaAlaValIleSerIleAspAsnSer | |
| | GACGATGCTGCCACTTACTACTGTCAACAGGATGCTGCCGTTATTAGTATTGATAACAGT | |
| 241 | ---------!---------!---------!---------!---------!---------! | 300 |
| | CTGCTACGACGGTGAATGATGACAGTTGTCCTACGACGGCAATAATCATAACTATTGTCA | |
| 1: | PheGlyGlyGlyThrGluValValValLys | |
| | TTCGGCGGAGGGACCGAGGTGGTCGTCAAA | |
| 301 | ---------!---------!---------! | 330 |
| | AAGCCGCCTCCCTGGCTCCACCAGCAGTTT | |
| 21626B-A8 SEQ ID No. 46, 303, 304 | | |
| 1: | AlaLeuValMetThrGlnThrProSerSerValSerAlaValValGlyGlyThrValThr | |
| | GCGCTTGTGATGACCCAGACTCCATCCTCCGTGTCTGCAGTTGTGGGAGGCACAGTCACC | |
| 1 | ---------!---------!---------!---------!---------!---------! | 60 |
| | CGCGAACACTACTGGGTCTGAGGTAGGAGGCACAGACGTCAACACCCTCCGTGTCAGTGG | |
| 1: | IleAsnCysGlnAlaSerGlnSerIleSerSerTyrLeuSerTrpTyrGlnGlnLysPro | |
| | ATCAATTGCCAGGCAAGTCAGAGCATTAGTAGTTACTTGTCCTGGTATCAGCAGAAACCA | |
| 61 | ---------!---------!---------!---------!---------!---------! | 120 |
| | TAGTTAACGGTCCGTTCAGTCTCGTAATCATCAATGAACAGGACCATAGTCGTCTTTGGT | |
| 1: | GlyGlnArgProLysProLeuIleTyrGluAlaSerLysLeuAlaSerGlyValSerSer | |
| | GGGCAGCGTCCCAAGCCCCTGATCTACGAAGCATCCAAACTGGCATCTGGGGTCTCATCG | |
| 121 | ---------!---------!---------!---------!---------!---------! | 180 |
| | CCCGTCGCAGGGTTCGGGGACTAGATGCTTCGTAGGTTTGACCGTAGACCCCAGAGTAGC | |
| 1: | ArgPheLysGlySerGlySerGlyThrGluPheThrLeuThrIleSerGlyValGluCys | |
| | CGGTTCAAAGGCAGTGGATCTGGGACAGAGTTCACTCTCACCATCAGCGGCGTGGAGTGT | |
| 181 | ---------!---------!---------!---------!---------!---------! | 240 |
| | GCCAAGTTTCCGTCACCTAGACCCTGTCTCAAGTGAGAGTGGTAGTCGCCGCACCTCACA | |
| 1: | AlaAspAlaAlaThrTyrTyrCysGlnGlnAspAlaAlaValMetAsnValAspAsnThr | |
| | GCCGATGCTGCCACTTACTACTGTCAACAGGATGCTGCTGTTATGAATGTTGATAATACT | |
| 241 | ---------!---------!---------!---------!---------!---------! | 300 |
| | CGGCTACGACGGTGAATGATGACAGTTGTCCTACGACGACAATACTTACAACTATTATGA | |
| 1: | PheGlyGlyGlyThrGluValValValLys | |
| | TTCGGCGGAGGGACCGAGGTGGTGGTCAAG | |
| 301 | ---------!---------!---------! | 330 |
| | AAGCCGCCTCCCTGGCTCCACCACCAGTTC | |
| 21626B-D1 SEQ ID No. 47, 305, 306 | | |
| 1: | AlaLeuValMetThrGlnThrProAlaSerValSerAlaAlaValGlyGlyThrValThr | |
| | GCCCTTGTGATGACCCAGACTCCAGCCTCCGTGTCTGCAGCTGTGGGAGGCACAGTCACC | |
| 1 | ---------!---------!---------!---------!---------!---------! | 60 |
| | CGGGAACACTACTGGGTCTGAGGTCGGAGGCACAGACGTCGACACCCTCCGTGTCAGTGG | |
| 1: | IleAsnCysGlnAlaSerArgSerIleHisLysTyrLeuSerTrpTyrGlnGlnLysPro | |
| | ATCAATTGCCAGGCCAGTCGGAGCATTCATAAGTACTTATCCTGGTATCAGCAGAAACCA | |
| 61 | ---------!---------!---------!---------!---------!---------! | 120 |
| | TAGTTAACGGTCCGGTCAGCCTCGTAAGTATTCATGAATAGGACCATAGTCGTCTTTGGT | |
| 1: | GlyGlnProProLysLeuLeuIleTyrArgAlaSerThrLeuAlaSerGlyValProSer | |
| | GGGCAGCCTCCCAAACTCCTGATTTACAGGGCATCCACTCTGGCATCTGGGGTCCCGTCG | |
| 121 | ---------!---------!---------!---------!---------!---------! | 180 |
| | CCCGTCGGAGGGTTTGAGGACTAAATGTCCCGTAGGTGAGACCGTAGACCCCAGGGCAGC | |
| 1: | ArgPheLysGlySerGlySerGlyThrGluPheThrLeuThrIleSerAspLeuGluCys | |
| | CGGTTCAAAGGCAGTGGATCTGGGACAGAGTTCACTCTCACCATTAGCGACCTGGAGTGT | |
| 181 | ---------!---------!---------!---------!---------!---------! | 240 |
| | GCCAAGTTTCCGTCACCTAGACCCTGTCTCAAGTGAGAGTGGTAATCGCTGGACCTCACA | |
| 1: | AlaAspAlaAlaThrTyrTyrCysGlnGlnAspAlaAlaValIleAsnIleAspAsnSer | |
| | GCCGATGCTGCCACTTACTACTGTCAACAGGATGCTGCCGTTATTAATATTGATAACAGT | |
| 241 | ---------!---------!---------!---------!---------!---------! | 300 |
| | CGGCTACGACGGTGAATGATGACAGTTGTCCTACGACGGCAATAATTATAACTATTGTCA | |
| 1: | PheGlyGlyGlyThrGluValValValLys | |
| | TTCGGCGGAGGGACCGAGGTGGTCGTCAAA | |
| 301 | ---------!---------!---------! | 330 |
| | AAGCCGCCTCCCTGGCTCCACCAGCAGTTT | |
| 21626B-G1 SEQ ID No. 48, 307, 308 | | |
| 1: | AlaLeuValMetThrGlnThrAlaSerProValSerAlaAlaValGlyGlyThrValThr | |
| | GCGCTGGTGATGACCCAGACTGCATCCCCCGTGTCTGCGGCTGTTGGAGGCACAGTCACC | |
| 1 | ---------!---------!---------!---------!---------!---------! | 60 |
| | CGCGACCACTACTGGGTCTGACGTAGGGGGCACAGACGCCGACAACCTCCGTGTCAGTGG | |
| 1: | IleAsnCysGlnAlaSerGlnSerIleHisLysTyrLeuSerTrpTyrGlnGlnLysPro | |
| | ATCAATTGCCAGGCCAGTCAGAGCATTCATAAGTACTTATCCTGGTATCAGCAGAAACCA | |
| 61 | ---------!---------!---------!---------!---------!---------! | 120 |
| | TAGTTAACGGTCCGGTCAGTCTCGTAAGTATTCATGAATAGGACCATAGTCGTCTTTGGT | |
| 1: | GlyGlnProProLysLeuLeuIleTyrArgAlaSerThrLeuAlaSerGlyValProSer | |
| | GGGCAGCCTCCCAAACTCCTGATTTACAGGGCATCCACTCTGGCATCTGGGGTCCCGTCG | |
| 121 | ---------!---------!---------!---------!---------!---------! | 180 |
| | CCCGTCGGAGGGTTTGAGGACTAAATGTCCCGTAGGTGAGACCGTAGACCCCAGGGCAGC | |
| 1: | ArgPheLysGlySerGlySerGlyThrGluPheThrLeuThrIleSerAspLeuGluCys | |
| | CGGTTCAAAGGCAGTGGATCTGGGACAGAGTTCACTCTCACCATTAGCGACCTGGAGTGT | |
| 181 | ---------!---------!---------!---------!---------!---------! | 240 |
| | GCCAAGTTTCCGTCACCTAGACCCTGTCTCAAGTGAGAGTGGTAATCGCTGGACCTCACA | |
| 1: | AlaAspAlaAlaThrTyrTyrCysGlnGlnAspAlaAlaValIleAsnIleAspAsnSer | |
| | GCCGATGCTGCCACTTACTACTGTCAACAGGATGCTGCCGTTATTAATATTGATAACAGT | |
| 241 | ---------!---------!---------!---------!---------!---------! | 300 |
| | CGGCTACGACGGTGAATGATGACAGTTGTCCTACGACGGCAATAATTATAACTATTGTCA | |
| 1: | PheGlyGlyGlyThrGluValValValLys | |
| | TTCGGCGGAGGGACCGAGGTGGTCGTCAAA | |
| 301 | ---------!---------!---------! | 330 |
| | AAGCCGCCTCCCTGGCTCCACCAGCAGTTT | |
| 21626B-C4-8 SEQ ID No. 49, 309, 310 | | |
| 1: | AlaLeuValMetThrGlnThrProSerSerValSerAlaAlaValGlyGlyThrValThr | |
| | GCGCTTGTGATGACCCAGACTCCATCCTCCGTGTCTGCAGCTGTGGGAGGCACAGTCACC | |
| 1 | ---------!---------!---------!---------!---------!---------! | 60 |
| | CGCGAACACTACTGGGTCTGAGGTAGGAGGCACAGACGTCGACACCCTCCGTGTCAGTGG | |
| 1: | IleAsnCysGlnAlaSerGlnSerIleSerSerTyrLeuSerTrpTyrGlnGlnLysPro | |
| | ATCAATTGCCAGGCAAGTCAGAGCATTAGTAGTTACTTATCCTGGTATCAGCAGAAACCA | |
| 61 | ---------!---------!---------!---------!---------!---------! | 120 |
| | TAGTTAACGGTCCGTTCAGTCTCGTAATCATCAATGAATAGGACCATAGTCGTCTTTGGT | |
| 1: | GlyGlnArgProLysProLeuIleTyrGluThrSerLysLeuAlaSerGlyValSerSer | |
| | GGGCAGCGTCCCAAGCCCCTGATCTACGAAACATCCAAACTGGCATCTGGGGTCTCATCG | |
| 121 | ---------!---------!---------!---------!---------!---------! | 180 |
| | CCCGTCGCAGGGTTCGGGGACTAGATGCTTTGTAGGTTTGACCGTAGACCCCAGAGTAGC | |
| 1: | ArgPheSerGlySerGlySerGlyThrGluPheThrLeuThrIleSerGlyValGluCys | |
| | CGGTTCAGTGGCAGTGGATCTGGGACAGAGTTCACTCTCACCATCAGCGGCGTGGAGTGT | |
| 181 | ---------!---------!---------!---------!---------!---------! | 240 |
| | GCCAAGTCACCGTCACCTAGACCCTGTCTCAAGTGAGAGTGGTAGTCGCCGCACCTCACA | |
| 1: | AlaAspAlaAlaThrTyrTyrCysGlnGlnAspAlaGlyValIleAsnValHisAsnThr | |
| | GCCGATGCTGCCACTTACTACTGTCAACAGGATGCTGGTGTTATTAATGTTCATAATACT | |
| 241 | ---------!---------!---------!---------!---------!---------! | 300 |
| | CGGCTACGACGGTGAATGATGACAGTTGTCCTACGACCACAATAATTACAAGTATTATGA | |
| 1: | PheGlyGlyGlyThrGluValValValLys | |
| | TTCGGCGGAGGGACCGAGGTGGTGGTCAAG | |
| 301 | ---------!---------!---------! | 330 |
| | AAGCCGCCTCCCTGGCTCCACCACCAGTTC | |
| 21626S-A1 SEQ ID No. 50, 311, 312 | | |
| 1: | AspValValMetThrGlnThrProAlaSerValSerGluProValGlyGlyThrValThr | |
| | GATGTTGTGATGACCCAGACTCCAGCCTCCGTGTCTGAACCTGTGGGAGGCACAGTCACC | |
| 1 | ---------!---------!---------!---------!---------!---------! | 60 |
| | CTACAACACTACTGGGTCTGAGGTCGGAGGCACAGACTTGGACACCCTCCGTGTCAGTGG | |
| 1: | IleLysCysGlnAlaSerGlnSerIleGlyAsnAlaLeuAlaTrpTyrGlnGlnLysPro | |
| | ATCAAGTGCCAGGCCAGTCAGAGCATTGGCAATGCATTAGCCTGGTATCAGCAGAAACCA | |
| 61 | ---------!---------!---------!---------!---------!---------! | 120 |
| | TAGTTCACGGTCCGGTCAGTCTCGTAACCGTTACGTAATCGGACCATAGTCGTCTTTGGT | |
| 1: | GlyGlnArgProSerLeuLeuIleTyrAspAlaSerThrLeuAlaSerGlyValSerSer | |
| | GGGCAGCGTCCCTCGCTCCTGATCTATGATGCATCCACTCTGGCATCTGGGGTCTCATCG | |
| 121 | ---------!---------!---------!---------!---------!---------! | 180 |
| | CCCGTCGCAGGGAGCGAGGACTAGATACTACGTAGGTGAGACCGTAGACCCCAGAGTAGC | |
| 1: | ArgPheLysGlySerArgSerGlyThrGluTyrThrLeuThrIleSerAspLeuGluCys | |
| | CGGTTCAAAGGCAGTAGATCTGGGACAGAGTACACTCTCACCATCAGCGACCTGGAGTGT | |
| 181 | ---------!---------!---------!---------!---------!---------! | 240 |
| | GCCAAGTTTCCGTCATCTAGACCCTGTCTCATGTGAGAGTGGTAGTCGCTGGACCTCACA | |
| 1: | AlaAspAlaAlaThrTyrTyrCysGlnCysAlaTyrTyrGlySerArgTyrAlaGlyGly | |
| | GCCGATGCTGCCACTTACTACTGTCAGTGTGCTTACTATGGTAGCCGTTATGCTGGAGGG | |
| 241 | ---------!---------!---------!---------!---------!---------! | 300 |
| | CGGCTACGACGGTGAATGATGACAGTCACACGAATGATACCATCGGCAATACGACCTCCC | |
| 1: | SerAlaPheGlyGlyGlyThrGluValValValLys | |
| | AGTGCTTTCGGCGGAGGGACCGAGGTGGTGGTCAAA | |
| 301 | ---------!---------!---------!------ | 336 |
| | TCACGAAAGCCGCCTCCCTGGCTCCACCACCAGTTT | |

### CDR Sequences

Listed below are sequences listing of certain non-limiting anti-IgE antibodies, identified by name (e.g. “21626S-D5 . . . ”) and, where appropriate, ID number (e.g. “SEQ ID No. 51 . . . ”).

| | HCDR1 | | HCDR2 | | HCDR3 | | LCDR1 | | LCDR2 | | LCDR3 | | [symbol] | |
|---|---|---|---|---|---|---|---|---|---|---|---|---|---|---|
| Clone | Sequence | SEQ ID No. | Sequence | SEQ ID No. | Sequence | SEQ ID No. | Sequence | SEQ ID No. | Sequence | SEQ ID No. | Sequence | SEQ ID No. | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |

[symbol]

indicates data missing or illegible when filed

### Humanized Heavy Chain Sequences (Variable Region)

Listed below are humanized heavy chain sequences (variable region) of certain non-limiting anti-IgE antibodies, identified by name (e.g. “h-21626B-C4-8”) and ID number (e.g. “SEQ ID No. 201”).

| h-21626B-C4-8 SEQ ID No. 201, 313, 314 | | | |
|---|---|---|---|
| 1: | 1 | LeuGluGluValGlnLeuValGluSerGlyGlyGlyLeuValGlnProGlyGlySerLeu | 60 |
| | | CTCGAGGAGGTCCAGCTCGTTGAGTCTGGAGGAGGCTTGGTCCAGCCTGGAGGGTCCCTG | |
| | | ---------!---------!---------!---------!---------!---------! | |
| | | GAGCTCCTCCAGGTCGAGCAACTCAGACCTCCTCCGAACCAGGTCGGACCTCCCAGGGAC | |
| 1: | 61 | ArgLeuSerCysThrAlaSerGlyPheSerLeuSerSerTyrTyrMetSerTrpValArg | 120 |
| | | AGGCTCTCCTGTACAGCCTCTGGATTCTCCCTCAGTAGCTACTACATGAGCTGGGTAAGA | |
| | | ---------!---------!---------!---------!---------!---------! | |
| | | TCCGAGAGGACATGTCGGAGACCTAAGAGGGAGTCATCGATGATGTACTCGACCCATTCT | |
| 1: | 121 | GlnAlaProGlyLysGlyLeuGluTrpIleGlyIleIleTyrProSerGlyAsnThrTyr | 180 |
| | | CAGGCACCAGGGAAGGGACTGGAGTGGATCGGCATCATTTATCCTAGTGGTAACACATAC | |
| | | ---------!---------!---------!---------!---------!---------! | |
| | | GTCCGTGGTCCCTTCCCTGACCTCACCTAGCCGTAGTAAATAGGATCACCATTGTGTATG | |
| 1: | 181 | TyrAlaAsnTrpAlaAsnGlyArgPheThrIleSerArgHisAsnSerLysAsnThrLeu | 240 |
| | | TACGCGAACTGGGCAAATGGTCGATTCACCATCTCCAGACACAATTCCAAGAACACGCTG | |
| | | ---------!---------!---------!---------!---------!---------! | |
| | | ATGCGCTTGACCCGTTTACCAGCTAAGTGGTAGAGGTCTGTGTTAAGGTTCTTGTGCGAC | |
| 1: | 241 | TyrLeuGlnMetAsnSerLeuArgAlaGluAspThrAlaValTyrTyrCysAlaArgAsp | 300 |
| | | TATCTTCAAATGAACAGCCTGAGAGCGGAGGACACGGCCGTGTACTACTGTGCCAGAGAT | |
| | | ---------!---------!---------!---------!---------!---------! | |
| | | ATAGAAGTTTACTTGTCGGACTCTCGCCTCCTGTGCCGGCACATGATGACACGGTCTCTA | |
| 1: | 301 | SerGlyTyrProAspIleTrpGlyGlnGlyThrLeuValThrValSerSer | 351 |
| | | TCTGGTTATCCTGACATCTGGGGTCAAGGAACCCTGGTCACCGTCTCCTCA | |
| | | ---------!---------!---------!---------!---------!- | |
| | | AGACCAATAGGACTGTAGACCCCAGTTCCTTGGGACCAGTGGCAGAGGAGT | |
| h-21626B-H3 SEQ ID No. 202, 315, 316 | | | |
| 1: | 1 | LeuGluGlnValGlnLeuValGluSerGlyGlyGlyLeuValGlnProGlyGlySerLeu | 60 |
| | | CTCGAGCAGGTCCAGCTGGTTGAGTCTGGGGGAGGCTTGGTCCAGCCTGGAGGGTCCCTG | |
| | | ---------!---------!---------!---------!---------!---------! | |
| | | GAGCTCGTCCAGGTCGACCAACTCAGACCCCCTCCGAACCAGGTCGGACCTCCCAGGGAC | |
| 1: | 61 | ArgLeuSerCysThrAlaSerGlyPheSerLeuSerSerTyrAlaMetGlyTrpValArg | 120 |
| | | AGGCTCTCCTGTACAGCCTCTGGATTCTCCCTCAGTAGCTATGCAATGGGCTGGGTAAGA | |
| | | ---------!---------!---------!---------!---------!---------! | |
| | | TCCGAGAGGACATGTCGGAGACCTAAGAGGGAGTCATCGATACGTTACCCGACCCATTCT | |
| 1: | 121 | GlnAlaProGlyLysGlyLeuGluTyrIleGlyTrpIleSerAlaGlyGlyThrThrTyr | 180 |
| | | CAGGCACCAGGGAAGGGACTTGAGTACATCGGCTGGATTAGTGCTGGTGGTACCACATAC | |
| | | ---------!---------!---------!---------!---------!---------! | |
| | | GTCCGTGGTCCCTTCCCTGAACTCATGTAGCCGACCTAATCACGACCACCATGGTGTATG | |
| 1: | 181 | TyrAlaSerTrpValAsnSerArgPheThrIleSerArgAspAsnSerLysAsnThrLeu | 240 |
| | | TACGCGAGCTGGGTGAATAGCAGATTCACCATCTCCAGAGACAATTCCAAGAACACGCTG | |
| | | ---------!---------!---------!---------!---------!---------! | |
| | | ATGCGCTCGACCCACTTATCGTCTAAGTGGTAGAGGTCTCTGTTAAGGTTCTTGTGCGAC | |
| 1: | 241 | TyrLeuGlnMetAsnSerLeuArgAlaGluAspThrAlaValTyrTyrCysAlaArgGlu | 300 |
| | | TATCTTCAAATGAACAGCCTGAGAGCGGAGGACACGGCCGTGTACTACTGTGCCAGAGAG | |
| | | ---------!---------!---------!---------!---------!---------! | |
| | | ATAGAAGTTTACTTGTCGGACTCTCGCCTCCTGTGCCGGCACATGATGACACGGTCTCTC | |
| 1: | 301 | GlyThrGlyTrpGlyAlaTyrAspIleTrpGlyGlnGlyThrLeuValThrValSerSer | 360 |
| | | GGTACTGGCTGGGGTGCCTATGACATCTGGGGTCAAGGAACCCTGGTCACCGTCTCCTCA | |
| | | ---------!---------!---------!---------!---------!---------! | |
| | | CCATGACCGACCCCACGGATACTGTAGACCCCAGTTCCTTGGGACCAGTGGCAGAGGAGT | |

### Humanized Light Chain Sequences (Variable Region)

Listed below are humanized light chain sequences (variable region) of certain non-limiting anti-IgE antibodies, identified by name (e.g. “h-21626B-C4-8”) and ID number (e.g. “SEQ ID No. 203”).

| h-21626B-C4-8 SEQ ID No. 203, 315, 316 | | | |
|---|---|---|---|
| 1: | 1 | SerArgAspIleGlnMetThrGlnSerProSerSerLeuSerAlaSerValGlyAspArg | 60 |
| | | TCTAGAGACATCCAGATGACGCAGTCCCCATCCTCCCTGTCTGCATCTGTAGGAGACAGG | |
| | | ---------!---------!---------!---------!---------!---------! | |
| | | AGATCTCTGTAGGTCTACTGCGTCAGGGGTAGGAGGGACAGACGTAGACATCCTCTGTCC | |
| 1: | 61 | ValThrIleThrCysGlnAlaSerGlnSerIleSerSerTyrLeuSerTrpTyrGlnGln | 120 |
| | | GTCACCATCACTTGCCAGGCAAGTCAGAGCATTAGTAGTTACTTATCCTGGTATCAGCAG | |
| | | ---------!---------!---------!---------!---------!---------! | |
| | | CAGTGGTAGTGAACGGTCCGTTCAGTCTCGTAATCATCAATGAATAGGACCATAGTCGTC | |
| 1: | 121 | LysProGlyLysAlaProLysLeuLeuIleTyrGluThrSerLysLeuAlaSerGlyVal | 180 |
| | | AAGCCAGGCAAGGCACCTAAGCTCCTGATCTATGAAACATCCAAACTGGCATCTGGGGTC | |
| | | ---------!---------!---------!---------!---------!---------! | |
| | | TTCGGTCCGTTCCGTGGATTCGAGGACTAGATACTTTGTAGGTTTGACCGTAGACCCCAG | |
| 1: | 181 | ProSerArgPheSerGlySerGlySerGlyThrAspPheThrLeuThrIleSerSerLeu | 240 |
| | | CCCTCAAGGTTTAGTGGAAGTGGATCTGGGACAGATTTCACCCTCACCATCAGCAGTCTG | |
| | | ---------!---------!---------!---------!---------!---------! | |
| | | GGGAGTTCCAAATCACCTTCACCTAGACCCTGTCTAAAGTGGGAGTGGTAGTCGTCAGAC | |
| 1: | 241 | GlnProGluAspPheAlaThrTyrTyrCysGlnGlnAspAlaGlyValIleAsnValHis | 300 |
| | | CAACCGGAGGACTTCGCGACGTACTACTGCCAACAGGATGCTGGTGTTATTAATGTTCAT | |
| | | ---------!---------!---------!---------!---------!---------! | |
| | | GTTGGCCTCCTGAAGCGCTGCATGATGACGGTTGTCCTACGACCACAATAATTACAAGTA | |
| 1: | 301 | AsnThrPheGlyGlyGlyThrLysValGluIleLys | 336 |
| | | AATACTTTCGGTGGTGGTACAAAGGTCGAGATCAAA | |
| | | ---------!---------!---------!------ | |
| | | TTATGAAAGCCACCACCATGTTTCCAGCTCTAGTTT | |
| h-21626B-C4-8E SEQ ID No. 204, 315, 316 | | | |
| 1: | 1 | SerArgAspIleGlnMetThrGlnSerProSerSerLeuSerAlaSerValGlyAspArg | 60 |
| | | TCTAGAGACATCCAGATGACGCAGTCCCCATCCTCCCTGTCTGCATCTGTAGGAGACAGG | |
| | | ---------!---------!---------!---------!---------!---------! | |
| | | AGATCTCTGTAGGTCTACTGCGTCAGGGGTAGGAGGGACAGACGTAGACATCCTCTGTCC | |
| 1: | 61 | ValThrIleThrCysGlnAlaSerGlnSerIleSerSerTyrLeuSerTrpTyrGlnGln | 120 |
| | | GTCACCATCACTTGCCAGGCAAGTCAGAGCATTAGTAGTTACTTATCCTGGTATCAGCAG | |
| | | ---------!---------!---------!---------!---------!---------! | |
| | | CAGTGGTAGTGAACGGTCCGTTCAGTCTCGTAATCATCAATGAATAGGACCATAGTCGTC | |
| 1: | 121 | LysProGlyLysAlaProLysLeuLeuIleTyrGluThrSerLysLeuAlaSerGlyVal | 180 |
| | | AAGCCAGGCAAGGCACCTAAGCTCCTGATCTATGAAACATCCAAACTGGCATCTGGGGTC | |
| | | ---------!---------!---------!---------!---------!---------! | |
| | | TTCGGTCCGTTCCGTGGATTCGAGGACTAGATACTTTGTAGGTTTGACCGTAGACCCCAG | |
| 1: | 181 | ProSerArgPheSerGlySerGlySerGlyThrGluPheThrLeuThrIleSerSerLeu | 240 |
| | | CCCTCAAGGTTTAGTGGAAGTGGATCTGGGACAGAATTCACCCTCACCATCAGCAGTCTG | |
| | | ---------!---------!---------!---------!---------!---------! | |
| | | GGGAGTTCCAAATCACCTTCACCTAGACCCTGTCTTAAGTGGGAGTGGTAGTCGTCAGAC | |
| 1: | 241 | GlnProGluAspPheAlaThrTyrTyrCysGlnGlnAspAlaGlyValIleAsnValHis | 300 |
| | | CAACCGGAGGACTTCGCGACGTACTACTGCCAACAGGATGCTGGTGTTATTAATGTTCAT | |
| | | ---------!---------!---------!---------!---------!---------! | |
| | | GTTGGCCTCCTGAAGCGCTGCATGATGACGGTTGTCCTACGACCACAATAATTACAAGTA | |
| 1: | 301 | AsnThrPheGlyGlyGlyThrLysValGluIleLys | 336 |
| | | AATACTTTCGGTGGTGGTACAAAGGTCGAGATCAAA | |
| | | ---------!---------!---------!------ | |
| | | TTATGAAAGCCACCACCATGTTTCCAGCTCTAGTTT | |
| h-21626B-H3 SEQ ID No. 205, 321, 322 | | | |
| 1: | 1 | SerArgAspIleGlnMetThrGlnSerProSerSerLeuSerAlaSerValGlyAspArg | 60 |
| | | TCTAGAGACATCCAGATGACGCAGTCCCCATCCTCCCTGTCTGCATCTGTAGGAGACAGG | |
| | | ---------!---------!---------!---------!---------!---------! | |
| | | AGATCTCTGTAGGTCTACTGCGTCAGGGGTAGGAGGGACAGACGTAGACATCCTCTGTCC | |
| 1: | 61 | ValThrIleThrCysGlnAlaSerGlnSerIleHisLysTyrLeuSerTrpTyrGlnGln | 120 |
| | | GTCACCATCACTTGCCAGGCCAGTCAGAGCATTCATAAGTACTTATCCTGGTATCAGCAG | |
| | | ---------!---------!---------!---------!---------!---------! | |
| | | CAGTGGTAGTGAACGGTCCGGTCAGTCTCGTAAGTATTCATGAATAGGACCATAGTCGTC | |
| 1: | 121 | LysProGlyLysAlaProLysLeuLeuIleTyrArgAlaSerThrLeuAlaSerGlyVal | 180 |
| | | AAGCCAGGCAAGGCACCTAAGCTCCTGATCTATAGGGCATCCACTCTGGCATCTGGGGTC | |
| | | ---------!---------!---------!---------!---------!---------! | |
| | | TTCGGTCCGTTCCGTGGATTCGAGGACTAGATATCCCGTAGGTGAGACCGTAGACCCCAG | |
| 1: | 181 | ProSerArgPheSerGlySerGlySerGlyThrAspPheThrLeuThr IleSerSerLeu | 240 |
| | | CCCTCAAGGTTTAGTGGAAGTGGATCTGGGACAGATTTCACCCTCACCATCAGCAGTCTG | |
| | | ---------!---------!---------!---------!---------!---------! | |
| | | GGGAGTTCCAAATCACCTTCACCTAGACCCTGTCTAAAGTGGGAGTGGTAGTCGTCAGAC | |
| 1: | 241 | GlnProGluAspPheAlaThrTyrTyrCysGlnGlnAspAlaAlaValIleSerIleAsp | 300 |
| | | CAACCGGAGGACTTCGCGACGTACTACTGCCAACAGGATGCTGCCGTTATTAGTATTGAT | |
| | | ---------!---------!---------!---------!---------!---------! | |
| | | GTTGGCCTCCTGAAGCGCTGCATGATGACGGTTGTCCTACGACGGCAATAATCATAACTA | |
| 1: | 301 | AsnSerPheGlyGlyGlyThrLysValGluIleLys | 336 |
| | | AACAGTTTCGGTGGTGGTACAAAGGTCGAGATCAAA | |
| | | ---------!---------!---------!------ | |
| | | TTGTCAAAGCCACCACCATGTTTCCAGCTCTAGTTT | |
| h-21626B-H3KQ SEQ ID No. 206, 323, 324 | | | |
| 1: | 1 | SerArgAspIleGlnMetThrGlnSerProSerSerLeuSerAlaSerValGlyAspArg | 60 |
| | | TCTAGAGACATCCAGATGACGCAGTCCCCATCCTCCCTGTCTGCATCTGTAGGAGACAGG | |
| | | ---------!---------!---------!---------!---------!---------! | |
| | | AGATCTCTGTAGGTCTACTGCGTCAGGGGTAGGAGGGACAGACGTAGACATCCTCTGTCC | |
| 1: | 60 | ValThrIleThrCysGlnAlaSerGlnSerIleHisLysTyrLeuSerTrpTyrGlnGln | 120 |
| | | GTCACCATCACTTGCCAGGCCAGTCAGAGCATTCATAAGTACTTATCCTGGTATCAGCAG | |
| | | ---------!---------!---------!---------!---------!---------! | |
| | | CAGTGGTAGTGAACGGTCCGGTCAGTCTCGTAAGTATTCATGAATAGGACCATAGTCGTC | |
| 1: | 121 | LysProGlyLysAlaProLysLeuLeuIleTyrArgAlaSerThrLeuAlaSerGlyVal | 180 |
| | | AAGCCAGGCAAGGCACCTAAGCTCCTGATCTATAGGGCATCCACTCTGGCATCTGGGGTC | |
| | | ---------!---------!---------!---------!---------!---------! | |
| | | TTCGGTCCGTTCCGTGGATTCGAGGACTAGATATCCCGTAGGTGAGACCGTAGACCCCAG | |
| 1: | 181 | ProSerArgPheLysGlySerGlySerGlyThrGlnPheThrLeuThrIleSerSerLeu | 240 |
| | | CCCTCAAGGTTTAAGGGAAGTGGATCTGGGACACAGTTCACCCTCACCATCAGCAGTCTG | |
| | | ---------!---------!---------!---------!---------!---------! | |
| | | GGGAGTTCCAAATTCCCTTCACCTAGACCCTGTGTCAAGTGGGAGTGGTAGTCGTCAGAC | |
| 1: | 241 | GlnProGluAspPheAlaThrTyrTyrCysGlnGlnAspAlaAlaValIleSerIleAsp | 300 |
| | | CAACCGGAGGACTTCGCGACGTACTACTGCCAACAGGATGCTGCCGTTATTAGTATTGAT | |
| | | ---------!---------!---------!---------!---------!---------! | |
| | | GTTGGCCTCCTGAAGCGCTGCATGATGACGGTTGTCCTACGACGGCAATAATCATAACTA | |
| 1: | 301 | AsnSerPheGlyGlyGlyThrLysValGluIleLys | 336 |
| | | AACAGTTTCGGTGGTGGTACAAAGGTCGAGATCAAA | |
| | | ---------!---------!---------!------ | |
| | | TTGTCAAAGCCACCACCATGTTTCCAGCTCTAGTTT | |

### Humanized Heavy Chain Sequences (Full Sequence)

Listed below are humanized heavy chain sequences (full sequence) of certain non-limiting anti-IgE antibodies, identified by name (e.g. “h-21626B-C4-8”) and ID number (e.g. “SEQ ID No. 207”).

| h-21626B-C4-8 SEQ ID No. 207, 325, 326 | | | |
|---|---|---|---|
| 1: | 1 | LeuGluGluValGlnLeuValGluSerGlyGlyGlyLeuValGlnProGlyGlySerLeu | 60 |
| | | CTCGAGGAGGTCCAGCTCGTTGAGTCTGGAGGAGGCTTGGTCCAGCCTGGAGGGTCCCTG | |
| | | ---------!---------!---------!---------!---------!---------! | |
| | | GAGCTCCTCCAGGTCGAGCAACTCAGACCTCCTCCGAACCAGGTCGGACCTCCCAGGGAC | |
| 1: | 60 | ArgLeuSerCysThrAlaSerGlyPheSerLeuSerSerTyrTyrMetSerTrpValArg | 120 |
| | | AGGCTCTCCTGTACAGCCTCTGGATTCTCCCTCAGTAGCTACTACATGAGCTGGGTAAGA | |
| | | ---------!---------!---------!---------!---------!---------! | |
| | | TCCGAGAGGACATGTCGGAGACCTAAGAGGGAGTCATCGATGATGTACTCGACCCATTCT | |
| 1: | 121 | GlnAlaProGlyLysGlyLeuGluTrpIleGlyIleIleTyrProSerGlyAsnThrTyr | 180 |
| | | CAGGCACCAGGGAAGGGACTGGAGTGGATCGGCATCATTTATCCTAGTGGTAACACATAC | |
| | | ---------!---------!---------!---------!---------!---------! | |
| | | GTCCGTGGTCCCTTCCCTGACCTCACCTAGCCGTAGTAAATAGGATCACCATTGTGTATG | |
| 1: | 181 | TyrAlaAsnTrpAlaAsnGlyArgPheThrIleSerArgHisAsnSerLysAsnThrLeu | 240 |
| | | TACGCGAACTGGGCAAATGGTCGATTCACCATCTCCAGACACAATTCCAAGAACACGCTG | |
| | | ---------!---------!---------!---------!---------!---------! | |
| | | ATGCGCTTGACCCGTTTACCAGCTAAGTGGTAGAGGTCTGTGTTAAGGTTCTTGTGCGAC | |
| 1: | 241 | TyrLeuGlnMetAsnSerLeuArgAlaGluAspThrAlaValTyrTyrCysAlaArgAsp | 300 |
| | | TATCTTCAAATGAACAGCCTGAGAGCGGAGGACACGGCCGTGTACTACTGTGCCAGAGAT | |
| | | ---------!---------!---------!---------!---------!---------! | |
| | | ATAGAAGTTTACTTGTCGGACTCTCGCCTCCTGTGCCGGCACATGATGACACGGTCTCTA | |
| 1: | 301 | SerGlyTyrProAspIleTrpGlyGlnGlyThrLeuValThrValSerSerAlaSerThr | 360 |
| | | TCTGGTTATCCTGACATCTGGGGTCAAGGAACCCTGGTCACCGTCTCCTCAGCCTCCACC | |
| | | ---------!---------!---------!---------!---------!---------! | |
| | | AGACCAATAGGACTGTAGACCCCAGTTCCTTGGGACCAGTGGCAGAGGAGTCGGAGGTGG | |
| 1: | 361 | LysGlyProSerValPheProLeuAlaProSerSerLysSerThrSerGlyGlyThrAla | 420 |
| | | AAGGGCCCATCGGTCTTCCCCCTGGCACCCTCCTCCAAGAGCACCTCTGGGGGCACAGCG | |
| | | ---------!---------!---------!---------!---------!---------! | |
| | | TTCCCGGGTAGCCAGAAGGGGGACCGTGGGAGGAGGTTCTCGTGGAGACCCCCGTGTCGC | |
| 1: | 421 | AlaLeuGlyCysLeuValLysAspTyrPheProGluProValThrValSerTrpAsnSer | 480 |
| | | GCCCTGGGCTGCCTGGTCAAGGACTACTTCCCCGAACCGGTGACGGTGTCGTGGAACTCA | |
| | | ---------!---------!---------!---------!---------!---------! | |
| | | CGGGACCCGACGGACCAGTTCCTGATGAAGGGGCTTGGCCACTGCCACAGCACCTTGAGT | |
| 1: | 481 | GlyAlaLeuThrSerGlyValHisThrPheProAlaValLeuGlnSerSerGlyLeuTyr | 540 |
| | | GGCGCCCTGACCAGCGGCGTGCACACCTTCCCGGCTGTCCTACAGTCCTCAGGACTCTAC | |
| | | ---------!---------!---------!---------!---------!---------! | |
| | | CCGCGGGACTGGTCGCCGCACGTGTGGAAGGGCCGACAGGATGTCAGGAGTCCTGAGATG | |
| 1: | 541 | SerLeuSerSerValValThrValProSerSerSerLeuGlyThrGlnThrTyrIleCys | 600 |
| | | TCCCTCAGCAGCGTGGTGACCGTGCCCTCCAGCAGCTTGGGCACCCAGACCTACATCTGC | |
| | | ---------!---------!---------!---------!---------!---------! | |
| | | AGGGAGTCGTCGCACCACTGGCACGGGAGGTCGTCGAACCCGTGGGTCTGGATGTAGACG | |
| 1: | 601 | AsnValAsnHisLysProSerAsnThrLysValAspLysLysValGluProLysSerCys | 660 |
| | | AACGTGAATCACAAGCCCAGCAACACCAAGGTGGACAAGAAAGTTGAGCCCAAATCTTGT | |
| | | ---------!---------!---------!---------!---------!---------! | |
| | | TTGCACTTAGTGTTCGGGTCGTTGTGGTTCCACCTGTTCTTTCAACTCGGGTTTAGAACA | |
| 1: | 661 | AspLys | 666 |
| | | GACAAA | |
| | | ------ | |
| | | CTGTTT | |
| h-21626B-H3 SEQ ID No. 208, 327, 328 | | | |
| 1: | 1 | LeuGluGlnValGlnLeuValGluSerGlyGlyGlyLeuValGlnProGlyGlySerLeu | 60 |
| | | CTCGAGCAGGTCCAGCTGGTTGAGTCTGGGGGAGGCTTGGTCCAGCCTGGAGGGTCCCTG | |
| | | ---------!---------!---------!---------!---------!---------! | |
| | | GAGCTCGTCCAGGTCGACCAACTCAGACCCCCTCCGAACCAGGTCGGACCTCCCAGGGAC | |
| 1: | 61 | ArgLeuSerCysThrAlaSerGlyPheSerLeuSerSerTyrAlaMetGlyTrpValArg | 120 |
| | | AGGCTCTCCTGTACAGCCTCTGGATTCTCCCTCAGTAGCTATGCAATGGGCTGGGTAAGA | |
| | | ---------!---------!---------!---------!---------!---------! | |
| | | TCCGAGAGGACATGTCGGAGACCTAAGAGGGAGTCATCGATACGTTACCCGACCCATTCT | |
| 1: | 121 | GlnAlaProGlyLysGlyLeuGluTyrIleGlyTrpIleSerAlaGlyGlyThrThrTyr | 180 |
| | | CAGGCACCAGGGAAGGGACTTGAGTACATCGGCTGGATTAGTGCTGGTGGTACCACATAC | |
| | | ---------!---------!---------!---------!---------!---------! | |
| | | GTCCGTGGTCCCTTCCCTGAACTCATGTAGCCGACCTAATCACGACCACCATGGTGTATG | |
| 1: | 181 | TyrAlaSerTrpValAsnSerArgPheThrIleSerArgAspAsnSerLysAsnThrLeu | 240 |
| | | TACGCGAGCTGGGTGAATAGCAGATTCACCATCTCCAGAGACAATTCCAAGAACACGCTG | |
| | | ---------!---------!---------!---------!---------!---------! | |
| | | ATGCGCTCGACCCACTTATCGTCTAAGTGGTAGAGGTCTCTGTTAAGGTTCTTGTGCGAC | |
| 1: | 241 | TyrLeuGlnMetAsnSerLeuArgAlaGluAspThrAlaValTyrTyrCysAlaArgGlu | 300 |
| | | TATCTTCAAATGAACAGCCTGAGAGCGGAGGACACGGCCGTGTACTACTGTGCCAGAGAG | |
| | | ---------!---------!---------!---------!---------!---------! | |
| | | ATAGAAGTTTACTTGTCGGACTCTCGCCTCCTGTGCCGGCACATGATGACACGGTCTCTC | |
| 1: | 301 | GlyThrGlyTrpGlyAlaTyrAspIleTrpGlyGlnGlyThrLeuValThrValSerSer | 360 |
| | | GGTACTGGCTGGGGTGCCTATGACATCTGGGGTCAAGGAACCCTGGTCACCGTCTCCTCA | |
| | | ---------!---------!---------!---------!---------!---------! | |
| | | CCATGACCGACCCCACGGATACTGTAGACCCCAGTTCCTTGGGACCAGTGGCAGAGGAGT | |
| 1: | 361 | AlaSerThrLysGlyProSerValPheProLeuAlaProSerSerLysSerThrSerGly | 420 |
| | | GCCTCCACCAAGGGCCCATCGGTCTTCCCCCTGGCACCCTCCTCCAAGAGCACCTCTGGG | |
| | | ---------!---------!---------!---------!---------!---------! | |
| | | CGGAGGTGGTTCCCGGGTAGCCAGAAGGGGGACCGTGGGAGGAGGTTCTCGTGGAGACCC | |
| 1: | 421 | GlyThrAlaAlaLeuGlyCysLeuValLysAspTyrPheProGluProValThrValSer | 480 |
| | | GGCACAGCGGCCCTGGGCTGCCTGGTCAAGGACTACTTCCCCGAACCGGTGACGGTGTCG | |
| | | ---------!---------!---------!---------!---------!---------! | |
| | | CCGTGTCGCCGGGACCCGACGGACCAGTTCCTGATGAAGGGGCTTGGCCACTGCCACAGC | |
| 1: | 481 | TrpAsnSerGlyAlaLeuThrSerGlyValHisThrPheProAlaValLeuGlnSerSer | 540 |
| | | TGGAACTCAGGCGCCCTGACCAGCGGCGTGCACACCTTCCCGGCTGTCCTACAGTCCTCA | |
| | | ---------!---------!---------!---------!---------!---------! | |
| | | ACCTTGAGTCCGCGGGACTGGTCGCCGCACGTGTGGAAGGGCCGACAGGATGTCAGGAGT | |
| 1: | 541 | GlyLeuTyrSerLeuSerSerValValThrValProSerSerSerLeuGlyThrGlnThr | 600 |
| | | GGACTCTACTCCCTCAGCAGCGTGGTGACCGTGCCCTCCAGCAGCTTGGGCACCCAGACC | |
| | | ---------!---------!---------!---------!---------!---------! | |
| | | CCTGAGATGAGGGAGTCGTCGCACCACTGGCACGGGAGGTCGTCGAACCCGTGGGTCTGG | |
| 1: | 601 | TyrIleCysAsnValAsnHisLysProSerAsnThrLysValAspLysLysValGluPro | 660 |
| | | TACATCTGCAACGTGAATCACAAGCCCAGCAACACCAAGGTGGACAAGAAAGTTGAGCCC | |
| | | ---------!---------!---------!---------!---------!---------! | |
| | | ATGTAGACGTTGCACTTAGTGTTCGGGTCGTTGTGGTTCCACCTGTTCTTTCAACTCGGG | |
| 1: | 661 | LysSerCysAspLys | 675 |
| | | AAATCTTGTGACAAA | |
| | | ---------!----- | |
| | | TTTAGAACACTGTTT | |

### Humanized Light Chain Sequences (Full Sequence)

Listed below are humanized light chain sequences (full sequence) of certain non-limiting anti-IgE antibodies, identified by name (e.g. “h-21626B-C4-8”) and ID number (e.g. “SEQ ID No. 209”).

| h-21626B-C4-8 SEQ ID No. 209, 329, 330 | | | |
|---|---|---|---|
| 1: | 1 | SerArgAspIleGlnMetThrGlnSerProSerSerLeuSerAlaSerValGlyAspArg | 60 |
| | | TCTAGAGACATCCAGATGACGCAGTCCCCATCCTCCCTGTCTGCATCTGTAGGAGACAGG | |
| | | ---------!---------!---------!---------!---------!---------! | |
| | | AGATCTCTGTAGGTCTACTGCGTCAGGGGTAGGAGGGACAGACGTAGACATCCTCTGTCC | |
| 1: | 61 | ValThrIleThrCysGlnAlaSerGlnSerIleSerSerTyrLeuSerTrpTyrGlnGln | 120 |
| | | GTCACCATCACTTGCCAGGCAAGTCAGAGCATTAGTAGTTACTTATCCTGGTATCAGCAG | |
| | | ---------!---------!---------!---------!---------!---------! | |
| | | CAGTGGTAGTGAACGGTCCGTTCAGTCTCGTAATCATCAATGAATAGGACCATAGTCGTC | |
| 1: | 121 | LysProGlyLysAlaProLysLeuLeuIleTyrGluThrSerLysLeuAlaSerGlyVal | 180 |
| | | AAGCCAGGCAAGGCACCTAAGCTCCTGATCTATGAAACATCCAAACTGGCATCTGGGGTC | |
| | | ---------!---------!---------!---------!---------!---------! | |
| | | TTCGGTCCGTTCCGTGGATTCGAGGACTAGATACTTTGTAGGTTTGACCGTAGACCCCAG | |
| 1: | 181 | ProSerArgPheSerGlySerGlySerGlyThrAspPheThrLeuThrIleSerSerLeu | 240 |
| | | CCCTCAAGGTTTAGTGGAAGTGGATCTGGGACAGATTTCACCCTCACCATCAGCAGTCTG | |
| | | ---------!---------!---------!---------!---------!---------! | |
| | | GGGAGTTCCAAATCACCTTCACCTAGACCCTGTCTAAAGTGGGAGTGGTAGTCGTCAGAC | |
| 1: | 241 | GlnProGluAspPheAlaThrTyrTyrCysGlnGlnAspAlaGlyValIleAsnValHis | 300 |
| | | CAACCGGAGGACTTCGCGACGTACTACTGCCAACAGGATGCTGGTGTTATTAATGTTCAT | |
| | | ---------!---------!---------!---------!---------!---------! | |
| | | GTTGGCCTCCTGAAGCGCTGCATGATGACGGTTGTCCTACGACCACAATAATTACAAGTA | |
| 1: | 301 | AsnThrPheGlyGlyGlyThrLysValGluIleLysArgThrValAlaAlaProSerVal | 360 |
| | | AATACTTTCGGTGGTGGTACAAAGGTCGAGATCAAACGGACTGTGGCTGCACCATCTGTC | |
| | | ---------!---------!---------!---------!---------!---------! | |
| | | TTATGAAAGCCACCACCATGTTTCCAGCTCTAGTTTGCCTGACACCGACGTGGTAGACAG | |
| 1: | 361 | PheIlePheProProSerAspGluGlnLeuLysSerGlyThrAlaSerValValCysLeu | 420 |
| | | TTCATCTTCCCGCCATCTGATGAGCAGTTGAAATCTGGAACTGCCTCTGTTGTGTGCCTG | |
| | | ---------!---------!---------!---------!---------!---------! | |
| | | AAGTAGAAGGGCGGTAGACTACTCGTCAACTTTAGACCTTGACGGAGACAACACACGGAC | |
| 1: | 421 | LeuAsnAsnPheTyrProArgGluAlaLysValGlnTrpLysValAspAsnAlaLeuGln | 480 |
| | | CTGAATAACTTCTATCCCAGAGAGGCCAAAGTACAGTGGAAGGTGGATAACGCCCTCCAA | |
| | | ---------!---------!---------!---------!---------!---------! | |
| | | GACTTATTGAAGATAGGGTCTCTCCGGTTTCATGTCACCTTCCACCTATTGCGGGAGGTT | |
| 1: | 481 | SerGlyAsnSerGlnGluSerValThrGluGInAspSerLysAspSerThrTyrSerLeu | 540 |
| | | TCGGGTAACTCCCAGGAGAGTGTCACAGAGCAGGACAGCAAGGACAGCACCTACAGCCTC | |
| | | ---------!---------!---------!---------!---------!---------! | |
| | | AGCCCATTGAGGGTCCTCTCACAGTGTCTCGTCCTGTCGTTCCTGTCGTGGATGTCGGAG | |
| 1: | 541 | SerSerThrLeuThrLeuSerLysAlaAspTyrGluLysHisLysValTyrAlaCysGlu | 600 |
| | | AGCAGCACCCTGACGCTGAGCAAAGCAGACTACGAGAAACACAAAGTCTACGCCTGCGAA | |
| | | ---------!---------!---------!---------!---------!---------! | |
| | | TCGTCGTGGGACTGCGACTCGTTTCGTCTGATGCTCTTTGTGTTTCAGATGCGGACGCTT | |
| 1: | 601 | ValThrHisGlnGlyLeuSerSerProValThrLysSerPheAsnArgGlyGluCys | 657 |
| | | GTCACCCATCAGGGCCTGAGCTCGCCCGTCACAAAGAGCTTCAACAGGGGAGAGTGT | |
| | | ---------!---------!---------!---------!---------!------- | |
| | | CAGTGGGTAGTCCCGGACTCGAGCGGGCAGTGTTTCTCGAAGTTGTCCCCTCTCACA | |
| h-21626C-C4-8E SEQ ID No. 210, 331, 332 | | | |
| 1: | 1 | SerArgAspIleGlnMetThrGlnSerProSerSerLeuSerAlaSerValGlyAspArg | 60 |
| | | TCTAGAGACATCCAGATGACGCAGTCCCCATCCTCCCTGTCTGCATCTGTAGGAGACAGG | |
| | | ---------!---------!---------!---------!---------!---------! | |
| | | AGATCTCTGTAGGTCTACTGCGTCAGGGGTAGGAGGGACAGACGTAGACATCCTCTGTCC | |
| 1: | 61 | ValThrIleThrCysGlnAlaSerGlnSerIleSerSerTyrLeuSerTrpTyrGlnGln | 120 |
| | | GTCACCATCACTTGCCAGGCAAGTCAGAGCATTAGTAGTTACTTATCCTGGTATCAGCAG | |
| | | ---------!---------!---------!---------!---------!---------! | |
| | | CAGTGGTAGTGAACGGTCCGTTCAGTCTCGTAATCATCAATGAATAGGACCATAGTCGTC | |
| 1: | 121 | LysProGlyLysAlaProLysLeuLeuIleTyrGluThrSerLysLeuAlaSerGlyVal | 180 |
| | | AAGCCAGGCAAGGCACCTAAGCTCCTGATCTATGAAACATCCAAACTGGCATCTGGGGTC | |
| | | ---------!---------!---------!---------!---------!---------! | |
| | | TTCGGTCCGTTCCGTGGATTCGAGGACTAGATACTTTGTAGGTTTGACCGTAGACCCCAG | |
| 1: | 181 | ProSerArgPheSerGlySerGlySerGlyThrGluPheThrLeuThrIleSerSerLeu | 240 |
| | | CCCTCAAGGTTTAGTGGAAGTGGATCTGGGACAGAATTCACCCTCACCATCAGCAGTCTG | |
| | | ---------!---------!---------!---------!---------!---------! | |
| | | GGGAGTTCCAAATCACCTTCACCTAGACCCTGTCTTAAGTGGGAGTGGTAGTCGTCAGAC | |
| 1: | 241 | GlnProGluAspPheAlaThrTyrTyrCysGlnGlnAspAlaGlyValIleAsnValHis | 300 |
| | | CAACCGGAGGACTTCGCGACGTACTACTGCCAACAGGATGCTGGTGTTATTAATGTTCAT | |
| | | ---------!---------!---------!---------!---------!---------! | |
| | | GTTGGCCTCCTGAAGCGCTGCATGATGACGGTTGTCCTACGACCACAATAATTACAAGTA | |
| 1: | 301 | AsnThrPheGlyGlyGlyThrLysValGluIleLysArgThrValAlaAlaProSerVal | 360 |
| | | AATACTTTCGGTGGTGGTACAAAGGTCGAGATCAAACGGACTGTGGCTGCACCATCTGTC | |
| | | ---------!---------!---------!---------!---------!---------! | |
| | | TTATGAAAGCCACCACCATGTTTCCAGCTCTAGTTTGCCTGACACCGACGTGGTAGACAG | |
| 1: | 361 | PheIlePheProProSerAspGluGlnLeuLysSerGlyThrAlaSerValValCysLeu | 420 |
| | | TTCATCTTCCCGCCATCTGATGAGCAGTTGAAATCTGGAACTGCCTCTGTTGTGTGCCTG | |
| | | ---------!---------!---------!---------!---------!---------! | |
| | | AAGTAGAAGGGCGGTAGACTACTCGTCAACTTTAGACCTTGACGGAGACAACACACGGAC | |
| 1: | 421 | LeuAsnAsnPheTyrProArgGluAlaLysValGlnTrpLysValAspAsnAlaLeuGln | 480 |
| | | CTGAATAACTTCTATCCCAGAGAGGCCAAAGTACAGTGGAAGGTGGATAACGCCCTCCAA | |
| | | ---------!---------!---------!---------!---------!---------! | |
| | | GACTTATTGAAGATAGGGTCTCTCCGGTTTCATGTCACCTTCCACCTATTGCGGGAGGTT | |
| 1: | 481 | SerGlyAsnSerGlnGluSerValThrGluGlnAspSerLysAspSerThrTyrSerLeu | 540 |
| | | TCGGGTAACTCCCAGGAGAGTGTCACAGAGCAGGACAGCAAGGACAGCACCTACAGCCTC | |
| | | ---------!---------!---------!---------!---------!---------! | |
| | | AGCCCATTGAGGGTCCTCTCACAGTGTCTCGTCCTGTCGTTCCTGTCGTGGATGTCGGAG | |
| 1: | 541 | SerSerThrLeuThrLeuSerLysAlaAspTyrGluLysHisLysValTyrAlaCysGlu | 600 |
| | | AGCAGCACCCTGACGCTGAGCAAAGCAGACTACGAGAAACACAAAGTCTACGCCTGCGAA | |
| | | ---------!---------!---------!---------!---------!---------! | |
| | | TCGTCGTGGGACTGCGACTCGTTTCGTCTGATGCTCTTTGTGTTTCAGATGCGGACGCTT | |
| 1: | 601 | ValThrHisGlnGlyLeuSerSerProValThrLysSerPheAsnArgGlyGluCys | 657 |
| | | GTCACCCATCAGGGCCTGAGCTCGCCCGTCACAAAGAGCTTCAACAGGGGAGAGTGT | |
| | | ---------!---------!---------!---------!---------!------- | |
| | | CAGTGGGTAGTCCCGGACTCGAGCGGGCAGTGTTTCTCGAAGTTGTCCCCTCTCACA | |
| h-21626B-H3 SEQ ID No. 211, 333, 334 | | | |
| 1: | 1 | SerArgAspIleGlnMetThrGlnSerProSerSerLeuSerAlaSerValGlyAspArg | 60 |
| | | TCTAGAGACATCCAGATGACGCAGTCCCCATCCTCCCTGTCTGCATCTGTAGGAGACAGG | |
| | | ---------!---------!---------!---------!---------!---------! | |
| | | AGATCTCTGTAGGTCTACTGCGTCAGGGGTAGGAGGGACAGACGTAGACATCCTCTGTCC | |
| 1: | 61 | ValThrIleThrCysGlnAlaSerGlnSerIleHisLysTyrLeuSerTrpTyrGlnGln | 120 |
| | | GTCACCATCACTTGCCAGGCCAGTCAGAGCATTCATAAGTACTTATCCTGGTATCAGCAG | |
| | | ---------!---------!---------!---------!---------!---------! | |
| | | CAGTGGTAGTGAACGGTCCGGTCAGTCTCGTAAGTATTCATGAATAGGACCATAGTCGTC | |
| 1: | 121 | LysProGlyLysAlaProLysLeuLeuIleTyrArgAlaSerThrLeuAlaSerGlyVal | 180 |
| | | AAGCCAGGCAAGGCACCTAAGCTCCTGATCTATAGGGCATCCACTCTGGCATCTGGGGTC | |
| | | ---------!---------!---------!---------!---------!---------! | |
| | | TTCGGTCCGTTCCGTGGATTCGAGGACTAGATATCCCGTAGGTGAGACCGTAGACCCCAG | |
| 1: | 181 | ProSerArgPheSerGlySerGlySerGlyThrAspPheThrLeuThrIleSerSerLeu | 240 |
| | | CCCTCAAGGTTTAGTGGAAGTGGATCTGGGACAGATTTCACCCTCACCATCAGCAGTCTG | |
| | | ---------!---------!---------!---------!---------!---------! | |
| | | GGGAGTTCCAAATCACCTTCACCTAGACCCTGTCTAAAGTGGGAGTGGTAGTCGTCAGAC | |
| 1: | 241 | GlnProGluAspPheAlaThrTyrTyrCysGlnGlnAspAlaAlaValIleSerIleAsp | 300 |
| | | CAACCGGAGGACTTCGCGACGTACTACTGCCAACAGGATGCTGCCGTTATTAGTATTGAT | |
| | | ---------!---------!---------!---------!---------!---------! | |
| | | GTTGGCCTCCTGAAGCGCTGCATGATGACGGTTGTCCTACGACGGCAATAATCATAACTA | |
| 1: | 301 | AsnSerPheGlyGlyGlyThrLysValGluIleLysArgThrValAlaAlaProSerVal | 360 |
| | | AACAGTTTCGGTGGTGGTACAAAGGTCGAGATCAAACGGACTGTGGCTGCACCATCTGTC | |
| | | ---------!---------!---------!---------!---------!---------! | |
| | | TTGTCAAAGCCACCACCATGTTTCCAGCTCTAGTTTGCCTGACACCGACGTGGTAGACAG | |
| 1: | 361 | PheIlePheProProSerAspGluGlnLeuLysSerGlyThrAlaSerValValCysLeu | 420 |
| | | TTCATCTTCCCGCCATCTGATGAGCAGTTGAAATCTGGAACTGCCTCTGTTGTGTGCCTG | |
| | | ---------!---------!---------!---------!---------!---------! | |
| | | AAGTAGAAGGGCGGTAGACTACTCGTCAACTTTAGACCTTGACGGAGACAACACACGGAC | |
| 1: | 421 | LeuAsnAsnPheTyrProArgGluAlaLysValGlnTrpLysValAspAsnAlaLeuGln | 480 |
| | | CTGAATAACTTCTATCCCAGAGAGGCCAAAGTACAGTGGAAGGTGGATAACGCCCTCCAA | |
| | | ---------!---------!---------!---------!---------!---------! | |
| | | GACTTATTGAAGATAGGGTCTCTCCGGTTTCATGTCACCTTCCACCTATTGCGGGAGGTT | |
| 1: | 481 | SerGlyAsnSerGlnGluSerValThrGluGInAspSerLysAspSerThrTyrSerLeu | 540 |
| | | TCGGGTAACTCCCAGGAGAGTGTCACAGAGCAGGACAGCAAGGACAGCACCTACAGCCTC | |
| | | ---------!---------!---------!---------!---------!---------! | |
| | | AGCCCATTGAGGGTCCTCTCACAGTGTCTCGTCCTGTCGTTCCTGTCGTGGATGTCGGAG | |
| 1: | 541 | SerSerThrLeuThrLeuSerLysAlaAspTyrGluLysHisLysValTyrAlaCysGlu | 600 |
| | | AGCAGCACCCTGACGCTGAGCAAAGCAGACTACGAGAAACACAAAGTCTACGCCTGCGAA | |
| | | ---------!---------!---------!---------!---------!---------! | |
| | | TCGTCGTGGGACTGCGACTCGTTTCGTCTGATGCTCTTTGTGTTTCAGATGCGGACGCTT | |
| 1: | 601 | ValThrHisGlnGlyLeuSerSerProValThrLysSerPheAsnArgGlyGluCys | 657 |
| | | GTCACCCATCAGGGCCTGAGCTCGCCCGTCACAAAGAGCTTCAACAGGGGAGAGTGT | |
| | | ---------!---------!---------!---------!---------!------- | |
| | | CAGTGGGTAGTCCCGGACTCGAGCGGGCAGTGTTTCTCGAAGTTGTCCCCTCTCACA | |
| h-21626B-H3KQ SEQ ID No. 212, 335, 336 | | | |
| 1: | 1 | SerArgAspIleGlnMetThrGlnSerProSerSerLeuSerAlaSerValGlyAspArg | 60 |
| | | TCTAGAGACATCCAGATGACGCAGTCCCCATCCTCCCTGTCTGCATCTGTAGGAGACAGG | |
| | | ---------!---------!---------!---------!---------!---------! | |
| | | AGATCTCTGTAGGTCTACTGCGTCAGGGGTAGGAGGGACAGACGTAGACATCCTCTGTCC | |
| 1: | 61 | ValThrIleThrCysGlnAlaSerGlnSerIleHisLysTyrLeuSerTrpTyrGlnGln | 120 |
| | | GTCACCATCACTTGCCAGGCCAGTCAGAGCATTCATAAGTACTTATCCTGGTATCAGCAG | |
| | | ---------!---------!---------!---------!---------!---------! | |
| | | CAGTGGTAGTGAACGGTCCGGTCAGTCTCGTAAGTATTCATGAATAGGACCATAGTCGTC | |
| 1: | 121 | LysProGlyLysAlaProLysLeuLeuIleTyrArgAlaSerThrLeuAlaSerGlyVal | 180 |
| | | AAGCCAGGCAAGGCACCTAAGCTCCTGATCTATAGGGCATCCACTCTGGCATCTGGGGTC | |
| | | ---------!---------!---------!---------!---------!---------! | |
| | | TTCGGTCCGTTCCGTGGATTCGAGGACTAGATATCCCGTAGGTGAGACCGTAGACCCCAG | |
| 1: | 181 | ProSerArgPheLysGlySerGlySerGlyThrGlnPheThrLeuThrIleSerSerLeu | 240 |
| | | CCCTCAAGGTTTAAGGGAAGTGGATCTGGGACACAGTTCACCCTCACCATCAGCAGTCTG | |
| | | ---------!---------!---------!---------!---------!---------! | |
| | | GGGAGTTCCAAATTCCCTTCACCTAGACCCTGTGTCAAGTGGGAGTGGTAGTCGTCAGAC | |
| 1: | 241 | GlnProGluAspPheAlaThrTyrTyrCysGlnGlnAspAlaAlaValIleSerIleAsp | 300 |
| | | CAACCGGAGGACTTCGCGACGTACTACTGCCAACAGGATGCTGCCGTTATTAGTATTGAT | |
| | | ---------!---------!---------!---------!---------!---------! | |
| | | GTTGGCCTCCTGAAGCGCTGCATGATGACGGTTGTCCTACGACGGCAATAATCATAACTA | |
| 1: | 301 | AsnSerPheGlyGlyGlyThrLysValGluIleLysArgThrValAlaAlaProSerVal | 360 |
| | | AACAGTTTCGGTGGTGGTACAAAGGTCGAGATCAAACGGACTGTGGCTGCACCATCTGTC | |
| | | ---------!---------!---------!---------!---------!---------! | |
| | | TTGTCAAAGCCACCACCATGTTTCCAGCTCTAGTTTGCCTGACACCGACGTGGTAGACAG | |
| 1: | 361 | PheIlePheProProSerAspGluGlnLeuLysSerGlyThrAlaSerValValCysLeu | 420 |
| | | TTCATCTTCCCGCCATCTGATGAGCAGTTGAAATCTGGAACTGCCTCTGTTGTGTGCCTG | |
| | | ---------!---------!---------!---------!---------!---------! | |
| | | AAGTAGAAGGGCGGTAGACTACTCGTCAACTTTAGACCTTGACGGAGACAACACACGGAC | |
| 1: | 421 | LeuAsnAsnPheTyrProArgGluAlaLysValGlnTrpLysValAspAsnAlaLeuGln | 480 |
| | | CTGAATAACTTCTATCCCAGAGAGGCCAAAGTACAGTGGAAGGTGGATAACGCCCTCCAA | |
| | | ---------!---------!---------!---------!---------!---------! | |
| | | GACTTATTGAAGATAGGGTCTCTCCGGTTTCATGTCACCTTCCACCTATTGCGGGAGGTT | |
| 1: | 481 | SerGlyAsnSerGlnGluSerValThrGluGlnAspSerLysAspSerThrTyrSerLeu | 540 |
| | | TCGGGTAACTCCCAGGAGAGTGTCACAGAGCAGGACAGCAAGGACAGCACCTACAGCCTC | |
| | | ---------!---------!---------!---------!---------!---------! | |
| | | AGCCCATTGAGGGTCCTCTCACAGTGTCTCGTCCTGTCGTTCCTGTCGTGGATGTCGGAG | |
| 1: | 541 | SerSerThrLeuThrLeuSerLysAlaAspTyrGluLysHisLysValTyrAlaCysGlu | 600 |
| | | AGCAGCACCCTGACGCTGAGCAAAGCAGACTACGAGAAACACAAAGTCTACGCCTGCGAA | |
| | | ---------!---------!---------!---------!---------!---------! | |
| | | TCGTCGTGGGACTGCGACTCGTTTCGTCTGATGCTCTTTGTGTTTCAGATGCGGACGCTT | |
| 1: | 601 | ValThrHisGlnGlyLeuSerSerProValThrLysSerPheAsnArgGlyGluCys | 657 |
| | | GTCACCCATCAGGGCCTGAGCTCGCCCGTCACAAAGAGCTTCAACAGGGGAGAGTGT | |
| | | ---------!---------!---------!---------!---------!------- | |
| | | CAGTGGGTAGTCCCGGACTCGAGCGGGCAGTGTTTCTCGAAGTTGTCCCCTCTCACA | |

### INCORPORATION BY REFERENCE

All publications, patents, and patent applications mentioned in this specification are herein incorporated by reference to the same extent as if each individual publication, patent, or patent application was specifically and individually indicated to be incorporated by reference.
1. Strunk R C, Bloomberg G R. Omalizumab for asthma. N Engl J Med 2006; 354:2689-95.
2. Busse W, Corren J, Lanier B Q, McAlary M, Fowler-Taylor A, Cioppa G D, et al. Omalizumab, anti-IgE recombinant humanized monoclonal antibody, for the treatment of severe allergic asthma. J Allergy Clin Immunol 2001; 108:184-90.
3. Holgate S T, Chuchalin A G, Hebert J, Lotvall J, Persson G B, Chung K F, et al. Efficacy and safety of a recombinant anti-immunoglobulin E antibody (omalizumab) in severe allergic asthma. Clin Exp Allergy 2004; 34:632-8.
4. Soler M, Matz J, Townley R, Buhl R, O'Brien J, Fox H, et al. The anti-IgE antibody omalizumab reduces exacerbations and steroid requirement in allergic asthmatics. Eur Respir J 2001; 18:254-61.
5. Milgrom H, Berger W, Nayak A, Gupta N, Pollard S, McAlary M, et al. Treatment of childhood asthma with anti-immunoglobulin E antibody (omalizumab). Pediatrics 2001; 108:E36.
6. Saini S, Rosen K E, Hsieh H J, Wong D A, Conner E, Kaplan A, et al. A randomized, placebo-controlled, dose-ranging study of single-dose omalizumab in patients with H1-antihistamine-refractory chronic idiopathic urticaria. J Allergy Clin Immunol 2011; 128:567-73 e1.
7. Maurer M, Rosen K, Hsieh H J, Saini S, Grattan C, Gimenez-Arnau A, et al. Omalizumab for the treatment of chronic idiopathic or spontaneous urticaria. N Engl J Med 2013; 368:924-35.
8. Gevaert P, Calus L, Van Zele T, Blomme K, De Ruyck N, Bauters W, et al. Omalizumab is effective in allergic and nonallergic patients with nasal polyps and asthma. J Allergy Clin Immunol 2013; 131:110-6 e1.
9. Gevaert P, Omachi T A, Corren J, Mullol J, Han J, Lee S E, et al. Efficacy and safety of omalizumab in nasal polyposis: 2 randomized phase 3 trials. J Allergy Clin Immunol 2020; 146:595-605.
10. Okubo K, Okano M, Sato N, Tamaki Y, Suzuki H, Uddin A, et al. Add-On Omalizumab for Inadequately Controlled Severe Pollinosis Despite Standard-of-Care: A Randomized Study. J Allergy Clin Immunol Pract 2020; 8:3130-40 e2.
11. Dantzer J A, Wood R A. The use of omalizumab in allergen immunotherapy. Clin Exp Allergy 2018; 48:232-40.
12. Beck L A, Marcotte G V, MacGlashan D, Togias A, Saini S. Omalizumab-induced reductions in mast cell Fce psilon RI expression and function. J Allergy Clin Immunol 2004; 114:527-30.
13. Kaplan A P, Gimenez-Arnau A M, Saini S S. Mechanisms of action that contribute to efficacy of omalizumab in chronic spontaneous urticaria. Allergy 2017; 72:519-33.
14. Trischler J, Bottoli I, Janocha R, Heusser C, Jaumont X, Lowe P, et al. Ligelizumab treatment for severe asthma: learnings from the clinical development programme. Clin Transl Immunology 2021; 10:e1255.
15. Maurer M, Gimenez-Arnau A M, Sussman G, Metz M, Baker D R, Bauer A, et al. Ligelizumab for Chronic Spontaneous Urticaria. N Engl J Med 2019; 381:1321-32.
16. Gauvreau G M, Arm J P, Boulet L P, Leigh R, Cockcroft D W, Davis B E, et al. Efficacy and safety of multiple doses of QGE031 (ligelizumab) versus omalizumab and placebo in inhibiting allergen-induced early asthmatic responses. J Allergy Clin Immunol 2016; 138:1051-9.
17. Gasser P, Tarchevskaya S S, Guntern P, Brigger D, Ruppli R, Zbaren N, et al. The mechanistic and functional profile of the therapeutic anti-IgE antibody ligelizumab differs from omalizumab. Nat Commun 2020; 11:165.
18. Jensen R K, Jabs F, Miehe M, Molgaard B, Pfutzner W, Mobs C, et al. Structure of intact IgE and the mechanism of ligelizumab revealed by electron microscopy. Allergy 2020; 75:1956-65.
19. Drinkwater N, Cossins B, Keeble A H, Wright M, Cain K, Hailu H, et al. Human immunoglobulin E flexes between acutely bent and extended conformations. Nat Struct Mol Biol 2014; 21:397-404.
20. Cohen E S, Dobson C L, Kack H, Wang B, Sims D A, Lloyd C O, et al. A novel IgE-neutralizing antibody for the treatment of severe uncontrolled asthma. MAbs 2014; 6:756-64.
21. Sheldon E, Schwickart M, Li J, Kim K, Crouch S, Parveen S, et al. Pharmacokinetics, Pharmacodynamics, and Safety of MEDI4212, an Anti-IgE Monoclonal Antibody, in Subjects with Atopy: A Phase I Study. Adv Ther 2016; 33:225-51.
22. Baumann M J, Eggel A, Amstutz P, Stadler B M, Vogel M. DARPins against a functional IgE epitope. Immunol Lett 2010; 133:78-84.
23. Kim B, Eggel A, Tarchevskaya S S, Vogel M, Prinz H, Jardetzky T S. Accelerated disassembly of IgE-receptor complexes by a disruptive macromolecular inhibitor. Nature 2012; 491:613-7.
24. Prinz H, Striessnig J. Ligand-induced accelerated dissociation of (+)-cis-diltiazem from L-type Ca2+ channels is simply explained by competition for individual attachment points. J Biol Chem 1993; 268:18580-5.
25. Eggel A, Baravalle G, Hobi G, Kim B, Buschor P, Forrer P, et al. Accelerated dissociation of IgE-FcepsilonRI complexes by disruptive inhibitors actively desensitizes allergic effector cells. J Allergy Clin Immunol 2014; 133:1709-19 e8.
26. Jabs F, Plum M, Laursen N S, Jensen R K, Molgaard B, Miehe M, et al. Trapping IgE in a closed conformation by mimicking CD23 binding prevents and disrupts FcepsilonRI interaction. Nat Commun 2018; 9:7.
27. Harris J M, Cabanski C R, Scheerens H, Samineni D, Bradley M S, Cochran C, et al.
A randomized trial of quilizumab in adults with refractory chronic spontaneous urticaria. J Allergy Clin Immunol 2016; 138:1730-2.
28. Harris J M, Maciuca R, Bradley M S, Cabanski C R, Scheerens H, Lim J, et al. A randomized trial of the efficacy and safety of quilizumab in adults with inadequately controlled allergic asthma. Respir Res 2016; 17:29.
29. Gauvreau G M, Harris J M, Boulet L P, Scheerens H, Fitzgerald J M, Putnam W S, et al. Targeting membrane-expressed IgE B cell receptor with an antibody to the M1 prime epitope reduces IgE production. Sci Transl Med 2014; 6:243ra85.
30. Chu S Y, Horton H M, Pong E, Leung I W, Chen H, Nguyen D H, et al. Reduction of total IgE by targeted coengagement of IgE B-cell receptor and FcgammaRIIb with Fc-engineered antibody. J Allergy Clin Immunol 2012; 129:1102-15.
31. Kirak O, Riethmuller G. A novel, nonanaphylactogenic, bispecific IgE-CD3 antibody eliminates IgE(+) B cells. J Allergy Clin Immunol 2015; 136:800-2 e3.
32. Wiegand T W, Williams P B, Dreskin S C, Jouvin M H, Kinet J P, Tasset D. High-affinity oligonucleotide ligands to human IgE inhibit binding to Fe epsilon receptor I. J Immunol 1996; 157:221-30.
33. Mendonsa S D, Bowser M T. In vitro selection of high-affinity DNA ligands for human IgE using capillary electrophoresis. Anal Chem 2004; 76:5387-92.
34. Poongavanam M V, Kisley L, Kourentzi K, Landes C F, Willson R C. Ensemble and single-molecule biophysical characterization of D17.4 DNA aptamer-IgE interactions. Biochim Biophys Acta 2016; 1864:154-64.
35. Ando T, Kitaura J. Tuning IgE: IgE-Associating Molecules and Their Effects on IgE-Dependent Mast Cell Reactions. Cells 2021; 10.
36. Pennington L F, Tarchevskaya S, Brigger D, Sathiyamoorthy K, Graham M T, Nadeau K C, et al. Structural basis of omalizumab therapy and omalizumab-mediated IgE exchange. Nat Commun 2016; 7:11610.
37. Davies A M, Allan E G, Keeble A H, Delgado J, Cossins B P, Mitropoulou A N, et al. Allosteric mechanism of action of the therapeutic anti-IgE antibody omalizumab. J Biol Chem 2017; 292:9975-87.
38. McDonnell J M, Calvert R, Beavil R L, Beavil A J, Henry A J, Sutton B J, et al. The structure of the IgE Cepsilon2 domain and its role in stabilizing the complex with its high-affinity receptor FcepsilonRIalpha. Nat Struct Biol 2001; 8:437-41.
39. Hirano T, Koyanagi A, Kotoshiba K, Shinkai Y, Kasai M, Ando T, et al. The Fab fragment of anti-IgE Cepsilon2 domain prevents allergic reactions through interacting with IgE-FcepsilonRIalpha complex on rat mast cells. Sci Rep 2018; 8:14237.

What is claimed is:

1. An antibody or a fragment thereof, comprising a Fab region that specifically binds to an IgE, wherein the binding of the Fab to the IgE disrupts interaction between the IgE and at least one Fcε receptor.

2. The antibody or fragment of claim 1, wherein the disrupted interaction comprises blocking an unbound IgE from binding to the at least one Fcε receptor.

3. The antibody or fragment of claim 1, wherein the disrupted interaction comprises dissociating a bound IgE from the at least one Fcε receptor.

4. The antibody or fragment of claim 1, wherein the disrupted interaction results in suppression of degranulation.

5. The antibody or fragment of claim 1, wherein the at least one Fcε receptor comprises FcεRI.

6. The antigen binding polypeptide of claim 1, wherein the at least one Fcε receptor comprises CD23.

7. The antibody or fragment of claim 1, wherein the at least one Fcε receptor comprises FcεRI and CD23.

8. The antibody or fragment of claim 1, wherein the Fab region specifically binds to at least one amino acid residue in β5-helix region of the Cε2, wherein the β5-helix region is the combination of helix, β5-helix joint, and lower half of β5 that connects to helix of Cε2.

9. The antibody or fragment of claim 8, wherein the Fab region specifically binds to at least one amino acid residue in the helix of Cε2.

10. The antibody or fragment of claim 8, wherein the Fab region specifically binds to at least one amino acid residue in the β5-helix joint of Cε2.

11. The antibody or fragment of claim 8, wherein the Fab region specifically binds to at least one amino acid residue in the lower half of β5 that connects to helix of Cε2.

12. The antibody or fragment of claim 8, wherein the Fab region does not bind to amino acid residue T298 of Cε2.

13. The antibody or fragment of claim 8, wherein the Fab region binds to at least two amino acid residues in the β5-Cε2 helix region.

14. The antibody or fragment of claim 8, wherein the Fab region does not bind to β3 region of Cε2.

15. The antibody or fragment of claim 8, wherein the Fab region does not bind to β4 region of Cε2.

16. The antibody or fragment of claim 8, wherein the Fab region does not bind to β3 or β4 region of Cε2.

17. The antibody or fragment of claim 1, wherein the antibody is a bispecific antibody or a binding fragment thereof.

18. The antibody or fragment of claim 1, wherein the antibody comprises a monovalent Fab′, a divalent Fab2, a single-chain variable fragment (scFv), a diabody, a minibody, a nanobody, a single-domain antibody (sdAb), or a camelid antibody or binding fragment thereof.

19. The antibody or fragment of claim 1, wherein the Fab region comprises at least one heavy chain sequence selected from SEQ ID Nos 1-25.

20. The antibody or fragment of claim 1, wherein the Fab region comprises at least one light chain sequence selected from SEQ ID Nos 26-50.

21. The antibody or fragment of claim 1, wherein the Fab region comprises at least one complementarity determining region (CDR) selected from SEQ ID Nos 51-200.

22. The antibody or fragment of claim 1, wherein the antibody exhibits a K_{D} of less than 1 nM, 1.2 nM, 2 nM, 5 nM, 10 nM, 13.5 nM, 15 nM, 20 nM, 25 nM, or 30 nM.

23. The antibody or fragment of claim 1, wherein the antibody is a humanized antibody.

24. The antibody or a fragment thereof of claim 23, wherein the humanized antibody comprises a heavy chain variable region selected from SEQ ID Nos 201-202.

25. The antibody or a fragment thereof of claim 23, wherein the humanized antibody comprises a light chain variable region selected from SEQ ID Nos 203-206.

26. The antibody or a fragment thereof of claim 23, wherein the humanized antibody comprises a heavy chain variable region selected from SEQ ID Nos 201-202, and a light chain variable region selected from SEQ ID Nos 203-206.

27. The antibody or a fragment thereof of claim 23, wherein the humanized antibody comprises a heavy chain full sequence selected from SEQ ID Nos 207-208.

28. The antibody or a fragment thereof of claim 23, wherein the humanized antibody comprises a light chain full sequence selected from SEQ ID Nos 209-212.

29. The antibody or a fragment thereof of claim 23, wherein the humanized antibody comprises a heavy chain full sequence selected from SEQ ID Nos 207-208, and a light chain variable region selected from SEQ ID Nos 209-212.

30. A complex comprising the antibody or fragment of claim 1, wherein the complex comprises the polypeptide or antibody bound to IgE protein.


SEQUENCE LISTING (342 sequences)

SEQ ID NO: 1 - protein, 118 aa, synthetic construct
  REGION 1..118: note = 21626S-D5
VQLQQPGAELVKPGASVKLSCKASGDTFTEYIIHWVKQRSGQGLEWIGWLSPGSGNIKYNEKFKDKATLTADKSSSTVYMELSRLTSEDSAVYFCARHEVGSYAMDYWGQGTSVTVSS

SEQ ID NO: 2 - protein, 118 aa, synthetic construct
  REGION 1..118: note = 21626B-B9
QTVKESEGRLVTPGTPLTLTCTVSGFSLSNYAMTWVRQAPGKGLEWIGTISSGGSTYYASGAKGRFTISKTSTTVDLKITSPTTEDTATYFCARGVGGGNYYEHFNIWGPGTLVTVSL

SEQ ID NO: 3 - protein, 118 aa, synthetic construct
  REGION 1..118: note = 21626B-H8
QSVEESGGRLVTPGTPLTLTCTASGFSLSTYSMSWVRQAPGKGLEWIGIISSSGNTYYASWAKGRFTISKSSTTVDLKITSPTTEDTATYFCARGVGYNNYYEHFNIWGPGTLVTVSL

SEQ ID NO: 4 - protein, 118 aa, synthetic construct
  REGION 1..118: note = 21626S-E6
QSVKESGGRLVTPGTPLTLTCTVSGFSLSNYAMSWVRQAPGKGLEWIGIISSGASTYYASWAKGRFTISRTSTTVDLKITSPTTEDTATYFCARGYGGANYYEHFNIWGPGTLVTVSL

SEQ ID NO: 5 - protein, 118 aa, synthetic construct
  REGION 1..118: note = 21626S-E12
QTVKESGGRLVTPGTPLTLTCTVSGFSLSNYAMTWVRQAPGKGLEWIGIISSGGYTYYASWAKGRFTISKTSTTVDLKITSPTTEDTATYFCARGYGGGNYYEHYNIWGPGTLVTVSL

SEQ ID NO: 6 - protein, 118 aa, synthetic construct
  REGION 1..118: note = 21626B-F7
QSLEESGGRLVTPGTPLTLTCTVSGFSLSAYTMTWVRQAPGKGLEWIGAISTGGSTYYANWAKGRFTISKTSTTVDLKITSPTTEDTATYFCARGYGSNNYYEHFKIWGPGTLVTVSL

SEQ ID NO: 7 - protein, 119 aa, synthetic construct
  REGION 1..119: note = 21626B-F5
QEQLMESGGRLVTPGTPLTLTCTVSGFSLSSYAMTWVRQAPGKGLEWIGIISTNGNTYYASWAKGRFAISKTSTTVDLKITSPTTEDTATYFCARGYGSNNYYEHFNIWGPGTLVTVSL

SEQ ID NO: 8 - protein, 119 aa, synthetic construct
  REGION 1..119: note = 21626B-C5
QEQLKESGGRLVTPGTPLTLTCTVSGFSLSNYAMSWVRQAPGKGLEWIGIISSSGNTYYASWAKGRFTISKTSTTVDLKITSPTTEDTATYFCARGYGYNNYYEHFNIWGPGTLVTVSL

SEQ ID NO: 9 - protein, 119 aa, synthetic construct
  REGION 1..119: note = 21626S-G9
QEQLEESGGRLVTPGTPLTLTCTASGFSLSAYSMSWVRQAPGKGLEWIGIISSSGYTYYASWAKGRFTISKTSTTVDLEITSPTTEDTATYFCARGYGYNNYYEHFNMWGPGTLVTVSL

SEQ ID NO: 10 - protein, 118 aa, synthetic construct
  REGION 1..118: note = 21626S-F8
QSVKESEGRLVTPGTPLTLTCTVAGFSLSHYHMSWVRQAPGKGLEWIGIIGSSGNTWYASWAKGRFTISKTSTTVDLRITSPTTEDTATYFCARAGASNNYYNYFNVWGPGTLVTVSL

SEQ ID NO: 11 - protein, 118 aa, synthetic construct
  REGION 1..118: note = 21626B-F10
QSVKESGGRLVTPGTPLTLTCTVAGFSLSRYYMSWVRQAPGKGLEWIGIISSSGSTYYASWAKGRFTISKTSTTVDLRITSPTTEDTATYFCARGGASNNYYNYFNIWGPGTLVTVSL

SEQ ID NO: 12 - protein, 124 aa, synthetic construct
  REGION 1..124: note = 21626B-G12
QSLEESGGGLFKPTDTLTLTCTVSGFPLSTYGITWVRQAPGNGLEWIGIISSGGSTHYASWAKSRSTITRDTNENTVTLKMTSLTVADTATYFCARAPYFRWDFNWDLSAFDPWGPGTLVTVSS

SEQ ID NO: 13 - protein, 117 aa, synthetic construct
  REGION 1..117: note = 21626B-A11
QSLGESGGRLVKPDETLTLTCTASGFSLSNYDMGWVRQAPGEGLEYIGWITTGGSAYYANWAKGRFTISRTSTTVDLKISSPTSEDTATYFCARDGGRGYGYSFSIWGPGTLVTVSS

SEQ ID NO: 14 - protein, 119 aa, synthetic construct
  REGION 1..119: note = 21626B-B5
QTVKESGGRLVTPGTPLTLTCTASGFSFSGWSVTWVRQAPGKGLEWIGYIYPRSGSTNYARWVRGRFTISATSTTVDLKLTSPTTEDTATYFCARGGYGGIDWGSMDIWGPGTLVTVSL

SEQ ID NO: 15 - protein, 115 aa, synthetic construct
  REGION 1..115: note = 21626B-C2
QTVKESGGRLVTPGTPLTLTCTASGFSLNSDDMTWVRQAPGMGLEWIGTMHASGFTYYASWAQGRFTISGTSTTVDLKITSPTTEDTATYFCARGAPGYTIDNIWGPGTLVTVSL

SEQ ID NO: 16 - protein, 115 aa, synthetic construct
  REGION 1..115: note = 21626B-H4
QSLEESGGRLVTPGTPLTLTCTVSGFSLSDHAMIWVRQAPGEGLEWVGIIYPSGSTYYANGAKGRFTISKTSTTVDLKMTSLIIEDTATYFCARDYDSGWGADIWGPGTPVTVSL

SEQ ID NO: 17 - protein, 116 aa, synthetic construct
  REGION 1..116: note = 21626B-C8
QTVKESEGGLFKPTDTLTLTCTVSGFSLSIYGVSWVRQAPGNGLEWIGSISATGITYYASWAKSRSTVTRNTNLNTVTLKMTSLTAADTATYFCTRGHTYSTPDIWGPGTLVTVSS

SEQ ID NO: 18 - protein, 118 aa, synthetic construct
  REGION 1..118: note = 21626B-E6
QSLEESGGGLIKPTDTLTLTCTVSGFSLSRYGVIWVRQAPGSGLEWIGAITATGITYYASWAKSRSTITRNTNLNTVTLKMTSLTAADTATYFCAGGHTFSTDVGDIWGPGTPVTVSS

SEQ ID NO: 19 - protein, 115 aa, synthetic construct
  REGION 1..115: note = 21626B-G5
QSVKESEGGLFKPADTLTLTCTVSGFSLNSYTISWVRQAPGNGLEWIGIINTYGTTNYASWAKSRSTITRNTNENTVTLKMTSLTAADTATYFCASLYTTQTNIWGPGTLVTVSS

SEQ ID NO: 20 - protein, 115 aa, synthetic construct
  REGION 1..115: note = 21626B-H3
QSLGESRGRLVTPGGSLTLTCTASGFSLSSYAMGWVRQAPGKGLEYIGWISAGGTTYYASWVNSRFTISRTSTTVDLEMTSLTTEDTATYFCAREGTGWGAYDIWGPGTLVTVSL

SEQ ID NO: 21 - protein, 113 aa, synthetic construct
  REGION 1..113: note = 21626B-A8
QLEESGGRLVTPGTPLTLTCTVSGIDLSSDSIGWVRQAPGKGLEWIGIIYPSGNVYYASWAKGRLTISKTSTTVELKMTSLTTEDTATYFCARDYYSTTLDIWGPGTLVTVSL

SEQ ID NO: 22 - protein, 114 aa, synthetic construct
  REGION 1..114: note = 21626B-D1
QEQLKESRGRLVTPGGSLTLTCTVSGIDLSSNAMSWVRQAPGEGLEWIGTISGGSGSTWYASWAKGRFTISKTSTTVYLKMTSPTTEDTATYFCARGWDGFSTWGPGTLVTVSL

SEQ ID NO: 23 - protein, 113 aa, synthetic construct
  REGION 1..113: note = 21626B-G1
QEQLVESGGRLVTPGSPLTLTCTVSGFSLSSYWMSWVRQAPGKGLEWMGDISNTGKIYYANWAKGRFTISKTSTTVDLTITSPTTEDTATYFCARGMNVYDIWGPGTLVTVSL

SEQ ID NO: 24 - protein, 112 aa, synthetic construct
  REGION 1..112: note = 21626B-C4-
QSLEESGGRLVTPGTPLTLTCTASGFSLSSYYMSWVRQAPEKGLEWIGIIYPSGNTYYANWANGRFAISRTSTTVDLKITSPTTEDTATYFCARDSGYPDIWGPGTLVTVSL

SEQ ID NO: 25 - protein, 119 aa, synthetic construct
  REGION 1..119: note = 21626S-A1
QEQLKESGGRLVTPGTPLTLTCTASGFSLSAYSMSWVRQAPGKGLEWIGIISSSGYTYYASWAKGRFTISKTSTTVDLEITSPTTEDTATYFCARGYGYNNYYEHFNMWGPGTLVTVSL

SEQ ID NO: 26 - protein, 112 aa, synthetic construct
  REGION 1..112: note = 21626S-D5
AQVMAQTPASVSEPVGGTVTIKCQASQSIGNALAWYQQKPGQRPSLLIYDASTLASGVSSRFKGSRSGTEYTLTISDLECADAATYYCQCAYYGSRYAGGSAFGGGTEVVVK

SEQ ID NO: 27 - protein, 112 aa, synthetic construct
  REGION 1..112: note = 21626B-B9
AIDLTQTPASVSEPVGGTVTIKCQASQSIGNALAWYQQKPGQPPKLLIHDASTLASGVSSRFKGSRSGTEYTLTISDLECADAATYYCQCAYYGSRYAGGNAFGGGTEVVVK

SEQ ID NO: 28 - protein, 112 aa, synthetic construct
  REGION 1..112: note = 21626B-H8
DGVMTQTPASVSEPVGGTVTIKCQASQSIGNALAWYQQKPGQPPKLLIHDASTLASGVSSRFKGSRSGTEYTLTISDLECADAATYYCQCAYYGSRYAGGNAFGGGTEVVVK

SEQ ID NO: 29 - protein, 112 aa, synthetic construct
  REGION 1..112: note = 21626S-E6
DVVMTQTPASASEPVGGTVTIKCQASQSIGNALAWYQQKPGQPPKLLIHDASTLASGVSSRFKGSRSGTEYTLTISDLECADAATYYCQCAYYGSRYAGGNAFGGGTEVVVK

SEQ ID NO: 30 - protein, 112 aa, synthetic construct
  REGION 1..112: note = 21626S-E12
DGVMTQTPASVSEPVGGTVTIKCQASQSIGNALAWYQQKPGQRPSLLIYDASTLASGVSSRFKGSRSGTEYTLTISDLECADAATYYCQCAYYGSRYAGGNAFGGGTEVVVK

SEQ ID NO: 31 - protein, 112 aa, synthetic construct
  REGION 1..112: note = 21626B-F7
DVVMTQTPSSVSEPVGGTVTIKCQASQSIGNALAWYQQKPGQRPKLLIHDASTLASGVSSRFKGIRSGTEYTLTISDLECADAATYYCQCAYYGNRYAGGNAFGGGTEVVVK

SEQ ID NO: 32 - protein, 112 aa, synthetic construct; identical to SEQ ID NO: 28
  REGION 1..112: note = 21626B-F5

SEQ ID NO: 33 - protein, 112 aa, synthetic construct
  REGION 1..112: note = 21626B-C5
AQGMTQTPSSVSEPVGGTVTIKCQASQSIGNALAWYQQKPGQRPKLLIHDASTLASGVSSRFKGIRSGTEYTLTISDLECADAATYYCQCAYYGNRYAGGNAFGGGTEVVVK

SEQ ID NO: 34 - protein, 112 aa, synthetic construct
  REGION 1..112: note = 21626S-G9
AQGMTQTPASVSEPVGGTVTIKCQASQSIGNALAWYQQKPGQPPKLLIHDASTLASGVSSRFKGSRSGTEYTLTISDLECADAATYYCQCAYYGSRYAGGNAFGGGTEVVVK

SEQ ID NO: 35 - protein, 112 aa, synthetic construct
  REGION 1..112: note = 21626S-F8
DVVMTQTPASVSEPVGGTATIKCQASQSIGNALAWYQQKPGQPPKLLMHDVSTLASGVSSRFKGSRSGTEFTLTISDLECADAATYYCQCAYYGSRYAGGNAFGGGTEVVVK

SEQ ID NO: 36 - protein, 112 aa, synthetic construct
  REGION 1..112: note = 21626B-F10
AAVMTQTPASVSEPVGGTVTIKCQASQSIGNALAWYQQKPGQPPKLLIHDASTLASGVSSRFKGSRSGTEYTLTISDLECADAATYYCQCAYYGSRYAGGNAFGGGTEVVVK

SEQ ID NO: 37 - protein, 110 aa, synthetic construct
  REGION 1..110: note = 21626B-G12
AQVLTQTPASVSAAVGGTVTISCQSSESVYKNNYLSWYQQKPGQPPKGLIYLSSTLASGVPSRFSGSGSGTQFTLTISDLECSDAATYYCAGGYSGNIGAFGGGTEVVVK

SEQ ID NO: 38 - protein, 110 aa, synthetic construct
  REGION 1..110: note = 21626B-A11
ALVMTQTPASVEAAVGGTVTINCQASQNIGNYLSWFQQKPGQRPKLLIYKASTLASGVSSRFKGSGSGTQFTLTISGVECADAATYYCQQDAAVINVDNTFGGGTEVVVK

SEQ ID NO: 39 - protein, 114 aa, synthetic construct
  REGION 1..114: note = 21626B-B5
AVVLTQTPSSSSAAVGGTVTINCQASQSVYNNNYLSWYQQKPGQPPKLLIYGASKLASGVPARFSGSRSGTDFTLTINDLECADAATYYCQSTYGSNSNGWDNAFGGGTEVVVR

SEQ ID NO: 40 - protein, 113 aa, synthetic construct
  REGION 1..113: note = 21626B-C2
AQVLTQTASPVSAAVGGTVTINCQSSQSIYGNNWLGWYQQKPGQPPKQLIYKASTLASGVSSRFKGSGSGTQFTLTISDVQCDDAATYYCLGEFSCTSGDCYTFGGGTEVVVK

SEQ ID NO: 41 - protein, 110 aa, synthetic construct
  REGION 1..110: note = 21626B-H4
ALVMTQTPASVSAAVGGTVTINCQASQSIHKYLSWYQQKPGQPPKLLIYRASTLASGVPSRFKGSGSGTEFTLTISDLECADAATYYCQQDAAVINIDNSFGGGTEVVVK

SEQ ID NO: 42 - protein, 112 aa, synthetic construct
  REGION 1..112: note = 21626B-C8
ALVLTQTPSSKSVPVGDTVTINCQASESVYNKNYLAWFQQKPGQPPKLLIYKASTLASGVPSRFKGSGSGTQFTLTINDVVCDDAATYFCAGYKDAATDGNAFGGGTEVVVK

SEQ ID NO: 43 - protein, 112 aa, synthetic construct
  REGION 1..112: note = 21626B-E6
ALVLTQTPSSKSVPVGDTVTINCQASESVWNKNYLAWFQQKPGQPPKLLIYKASTLASGVPSRFKGSGSGTQFTLTINDVVCGDAATYFCAGYKDIDIDGNAFGGGTEVVVK

SEQ ID NO: 44 - protein, 110 aa, synthetic construct
  REGION 1..110: note = 21626B-G5
DPVMTQTPSSTSAAVGGTVTINCQASQSVATKNQLAWYQQKPGQPPKLLIYTTSRLASGVPSRFSGSGSGAQFTLTISGVQCDDAATYYCLGTYANPIYTFGGGTEVVVK

SEQ ID NO: 45 - protein, 110 aa, synthetic construct
  REGION 1..110: note = 21626B-H3
ALVMTQTPASVSAAVGGTVTINCQASQSIHKYLSWYQQKPGQPPKLLIYRASTLASGVPSRFKGSGSGTQFTLTISDLECDDAATYYCQQDAAVISIDNSFGGGTEVVVK

SEQ ID NO: 46 - protein, 110 aa, synthetic construct
  REGION 1..110: note = 21626B-A8
ALVMTQTPSSVSAVVGGTVTINCQASQSISSYLSWYQQKPGQRPKPLIYEASKLASGVSSRFKGSGSGTEFTLTISGVECADAATYYCQQDAAVMNVDNTFGGGTEVVVK

SEQ ID NO: 47 - protein, 110 aa, synthetic construct
  REGION 1..110: note = 21626B-D1
ALVMTQTPASVSAAVGGTVTINCQASRSIHKYLSWYQQKPGQPPKLLIYRASTLASGVPSRFKGSGSGTEFTLTISDLECADAATYYCQQDAAVINIDNSFGGGTEVVVK

SEQ ID NO: 48 - protein, 110 aa, synthetic construct
  REGION 1..110: note = 21626B-G1
ALVMTQTASPVSAAVGGTVTINCQASQSIHKYLSWYQQKPGQPPKLLIYRASTLASGVPSRFKGSGSGTEFTLTISDLECADAATYYCQQDAAVINIDNSFGGGTEVVVK

SEQ ID NO: 49 - protein, 110 aa, synthetic construct
  REGION 1..110: note = 21626B-C4-8
ALVMTQTPSSVSAAVGGTVTINCQASQSISSYLSWYQQKPGQRPKPLIYETSKLASGVSSRFSGSGSGTEFTLTISGVECADAATYYCQQDAGVINVHNTFGGGTEVVVK

SEQ ID NO: 50 - protein, 112 aa, synthetic construct
  REGION 1..112: note = 21626S-A1
DVVMTQTPASVSEPVGGTVTIKCQASQSIGNALAWYQQKPGQRPSLLIYDASTLASGVSSRFKGSRSGTEYTLTISDLECADAATYYCQCAYYGSRYAGGSAFGGGTEVVVK

SEQ ID NO: 51 - protein, 8 aa, synthetic construct
  REGION 1..8: note = 21626S-D5 HCDR1
GFSLSNYA

SEQ ID NO: 52 - protein, 8 aa, synthetic construct
  REGION 1..8: note = 21626B-B9 HCDR1
GFSLSNYA

SEQ ID NO: 53 - protein, 8 aa, synthetic construct
  REGION 1..8: note = 21626B-H8 HCDR1
GFSLSTYS

SEQ ID NO: 54 - protein, 8 aa, synthetic construct
  REGION 1..8: note = 21626S-E6 HCDR1
GFSLSNYA

SEQ ID NO: 55 - protein, 8 aa, synthetic construct
  REGION 1..8: note = 21626S-E12 HCDR1
GFSLSNYA

SEQ ID NO: 56 - protein, 8 aa, synthetic construct
  REGION 1..8: note = 21626B-F7 HCDR1
GFSLSAYT

SEQ ID NO: 57 - protein, 8 aa, synthetic construct
  REGION 1..8: note = 21626B-F5 HCDR1
GFSLSSYA

SEQ ID NO: 58 - protein, 8 aa, synthetic construct
  REGION 1..8: note = 21626B-C5 HCDR1
GFSLSNYA

SEQ ID NO: 59 - protein, 8 aa, synthetic construct
  REGION 1..8: note = 21626S-G9 HCDR1
GFSLSAYS

SEQ ID NO: 60 - protein, 8 aa, synthetic construct
  REGION 1..8: note = 21626S-F8 HCDR1
GFSLSHYH

SEQ ID NO: 61 - protein, 8 aa, synthetic construct
  REGION 1..8: note = 21626B-F10 HCDR1
GFSLSRYY

SEQ ID NO: 62 - protein, 8 aa, synthetic construct
  REGION 1..8: note = 21626B-G12 HCDR1
GFPLSTYG

SEQ ID NO: 63 - protein, 8 aa, synthetic construct
  REGION 1..8: note = 21626B-A11 HCDR1
GFSLSNYD

SEQ ID NO: 64 - protein, 8 aa, synthetic construct
  REGION 1..8: note = 21626B-B5 HCDR1
GFSFSGWS

SEQ ID NO: 65 - protein, 8 aa, synthetic construct
  REGION 1..8: note = 21626B-C2 HCDR1
GFSLNSDD

SEQ ID NO: 66 - protein, 8 aa, synthetic construct
  REGION 1..8: note = 21626B-H4 HCDR1
GFSLSDHA

SEQ ID NO: 67 - protein, 8 aa, synthetic construct
  REGION 1..8: note = 21626B-C8 HCDR1
GFSLSIYG

SEQ ID NO: 68 - protein, 8 aa, synthetic construct
  REGION 1..8: note = 21626B-E6 HCDR1
GFSLSRYG

SEQ ID NO: 69 - protein, 8 aa, synthetic construct
  REGION 1..8: note = 21626B-G5 HCDR1
GFSLNSYT

SEQ ID NO: 70 - protein, 8 aa, synthetic construct
  REGION 1..8: note = 21626B-H3 HCDR1
GFSLSSYA

SEQ ID NO: 71 - protein, 8 aa, synthetic construct
  REGION 1..8: note = 21626B-A8 HCDR1
GIDLSSDS

SEQ ID NO: 72 - protein, 8 aa, synthetic construct
  REGION 1..8: note = 21626B-D1 HCDR1
GIDLSSNA

SEQ ID NO: 73 - protein, 8 aa, synthetic construct
  REGION 1..8: note = 21626B-G1 HCDR1
GFSLSSYW

SEQ ID NO: 74 - protein, 8 aa, synthetic construct
  REGION 1..8: note = 21626B-C4-8 HCDR1
GFSLSSYY

SEQ ID NO: 75 - protein, 8 aa, synthetic construct
  REGION 1..8: note = 26126S-A1 HCDR1
GFSLSAYS

SEQ ID NO: 76 - protein, 7 aa, synthetic construct
  REGION 1..7: note = 21626S-D5 HCDR2
IGSGGYT

SEQ ID NO: 77 - protein, 7 aa, synthetic construct
  REGION 1..7: note = 21626B-B9 HCDR2
ISSGGST

SEQ ID NO: 78 - protein, 7 aa, synthetic construct
  REGION 1..7: note = 21626B-H8 HCDR2
ISSSGNT

SEQ ID NO: 79 - protein, 7 aa, synthetic construct
  REGION 1..7: note = 21626S-E6 HCDR2
ISSGAST

SEQ ID NO: 80 - protein, 7 aa, synthetic construct
  REGION 1..7: note = 21626S-E12 HCDR2
ISSGGYT

SEQ ID NO: 81 - protein, 7 aa, synthetic construct
  REGION 1..7: note = 21626B-F7 HCDR2
ISTGGST

SEQ ID NO: 82 - protein, 7 aa, synthetic construct
  REGION 1..7: note = 21626B-F5 HCDR2
ISTNGNT

SEQ ID NO: 83 - protein, 7 aa, synthetic construct
  REGION 1..7: note = 21626B-C5 HCDR2
ISSSGNT

SEQ ID NO: 84 - protein, 7 aa, synthetic construct
  REGION 1..7: note = 21626S-G9 HCDR2
ISSSGYT

SEQ ID NO: 85 - protein, 7 aa, synthetic construct
  REGION 1..7: note = 21626S-F8 HCDR2
IGSSGNT

SEQ ID NO: 86 - protein, 7 aa, synthetic construct
  REGION 1..7: note = 21626B-F10 HCDR2
ISSSGST

SEQ ID NO: 87 - protein, 7 aa, synthetic construct
  REGION 1..7: note = 21626B-G12 HCDR2
ISSGGST

SEQ ID NO: 88 - protein, 7 aa, synthetic construct
  REGION 1..7: note = 21626B-A11 HCDR2
ITTGGSA

SEQ ID NO: 89 - protein, 8 aa, synthetic construct
  REGION 1..8: note = 21626B-B5 HCDR2
IYPRSGST

SEQ ID NO: 90 - protein, 7 aa, synthetic construct
  REGION 1..7: note = 21626B-C2 HCDR2
MHASGFT

SEQ ID NO: 91 - protein, 7 aa, synthetic construct
  REGION 1..7: note = 21626B-H4 HCDR2
IYPSGST

SEQ ID NO: 92 - protein, 7 aa, synthetic construct
  REGION 1..7: note = 21626B-C8 HCDR2
ISATGIT

SEQ ID NO: 93 - protein, 7 aa, synthetic construct
  REGION 1..7: note = 21626B-E6 HCDR2
ITATGIT

SEQ ID NO: 94 - protein, 7 aa, synthetic construct
  REGION 1..7: note = 21626B-G5 HCDR2
INTYGTT

SEQ ID NO: 95 - protein, 7 aa, synthetic construct
  REGION 1..7: note = 21626B-H3 HCDR2
ISAGGTT

SEQ ID NO: 96 - protein, 7 aa, synthetic construct
  REGION 1..7: note = 21626B-A8 HCDR2
IYPSGNV

SEQ ID NO: 97 - protein, 8 aa, synthetic construct
  REGION 1..8: note = 21626B-D1 HCDR2
ISGGSGST

SEQ ID NO: 98 - protein, 7 aa, synthetic construct
  REGION 1..7: note = 21626B-G1 HCDR2
ISNTGKI

SEQ ID NO: 99 - protein, 7 aa, synthetic construct
  REGION 1..7: note = 21626B-C4-8 HCDR2
IYPSGNT

SEQ ID NO: 100 - protein, 7 aa, synthetic construct
  REGION 1..7: note = 26126S-A1 HCDR2
ISSSGYT

SEQ ID NO: 101 - protein, 15 aa, synthetic construct
  REGION 1..15: note = 21626S-D5 HCDR3
ARGVGGANYYEHFNI

SEQ ID NO: 102 - protein, 15 aa, synthetic construct
  REGION 1..15: note = 21626B-B9 HCDR3
ARGVGGGNYYEHFNI

SEQ ID NO: 103 - protein, 15 aa, synthetic construct
  REGION 1..15: note = 21626B-H8 HCDR3
ARGVGYNNYYEHFNI

SEQ ID NO: 104 - protein, 15 aa, synthetic construct
  REGION 1..15: note = 21626S-E6 HCDR3
ARGYGGANYYEHFNI

SEQ ID NO: 105 - protein, 15 aa, synthetic construct
  REGION 1..15: note = 21626S-E12 HCDR3
ARGYGGGNYYEHYNI

SEQ ID NO: 106 - protein, 15 aa, synthetic construct
  REGION 1..15: note = 21626B-F7 HCDR3
ARGYGSNNYYEHFKI

SEQ ID NO: 107 - protein, 15 aa, synthetic construct
  REGION 1..15: note = 21626B-F5 HCDR3
ARGYGSNNYYEHFNI

SEQ ID NO: 108 - protein, 15 aa, synthetic construct
  REGION 1..15: note = 21626B-C5 HCDR3
ARGYGYNNYYEHFNI

SEQ ID NO: 109 - protein, 15 aa, synthetic construct
  REGION 1..15: note = 21626S-G9 HCDR3
ARGYGYNNYYEHFNM

SEQ ID NO: 110 - protein, 15 aa, synthetic construct
  REGION 1..15: note = 21626S-F8 HCDR3
ARAGASNNYYNYFNV

SEQ ID NO: 111 - protein, 15 aa, synthetic construct
  REGION 1..15: note = 21626B-F10 HCDR3
ARGGASNNYYNYFNI

SEQ ID NO: 112 - protein, 19 aa, synthetic construct
  REGION 1..19: note = 21626B-G12 HCDR3
ARAPYFRWDFNWDLSAFDP

SEQ ID NO: 113 - protein, 14 aa, synthetic construct
  REGION 1..14: note = 21626B-A11 HCDR3
ARDGGRGYGYSFSI

SEQ ID NO: 114 - protein, 15 aa, synthetic construct
  REGION 1..15: note = 21626B-B5 HCDR3
ARGGYGGIDWGSMDV

SEQ ID NO: 115 - protein, 12 aa, synthetic construct
  REGION 1..12: note = 21626B-C2 HCDR3
ARGAPGYTIDNI

SEQ ID NO: 116 - protein, 12 aa, synthetic construct
  REGION 1..12: note = 21626B-H4 HCDR3
ARDYDSGWGADI

SEQ ID NO: 117 - protein, 11 aa, synthetic construct
  REGION 1..11: note = 21626B-C8 HCDR3
TRGHTYSTPDI

SEQ ID NO: 118 - protein, 13 aa, synthetic construct
  REGION 1..13: note = 21626B-E6 HCDR3
AGGHTFSTDVGDI

SEQ ID NO: 119 - protein, 10 aa, synthetic construct
  REGION 1..10: note = 21626B-G5 HCDR3
ASLYTTQTNI

SEQ ID NO: 120 - protein, 12 aa, synthetic construct
  REGION 1..12: note = 21626B-H3 HCDR3
AREGTGWGAYDI

SEQ ID NO: 121 - protein, 11 aa, synthetic construct
  REGION 1..11: note = 21626B-A8 HCDR3
ARDYYSTTLDI

SEQ ID NO: 122 - protein, 9 aa, synthetic construct
  REGION 1..9: note = 21626B-D1 HCDR3
ARGWDGFST

SEQ ID NO: 123 - protein, 9 aa, synthetic construct
  REGION 1..9: note = 21626B-G1 HCDR3
ARGMNVYDI

SEQ ID NO: 124 - protein, 9 aa, synthetic construct
  REGION 1..9: note = 21626B-C4-8 HCDR3
ARDSGYPDI

SEQ ID NO: 125 - protein, 15 aa, synthetic construct
  REGION 1..15: note = 26126S-A1 HCDR3
ARGYGYNNYYEHFNM

SEQ ID NO: 126 - protein, 6 aa, synthetic construct
  REGION 1..6: note = 21626S-D5 LCDR1
QSIGNA

SEQ ID NO: 127 - protein, 6 aa, synthetic construct
  REGION 1..6: note = 21626B-B9 LCDR1
QSIGNA

SEQ ID NO: 128 - protein, 6 aa, synthetic construct
  REGION 1..6: note = 21626B-H8 LCDR1
QSIGNA

SEQ ID NO: 129 - protein, 6 aa, synthetic construct
  REGION 1..6: note = 21626S-E6 LCDR1
QSIGNA

SEQ ID NO: 130 - protein, 6 aa, synthetic construct
  REGION 1..6: note = 21626S-E12 LCDR1
QSIGNA

SEQ ID NO: 131 - protein, 6 aa, synthetic construct
  REGION 1..6: note = 21626B-F7 LCDR1
QSIGNA

SEQ ID NO: 132 - protein, 6 aa, synthetic construct
  REGION 1..6: note = 21626B-F5 LCDR1
QSIGNA

SEQ ID NO: 133 - protein, 6 aa, synthetic construct
  REGION 1..6: note = 21626B-C5 LCDR1
QSIGNA

SEQ ID NO: 134 - protein, 6 aa, synthetic construct
  REGION 1..6: note = 21626S-G9 LCDR1
QSIGNA

SEQ ID NO: 135 - protein, 6 aa, synthetic construct
  REGION 1..6: note = 21626S-F8 LCDR1
QSIGNA

SEQ ID NO: 136 - protein, 6 aa, synthetic construct
  REGION 1..6: note = 21626B-F10 LCDR1
QSIGNA

SEQ ID NO: 137 - protein, 8 aa, synthetic construct
  REGION 1..8: note = 21626B-G12 LCDR1
ESVYKNNY

SEQ ID NO: 138 - protein, 6 aa, synthetic construct
  REGION 1..6: note = 21626B-A11 LCDR1
QNIGNY

SEQ ID NO: 139 - protein, 8 aa, synthetic construct
  REGION 1..8: note = 21626B-B5 LCDR1
QSVYNNNY

SEQ ID NO: 140 - protein, 8 aa, synthetic construct
  REGION 1..8: note = 21626B-C2 LCDR1
QSIYGNNW

SEQ ID NO: 141 - protein, 6 aa, synthetic construct
  REGION 1..6: note = 21626B-H4 LCDR1
QSIHKY

SEQ ID NO: 142 - protein, 8 aa, synthetic construct
  REGION 1..8: note = 21626B-C8 LCDR1
ESVYNKNY

SEQ ID NO: 143 - protein, 8 aa, synthetic construct
  REGION 1..8: note = 21626B-E6 LCDR1
ESVWNKNY

SEQ ID NO: 144 - protein, 8 aa, synthetic construct
  REGION 1..8: note = 21626B-G5 LCDR1
QSVATKNQ

SEQ ID NO: 145 - protein, 6 aa, synthetic construct
  REGION 1..6: note = 21626B-H3 LCDR1
QSIHKY

SEQ ID NO: 146 - protein, 6 aa, synthetic construct
  REGION 1..6: note = 21626B-A8 LCDR1
QSISSY

SEQ ID NO: 147 - protein, 6 aa, synthetic construct
  REGION 1..6: note = 21626B-D1 LCDR1
RSIHKY

SEQ ID NO: 148 - protein, 6 aa, synthetic construct
  REGION 1..6: note = 21626B-G1 LCDR1
QSIHKY

SEQ ID NO: 149 - protein, 6 aa, synthetic construct
  REGION 1..6: note = 21626B-C4-8 LCDR1
QSVNNN

SEQ ID NO: 150 - protein, 6 aa, synthetic construct
  REGION 1..6: note = 26126S-A1 LCDR1
QSIGNA

SEQ ID NO: 151-175 - skipped (25 entries)

SEQ ID NO: 176 - protein, 14 aa, synthetic construct
  REGION 1..14: note = 21626S-D5 LCDR3
QCAYYGSRYAGGSA

SEQ ID NO: 177 - protein, 14 aa, synthetic construct
  REGION 1..14: note = 21626B-B9 LCDR3
QCAYYGSRYAGGNA

SEQ ID NO: 178 - protein, 14 aa, synthetic construct
  REGION 1..14: note = 21626B-H8 LCDR3
QCAYYGSRYAGGNA

SEQ ID NO: 179 - protein, 14 aa, synthetic construct
  REGION 1..14: note = 21626S-E6 LCDR3
QCAYYGSRYAGGNA

SEQ ID NO: 180 - protein, 14 aa, synthetic construct
  REGION 1..14: note = 21626S-E12 LCDR3
QCAYYGSRYAGGNA

SEQ ID NO: 181 - protein, 14 aa, synthetic construct
  REGION 1..14: note = 21626B-F7 LCDR3
QCAYYGNRYAGGNA

SEQ ID NO: 182 - protein, 14 aa, synthetic construct
  REGION 1..14: note = 21626B-F5 LCDR3
QCAYYGSRYAGGNA

SEQ ID NO: 183 - protein, 14 aa, synthetic construct
  REGION 1..14: note = 21626B-C5 LCDR3
QCAYYGNRYAGGNA

SEQ ID NO: 184 - protein, 14 aa, synthetic construct
  REGION 1..14: note = 21626S-G9 LCDR3
QCAYYGSRYAGGNA

SEQ ID NO: 185 - protein, 14 aa, synthetic construct
  REGION 1..14: note = 21626S-F8 LCDR3
QCAYYGSRYAGGNA

SEQ ID NO: 186 - protein, 14 aa, synthetic construct
  REGION 1..14: note = 21626B-F10 LCDR3
QCAYYGSRYAGGNA

SEQ ID NO: 187 - protein, 10 aa, synthetic construct
  REGION 1..10: note = 21626B-G12 LCDR3
AGGYSGNIGA

SEQ ID NO: 188 - protein, 12 aa, synthetic construct
  REGION 1..12: note = 21626B-A11 LCDR3
QQDAAVINVDNT

SEQ ID NO: 189 - protein, 14 aa, synthetic construct
  REGION 1..14: note = 21626B-B5 LCDR3
QSTYGSNSNGWDNA

SEQ ID NO: 190 - protein, 13 aa, synthetic construct
  REGION 1..13: note = 21626B-C2 LCDR3
LGEFSCTSGDCYT

SEQ ID NO: 191 - protein, 12 aa, synthetic construct
  REGION 1..12: note = 21626B-H4 LCDR3
QQDAAVINIDNS

SEQ ID NO: 192 - protein, 12 aa, synthetic construct
  REGION 1..12: note = 21626B-C8 LCDR3
AGYKDAATDGNA

SEQ ID NO: 193 - protein, 12 aa, synthetic construct
  REGION 1..12: note = 21626B-E6 LCDR3
AGYKDIDIDGNA

SEQ ID NO: 194 - protein, 10 aa, synthetic construct
  REGION 1..10: note = 21626B-G5 LCDR3
LGTYANPIYT

SEQ ID NO: 195 - protein, 12 aa, synthetic construct
  REGION 1..12: note = 21626B-H3 LCDR3
QQDAAVISIDNS

SEQ ID NO: 196 - protein, 12 aa, synthetic construct
  REGION 1..12: note = 21626B-A8 LCDR3
QQDAAVMNVDNT

SEQ ID NO: 197 - protein, 12 aa, synthetic construct
  REGION 1..12: note = 21626B-D1 LCDR3
QQDAAVINIDNS

SEQ ID NO: 198 - protein, 12 aa, synthetic construct
  REGION 1..12: note = 21626B-G1 LCDR3
QQDAAVINIDNS

SEQ ID NO: 199 - protein, 12 aa, synthetic construct
  REGION 1..12: note = 21626B-C4-8 LCDR3
QQDAGVINVHNN

SEQ ID NO: 200 - protein, 14 aa, synthetic construct
  REGION 1..14: note = 26126S-A1 LCDR3
QCAYYGSRYAGGSA

SEQ ID NO: 201 - protein, 117 aa, synthetic construct
  REGION 1..117: note = h-21626B-C4-8
LEEVQLVESGGGLVQPGGSLRLSCTASGFSLSSYYMSWVRQAPGKGLEWIGIIYPSGNTYYANWANGRFTISRHNSKNTLYLQMNSLRAEDTAVYYCARDSGYPDIWGQGTLVTVSS

SEQ ID NO: 202 - protein, 120 aa, synthetic construct
  REGION 1..120: note = h-21626B-H3
LEQVQLVESGGGLVQPGGSLRLSCTASGFSLSSYAMGWVRQAPGKGLEYIGWISAGGTTYYASWVNSRFTISRDNSKNTLYLQMNSLRAEDTAVYYCAREGTGWGAYDIWGQGTLVTVSS

SEQ ID NO: 203 - protein, 112 aa, synthetic construct
  REGION 1..112: note = h-21626B-C4-8
SRDIQMTQSPSSLSASVGDRVTITCQASQSISSYLSWYQQKPGKAPKLLIYETSKLASGVPSRFSGSGSGTDFTLTISSLQPEDFATYYCQQDAGVINVHNTFGGGTKVEIK

SEQ ID NO: 204 - protein, 112 aa, synthetic construct
  REGION 1..112: note = h-21626B-C4-8E
SRDIQMTQSPSSLSASVGDRVTITCQASQSISSYLSWYQQKPGKAPKLLIYETSKLASGVPSRFSGSGSGTEFTLTISSLQPEDFATYYCQQDAGVINVHNTFGGGTKVEIK

SEQ ID NO: 205 - protein, 112 aa, synthetic construct
  REGION 1..112: note = h-21626B-H3
SRDIQMTQSPSSLSASVGDRVTITCQASQSIHKYLSWYQQKPGKAPKLLIYRASTLASGVPSRFSGSGSGTDFTLTISSLQPEDFATYYCQQDAAVISIDNSFGGGTKVEIK

SEQ ID NO: 206 - protein, 112 aa, synthetic construct
  REGION 1..112: note = h-21626B-H3KQ
SRDIQMTQSPSSLSASVGDRVTITCQASQSIHKYLSWYQQKPGKAPKLLIYRASTLASGVPSRFKGSGSGTQFTLTISSLQPEDFATYYCQQDAAVISIDNSFGGGTKVEIK

SEQ ID NO: 207 - protein, 222 aa, synthetic construct
  REGION 1..222: note = h-21626B-C4-8
LEEVQLVESGGGLVQPGGSLRLSCTASGFSLSSYYMSWVRQAPGKGLEWIGIIYPSGNTYYANWANGRFTISRHNSKNTLYLQMNSLRAEDTAVYYCARDSGYPDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 208 - protein, 225 aa, synthetic construct
  REGION 1..225: note = h-21626B-H3
LEQVQLVESGGGLVQPGGSLRLSCTASGFSLSSYAMGWVRQAPGKGLEYIGWISAGGTTYYASWVNSRFTISRDNSKNTLYLQMNSLRAEDTAVYYCAREGTGWGAYDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 209 - protein, 219 aa, synthetic construct
  REGION 1..219: note = h-21626B-C4-8
SRDIQMTQSPSSLSASVGDRVTITCQASQSISSYLSWYQQKPGKAPKLLIYETSKLASGVPSRFSGSGSGTDFTLTISSLQPEDFATYYCQQDAGVINVHNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 210 - protein, 219 aa, synthetic construct
  REGION 1..219: note = h-21626B-C4-8E
SRDIQMTQSPSSLSASVGDRVTITCQASQSISSYLSWYQQKPGKAPKLLIYETSKLASGVPSRFSGSGSGTEFTLTISSLQPEDFATYYCQQDAGVINVHNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - protein, 219 aa, synthetic construct
  REGION 1..219: note = h-21626B-H3
SRDIQMTQSPSSLSASVGDRVTITCQASQSIHKYLSWYQQKPGKAPKLLIYRASTLASGVPSRFSGSGSGTDFTLTISSLQPEDFATYYCQQDAAVISIDNSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 212 - protein, 219 aa, synthetic construct
  REGION 1..219: note = h-21626B-H3KQ
SRDIQMTQSPSSLSASVGDRVTITCQASQSIHKYLSWYQQKPGKAPKLLIYRASTLASGVPSRFKGSGSGTQFTLTISSLQPEDFATYYCQQDAAVISIDNSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 213 - DNA, 354 bp, synthetic construct
  misc_feature 1..354: note = 21626S-D5 Coding Sequence
  source 1..354: mol_type = other DNA
gtccaactgcagcagcctggagctgaactggtgaaacccggggcatcagtgaagctgtcctgcaaggcttctggcgacaccttcactgagtatattatacactgggtaaagcagaggtctggacagggtcttgagtggattgggtggttatcccctggaagtggtaatataaagtataatgagaaattcaaggacaaggccacattgactgcggacaaatcctccagcacagtctatatggagcttagtagattgacatctgaagactctgcggtctatttctgtgcaagacacgaagtggggtcttatgctatggactactggggtcaaggaacctcagtcaccgtctcctca

SEQ ID NO: 214 - DNA, 354 bp, synthetic construct
  misc_feature 1..354: note = 21626S-D5 Coding Complementary Sequence
  source 1..354: mol_type = other DNA
tgaggagacggtgactgaggttccttgaccccagtagtccatagcataagaccccacttcgtgtcttgcacagaaatagaccgcagagtcttcagatgtcaatctactaagctccatatagactgtgctggaggatttgtccgcagtcaatgtggccttgtccttgaatttctcattatactttatattaccacttccaggggataaccacccaatccactcaagaccctgtccagacctctgctttacccagtgtataatatactcagtgaaggtgtcgccagaagccttgcaggacagcttcactgatgccccgggtttcaccagttcagctccaggctgctgcagttggac

SEQ ID NO: 215 - DNA, 354 bp, synthetic construct
  misc_feature 1..354: note = 21626B-B9 Coding Sequence
  source 1..354: mol_type = other DNA
cagacggtgaaggagtccgagggtcgcctggtcacgcctgggacacccctgacactgacctgcacagtctctggattctccctcagtaactatgcaatgacctgggtccgccaggctccagggaaggggctggaatggatcggaaccattagtagtggtggtagtacatactacgcgagcggggcgaaaggccgattcaccatctccaagacctcgaccacggtggatctgaagatcaccagtccgacaaccgaggacacggccacctatttctgtgccagaggtgttggtggtggtaattattatgagcactttaacatctggggcccaggcaccctggtcaccgtctcctta

SEQ ID NO: 216 - DNA, 354 bp, synthetic construct
  misc_feature 1..354: note = 21626B-B9 Coding Complementary Sequence
  source 1..354: mol_type = other DNA
taaggagacggtgaccagggtgcctgggccccagatgttaaagtgctcataataattaccaccaccaacacctctggcacagaaataggtggccgtgtcctcggttgtcggactggtgatcttcagatccaccgtggtcgaggtcttggagatggtgaatcggcctttcgccccgctcgcgtagtatgtactaccaccactactaatggttccgatccattccagccccttccctggagcctggcggacccaggtcattgcatagttactgagggagaatccagagactgtgcaggtcagtgtcaggggtgtcccaggcgtgaccaggcgaccctcggactccttcaccgtctg

SEQ ID NO: 217 - DNA, 354 bp, synthetic construct
  misc_feature 1..354: note = 21626B-H8 Coding Sequence
  source 1..354: mol_type = other DNA
cagtcggtggaggagtccgggggtcgcctggtcacgcctgggacacccctgacactcacctgcacagcctctggattctccctcagtacctactccatgagctgggtccgccaggctccagggaaggggctggaatggatcggaatcattagtagtagtggtaacacatactacgcgagctgggcgaaaggccgattcaccatctccaagtcctcgaccacggtggatctgaaaatcaccagtccgacaaccgaggacacggccacctatttctgtgccagaggtgttggttataataattattatgagcactttaacatctggggcccaggcaccctggtcaccgtctcctta

SEQ ID NO: 218 - DNA, 354 bp, synthetic construct
  misc_feature 1..354: note = 21626B-H8 Coding Complementary Sequence
  source 1..354: mol_type = other DNA
taaggagacggtgaccagggtgcctgggccccagatgttaaagtgctcataataattattataaccaacacctctggcacagaaataggtggccgtgtcctcggttgtcggactggtgattttcagatccaccgtggtcgaggacttggagatggtgaatcggcctttcgcccagctcgcgtagtatgtgttaccactactactaatgattccgatccattccagccccttccctggagcctggcggacccagctcatggagtaggtactgagggagaatccagaggctgtgcaggtgagtgtcaggggtgtcccaggcgtgaccaggcgacccccggactcctccaccgactg

SEQ ID NO: 219 - DNA, 354 bp, synthetic construct
  misc_feature 1..354: note = 21626S-E6 Coding Sequence
  source 1..354: mol_type = other DNA
cagtcggtgaaggagtccgggggtcgcctggtcacgcctgggacacccctgacactcacctgcacagtctctggattctccctcagtaactatgcaatgagctgggtccgccaggctccagggaaggggctggaatggatcggaatcattagtagtggtgctagcacatactacgcgagctgggcgaaaggccgattcaccatctccagaacctcgaccacggtggatctgaaaatcaccagtccgacaaccgaggacacggccacctatttctgtgccagaggttatggtggtgctaattattatgagcactttaacatctggggcccaggcaccctggtcaccgtctcctta

SEQ ID NO: 220 - DNA, 354 bp, synthetic construct
  misc_feature 1..354: note = 21626S-E6 Coding Complementary Sequence
  source 1..354: mol_type = other DNA
taaggagacggtgaccagggtgcctgggccccagatgttaaagtgctcataataattagcaccaccataacctctggcacagaaataggtggccgtgtcctcggttgtcggactggtgattttcagatccaccgtggtcgaggttctggagatggtgaatcggcctttcgcccagctcgcgtagtatgtgctagcaccactactaatgattccgatccattccagccccttccctggagcctggcggacccagctcattgcatagttactgagggagaatccagagactgtgcaggtgagtgtcaggggtgtcccaggcgtgaccaggcgacccccggactccttcaccgactg

SEQ ID NO: 221 - DNA, 354 bp, synthetic construct
  misc_feature 1..354: note = 21626S-E12 Coding Sequence
  source 1..354: mol_type = other DNA
cagacagtgaaggagtccgggggtcgcctggtcacgcctgggacacccctgacactcacctgcacagtctctggattctccctcagtaactatgcaatgacctgggtccgccaggctccggggaaggggctggaatggatcggaatcattagtagtggtggttatacatactacgcgagctgggcgaaaggccgattcaccatctccaaaacctcgaccacggtggatctgaaaatcaccagtccgacaaccgaggacacggccacctatttctgtgccagaggttatggtggtggtaactattatgagcactataacatctggggcccaggcaccctggtcaccgtctcctta

SEQ ID NO: 222 - DNA, 354 bp, synthetic construct
  misc_feature 1..354: note = 21626S-E12 Coding Complementary Sequence
  source 1..354: mol_type = other DNA
taaggagacggtgaccagggtgcctgggccccagatgttatagtgctcataatagttaccaccaccataacctctggcacagaaataggtggccgtgtcctcggttgtcggactggtgattttcagatccaccgtggtcgaggttttggagatggtgaatcggcctttcgcccagctcgcgtagtatgtataaccaccactactaatgattccgatccattccagccccttccccggagcctggcggacccaggtcattgcatagttactgagggagaatccagagactgtgcaggtgagtgtcaggggtgtcccaggcgtgaccaggcgacccccggactccttcactgtctg

SEQ ID NO: 223 - DNA, 354 bp, synthetic construct
  misc_feature 1..354: note = 21626B-F7 Coding Sequence
  source 1..354: mol_type = other DNA
cagtcgctggaggagtccgggggtcgcctggtcacgcctgggacacccctgacactcacctgcacagtctctggattctccctcagtgcctatacaatgacctgggtccgccaggctccagggaaggggctggaatggatcggagccattagtactggtggtagcacatactacgcgaactgggcgaaaggccgattcaccatctccaaaacctcgaccacggtggatctgaaaatcaccagtccgacaaccgaggacacggccacctatttctgtgccagaggttatggtagtaataattattatgagcactttaagatctggggcccaggcaccctggtcaccgtctcctta

SEQ ID NO: 224 - DNA, 354 bp, synthetic construct
  misc_feature 1..354: note = 21626B-F7 Coding Complementary Sequence
  source 1..354: mol_type = other DNA
taaggagacggtgaccagggtgcctgggccccagatcttaaagtgctcataataattattactaccataacctctggcacagaaataggtggccgtgtcctcggttgtcggactggtgattttcagatccaccgtggtcgaggttttggagatggtgaatcggcctttcgcccagttcgcgtagtatgtgctaccaccagtactaatggctccgatccattccagccccttccctggagcctggcggacccaggtcattgtataggcactgagggagaatccagagactgtgcaggtgagtgtcaggggtgtcccaggcgtgaccaggcgacccccggactcctccagcgactg

SEQ ID NO: 225 - DNA, 357 bp, synthetic construct
  misc_feature 1..357: note = 21626B-F5 Coding Sequence
  source 1..357: mol_type = other DNA
caggagcagctgatggagtccgggggtcgcctggtcacgcctgggacacccctgacactcacctgcacagtctctggattctccctcagtagctatgcaatgacctgggtccgccaggctccagggaaggggctggaatggatcggaatcattagtactaatggtaacacatactacgcgagctgggcgaaaggccgattcgccatctccaaaacctcgaccacggtggatctgaaaatcaccagtccgacaaccgaggacacggccacctatttctgtgccagaggttatggtagtaataattattatgagcactttaacatctggggcccaggcaccctggtcaccgtctcctta

SEQ ID NO: 226 - DNA, 357 bp, synthetic construct
  misc_feature 1..357: note = 21626B-F5 Coding Complementary Sequence
  source 1..357: mol_type = other DNA
taaggagacggtgaccagggtgcctgggccccagatgttaaagtgctcataataattattactaccataacctctggcacagaaataggtggccgtgtcctcggttgtcggactggtgattttcagatccaccgtggtcgaggttttggagatggcgaatcggcctttcgcccagctcgcgtagtatgtgttaccattagtactaatgattccgatccattccagccccttccctggagcctggcggacccaggtcattgcatagctactgagggagaatccagagactgtgcaggtgagtgtcaggggtgtcccaggcgtgaccaggcgacccccggactccatcagctgctcctg

SEQ ID NO: 227 - DNA, 357 bp, synthetic construct
  misc_feature 1..357: note = 21626B-C5 Coding Sequence
  source 1..357: mol_type = other DNA
caggagcagctgaaggagtccgggggtcgcctggtcacgcctgggacacccctgacactcacctgcacagtctctggattctccctcagtaactatgcaatgagctgggtccgccaggctccagggaaggggctggaatggatcggaatcattagtagtagtggtaacacatactacgcgagctgggcgaaaggccgattcaccatctccaaaacctcgaccacggtggatctgaaaatcaccagtccgacaaccgaggacacggccacctatttctgtgccagaggttatggttataataattattatgagcactttaacatctggggcccaggcaccctggtcaccgtctcctta

SEQ ID NO: 228 - DNA, 357 bp, synthetic construct
  misc_feature 1..357: note = 21626B-C5 Coding Complementary Sequence
  source 1..357: mol_type = other DNA
taaggagacggtgaccagggtgcctgggccccagatgttaaagtgctcataataattattataaccataacctctggcacagaaataggtggccgtgtcctcggttgtcggactggtgattttcagatccaccgtggtcgaggttttggagatggtgaatcggcctttcgcccagctcgcgtagtatgtgttaccactactactaatgattccgatccattccagccccttccctggagcctggcggacccagctcattgcatagttactgagggagaatccagagactgtgcaggtgagtgtcaggggtgtcccaggcgtgaccaggcgacccccggactccttcagctgctcctg

SEQ ID NO: 229 - DNA, 357 bp, synthetic construct
  misc_feature 1..357: note = 21626S-G9 Coding Sequence
  source 1..357: mol_type = other DNA
caggagcagctggaggagtccgggggtcgcctggtcacgcctgggacacccctgacactcacctgcacagcctctggattctccctcagtgcctactccatgagctgggtccgccaggctccagggaaggggctggaatggatcggaatcattagtagcagtggttacacatactacgcgagctgggcgaaaggccgattcaccatctccaaaacctcgaccacggtggatctggagatcaccagtccgacaaccgaggacacggccacctatttctgtgccagaggttatggttataataattattatgagcactttaacatgtggggcccaggcaccctggtcaccgtctcctta

SEQ ID NO: 230 - DNA, 357 bp, synthetic construct
  misc_feature 1..357: note = 21626S-G9 Coding Complementary Sequence
  source 1..357: mol_type = other DNA
taaggagacggtgaccagggtgcctgggccccacatgttaaagtgctcataataattattataaccataacctctggcacagaaataggtggccgtgtcctcggttgtcggactggtgatctccagatccaccgtggtcgaggttttggagatggtgaatcggcctttcgcccagctcgcgtagtatgtgtaaccactgctactaatgattccgatccattccagccccttccctggagcctggcggacccagctcatggagtaggcactgagggagaatccagaggctgtgcaggtgagtgtcaggggtgtcccaggcgtgaccaggcgacccccggactcctccagctgctcctg

SEQ ID NO: 231 - DNA, 354 bp, synthetic construct
  misc_feature 1..354: note = 21626S-F8 Coding Sequence
  source 1..354: mol_type = other DNA
cagtcagtgaaggagtccgagggtcgcctggtcacgcctgggacacccctgacactcacctgcacagtcgctggattctccctcagccactaccatatgagctgggtccgccaggctccagggaaggggctggaatggatcggaatcattggtagtagtggaaacacatggtacgcgagctgggcgaaaggccgattcaccatctccaagacctcgaccacggtggatctgagaatcaccagtccgacaaccgaggacacggccacctatttctgtgccagagctggtgctagtaacaattattataattactttaacgtctggggcccaggcaccctggtcaccgtctcctta

SEQ ID NO: 232 - DNA, 354 bp, synthetic construct
  misc_feature 1..354: note = 21626S-F8 Coding Complementary Sequence
  source 1..354: mol_type = other DNA
taaggagacggtgaccagggtgcctgggccccagacgttaaagtaattataataattgttactagcaccagctctggcacagaaataggtggccgtgtcctcggttgtcggactggtgattctcagatccaccgtggtcgaggtcttggagatggtgaatcggcctttcgcccagctcgcgtaccatgtgtttccactactaccaatgattccgatccattccagccccttccctggagcctggcggacccagctcatatggtagtggctgagggagaatccagcgactgtgcaggtgagtgtcaggggtgtcccaggcgtgaccaggcgaccctcggactccttcactgactg

SEQ ID NO: 233 - DNA, 354 bp, synthetic construct
  misc_feature 1..354: note = 21626B-F10 Coding Sequence
  source 1..354: mol_type = other DNA
cagtcggtgaaggagtccgggggtcgcctggtcacgcctgggacacccctgacactcacctgcacagtcgctggattctccctcagccgctactatatgagctgggtccgccaggctccagggaaggggctggaatggatcggaatcattagtagtagtggtagcacatactacgcgagctgggcgaaaggccgattcaccatctccaaaacctcgaccacggtggatctgagaatcaccagtccgacaaccgaggacacggccacctatttctgtgccagaggtggtgctagtaataattattataattactttaacatctggggcccaggcaccctggtcaccgtctcctta

SEQ ID NO: 234 - DNA, 354 bp, synthetic construct
  misc_feature 1..354: note = 21626B-F10 Coding Complementary Sequence
  source 1..354: mol_type = other DNA
taaggagacggtgaccagggtgcctgggccccagatgttaaagtaattataataattattactagcaccacctctggcacagaaataggtggccgtgtcctcggttgtcggactggtgattctcagatccaccgtggtcgaggttttggagatggtgaatcggcctttcgcccagctcgcgtagtatgtgctaccactactactaatgattccgatccattccagccccttccctggagcctggcggacccagctcatatagtagcggctgagggagaatccagcgactgtgcaggtgagtgtcaggggtgtcccaggcgtgaccaggcgacccccggactccttcaccgactg

SEQ ID NO: 235 - DNA, 372 bp, synthetic construct
  misc_feature 1..372: note = 21626B-G12 Coding Sequence
  source 1..372: mol_type = other DNA
cagtcgttggaggagtccgggggaggtctcttcaagccaacggataccctgactctcacctgcacagtctctggattccccctcagtacctatggaataacctgggtccgccaggctccagggaacgggctggaatggatcggaatcattagtagtggtggtagcacacactacgcgagctgggcgaaaagccgatccaccatcaccagagacaccaacgagaacacggtgactctgaaaatgaccagtctgacagtcgcggacacggccacctatttctgtgcgagagccccctactttaggtgggattttaattgggatctttccgcttttgatccctggggcccaggcaccctggtcaccgtctcctca

SEQ ID NO: 236 - DNA, 372 bp, synthetic construct
  misc_feature 1..372: note = 21626B-G12 Coding Complementary Sequence
  source 1..372: mol_type = other DNA
tgaggagacggtgaccagggtgcctgggccccagggatcaaaagcggaaagatcccaattaaaatcccacctaaagtagggggctctcgcacagaaataggtggccgtgtccgcgactgtcagactggtcattttcagagtcaccgtgttctcgttggtgtctctggtgatggtggatcggcttttcgcccagctcgcgtagtgtgtgctaccaccactactaatgattccgatccattccagcccgttccctggagcctggcggacccaggttattccataggtactgagggggaatccagagactgtgcaggtgagagtcagggtatccgttggcttgaagagacctcccccggactcctccaacgactg

SEQ ID NO: 237 - DNA, 351 bp, synthetic construct
  misc_feature 1..351: note = 21626B-A11 Coding Sequence
  source 1..351: mol_type = other DNA
cagtcgctgggggagtccgggggtcgcctggtcaagcctgacgaaaccctgacactcacctgcacagcctctggattctccctcagtaactacgacatgggctgggtccgccaggctccaggggaggggctggaatatatcggatggattactactggtggtagcgcatactacgcgaactgggcaaaaggccgattcaccatctccagaacctcgaccacggtggatctgaaaatcagcagtccgacaagtgaggacacggccacctatttctgtgccagagatggtggccgtggttatggttattcatttagcatctggggcccaggcaccctggtcaccgtctcctca

SEQ ID NO: 238 - DNA, 351 bp, synthetic construct
  misc_feature 1..351: note = 21626B-A11 Coding Complementary Sequence
  source 1..351: mol_type = other DNA
tgaggagacggtgaccagggtgcctgggccccagatgctaaatgaataaccataaccacggccaccatctctggcacagaaataggtggccgtgtcctcacttgtcggactgctgattttcagatccaccgtggtcgaggttctggagatggtgaatcggccttttgcccagttcgcgtagtatgcgctaccaccagtagtaatccatccgatatattccagcccctcccctggagcctggcggacccagcccatgtcgtagttactgagggagaatccagaggctgtgcaggtgagtgtcagggtttcgtcaggcttgaccaggcgacccccggactcccccagcgactg

SEQ ID NO: 239 - DNA, 357 bp, synthetic construct
  misc_feature 1..357: note = 21626B-B5 Coding Sequence
  source 1..357: mol_type = other DNA
cagacggtgaaggagtccgggggtcgcctggtcacgcctgggacacccctgacactcacctgcacagcctctggattctccttcagtggctggtcggtgacctgggtccgccaggctccagggaaggggctggagtggatcggatacatttatcctcggagtggtagcacaaactacgcgaggtgggtgagaggccgattcaccatctccgcaacctcgaccacggtggatctgaaactcaccagtccgacaaccgaggacacggccacttatttctgtgccagaggaggttacggtggtattgactggggtagcatggacatctggggcccaggcaccctggtcaccgtctcctta

SEQ ID NO: 240 - DNA, 357 bp, synthetic construct
  misc_feature 1..357: note = 21626B-B5 Coding Complementary Sequence
  source 1..357: mol_type = other DNA
taaggagacggtgaccagggtgcctgggccccagatgtccatgctaccccagtcaataccaccgtaacctcctctggcacagaaataagtggccgtgtcctcggttgtcggactggtgagtttcagatccaccgtggtcgaggttgcggagatggtgaatcggcctctcacccacctcgcgtagtttgtgctaccactccgaggataaatgtatccgatccactccagccccttccctggagcctggcggacccaggtcaccgaccagccactgaaggagaatccagaggctgtgcaggtgagtgtcaggggtgtcccaggcgtgaccaggcgacccccggactccttcaccgtctg

SEQ ID NO: 241 - DNA, 345 bp, synthetic construct
  misc_feature 1..345: note = 21626B-C2 Coding Sequence
  source 1..345: mol_type = other DNA
cagacagtgaaggagtccgggggtcgcctggtcacgcctgggacacccctgacactcacctgcacagcctctggattctccctcaacagcgacgacatgacctgggtccgccaggctccagggatggggctggaatggattggaaccatgcatgctagcggtttcacatactacgcgagctgggcgcaaggccgattcaccatctccggaacctcgaccacggtggatctgaaaatcaccagtccgacaaccgaggacacggccacctatttctgtgccagaggtgctcctggttatactattgataacatctggggcccaggcaccctggtcaccgtctcctta

SEQ ID NO: 242 - DNA, 345 bp, synthetic construct
  misc_feature 1..345: note = 21626B-C2 Coding Complementary Sequence
  source 1..345: mol_type = other DNA
taaggagacggtgaccagggtgcctgggccccagatgttatcaatagtataaccaggagcacctctggcacagaaataggtggccgtgtcctcggttgtcggactggtgattttcagatccaccgtggtcgaggttccggagatggtgaatcggccttgcgcccagctcgcgtagtatgtgaaaccgctagcatgcatggttccaatccattccagccccatccctggagcctggcggacccaggtcatgtcgtcgctgttgagggagaatccagaggctgtgcaggtgagtgtcaggggtgtcccaggcgtgaccaggcgacccccggactccttcactgtctg

SEQ ID NO: 243 - DNA, 345 bp, synthetic construct
  misc_feature 1..345: note = 21626B-H4 Coding Sequence
  source 1..345: mol_type = other DNA
cagtcgttggaggagtccgggggtcgcctggtcacgcctgggacacccctgacactcacctgcacagtctctggattctccctcagtgaccatgcaatgatctgggtccgccaggctccaggcgaggggctggaatgggtcgggatcatttatcctagtggtagcacatactacgcgaacggggcgaaaggccgattcaccatctccaaaacctcgaccacggtggatctgaaaatgaccagcctgataatcgaggacacggccacgtatttctgtgccagagactatgatagtggctggggtgcggatatctggggcccaggcaccccggtcaccgtctccttg

SEQ ID NO: 244 - DNA, 345 bp, synthetic construct
  misc_feature 1..345: note = 21626B-H4 Coding Complementary Sequence
  source 1..345: mol_type = other DNA
caaggagacggtgaccggggtgcctgggccccagatatccgcaccccagccactatcatagtctctggcacagaaatacgtggccgtgtcctcgattatcaggctggtcattttcagatccaccgtggtcgaggttttggagatggtgaatcggcctttcgccccgttcgcgtagtatgtgctaccactaggataaatgatcccgacccattccagcccctcgcctggagcctggcggacccagatcattgcatggtcactgagggagaatccagagactgtgcaggtgagtgtcaggggtgtcccaggcgtgaccaggcgacccccggactcctccaacgactg

SEQ ID NO: 245 - DNA, 348 bp, synthetic construct
  misc_feature 1..348: note = 21626B-C8 Coding Sequence
  source 1..348: mol_type = other DNA
cagacagtgaaggagtccgagggaggtctcttcaagccaacggataccctgacactcacctgcacagtctctggattctccctcagtatctatggagtgagctgggtccgccaggctccagggaacgggctggaatggatcgggtccattagtgctactggtatcacatactacgcgagctgggcgaaaagccgatccaccgtcaccagaaacaccaacctgaacacggtgactctgaaaatgaccagtctgacagccgcggacacggccacctatttctgtactagaggtcatacttattctacccctgacatctggggcccaggcaccctggtcaccgtctcctca

SEQ ID NO: 246 - DNA, 348 bp, synthetic construct
  misc_feature 1..348: note = 21626B-C8 Coding Complementary Sequence
  source 1..348: mol_type = other DNA
tgaggagacggtgaccagggtgcctgggccccagatgtcaggggtagaataagtatgacctctagtacagaaataggtggccgtgtccgcggctgtcagactggtcattttcagagtcaccgtgttcaggttggtgtttctggtgacggtggatcggcttttcgcccagctcgcgtagtatgtgataccagtagcactaatggacccgatccattccagcccgttccctggagcctggcggacccagctcactccatagatactgagggagaatccagagactgtgcaggtgagtgtcagggtatccgttggcttgaagagacctccctcggactccttcactgtctg

SEQ ID NO: 247 - DNA, 354 bp, synthetic construct
  misc_feature 1..354: note = 21626B-E6 Coding Sequence
  source 1..354: mol_type = other DNA
cagtcgctggaggagtccgggggaggcctgatcaagccaacggataccctgacactcacctgcacagtctctggattctccctcagtaggtatggagtgatctgggtccgccaggctccagggagtggcctggaatggatcggagccattactgctactggtatcacatactacgcgagctgggcgaaaagccgatccaccatcaccagaaacaccaacctgaacacggtgactctgaaaatgaccagtctgacagccgcggacacggccacctatttctgtgcggggggtcatacttttagtactgatgtcggggacatctggggcccaggcaccccggtcaccgtctcctca

SEQ ID NO: 248 - DNA, 354 bp, synthetic construct
  misc_feature 1..354: note = 21626B-E6 Coding Complementary Sequence
  source 1..354: mol_type = other DNA
tgaggagacggtgaccggggtgcctgggccccagatgtccccgacatcagtactaaaagtatgaccccccgcacagaaataggtggccgtgtccgcggctgtcagactggtcattttcagagtcaccgtgttcaggttggtgtttctggtgatggtggatcggcttttcgcccagctcgcgtagtatgtgataccagtagcagtaatggctccgatccattccaggccactccctggagcctggcggacccagatcactccatacctactgagggagaatccagagactgtgcaggtgagtgtcagggtatccgttggcttgatcaggcctcccccggactcctccagcgactg

SEQ ID NO: 249 - DNA, 345 bp, synthetic construct
  misc_feature 1..345: note = 21626B-G5 Coding Sequence
  source 1..345: mol_type = other DNA
cagtcggtgaaggagtccgagggaggtctcttcaagccagcggataccctgacactcacctgcacagtctctggattctccctcaatagctatacaataagctgggtccgccaggctccagggaacgggctggaatggatcggaatcattaatacttatggtaccactaactacgcgagctgggcgaaaagccgatccaccatcaccagaaacaccaacgagaacacggtgactctgaaaatgaccagtctgacagccgcggacacggccacctatttctgtgcgagcttgtatacaacacagactaacatttggggcccaggcaccctggtcaccgtctcctca

SEQ ID NO: 250 - DNA, 345 bp, synthetic construct
  misc_feature 1..345: note = 21626B-G5 Coding Complementary Sequence
  source 1..345: mol_type = other DNA
tgaggagacggtgaccagggtgcctgggccccaaatgttagtctgtgttgtatacaagctcgcacagaaataggtggccgtgtccgcggctgtcagactggtcattttcagagtcaccgtgttctcgttggtgtttctggtgatggtggatcggcttttcgcccagctcgcgtagttagtggtaccataagtattaatgattccgatccattccagcccgttccctggagcctggcggacccagcttattgtatagctattgagggagaatccagagactgtgcaggtgagtgtcagggtatccgctggcttgaagagacctccctcggactccttcaccgactg

SEQ ID NO: 251 - DNA, 345 bp, synthetic construct
  misc_feature 1..345: note = 21626B-H3 Coding Sequence
  source 1..345: mol_type = other DNA
cagtcgctgggggagtccaggggtcgcctggtaacgcctggaggatccctgacactcacctgcacagcctctggattctccctcagtagctatgcaatgggctgggtccgccaggctccagggaaggggctggaatacatcggatggattagtgctggtggtaccacatactacgcgagctgggtgaatagtcgattcaccatctccagaacctcgaccacggtggatctggaaatgaccagtctgacaaccgaggacacggccacctatttctgtgccagagagggtactggctggggtgcctatgacatctggggcccaggcaccctggtcaccgtctcctta

SEQ ID NO: 252 - DNA, 345 bp, synthetic construct
  misc_feature 1..345: note = 21626B-H3 Coding Complementary Sequence
  source 1..345: mol_type = other DNA
taaggagacggtgaccagggtgcctgggccccagatgtcataggcaccccagccagtaccctctctggcacagaaataggtggccgtgtcctcggttgtcagactggtcatttccagatccaccgtggtcgaggttctggagatggtgaatcgactattcacccagctcgcgtagtatgtggtaccaccagcactaatccatccgatgtattccagccccttccctggagcctggcggacccagcccattgcatagctactgagggagaatccagaggctgtgcaggtgagtgtcagggatcctccaggcgttaccaggcgacccctggactcccccagcgactg

SEQ ID NO: 253 - DNA, 339 bp, synthetic construct
  misc_feature 1..339: note = 21626B-A8 Coding Sequence
  source 1..339: mol_type = other DNA
cagctggaggagtccgggggtcgcctggtcacgcctgggacacccctgacactcacctgcaccgtctctggaatcgacctcagtagcgacagtattggctgggtccgccaggctccagggaaggggctggaatggatcggaatcatttatcctagtggtaatgtatactacgcgagctgggcgaaaggccgactcaccatctccaaaacctcgaccacggtggagctgaaaatgaccagtctgacaaccgaggacacggccacctatttttgtgccagagattactatagtactaccttagacatctggggcccaggcaccctggtcaccgtctccttg

SEQ ID NO: 254 - DNA, 339 bp, synthetic construct
  misc_feature 1..339: note = 21626B-A8 Coding Complementary Sequence
  source 1..339: mol_type = other DNA
caaggagacggtgaccagggtgcctgggccccagatgtctaaggtagtactatagtaatctctggcacaaaaataggtggccgtgtcctcggttgtcagactggtcattttcagctccaccgtggtcgaggttttggagatggtgagtcggcctttcgcccagctcgcgtagtatacattaccactaggataaatgattccgatccattccagccccttccctggagcctggcggacccagccaatactgtcgctactgaggtcgattccagagacggtgcaggtgagtgtcaggggtgtcccaggcgtgaccaggcgacccccggactcctccagctg

SEQ ID NO: 255 - DNA, 342 bp, synthetic construct
  misc_feature 1..342: note = 21626B-D1 Coding Sequence
  source 1..342: mol_type = other DNA
caggagcagctgaaggagtcccggggtcgcctggtaacgcctggaggatccctgacactcacctgcacagtctctggaatcgacctcagtagcaatgcaatgagctgggtccgccaggctccaggggaggggctggaatggatcggaaccattagtggtggtagtggtagcacatggtacgcgagctgggcgaaaggccgattcaccatctccaagacctcgaccacggtgtatctgaaaatgaccagtccgacaaccgaggacacggccacctatttctgtgccagaggttgggacggctttagcacctggggcccaggcaccctggtcaccgtctcctta

SEQ ID NO: 256 - DNA, 342 bp, synthetic construct
  misc_feature 1..342: note = 21626B-D1 Coding Complementary Sequence
  source 1..342: mol_type = other DNA
taaggagacggtgaccagggtgcctgggccccaggtgctaaagccgtcccaacctctggcacagaaataggtggccgtgtcctcggttgtcggactggtcattttcagatacaccgtggtcgaggtcttggagatggtgaatcggcctttcgcccagctcgcgtaccatgtgctaccactaccaccactaatggttccgatccattccagcccctcccctggagcctggcggacccagctcattgcattgctactgaggtcgattccagagactgtgcaggtgagtgtcagggatcctccaggcgttaccaggcgaccccgggactccttcagctgctcctg

SEQ ID NO: 257 - DNA, 339 bp, synthetic construct
  misc_feature 1..339: note = 21626B-G1 Coding Sequence
  source 1..339: mol_type = other DNA
caggagcagctggtggagtccgggggtcgcctggtcacgcctgggtcacccctgacactcacctgcacagtctctggattctccctcagtagctactggatgagctgggtccgccaagctccagggaaggggctggagtggatgggagatattagtaatactggtaagatatattacgcgaactgggcgaaaggccgattcaccatctccaaaacctcgaccacggtggatctgacaatcaccagtccgacaacggaggacacggccacctatttctgtgccagaggtatgaatgtttatgacatctggggcccaggcaccctggtcaccgtctcctta

SEQ ID NO: 258 - DNA, 339 bp, synthetic construct
  misc_feature 1..339: note = 21626B-G1 Coding Complementary Sequence
  source 1..339: mol_type = other DNA
taaggagacggtgaccagggtgcctgggccccagatgtcataaacattcatacctctggcacagaaataggtggccgtgtcctccgttgtcggactggtgattgtcagatccaccgtggtcgaggttttggagatggtgaatcggcctttcgcccagttcgcgtaatatatcttaccagtattactaatatctcccatccactccagccccttccctggagcttggcggacccagctcatccagtagctactgagggagaatccagagactgtgcaggtgagtgtcaggggtgacccaggcgtgaccaggcgacccccggactccaccagctgctcctg

SEQ ID NO: 259 - DNA, 336 bp, synthetic construct
  misc_feature 1..336: note = 21626B-C4- Coding Sequence
  source 1..336: mol_type = other DNA
cagtcgttggaggagtccgggggtcgcctggtcacgcctgggacacccctgacactcacctgcacagcctctggattctccctcagtagctactacatgagctgggtccgccaggctccagaaaaggggctggaatggatcggaatcatttatcctagtggtaacacatactacgcgaactgggcgaatggtcgattcgccatctccagaacctcgaccacggtggatctgaagatcaccagtccgacaaccgaggacacggccacctatttctgcgccagagattctggttatcctgacatctggggcccaggcaccctggtcaccgtctcctta

SEQ ID NO: 260 - DNA, 336 bp, synthetic construct
  misc_feature 1..336: note = 21626B-C4- Coding Complementary Sequence
  source 1..336: mol_type = other DNA
taaggagacggtgaccagggtgcctgggccccagatgtcaggataaccagaatctctggcgcagaaataggtggccgtgtcctcggttgtcggactggtgatcttcagatccaccgtggtcgaggttctggagatggcgaatcgaccattcgcccagttcgcgtagtatgtgttaccactaggataaatgattccgatccattccagccccttttctggagcctggcggacccagctcatgtagtagctactgagggagaatccagaggctgtgcaggtgagtgtcaggggtgtcccaggcgtgaccaggcgacccccggactcctccaacgactg

SEQ ID NO: 261 - DNA, 357 bp, synthetic construct
  misc_feature 1..357: note = 21626S-A1 Coding Sequence
  source 1..357: mol_type = other DNA
caggagcagctgaaggagtccgggggtcgcctggtcacgcctgggacacccctgacactcacctgcacagcctctggattctccctcagtgcctactccatgagctgggtccgccaggctccagggaaggggctggaatggatcggaatcattagtagcagtggttacacatactacgcgagctgggcgaaaggccgattcaccatctccaaaacctcgaccacggtggatctggagatcaccagtccgacaaccgaggacacggccacctatttctgtgccagaggttatggttataataattattatgagcactttaacatgtggggcccaggcaccctggtcaccgtctcctta

SEQ ID NO: 262 - DNA, 357 bp, synthetic construct
  misc_feature 1..357: note = 21626S-A1 Coding Complementary Sequence
  source 1..357: mol_type = other DNA
taaggagacggtgaccagggtgcctgggccccacatgttaaagtgctcataataattattataaccataacctctggcacagaaataggtggccgtgtcctcggttgtcggactggtgatctccagatccaccgtggtcgaggttttggagatggtgaatcggcctttcgcccagctcgcgtagtatgtgtaaccactgctactaatgattccgatccattccagccccttccctggagcctggcggacccagctcatggagtaggcactgagggagaatccagaggctgtgcaggtgagtgtcaggggtgtcccaggcgtgaccaggcgacccccggactccttcagctgctcctg

SEQ ID NO: 263 - DNA, 336 bp, synthetic construct
  misc_feature 1..336: note = 21626S-D5 Coding Sequence
  source 1..336: mol_type = other DNA
gcccaagtgatggcccagactccagcctccgtgtctgaacctgtcggaggcacagtcaccatcaagtgccaggccagtcagagcattggcaatgcattagcctggtatcagcagaaaccagggcagcgtccctcgctcctgatctatgatgcatccactctggcatctggggtctcatcgcggttcaaaggcagtagatctgggacagagtacactctcaccatcagcgacctggagtgtgccgatgctgccacttactactgtcagtgtgcttactatggtagccgttatgctggagggagtgctttcggcggagggaccgaggtggtggtcaaa

SEQ ID NO: 264 - DNA, 336 bp, synthetic construct
  misc_feature 1..336: note = 21626S-D5 Coding Complementary Sequence
  source 1..336: mol_type = other DNA
tttgaccaccacctcggtccctccgccgaaagcactccctccagcataacggctaccatagtaagcacactgacagtagtaagtggcagcatcggcacactccaggtcgctgatggtgagagtgtactctgtcccagatctactgcctttgaaccgcgatgagaccccagatgccagagtggatgcatcatagatcaggagcgagggacgctgccctggtttctgctgataccaggctaatgcattgccaatgctctgactggcctggcacttgatggtgactgtgcctccgacaggttcagacacggaggctggagtctgggccatcacttgggc

SEQ ID NO: 265 - DNA, 336 bp, synthetic construct
  misc_feature 1..336: note = 21626B-B9 Coding Sequence
  source 1..336: mol_type = other DNA
gccatcgatctgacccagactccagcctccgtgtctgaacctgtgggaggcacagtcaccatcaagtgccaggccagtcagagcattggcaatgcattagcctggtatcagcagaaaccagggcagcctcccaagctcctgatccatgatgcatccactctggcatctggggtctcatcgcggttcaaaggcagtagatctgggacagagtacactctcaccatcagcgacctggagtgtgccgatgctgccacttactactgtcagtgtgcttattatggtagccgttatgctggagggaatgctttcggcggagggaccgaggtggtggtcaaa

SEQ ID NO: 266 - DNA, 336 bp, synthetic construct
  misc_feature 1..336: note = 21626B-B9 Coding Complementary Sequence
  source 1..336: mol_type = other DNA
tttgaccaccacctcggtccctccgccgaaagcattccctccagcataacggctaccataataagcacactgacagtagtaagtggcagcatcggcacactccaggtcgctgatggtgagagtgtactctgtcccagatctactgcctttgaaccgcgatgagaccccagatgccagagtggatgcatcatggatcaggagcttgggaggctgccctggtttctgctgataccaggctaatgcattgccaatgctctgactggcctggcacttgatggtgactgtgcctcccacaggttcagacacggaggctggagtctgggtcagatcgatggc

SEQ ID NO: 267 - DNA, 336 bp, synthetic construct
  misc_feature 1..336: note = 21626B-H8 Coding Sequence
  source 1..336: mol_type = other DNA
gatggtgtgatgacccagactccagcctccgtgtctgaacctgtgggaggcacagtcaccatcaagtgccaggccagtcagagcattggcaatgcattagcctggtatcagcagaaaccagggcagcctcccaagctcctgatccatgatgcatccactctggcatccggggtctcatcgcggttcaaaggcagtagatctgggacagagtacactctcaccatcagcgacctggagtgtgccgatgctgccacttactactgtcagtgtgcttattatggtagccgttatgctggagggaatgctttcggcggagggaccgaggtggtggtcaaa

SEQ ID NO: 268 - DNA, 336 bp, synthetic construct
  misc_feature 1..336: note = 21626B-H8 Coding Complementary Sequence
  source 1..336: mol_type = other DNA
tttgaccaccacctcggtccctccgccgaaagcattccctccagcataacggctaccataataagcacactgacagtagtaagtggcagcatcggcacactccaggtcgctgatggtgagagtgtactctgtcccagatctactgcctttgaaccgcgatgagaccccggatgccagagtggatgcatcatggatcaggagcttgggaggctgccctggtttctgctgataccaggctaatgcattgccaatgctctgactggcctggcacttgatggtgactgtgcctcccacaggttcagacacggaggctggagtctgggtcatcacaccatc

SEQ ID NO: 269 - DNA, 336 bp, synthetic construct
  misc_feature 1..336: note = 21626S-E6 Coding Sequence
  source 1..336: mol_type = other DNA
gatgttgtgatgacccagactccagcctccgcgtctgaacctgtgggaggcacagtcaccatcaagtgccaggccagtcagagcattggcaatgcattagcctggtatcagcagaaaccagggcagcctcccaagctcctgatccatgatgcatccactctggcatctggggtctcatcgcggttcaaaggcagtagatctgggacagagtacactctcaccatcagcgacctggagtgtgccgatgctgccacttactactgtcagtgtgcttattatggtagccgttatgctggagggaatgctttcggcggagggaccgaggtggtggtcaaa

SEQ ID NO: 270 - DNA, 336 bp, synthetic construct
  misc_feature 1..336: note = 21626S-E6 Coding Complementary Sequence
  source 1..336: mol_type = other DNA
tttgaccaccacctcggtccctccgccgaaagcattccctccagcataacggctaccataataagcacactgacagtagtaagtggcagcatcggcacactccaggtcgctgatggtgagagtgtactctgtcccagatctactgcctttgaaccgcgatgagaccccagatgccagagtggatgcatcatggatcaggagcttgggaggctgccctggtttctgctgataccaggctaatgcattgccaatgctctgactggcctggcacttgatggtgactgtgcctcccacaggttcagacgcggaggctggagtctgggtcatcacaacatc

SEQ ID NO: 271 - DNA, 336 bp, synthetic construct
  misc_feature 1..336: note = 21626S-E12 Coding Sequence
  source 1..336: mol_type = other DNA
gatggtgtgatgacccagactccagcctccgtgtctgaacctgtgggaggcacagtcaccatcaagtgccaggccagtcagagcattggcaatgcattagcctggtatcagcagaaaccagggcagcgtccctcgctcctgatctatgatgcatccactctggcatctggggtctcatcgcggttcaaaggcagtagatctgggacagagtacactctcaccatcagcgacctggagtgtgccgatgctgccacttactactgtcagtgtgcttactatggtagccgttatgctggagggaatgctttcggcggagggaccgaggtggtggtcaaa

SEQ ID NO: 272 - DNA, 336 bp, synthetic construct
  misc_feature 1..336: note = 21626S-E12 Coding Complementary Sequence
  source 1..336: mol_type = other DNA
tttgaccaccacctcggtccctccgccgaaagcattccctccagcataacggctaccatagtaagcacactgacagtagtaagtggcagcatcggcacactccaggtcgctgatggtgagagtgtactctgtcccagatctactgcctttgaaccgcgatgagaccccagatgccagagtggatgcatcatagatcaggagcgagggacgctgccctggtttctgctgataccaggctaatgcattgccaatgctctgactggcctggcacttgatggtgactgtgcctcccacaggttcagacacggaggctggagtctgggtcatcacaccatc

SEQ ID NO: 273 - DNA, 336 bp, synthetic construct
  misc_feature 1..336: note = 21626B-F7 Coding Sequence
  source 1..336: mol_type = other DNA
gatgtcgtgatgacccagactccgtcctccgtgtctgaacctgtgggaggcacagtcaccatcaagtgccaggccagtcagagcattggcaatgcattagcctggtatcagcagaaaccagggcagcgtcccaagctcctgatccatgatgcatccactctggcatctggggtctcatcgcggttcaaaggcattagatccgggacagagtacactctcaccatcagcgacctggagtgtgccgatgctgccacttactactgtcagtgtgcttattatggtaaccgttatgctggagggaatgctttcggcggagggaccgaggtggtggtcaaa

SEQ ID NO: 274 - DNA, 336 bp, synthetic construct
  misc_feature 1..336: note = 21626B-F7 Coding Complementary Sequence
  source 1..336: mol_type = other DNA
tttgaccaccacctcggtccctccgccgaaagcattccctccagcataacggttaccataataagcacactgacagtagtaagtggcagcatcggcacactccaggtcgctgatggtgagagtgtactctgtcccggatctaatgcctttgaaccgcgatgagaccccagatgccagagtggatgcatcatggatcaggagcttgggacgctgccctggtttctgctgataccaggctaatgcattgccaatgctctgactggcctggcacttgatggtgactgtgcctcccacaggttcagacacggaggacggagtctgggtcatcacgacatc

SEQ ID NO: 275 - DNA, 336 bp, synthetic construct
  misc_feature 1..336: note = 21626B-F5 Coding Sequence
  source 1..336: mol_type = other DNA
gatggcgtgatgacccagactccagcctccgtgtctgaacctgtgggaggcacagtcaccatcaagtgccaggccagtcagagcattggcaatgcattagcctggtatcagcagaaaccagggcagcctcccaagctcctgatccatgatgcatccactctggcatctggggtctcatcgcggttcaaaggcagtagatctgggacagagtacactctcaccatcagcgacctggagtgtgccgatgctgccacttactactgtcagtgtgcttattatggtagccgttatgctggagggaatgctttcggcggagggaccgaggtggtggtcaaa

SEQ ID NO: 276 - DNA, 336 bp, synthetic construct
  misc_feature 1..336: note = 21626B-F5 Coding Complementary Sequence
  source 1..336: mol_type = other DNA
tttgaccaccacctcggtccctccgccgaaagcattccctccagcataacggctaccataataagcacactgacagtagtaagtggcagcatcggcacactccaggtcgctgatggtgagagtgtactctgtcccagatctactgcctttgaaccgcgatgagaccccagatgccagagtggatgcatcatggatcaggagcttgggaggctgccctggtttctgctgataccaggctaatgcattgccaatgctctgactggcctggcacttgatggtgactgtgcctcccacaggttcagacacggaggctggagtctgggtcatcacgccatc

SEQ ID NO: 277 - DNA, 336 bp, synthetic construct
  misc_feature 1..336: note = 21626B-C5 Coding Sequence
  source 1..336: mol_type = other DNA
gcccaagggatgacccagactccatcctccgtgtctgaacctgtgggaggcacagtcaccatcaagtgccaggccagtcagagcattggcaatgcattagcctggtatcagcagaaaccagggcagcgtcccaagctcctgatccatgatgcatccactctggcatctggggtctcatcgcggttcaaaggcattagatctgggacagagtacactctcaccatcagcgacctggagtgtgccgatgctgccacttactactgtcagtgtgcttattatggtaaccgttatgctggagggaatgctttcggcggagggaccgaggtggtggtcaaa

SEQ ID NO: 278 - DNA, 336 bp, synthetic construct
  misc_feature 1..336: note = 21626B-C5 Coding Complementary Sequence
  source 1..336: mol_type = other DNA
tttgaccaccacctcggtccctccgccgaaagcattccctccagcataacggttaccataataagcacactgacagtagtaagtggcagcatcggcacactccaggtcgctgatggtgagagtgtactctgtcccagatctaatgcctttgaaccgcgatgagaccccagatgccagagtggatgcatcatggatcaggagcttgggacgctgccctggtttctgctgataccaggctaatgcattgccaatgctctgactggcctggcacttgatggtgactgtgcctcccacaggttcagacacggaggatggagtctgggtcatcccttgggc

SEQ ID NO: 279 - DNA, 336 bp, synthetic construct
  misc_feature 1..336: note = 21626S-G9 Coding Sequence
  source 1..336: mol_type = other DNA
gcccaagggatgacccagactccagcctccgtgtctgaacctgtgggaggcacagtcaccatcaagtgccaggccagtcagagcattggcaatgcattagcctggtatcagcagaaaccagggcagcctcccaagctcctgatccatgatgcatccactctggcatctggggtctcatcgcggttcaaaggcagtagatctgggacagagtacactctcaccatcagcgacctggagtgtgccgatgctgccacttactactgtcagtgtgcttattatggtagccgttatgctggagggaatgctttcggcggagggaccgaggtggtggtcaaa

SEQ ID NO: 280 - DNA, 336 bp, synthetic construct
  misc_feature 1..336: note = 21626S-G9 Coding Complementary Sequence
  source 1..336: mol_type = other DNA
tttgaccaccacctcggtccctccgccgaaagcattccctccagcataacggctaccataataagcacactgacagtagtaagtggcagcatcggcacactccaggtcgctgatggtgagagtgtactctgtcccagatctactgcctttgaaccgcgatgagaccccagatgccagagtggatgcatcatggatcaggagcttgggaggctgccctggtttctgctgataccaggctaatgcattgccaatgctctgactggcctggcacttgatggtgactgtgcctcccacaggttcagacacggaggctggagtctgggtcatcccttgggc

SEQ ID NO: 281 - DNA, 336 bp, synthetic construct
  misc_feature 1..336: note = 21626S-F8 Coding Sequence
  source 1..336: mol_type = other DNA
gatgttgtgatgacccagactccagcctccgtgtctgaacctgtgggaggcacagccaccatcaagtgccaggccagtcagagcattggcaatgcattagcctggtatcagcagaaaccagggcagcctcccaagctcctgatgcatgatgtatccactctggcatctggggtctcatcgcggttcaaaggcagtagatctgggacagagttcactctcaccatcagcgacctggagtgtgccgatgctgccacttactactgtcagtgtgcttattatggtagccgttatgctggagggaatgctttcggcggagggaccgaggtggtggtcaaa

SEQ ID NO: 282 - DNA, 336 bp, synthetic construct
  misc_feature 1..336: note = 21626S-F8 Coding Complementary Sequence
  source 1..336: mol_type = other DNA
tttgaccaccacctcggtccctccgccgaaagcattccctccagcataacggctaccataataagcacactgacagtagtaagtggcagcatcggcacactccaggtcgctgatggtgagagtgaactctgtcccagatctactgcctttgaaccgcgatgagaccccagatgccagagtggatacatcatgcatcaggagcttgggaggctgccctggtttctgctgataccaggctaatgcattgccaatgctctgactggcctggcacttgatggtggctgtgcctcccacaggttcagacacggaggctggagtctgggtcatcacaacatc

SEQ ID NO: 283 - DNA, 336 bp, synthetic construct
  misc_feature 1..336: note = 21626B-F10 Coding Sequence
  source 1..336: mol_type = other DNA
gcagccgtgatgacccagactccagcctccgtgtctgaacctgtgggaggcacagtcaccatcaagtgccaggccagtcagagcattggcaatgcattagcctggtatcagcagaaaccagggcagcctcccaagctcctgatccatgatgcatccactctggcatctggggtctcatcgcggttcaaaggcagtagatctgggacagagtacactctcaccatcagcgacctggagtgtgccgatgctgccacttactactgtcagtgtgcttattatggtagccgttatgctggagggaatgctttcggcggagggaccgaggtggtggtcaaa

SEQ ID NO: 284 - DNA, 336 bp, synthetic construct
  misc_feature 1..336: note = 21626B-F10 Coding Complementary Sequence
  source 1..336: mol_type = other DNA
tttgaccaccacctcggtccctccgccgaaagcattccctccagcataacggctaccataataagcacactgacagtagtaagtggcagcatcggcacactccaggtcgctgatggtgagagtgtactctgtcccagatctactgcctttgaaccgcgatgagaccccagatgccagagtggatgcatcatggatcaggagcttgggaggctgccctggtttctgctgataccaggctaatgcattgccaatgctctgactggcctggcacttgatggtgactgtgcctcccacaggttcagacacggaggctggagtctgggtcatcacggctgc

SEQ ID NO: 285 - DNA, 330 bp, synthetic construct
  misc_feature 1..330: note = 21626B-G12 Coding Sequence
  source 1..330: mol_type = other DNA
gcccaagtgctgacccagactccagcctccgtgtctgcagctgtgggaggcacagtcaccatcagttgccagtccagtgagagtgtttataagaacaactacttatcctggtatcagcagaaaccagggcagcctcccaaaggcctgatctatctttcatccactctggcatctggggtcccatcgcggttcagcggcagtggatctgggacacagttcactctcaccatcagcgacctggagtgtagcgatgctgccacttactactgtgcaggcggttatagtggtaatattggcgctttcggcggagggaccgaggtggtggtcaaa

SEQ ID NO: 286 - DNA, 330 bp, synthetic construct
  misc_feature 1..330: note = 21626B-G12 Coding Complementary Sequence
  source 1..330: mol_type = other DNA
tttgaccaccacctcggtccctccgccgaaagcgccaatattaccactataaccgcctgcacagtagtaagtggcagcatcgctacactccaggtcgctgatggtgagagtgaactgtgtcccagatccactgccgctgaaccgcgatgggaccccagatgccagagtggatgaaagatagatcaggcctttgggaggctgccctggtttctgctgataccaggataagtagttgttcttataaacactctcactggactggcaactgatggtgactgtgcctcccacagctgcagacacggaggctggagtctgggtcagcacttgggc

SEQ ID NO: 287 - DNA, 330 bp, synthetic construct
  misc_feature 1..330: note = 21626B-A11 Coding Sequence
  source 1..330: mol_type = other DNA
gcgctggtgatgacccagactccagcctccgtggaggcagctgtgggaggcacagtcaccatcaattgccaggccagccagaacattggtaattacttatcctggtttcaacagaaaccagggcagcgtcccaagctcctgatctacaaggcttccactctggcatctggggtctcatcgcggttcaaaggcagtggatctgggacacagttcactctcaccatcagcggcgtggagtgtgccgatgctgccacttactactgtcaacaagatgctgctgttattaatgttgataatactttcggcggagggaccgaggtggtggtcaaa

SEQ ID NO: 288 - DNA, 330 bp, synthetic construct
  misc_feature 1..330: note = 21626B-A11 Coding Complementary Sequence
  source 1..330: mol_type = other DNA
tttgaccaccacctcggtccctccgccgaaagtattatcaacattaataacagcagcatcttgttgacagtagtaagtggcagcatcggcacactccacgccgctgatggtgagagtgaactgtgtcccagatccactgcctttgaaccgcgatgagaccccagatgccagagtggaagccttgtagatcaggagcttgggacgctgccctggtttctgttgaaaccaggataagtaattaccaatgttctggctggcctggcaattgatggtgactgtgcctcccacagctgcctccacggaggctggagtctgggtcatcaccagcgc

SEQ ID NO: 289 - DNA, 342 bp, synthetic construct
  misc_feature 1..342: note = 21626B-B5 Coding Sequence
  source 1..342: mol_type = other DNA
gccgtcgtgctgacccagactccatcatcctcgtctgcagctgtgggaggcacagtcaccatcaattgccaggccagtcagagtgtttataataacaactacttatcctggtatcagcaaaaaccagggcagcctcccaagctcctgatctatggtgcatccaaactggcatctggggtcccagcgcggttcagcggcagtagatctgggacagacttcactctcaccatcaacgacctggagtgtgccgatgctgccacttactactgtcaaagtacttatggtagtaatagtaatggttgggataatgctttcggcggagggaccgaggtggtcgtcaga

SEQ ID NO: 290 - DNA, 342 bp, synthetic construct
  misc_feature 1..342: note = 21626B-B5 Coding Complementary Sequence
  source 1..342: mol_type = other DNA
tctgacgaccacctcggtccctccgccgaaagcattatcccaaccattactattactaccataagtactttgacagtagtaagtggcagcatcggcacactccaggtcgttgatggtgagagtgaagtctgtcccagatctactgccgctgaaccgcgctgggaccccagatgccagtttggatgcaccatagatcaggagcttgggaggctgccctggtttttgctgataccaggataagtagttgttattataaacactctgactggcctggcaattgatggtgactgtgcctcccacagctgcagacgaggatgatggagtctgggtcagcacgacggc

SEQ ID NO: 291 - DNA, 339 bp, synthetic construct
  misc_feature 1..339: note = 21626B-C2 Coding Sequence
  source 1..339: mol_type = other DNA
gcccaagtgctgacccagactgcatcccctgtgtctgcagctgtgggaggcacagtcaccatcaattgtcagtccagtcagagcatttatggtaacaactggttaggctggtatcagcagaaaccagggcagcctcccaagcaactgatctacaaggcttccactctggcatctggggtctcatcgcggttcaaaggcagtggatctgggacacagttcactctcaccatcagcgacgtgcagtgtgacgatgctgccacttactactgtctaggcgaatttagttgtactagtggtgattgttatactttcggcggagggaccgaggtggtggtcaaa

SEQ ID NO: 292 - DNA, 339 bp, synthetic construct
  misc_feature 1..339: note = 21626B-C2 Coding Complementary Sequence
  source 1..339: mol_type = other DNA
tttgaccaccacctcggtccctccgccgaaagtataacaatcaccactagtacaactaaattcgcctagacagtagtaagtggcagcatcgtcacactgcacgtcgctgatggtgagagtgaactgtgtcccagatccactgcctttgaaccgcgatgagaccccagatgccagagtggaagccttgtagatcagttgcttgggaggctgccctggtttctgctgataccagcctaaccagttgttaccataaatgctctgactggactgacaattgatggtgactgtgcctcccacagctgcagacacaggggatgcagtctgggtcagcacttgggc

SEQ ID NO: 293 - DNA, 330 bp, synthetic construct
  misc_feature 1..330: note = 21626B-H4 Coding Sequence
  source 1..330: mol_type = other DNA
gccctcgtgatgacccagactccagcctccgtgtctgcagctgtgggaggcacagtcaccatcaattgccaggccagtcagagcattcataagtacttatcctggtatcagcagaaaccagggcagcctcccaaactcctgatttacagggcatccactctggcatctggggtcccgtcgcggttcaaaggcagtggatctgggacagagttcactctcaccattagcgacctggagtgtgccgatgctgccacttactactgtcaacaggatgctgccgttattaatattgataacagtttcggcggagggaccgaggtggtcgtcaaa

SEQ ID NO: 294 - DNA, 330 bp, synthetic construct
  misc_feature 1..330: note = 21626B-H4 Coding Complementary Sequence
  source 1..330: mol_type = other DNA
tttgacgaccacctcggtccctccgccgaaactgttatcaatattaataacggcagcatcctgttgacagtagtaagtggcagcatcggcacactccaggtcgctaatggtgagagtgaactctgtcccagatccactgcctttgaaccgcgacgggaccccagatgccagagtggatgccctgtaaatcaggagtttgggaggctgccctggtttctgctgataccaggataagtacttatgaatgctctgactggcctggcaattgatggtgactgtgcctcccacagctgcagacacggaggctggagtctgggtcatcacgagggc

SEQ ID NO: 295 - DNA, 336 bp, synthetic construct
  misc_feature 1..336: note = 21626B-C8 Coding Sequence
  source 1..336: mol_type = other DNA
gcgctcgtgctgacccagactccatcttccaagtctgtccctgtgggagacacagtcaccatcaattgccaggccagtgagagtgtttataataagaactacttagcctggtttcagcagaaaccagggcagcctcccaagctcctgatctacaaggcttccactctggcatctggggtcccatcgcggttcaaaggcagtggatctgggacacagttcactctcaccatcaacgatgtggtgtgtgacgatgctgccacttacttttgtgcaggatataaagatgctgctactgatggtaatgctttcggcggagggaccgaggtggtggtcaaa

SEQ ID NO: 296 - DNA, 336 bp, synthetic construct
  misc_feature 1..336: note = 21626B-C8 Coding Complementary Sequence
  source 1..336: mol_type = other DNA
tttgaccaccacctcggtccctccgccgaaagcattaccatcagtagcagcatctttatatcctgcacaaaagtaagtggcagcatcgtcacacaccacatcgttgatggtgagagtgaactgtgtcccagatccactgcctttgaaccgcgatgggaccccagatgccagagtggaagccttgtagatcaggagcttgggaggctgccctggtttctgctgaaaccaggctaagtagttcttattataaacactctcactggcctggcaattgatggtgactgtgtctcccacagggacagacttggaagatggagtctgggtcagcacgagcgc

SEQ ID NO: 297 - DNA, 336 bp, synthetic construct
  misc_feature 1..336: note = 21626B-E6 Coding Sequence
  source 1..336: mol_type = other DNA
gcgctggtgctgacccagactccatcttccaagtctgtccctgtgggagacacagtcaccatcaattgccaggccagtgagagtgtttggaataagaactacttagcctggtttcaacagaaaccagggcagcctcccaagctcctgatctacaaggcttccactctggcatctggggtcccatcgcggttcaaaggcagtggatctgggacacaattcactctcaccatcaacgatgtggtgtgtggcgatgctgccacttacttttgtgcaggatataaagatattgatattgatggtaatgctttcggcggagggaccgaggtggtggtcaaa

SEQ ID NO: 298 - DNA, 336 bp, synthetic construct
  misc_feature 1..336: note = 21626B-E6 Coding Complementary Sequence
  source 1..336: mol_type = other DNA
tttgaccaccacctcggtccctccgccgaaagcattaccatcaatatcaatatctttatatcctgcacaaaagtaagtggcagcatcgccacacaccacatcgttgatggtgagagtgaattgtgtcccagatccactgcctttgaaccgcgatgggaccccagatgccagagtggaagccttgtagatcaggagcttgggaggctgccctggtttctgttgaaaccaggctaagtagttcttattccaaacactctcactggcctggcaattgatggtgactgtgtctcccacagggacagacttggaagatggagtctgggtcagcaccagcgc

SEQ ID NO: 299 - DNA, 330 bp, synthetic construct
  misc_feature 1..330: note = 21626B-G5 Coding Sequence
  source 1..330: mol_type = other DNA
gaccctgtgatgacccagactccatcttccacgtctgcagctgtgggaggcacagtcaccatcaattgccaggccagtcaaagtgttgctactaagaaccaattagcctggtatcagcagaaaccagggcagcctcccaagctcctgatctatactacatccagactggcatctggggtcccatcgcggttcagcggcagtggatctggggcgcagttcactctcaccatcagtggtgtgcagtgtgacgatgctgccacttactactgtctaggaacatatgctaatcctatttatactttcggcggagggactgaggtggtcgtcaag

SEQ ID NO: 300 - DNA, 330 bp, synthetic construct
  misc_feature 1..330: note = 21626B-G5 Coding Complementary Sequence
  source 1..330: mol_type = other DNA
cttgacgaccacctcagtccctccgccgaaagtataaataggattagcatatgttcctagacagtagtaagtggcagcatcgtcacactgcacaccactgatggtgagagtgaactgcgccccagatccactgccgctgaaccgcgatgggaccccagatgccagtctggatgtagtatagatcaggagcttgggaggctgccctggtttctgctgataccaggctaattggttcttagtagcaacactttgactggcctggcaattgatggtgactgtgcctcccacagctgcagacgtggaagatggagtctgggtcatcacagggtc

SEQ ID NO: 301 - DNA, 330 bp, synthetic construct
  misc_feature 1..330: note = 21626B-H3 Coding Sequence
  source 1..330: mol_type = other DNA
gcccttgtgatgacccagactccagcctccgtgtctgcagctgtgggaggcacagtcaccatcaactgccaggccagtcagagcattcataagtacttatcctggtatcagcagaaaccagggcagcctcccaaactcctgatttacagggcatccactctggcatctggggtcccgtcgcggttcaaaggcagtggatctgggacacagttcactctcaccattagcgacctggagtgtgacgatgctgccacttactactgtcaacaggatgctgccgttattagtattgataacagtttcggcggagggaccgaggtggtcgtcaaa

SEQ ID NO: 302 - DNA, 330 bp, synthetic construct
  misc_feature 1..330: note = 21626B-H3 Coding Complementary Sequence
  source 1..330: mol_type = other DNA
tttgacgaccacctcggtccctccgccgaaactgttatcaatactaataacggcagcatcctgttgacagtagtaagtggcagcatcgtcacactccaggtcgctaatggtgagagtgaactgtgtcccagatccactgcctttgaaccgcgacgggaccccagatgccagagtggatgccctgtaaatcaggagtttgggaggctgccctggtttctgctgataccaggataagtacttatgaatgctctgactggcctggcagttgatggtgactgtgcctcccacagctgcagacacggaggctggagtctgggtcatcacaagggc

SEQ ID NO: 303 - DNA, 330 bp, synthetic construct
  misc_feature 1..330: note = 21626B-A8 Coding Sequence
  source 1..330: mol_type = other DNA
gcgcttgtgatgacccagactccatcctccgtgtctgcagttgtgggaggcacagtcaccatcaattgccaggcaagtcagagcattagtagttacttgtcctggtatcagcagaaaccagggcagcgtcccaagcccctgatctacgaagcatccaaactggcatctggggtctcatcgcggttcaaaggcagtggatctgggacagagttcactctcaccatcagcggcgtggagtgtgccgatgctgccacttactactgtcaacaggatgctgctgttatgaatgttgataatactttcggcggagggaccgaggtggtggtcaag

SEQ ID NO: 304 - DNA, 330 bp, synthetic construct
  misc_feature 1..330: note = 21626B-A8 Coding Complementary Sequence
  source 1..330: mol_type = other DNA
cttgaccaccacctcggtccctccgccgaaagtattatcaacattcataacagcagcatcctgttgacagtagtaagtggcagcatcggcacactccacgccgctgatggtgagagtgaactctgtcccagatccactgcctttgaaccgcgatgagaccccagatgccagtttggatgcttcgtagatcaggggcttgggacgctgccctggtttctgctgataccaggacaagtaactactaatgctctgacttgcctggcaattgatggtgactgtgcctcccacaactgcagacacggaggatggagtctgggtcatcacaagcgc

SEQ ID NO: 305 - DNA, 330 bp, synthetic construct
  misc_feature 1..330: note = 21626B-D1 Coding Sequence
  source 1..330: mol_type = other DNA
gcccttgtgatgacccagactccagcctccgtgtctgcagctgtgggaggcacagtcaccatcaattgccaggccagtcggagcattcataagtacttatcctggtatcagcagaaaccagggcagcctcccaaactcctgatttacagggcatccactctggcatctggggtcccgtcgcggttcaaaggcagtggatctgggacagagttcactctcaccattagcgacctggagtgtgccgatgctgccacttactactgtcaacaggatgctgccgttattaatattgataacagtttcggcggagggaccgaggtggtcgtcaaa

SEQ ID NO: 306 - DNA, 330 bp, synthetic construct
  misc_feature 1..330: note = 21626B-D1 Coding Complementary Sequence
  source 1..330: mol_type = other DNA
tttgacgaccacctcggtccctccgccgaaactgttatcaatattaataacggcagcatcctgttgacagtagtaagtggcagcatcggcacactccaggtcgctaatggtgagagtgaactctgtcccagatccactgcctttgaaccgcgacgggaccccagatgccagagtggatgccctgtaaatcaggagtttgggaggctgccctggtttctgctgataccaggataagtacttatgaatgctccgactggcctggcaattgatggtgactgtgcctcccacagctgcagacacggaggctggagtctgggtcatcacaagggc

SEQ ID NO: 307 - DNA, 330 bp, synthetic construct
  misc_feature 1..330: note = 21626B-G1 Coding Sequence
  source 1..330: mol_type = other DNA
gcgctggtgatgacccagactgcatcccccgtgtctgcggctgttggaggcacagtcaccatcaattgccaggccagtcagagcattcataagtacttatcctggtatcagcagaaaccagggcagcctcccaaactcctgatttacagggcatccactctggcatctggggtcccgtcgcggttcaaaggcagtggatctgggacagagttcactctcaccattagcgacctggagtgtgccgatgctgccacttactactgtcaacaggatgctgccgttattaatattgataacagtttcggcggagggaccgaggtggtcgtcaaa

SEQ ID NO: 308 - DNA, 330 bp, synthetic construct
  misc_feature 1..330: note = 21626B-G1 Coding Complementary Sequence
  source 1..330: mol_type = other DNA
tttgacgaccacctcggtccctccgccgaaactgttatcaatattaataacggcagcatcctgttgacagtagtaagtggcagcatcggcacactccaggtcgctaatggtgagagtgaactctgtcccagatccactgcctttgaaccgcgacgggaccccagatgccagagtggatgccctgtaaatcaggagtttgggaggctgccctggtttctgctgataccaggataagtacttatgaatgctctgactggcctggcaattgatggtgactgtgcctccaacagccgcagacacgggggatgcagtctgggtcatcaccagcgc

SEQ ID NO: 309 - DNA, 330 bp, synthetic construct
  misc_feature 1..330: note = 21626B-C4-8 Coding Sequence
  source 1..330: mol_type = other DNA
gcgcttgtgatgacccagactccatcctccgtgtctgcagctgtgggaggcacagtcaccatcaattgccaggcaagtcagagcattagtagttacttatcctggtatcagcagaaaccagggcagcgtcccaagcccctgatctacgaaacatccaaactggcatctggggtctcatcgcggttcagtggcagtggatctgggacagagttcactctcaccatcagcggcgtggagtgtgccgatgctgccacttactactgtcaacaggatgctggtgttattaatgttcataatactttcggcggagggaccgaggtggtggtcaag

SEQ ID NO: 310 - DNA, 330 bp, synthetic construct
  misc_feature 1..330: note = 21626B-C4-8 Coding Complementary Sequence
  source 1..330: mol_type = other DNA
cttgaccaccacctcggtccctccgccgaaagtattatgaacattaataacaccagcatcctgttgacagtagtaagtggcagcatcggcacactccacgccgctgatggtgagagtgaactctgtcccagatccactgccactgaaccgcgatgagaccccagatgccagtttggatgtttcgtagatcaggggcttgggacgctgccctggtttctgctgataccaggataagtaactactaatgctctgacttgcctggcaattgatggtgactgtgcctcccacagctgcagacacggaggatggagtctgggtcatcacaagcgc

SEQ ID NO: 311 - DNA, 336 bp, synthetic construct
  misc_feature 1..336: note = 21626S-A1 Coding Sequence
  source 1..336: mol_type = other DNA
gatgttgtgatgacccagactccagcctccgtgtctgaacctgtgggaggcacagtcaccatcaagtgccaggccagtcagagcattggcaatgcattagcctggtatcagcagaaaccagggcagcgtccctcgctcctgatctatgatgcatccactctggcatctggggtctcatcgcggttcaaaggcagtagatctgggacagagtacactctcaccatcagcgacctggagtgtgccgatgctgccacttactactgtcagtgtgcttactatggtagccgttatgctggagggagtgctttcggcggagggaccgaggtggtggtcaaa

SEQ ID NO: 312 - DNA, 336 bp, synthetic construct
  misc_feature 1..336: note = 21626S-A1 Coding Complementary Sequence
  source 1..336: mol_type = other DNA
tttgaccaccacctcggtccctccgccgaaagcactccctccagcataacggctaccatagtaagcacactgacagtagtaagtggcagcatcggcacactccaggtcgctgatggtgagagtgtactctgtcccagatctactgcctttgaaccgcgatgagaccccagatgccagagtggatgcatcatagatcaggagcgagggacgctgccctggtttctgctgataccaggctaatgcattgccaatgctctgactggcctggcacttgatggtgactgtgcctcccacaggttcagacacggaggctggagtctgggtcatcacaacatc

SEQ ID NO: 313 - DNA, 351 bp, synthetic construct
  misc_feature 1..351: note = h-21626B-C4-8 Coding Sequence
  source 1..351: mol_type = other DNA
ctcgaggaggtccagctcgttgagtctggaggaggcttggtccagcctggagggtccctgaggctctcctgtacagcctctggattctccctcagtagctactacatgagctgggtaagacaggcaccagggaagggactggagtggatcggcatcatttatcctagtggtaacacatactacgcgaactgggcaaatggtcgattcaccatctccagacacaattccaagaacacgctgtatcttcaaatgaacagcctgagagcggaggacacggccgtgtactactgtgccagagattctggttatcctgacatctggggtcaaggaaccctggtcaccgtctcctca

SEQ ID NO: 314 - DNA, 351 bp, synthetic construct
  misc_feature 1..351: note = h-21626B-C4-8 Coding Complementary Sequence
  source 1..351: mol_type = other DNA
tgaggagacggtgaccagggttccttgaccccagatgtcaggataaccagaatctctggcacagtagtacacggccgtgtcctccgctctcaggctgttcatttgaagatacagcgtgttcttggaattgtgtctggagatggtgaatcgaccatttgcccagttcgcgtagtatgtgttaccactaggataaatgatgccgatccactccagtcccttccctggtgcctgtcttacccagctcatgtagtagctactgagggagaatccagaggctgtacaggagagcctcagggaccctccaggctggaccaagcctcctccagactcaacgagctggacctcctcgag

SEQ ID NO: 315 - DNA, 360 bp, synthetic construct
  misc_feature 1..360: note = h-21626B-H3 Coding Sequence
  source 1..360: mol_type = other DNA
ctcgagcaggtccagctggttgagtctgggggaggcttggtccagcctggagggtccctgaggctctcctgtacagcctctggattctccctcagtagctatgcaatgggctgggtaagacaggcaccagggaagggacttgagtacatcggctggattagtgctggtggtaccacatactacgcgagctgggtgaatagcagattcaccatctccagagacaattccaagaacacgctgtatcttcaaatgaacagcctgagagcggaggacacggccgtgtactactgtgccagagagggtactggctggggtgcctatgacatctggggtcaaggaaccctggtcaccgtctcctca

SEQ ID NO: 316 - DNA, 360 bp, synthetic construct
  misc_feature 1..360: note = h-21626B-H3 Coding Complementary Sequence
  source 1..360: mol_type = other DNA
tgaggagacggtgaccagggttccttgaccccagatgtcataggcaccccagccagtaccctctctggcacagtagtacacggccgtgtcctccgctctcaggctgttcatttgaagatacagcgtgttcttggaattgtctctggagatggtgaatctgctattcacccagctcgcgtagtatgtggtaccaccagcactaatccagccgatgtactcaagtcccttccctggtgcctgtcttacccagcccattgcatagctactgagggagaatccagaggctgtacaggagagcctcagggaccctccaggctggaccaagcctcccccagactcaaccagctggacctgctcgag

SEQ ID NO: 317 - DNA, 336 bp, synthetic construct
  misc_feature 1..336: note = h-21626B-C4-8 Coding Sequence
  source 1..336: mol_type = other DNA
tctagagacatccagatgacgcagtccccatcctccctgtctgcatctgtaggagacagggtcaccatcacttgccaggcaagtcagagcattagtagttacttatcctggtatcagcagaagccaggcaaggcacctaagctcctgatctatgaaacatccaaactggcatctggggtcccctcaaggtttagtggaagtggatctgggacagatttcaccctcaccatcagcagtctgcaaccggaggacttcgcgacgtactactgccaacaggatgctggtgttattaatgttcataatactttcggtggtggtacaaaggtcgagatcaaa

SEQ ID NO: 318 - DNA, 336 bp, synthetic construct
  misc_feature 1..336: note = h-21626B-C4-8 Coding Complementary Sequence
  source 1..336: mol_type = other DNA
tttgatctcgacctttgtaccaccaccgaaagtattatgaacattaataacaccagcatcctgttggcagtagtacgtcgcgaagtcctccggttgcagactgctgatggtgagggtgaaatctgtcccagatccacttccactaaaccttgaggggaccccagatgccagtttggatgtttcatagatcaggagcttaggtgccttgcctggcttctgctgataccaggataagtaactactaatgctctgacttgcctggcaagtgatggtgaccctgtctcctacagatgcagacagggaggatggggactgcgtcatctggatgtctctaga

SEQ ID NO: 319 - DNA, 336 bp, synthetic construct
  misc_feature 1..336: note = h-21626B-C4-8E Coding Sequence
  source 1..336: mol_type = other DNA
tctagagacatccagatgacgcagtccccatcctccctgtctgcatctgtaggagacagggtcaccatcacttgccaggcaagtcagagcattagtagttacttatcctggtatcagcagaagccaggcaaggcacctaagctcctgatctatgaaacatccaaactggcatctggggtcccctcaaggtttagtggaagtggatctgggacagaattcaccctcaccatcagcagtctgcaaccggaggacttcgcgacgtactactgccaacaggatgctggtgttattaatgttcataatactttcggtggtggtacaaaggtcgagatcaaa

SEQ ID NO: 320 - DNA, 336 bp, synthetic construct
  misc_feature 1..336: note = h-21626B-C4-8E Coding Complementary Sequence
  source 1..336: mol_type = other DNA
tttgatctcgacctttgtaccaccaccgaaagtattatgaacattaataacaccagcatcctgttggcagtagtacgtcgcgaagtcctccggttgcagactgctgatggtgagggtgaattctgtcccagatccacttccactaaaccttgaggggaccccagatgccagtttggatgtttcatagatcaggagcttaggtgccttgcctggcttctgctgataccaggataagtaactactaatgctctgacttgcctggcaagtgatggtgaccctgtctcctacagatgcagacagggaggatggggactgcgtcatctggatgtctctaga

SEQ ID NO: 321 - DNA, 336 bp, synthetic construct
  misc_feature 1..336: note = h-21626B-H3 Coding Sequence
  source 1..336: mol_type = other DNA
tctagagacatccagatgacgcagtccccatcctccctgtctgcatctgtaggagacagggtcaccatcacttgccaggccagtcagagcattcataagtacttatcctggtatcagcagaagccaggcaaggcacctaagctcctgatctatagggcatccactctggcatctggggtcccctcaaggtttagtggaagtggatctgggacagatttcaccctcaccatcagcagtctgcaaccggaggacttcgcgacgtactactgccaacaggatgctgccgttattagtattgataacagtttcggtggtggtacaaaggtcgagatcaaa

SEQ ID NO: 322 - DNA, 336 bp, synthetic construct
  misc_feature 1..336: note = h-21626B-H3 Coding Complementary Sequence
  source 1..336: mol_type = other DNA
tttgatctcgacctttgtaccaccaccgaaactgttatcaatactaataacggcagcatcctgttggcagtagtacgtcgcgaagtcctccggttgcagactgctgatggtgagggtgaaatctgtcccagatccacttccactaaaccttgaggggaccccagatgccagagtggatgccctatagatcaggagcttaggtgccttgcctggcttctgctgataccaggataagtacttatgaatgctctgactggcctggcaagtgatggtgaccctgtctcctacagatgcagacagggaggatggggactgcgtcatctggatgtctctaga

SEQ ID NO: 323 - DNA, 336 bp, synthetic construct
  misc_feature 1..336: note = h-21626B-H3KQ Coding Sequence
  source 1..336: mol_type = other DNA
tctagagacatccagatgacgcagtccccatcctccctgtctgcatctgtaggagacagggtcaccatcacttgccaggccagtcagagcattcataagtacttatcctggtatcagcagaagccaggcaaggcacctaagctcctgatctatagggcatccactctggcatctggggtcccctcaaggtttaagggaagtggatctgggacacagttcaccctcaccatcagcagtctgcaaccggaggacttcgcgacgtactactgccaacaggatgctgccgttattagtattgataacagtttcggtggtggtacaaaggtcgagatcaaa

SEQ ID NO: 324 - DNA, 336 bp, synthetic construct
  misc_feature 1..336: note = h-21626B-H3KQ Coding Complementary Sequence
  source 1..336: mol_type = other DNA
tttgatctcgacctttgtaccaccaccgaaactgttatcaatactaataacggcagcatcctgttggcagtagtacgtcgcgaagtcctccggttgcagactgctgatggtgagggtgaactgtgtcccagatccacttcccttaaaccttgaggggaccccagatgccagagtggatgccctatagatcaggagcttaggtgccttgcctggcttctgctgataccaggataagtacttatgaatgctctgactggcctggcaagtgatggtgaccctgtctcctacagatgcagacagggaggatggggactgcgtcatctggatgtctctaga

SEQ ID NO: 325 - DNA, 666 bp, synthetic construct
  misc_feature 1..666: note = h-21626B-C4-8 Coding Sequence
  source 1..666: mol_type = other DNA
ctcgaggaggtccagctcgttgagtctggaggaggcttggtccagcctggagggtccctgaggctctcctgtacagcctctggattctccctcagtagctactacatgagctgggtaagacaggcaccagggaagggactggagtggatcggcatcatttatcctagtggtaacacatactacgcgaactgggcaaatggtcgattcaccatctccagacacaattccaagaacacgctgtatcttcaaatgaacagcctgagagcggaggacacggccgtgtactactgtgccagagattctggttatcctgacatctggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaa

SEQ ID NO: 326 - DNA, 666 bp, synthetic construct
  misc_feature 1..666: note = h-21626B-C4-8 Coding Complementary Sequence
  source 1..666: mol_type = other DNA
tttgtcacaagatttgggctcaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcaccggttcggggaagtagtccttgaccaggcagcccagggccgctgtgcccccagaggtgctcttggaggagggtgccagggggaagaccgatgggcccttggtggaggctgaggagacggtgaccagggttccttgaccccagatgtcaggataaccagaatctctggcacagtagtacacggccgtgtcctccgctctcaggctgttcatttgaagatacagcgtgttcttggaattgtgtctggagatggtgaatcgaccatttgcccagttcgcgtagtatgtgttaccactaggataaatgatgccgatccactccagtcccttccctggtgcctgtcttacccagctcatgtagtagctactgagggagaatccagaggctgtacaggagagcctcagggaccctccaggctggaccaagcctcctccagactcaacgagctggacctcctcgag

SEQ ID NO: 327 - DNA, 675 bp, synthetic construct
  misc_feature 1..675: note = h-21626B-H3 Coding Sequence
  source 1..675: mol_type = other DNA
ctcgagcaggtccagctggttgagtctgggggaggcttggtccagcctggagggtccctgaggctctcctgtacagcctctggattctccctcagtagctatgcaatgggctgggtaagacaggcaccagggaagggacttgagtacatcggctggattagtgctggtggtaccacatactacgcgagctgggtgaatagcagattcaccatctccagagacaattccaagaacacgctgtatcttcaaatgaacagcctgagagcggaggacacggccgtgtactactgtgccagagagggtactggctggggtgcctatgacatctggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaa

SEQ ID NO: 328 - DNA, 675 bp, synthetic construct
  misc_feature 1..675: note = h-21626B-H3 Coding Complementary Sequence
  source 1..675: mol_type = other DNA
tttgtcacaagatttgggctcaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcaccggttcggggaagtagtccttgaccaggcagcccagggccgctgtgcccccagaggtgctcttggaggagggtgccagggggaagaccgatgggcccttggtggaggctgaggagacggtgaccagggttccttgaccccagatgtcataggcaccccagccagtaccctctctggcacagtagtacacggccgtgtcctccgctctcaggctgttcatttgaagatacagcgtgttcttggaattgtctctggagatggtgaatctgctattcacccagctcgcgtagtatgtggtaccaccagcactaatccagccgatgtactcaagtcccttccctggtgcctgtcttacccagcccattgcatagctactgagggagaatccagaggctgtacaggagagcctcagggaccctccaggctggaccaagcctcccccagactcaaccagctggacctgctcgag

SEQ ID NO: 329 - DNA, 657 bp, synthetic construct
  misc_feature 1..657: note = h-21626B-C4-8 Coding Sequence
  source 1..657: mol_type = other DNA
tctagagacatccagatgacgcagtccccatcctccctgtctgcatctgtaggagacagggtcaccatcacttgccaggcaagtcagagcattagtagttacttatcctggtatcagcagaagccaggcaaggcacctaagctcctgatctatgaaacatccaaactggcatctggggtcccctcaaggtttagtggaagtggatctgggacagatttcaccctcaccatcagcagtctgcaaccggaggacttcgcgacgtactactgccaacaggatgctggtgttattaatgttcataatactttcggtggtggtacaaaggtcgagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 330 - DNA, 657 bp, synthetic construct
  misc_feature 1..657: note = h-21626B-C4-8 Coding Complementary Sequence
  source 1..657: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctcgacctttgtaccaccaccgaaagtattatgaacattaataacaccagcatcctgttggcagtagtacgtcgcgaagtcctccggttgcagactgctgatggtgagggtgaaatctgtcccagatccacttccactaaaccttgaggggaccccagatgccagtttggatgtttcatagatcaggagcttaggtgccttgcctggcttctgctgataccaggataagtaactactaatgctctgacttgcctggcaagtgatggtgaccctgtctcctacagatgcagacagggaggatggggactgcgtcatctggatgtctctaga

SEQ ID NO: 331 - DNA, 657 bp, synthetic construct
  misc_feature 1..657: note = h-21626B-C4-8E Coding Sequence
  source 1..657: mol_type = other DNA
tctagagacatccagatgacgcagtccccatcctccctgtctgcatctgtaggagacagggtcaccatcacttgccaggcaagtcagagcattagtagttacttatcctggtatcagcagaagccaggcaaggcacctaagctcctgatctatgaaacatccaaactggcatctggggtcccctcaaggtttagtggaagtggatctgggacagaattcaccctcaccatcagcagtctgcaaccggaggacttcgcgacgtactactgccaacaggatgctggtgttattaatgttcataatactttcggtggtggtacaaaggtcgagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 332 - DNA, 657 bp, synthetic construct
  misc_feature 1..657: note = h-21626B-C4-8E Coding Complementary Sequence
  source 1..657: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctcgacctttgtaccaccaccgaaagtattatgaacattaataacaccagcatcctgttggcagtagtacgtcgcgaagtcctccggttgcagactgctgatggtgagggtgaattctgtcccagatccacttccactaaaccttgaggggaccccagatgccagtttggatgtttcatagatcaggagcttaggtgccttgcctggcttctgctgataccaggataagtaactactaatgctctgacttgcctggcaagtgatggtgaccctgtctcctacagatgcagacagggaggatggggactgcgtcatctggatgtctctaga

SEQ ID NO: 333 - DNA, 657 bp, synthetic construct
  misc_feature 1..657: note = h-21626B-H3 Coding Sequence
  source 1..657: mol_type = other DNA
tctagagacatccagatgacgcagtccccatcctccctgtctgcatctgtaggagacagggtcaccatcacttgccaggccagtcagagcattcataagtacttatcctggtatcagcagaagccaggcaaggcacctaagctcctgatctatagggcatccactctggcatctggggtcccctcaaggtttagtggaagtggatctgggacagatttcaccctcaccatcagcagtctgcaaccggaggacttcgcgacgtactactgccaacaggatgctgccgttattagtattgataacagtttcggtggtggtacaaaggtcgagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 334 - DNA, 657 bp, synthetic construct
  misc_feature 1..657: note = h-21626B-H3 Coding Complementary Sequence
  source 1..657: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctcgacctttgtaccaccaccgaaactgttatcaatactaataacggcagcatcctgttggcagtagtacgtcgcgaagtcctccggttgcagactgctgatggtgagggtgaaatctgtcccagatccacttccactaaaccttgaggggaccccagatgccagagtggatgccctatagatcaggagcttaggtgccttgcctggcttctgctgataccaggataagtacttatgaatgctctgactggcctggcaagtgatggtgaccctgtctcctacagatgcagacagggaggatggggactgcgtcatctggatgtctctaga

SEQ ID NO: 335 - DNA, 657 bp, synthetic construct
  misc_feature 1..657: note = h-21626B-H3KQ Coding Sequence
  source 1..657: mol_type = other DNA
tctagagacatccagatgacgcagtccccatcctccctgtctgcatctgtaggagacagggtcaccatcacttgccaggccagtcagagcattcataagtacttatcctggtatcagcagaagccaggcaaggcacctaagctcctgatctatagggcatccactctggcatctggggtcccctcaaggtttaagggaagtggatctgggacacagttcaccctcaccatcagcagtctgcaaccggaggacttcgcgacgtactactgccaacaggatgctgccgttattagtattgataacagtttcggtggtggtacaaaggtcgagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 336 - DNA, 657 bp, synthetic construct
  misc_feature 1..657: note = h-21626B-H3KQ Coding Complementary Sequence
  source 1..657: mol_type = other DNA
agatctctgtaggtctactgcgtcaggggtaggagggacagacgtagacatcctctgtcccagtggtagtgaacggtccggtcagtctcgtaagtattcatgaataggaccatagtcgtcttcggtccgttccgtggattcgaggactagatatcccgtaggtgagaccgtagaccccaggggagttccaaattcccttcacctagaccctgtgtcaagtgggagtggtagtcgtcagacgttggcctcctgaagcgctgcatgatgacggttgtcctacgacggcaataatcataactattgtcaaagccaccaccatgtttccagctctagtttgcctgacaccgacgtggtagacagaagtagaagggcggtagactactcgtcaactttagaccttgacggagacaacacacggacgacttattgaagatagggtctctccggtttcatgtcaccttccacctattgcgggaggttagcccattgagggtcctctcacagtgtctcgtcctgtcgttcctgtcgtggatgtcggagtcgtcgtgggactgcgactcgtttcgtctgatgctctttgtgtttcagatgcggacgcttcagtgggtagtcccggactcgagcgggcagtgtttctcgaagttgtcccctctcaca

SEQ ID NO: 337 - protein, 18 aa, synthetic construct
  REGION 1..18: note = Human
LASTQSELTLSQKHWLSD

SEQ ID NO: 338 - protein, 18 aa, synthetic construct
  REGION 1..18: note = Mouse
LASTCSKLNITEQQWMSE

SEQ ID NO: 339 - protein, 18 aa, synthetic construct
  REGION 1..18: note = B5-Helix
LASTCSKLNITEQQWMSD

SEQ ID NO: 340 - protein, 18 aa, synthetic construct
  REGION 1..18: note = B5
LASTCSKLNITQKHWLSD

SEQ ID NO: 341 - protein, 18 aa, synthetic construct
  REGION 1..18: note = Joint
LASTQSELNITEQQWLSD

SEQ ID NO: 342 - protein, 18 aa, synthetic construct
  REGION 1..18: note = Helix
LASTQSELTLSEQQWMSD